(19) United States
(12) Patent Application Publication
(10) Pub. No.: US 2023/0337531 A1
(43) Pub. Date: Oct. 19, 2023

(54) NOVEL COMPOUNDS AND ORGANIC LIGHT EMITTING DEVICE USING THE SAME

(71) Applicant: LG CHEM, LTD., Seoul (KR)

(72) Inventors: Minjun KIM, Daejeon (KR); Dong Hoon LEE, Daejeon (KR); Sang Duk SUH, Daejeon (KR); Donghee KIM, Daejeon (KR); Seung Won CHOI, Daejeon (KR); Jaehoon SIM, Daejeon (KR)

(21) Appl. No.: 17/922,645

(22) PCT Filed: May 18, 2021
(86) PCT No.: PCT/KR2021/006215

§ 371 Date: Nov. 1, 2022

(30) Foreign Application Priority Data
May 18, 2020 (KR) 10-2020-0059233
May 18, 2021 (KR) 10-2021-0063936

(51) Int. Cl. H10K 85/60; C07D 307/77; C07D 409/12; C07D 407/12; C07D 409/14; C07D 407/14; C07D 333/50

(52) U.S. Cl. CPC H10K 85/636; C07D 307/77; C07D 333/50; C07D 407/12; C07D 407/14; C07D 409/12; C07D 409/14; H10K 85/633; H10K 50/11; H10K 85/615; H10K 85/626; H10K 85/6574; H10K 85/6576

(57) ABSTRACT

Provided are novel compounds of Chemical Formula 1:

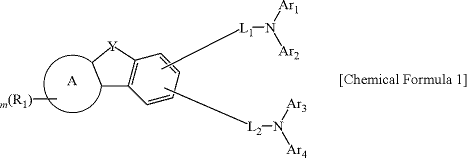

where Y is S or O, A is naphthalene ring, L₁ and L₂ are each independently, a direct bond or a substituted or unsubstituted C₆₋₆₀ arylene, Ar₁, Ar₂, Ar₃ and Ar₄ are each independently a substituted or unsubstituted C₆₋₆₀ and or a substituted or unsubstituted C₂₋₆₀ heteroaryl containing at least one of N, O, and S, R₁ is each independently, hydrogen, deuterium, a substituted or unsubstituted C₁₋₆₀ alkyl or a substituted or unsubstituted C₆₋₆₀ aryl, and m is an integer of 0 to 6. The compounds exhibit high stability to electrons and holes. Also provided is, an organic light emitting device including the compounds, and the devices exhibit greatly improved lifetime characteristics while maintaining high efficiency because of the stability of the compounds of Chemical Formula 1.

### CROSS-REFERENCE TO RELATED APPLICATION(S)

This application is a National Stage Application of International Application No. PCT/KR2021/006215 filed on May 18, 2021, which claims priority to and the benefit of Korean Patent Application No. 10-2020-0059233 filed on May 18, 2020 and Korean Patent Application No. 10-2021-0063936 filed on May 18, 2021 with the Korean Intellectual Property Office, the disclosures of which are incorporated herein by reference in their entirety.

### TECHNICAL FIELD

The present invention relates to a novel compound and an organic light emitting device comprising the same.

### BACKGROUND

In general, an organic light emitting phenomenon refers to a phenomenon where electric energy is converted into light energy by using an organic material. The organic light emitting device using the organic light emitting phenomenon has characteristics such as a wide viewing angle, excellent contrast, a fast response time, and excellent luminance, driving voltage, and response speed, and thus many studies about it have proceeded.

The organic light emitting device generally has a structure which includes an anode, a cathode, and an organic material layer interposed between the anode and the cathode. The organic material layer can have a multilayered structure that includes different materials in order to enhance efficiency and stability of the organic light emitting device, and for example, the organic material layer can be formed of a hole injection layer, a hole transport layer, a light emitting layer, an electron transport layer, an electron injection layer, and the like. In the structure of the organic light emitting device, if a voltage is applied between two electrodes, the holes are injected from an anode into the organic material layer and the electrons are injected from the cathode into the organic material layer, and when the injected holes and the electrons meet each other, an exciton is formed, and light is emitted when the exciton falls to a ground state.

There is a continuing need for the development of new materials for the organic materials used in such organic light emitting devices.

### PRIOR ART LITERATURE

### Patent Literature

(Patent Literature 0001) Korean Patent Laid-open Publication No. 10-2013-073537

### BRIEF DESCRIPTION

### Technical Problem

It is an object of the present invention to provide a novel compound and an organic light emitting device including the same.

### Technical Solution

The present invention provides a compound of Chemical Formula 1:

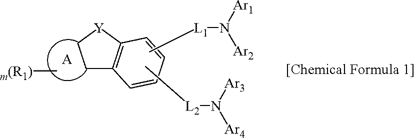

wherein in Chemical Formula 1:
Y is S or O;
A is naphthalene ring;
L₁ and L₂ are each independently a direct bond or a substituted or unsubstituted C₆-₆₀ arylene;
Ar₁, Ar₂, Ar₃ and Ar₄ are each independently a substituted or unsubstituted C₆₋₆₀ aryl or a substituted or unsubstituted C₂₋₆₀ heteroaryl containing at least one of N, O, and S;
R₁ is each independently hydrogen, deuterium, a substituted or unsubstituted C₁₋₆₀ alkyl or a substituted or unsubstituted C₆₋₆₀ aryl; and
m is an integer of 0 to 6.

In addition, the present invention also provides an organic light emitting device including: a first electrode; a second electrode provided at a side opposite to the first electrode; and at least one layer of the organic material layers provided between the first electrode and the second electrode, wherein the at least one layer of the organic material layers includes the compound of the present invention described above.

### Advantageous Effects

The compound of Formula 1 described above can be used as a material of an organic material layer of an organic light emitting device, and can allow improvement of the efficiency, the low driving voltage, and/or the lifetime characteristic when applied to the organic light emitting device. In particular, the compound of Chemical Formula 1 can be used as a material for hole injection, hole transport, hole injection and transport, light emission, electron transport, or electron injection.

### BRIEF DESCRIPTION OF THE DRAWINGS

FIG. 1 shows an example of an organic light emitting device including a substrate 1, an anode 2, a light emitting layer 4, and a cathode 6.

FIG. 2 shows an example of an organic light emitting device including a substrate 1, an anode 2, a hole injection layer 7, a hole transport layer 3, an electron blocking layer 8, a light emitting layer 4, a hole blocking layer 9, an electron injection and transport layer 5, and a cathode 6.

### DETAILED DESCRIPTION

Hereinafter, the present invention will be described in more detail to help understanding of the present invention.

### Definition of Terms

In the present specification,

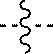

and

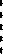

mean a bond connected to another substituent group.

As used herein, the term “substituted or unsubstituted” means that substitution is performed by one or more substituent groups selected from the group consisting of deuterium, a halogen group, a nitrile group, a nitro group, a hydroxyl group, a carbonyl group, an ester group, an imide group, an amino group, a phosphine oxide group, an alkoxy group, an aryloxy group, an alkylthioxy group, an arylthioxy group, an alkylsulfoxy group, an arylsulfoxy group, a silyl group, a boron group, an alkyl group, a cycloalkyl group, an alkenyl group, an aryl group, an aralkyl group, an aralkenyl group, an alkylaryl group, an alkylamine group, an aralkylamine group, a heteroarylamine group, an arylamine group, an arylphosphine group, or a heterocyclic group containing at least one of N, O, and S atoms, or there is no substituent group, or substitution is performed by a substituent group where two or more substituent groups of the exemplified substituent groups are linked or there is no substituent group. for example, the term “substituent group where two or more substituent groups are linked” can be a biphenyl group. That is, the biphenyl group can be an aryl group, or can be interpreted as a substituent group where two phenyl groups are connected.

In the present specification the number of carbon atoms in a carbonyl group is not particularly limited, but is preferably 1 to 40 carbon atoms. Specifically, the carbonyl group can be compounds having the following structures, but is not limited thereto:

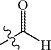

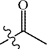

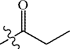

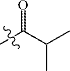

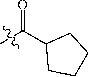

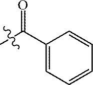

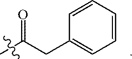

In the present specification, the ester group can have a structure in which oxygen of the ester group can be substituted by a straight-chain, branched-chain, or cyclic alkyl group having 1 to 25 carbon atoms, or an aryl group having 6 to 25 carbon atoms. Specifically, the ester group can be compounds having the following structures, but is not limited thereto:

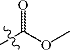

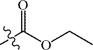

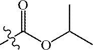

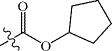

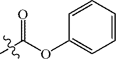

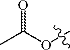

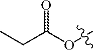

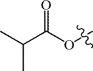

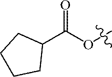

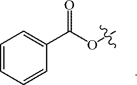

In the present specification, the number of carbon atoms in an imide group is not particularly limited, but is preferably 1 to 25. Specifically, the imide group can be compounds having the following structures, but is not limited thereto:

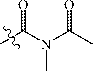

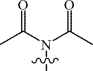

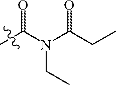

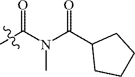

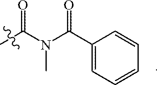

In the present specification, the silyl group specifically includes a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, and the like, but is not limited thereto.

In the present specification, the boron group specifically includes a trimethylboron group, a triethylboron group, a t-butyldimethylboron group, a triphenylboron group, a phenylboron group, and the like, but is not limited thereto.

In the present specification, examples of a halogen group include fluorine, chlorine, bromine, or iodine.

In the present specification, the alkyl group can be a straight chain or a branched chain, and the number of carbon atoms thereof is not particularly limited, but is preferably 1 to 40. According to one embodiment, the alkyl group has 1 to 20 carbon atoms. According to another embodiment, the alkyl group has 1 to 10 carbon atoms. Accoding to still another embodiment, the alkyl group has 1 to 6 carbon atoms. Specific examples of the alkyl group include methyl, ethyl, propyl, n-propyl, isopropyl, butyl, n-butyl, isobutyl, tert-butyl, sec-butyl, 1-methyl-butyl, 1-ethyl-butyl, pentyl, n-pentyl, isopentyl, neopentyl, tert-pentyl, hexyl, n-hexyl, 1-methylpentyl, 2-methylpentyl, 4-methyl-2-pentyl, 3,3-dimethylbutyl, 2-ethylbutyl, heptyl, n-heptyl, 1-methylhexyl, cyclopentylmethyl, cyclohexylmethyl, octyl, n-octyl, tert-octyl, 1-methylheptyl, 2-ethylhexyl, 2-propylpentyl, n-nonyl, 2,2-dimethylheptyl, 1-ethyl-propyl, 1,1-dimethyl-propyl, isohexyl, 2-methylpentyl, 4-methylhexyl, 5-methylhexyl, and the like, but are not limited thereto.

In the present specification, the alkenyl group can be a straight chain or a branched chain, and the number of carbon atoms thereof is not particularly limited, but is preferably 2 to 40. According to one embodiment, the alkenyl group has 2 to 20 carbon atoms. According to another embodiment, the alkenyl group has 2 to 10 carbon atoms. According to still another embodiment, the alkenyl group has 2 to 6 carbon atoms. Specific examples thereof include vinyl, 1-propenyl, isopropenyl, 1-butenyl, 2-butenyl, 3-butenyl, 1-pentenyl, 2-pentenyl, 3-pentenyl, 3-methyl-1-butenyl, 1,3-butadienyl, allyl, 1-phenylvinyl-1-yl, 2-phenylvinyl-1-yl, 2,2-diphenylvinyl-1-yl, 2-phenyl-2-(naphthyl-1-yl)vinyl-1-yl, 2,2-bis(diphenyl-1-yl)vinyl-1-yl, a stilbenyl group, a styrenyl group, and the like, but are not limited thereto.

In the present specification, a cycloalkyl group is not particularly limited, but the number of carbon atoms thereof is preferably 3 to 60. According to one embodiment, the cycloalkyl group has 3 to 30 carbon atoms. According to another embodiment, the cycloalkyl group has 3 to 20 carbon atoms. According to another embodiment, the cycloalkyl group has 3 to 6 carbon atoms. Specific examples thereof include cyclopropyl, cyclobutyl, cyclopentyl, 3-methylcyclopentyl, 2,3-dimethylcyclopentyl, cyclohexyl, 3-methylcyclohexyl, 4-methylcyclohexyl, 2,3-dimethylcyclohexyl, 3,4,5-trimethylcyclohexyl, 4-tert-butylcyclohexyl, cycloheptyl, cyclooctyl, and the like, but are not limited thereto.

In the present specification, the aryl group is not particularly limited, but preferably has 6 to 60 carbon atoms, and can be a monocyclic aryl group or a polycyclic aryl group. According to one embodiment, the aryl group has 6 to 30 carbon atoms. According to one embodiment, the aryl group has 6 to 20 carbon atoms. The aryl group can be a phenyl group, a biphenyl group, a terphenyl group or the like as the monocyclic aryl group, but is not limited thereto. Examples of the polycyclic aryl group include a naphthyl group, an anthracenyl group, a phenanthryl group, a pyrenyl group, a perylenyl group, a chrycenyl group, a fluorenyl group or the like, but is not limited thereto.

In the present specification, a fluorenyl group can be substituted, and two substituent groups can be combined with each other to form a spiro structure. In the case where the fluorenyl group is substituted,

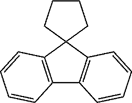 [SMILES: c1ccc2c(c1)-c1ccccc1C21CCCC1]

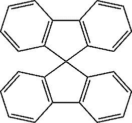 [SMILES: c1ccc2c(c1)-c1ccccc1C21c2ccccc2-c2ccccc21]

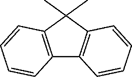

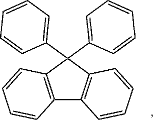

and the like can be formed. However, the structure is not limited thereto.

In the present specification, the heterocyclic group is a heterocyclic group containing at least one of O, N, Si, and S as a heteroatom, and the number of carbon atoms thereof is not particularly limited, but is preferably 2 to 60. Examples of the heterocyclic group include a thiophene group, a furan group, a pyrrole group, an imidazole group, a thiazole group, an oxazole group, an oxadiazole group, a triazole group, a pyridyl group, a bipyridyl group, a pyrimidyl group, a triazine group, a triazole group, an acridyl group, a pyridazine group, a pyrazinyl group, a quinolinyl group, a quinazoline group, a quinoxalinyl group, a phthalazinyl group, a pyridopyrimidinyl group, a pyridopyrazinyl group, a pyrazinopyrazinyl group, an isoquinoline group, an indole group, a carbazole group, a benzoxazole group, a benzimidazole group, a benzothiazole group, a benzo carbazole group, a benzothiophene group, a dibenzothiophene group, a benzofuranyl group, a phenanthroline group, a thiazolyl group, an isoxazolyl group, an oxadiazolyl group, a thiadiazolyl group, a benzothiazolyl group, a phenothiazinyl group, a dibenzofuranyl group, and the like, but are not limited thereto.

In the present specification, the aryl group in the aralkyl group, the aralkenyl group, the alkylaryl group, and the arylamine group is the same as the aforementioned examples of the aryl group. In the present specification, the alkyl group in the aralkyl group, the alkylaryl group and the alkylamine group is the same as the aforementioned examples of the alkyl group. In the present specification, the heteroaryl in the heteroarylamines can be applied to the aforementioned description of the heterocyclic group. In the present specification, the alkenyl group in the aralkenyl group is the same as the aforementioned examples of the alkenyl group. In the present specification, the aforementioned description of the aryl group can be applied except that the arylene is a divalent group. In the present specification, the aforementioned description of the heterocyclic group can be applied except that the heteroarylene is a divalent group. In the present specification, the aforementioned description of the aryl group or cycloalkyl group can be applied except that the hydrocarbon ring is not a monovalent group but formed by combining two substituent groups. In the present specification, the aforementioned description of the heterocyclic group can be applied, except that the heterocycle is not a monovalent group but formed by combining two substituent groups.

### Compound

The present invention provides a compound of Chemical Formula 1. Since the compound of Formula 1 has a core structure of naphthobenzofuran (or naphthobenzothiophene) and two amine groups which are directly bonded to the benzene ring of the core structure or are substituted by an arylene linker, energy transfer from the host to the red dopant can be easily performed when the compound is applied to the light emitting layer. In particular, the stability of electrons and holes is high compared to compounds having different substitution positions of amine groups, compounds in which amine groups are substituted by a heteroarylene linker, or compounds including a hetero group in addition to a naphthalene ring. Accordingly, excellent efficiency, low driving voltage, high brightness, and long lifetime can be realized when applied as a host compound for the light emitting layer of an organic light emitting device. The structure of Chemical Formula 1 is as follows.

Preferably, the compound of Chemical Formula 1 can be any one selected from the compounds of the following Chemical Formulas 1-1 to 1-3:

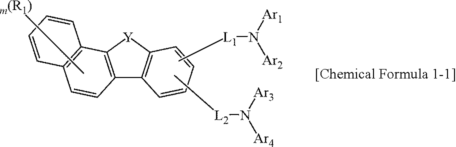

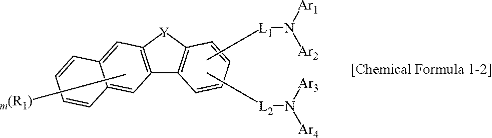

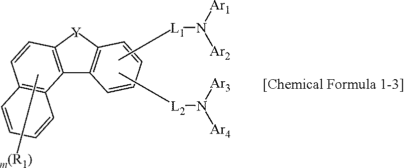

wherein in Chemical Formula 1-1 to 1-3:
Y, L₁, L₂, Ar₁, Ar₂, Ar₃, Ar₄, R₁ and m are as defined above.

Preferably, the compound of Chemical Formula 1 can be any one compound selected from the compounds of the following Chemical Formulas 2-1 to 1-6:

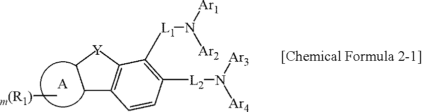

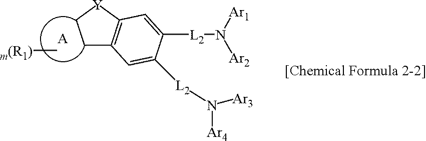

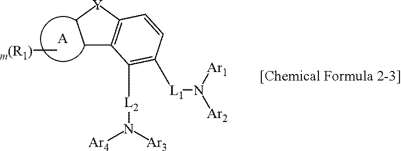

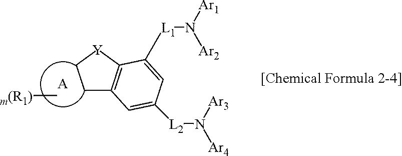

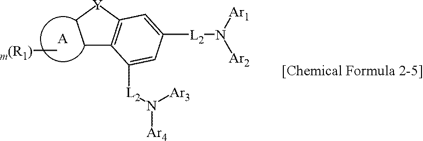

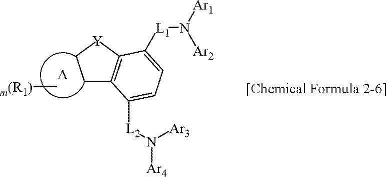

wherein in Chemical Formula 2-1 to 2-6:
Y, L₁, L₂, Ar₁, Ar₂, Ar₃, Ar₄, R₁ and m are as defined above.

Preferably, L₁ and L₂ are each independently a direct bond, phenylene, biphenylylene or naphthylene, more preferably, a direct bond or phenylene.

Preferably, Ar₁, Ar₂, Ar₃ and Ar₄ are each independently biphenylyl, terphenylyl, naphthyl, naphthylphenyl, phenylnaphthyl, phenanthrenyl, triphenylenyl, dimethylfluorenyl, diphenylfluorenyl, dibenzofuranyl, dibenzothiophenyl, carbazol-9-yl, or 9-phenyl-9H-carbazolyl.

More preferably, Ar₁ and Ar₂ are each independently biphenylyl, terphenylyl, naphthyl, naphthylphenyl, phenylnaphthyl, triphenylenyl, dimethylfluorenyl, dibenzofuranyl or dibenzothiophenyl.

Preferably, R₁ is each independently hydrogen, deuterium, C₁₋₁₀alkyl or phenyl, more preferably, R₁ is each independently hydrogen, or deuterium.

Preferably, m is an integer of 0 to 2, more preferably 0 or 1.

Preferably, the compound of Chemical Formula 1 can be any one compound selected from the group consisting of the following compounds:

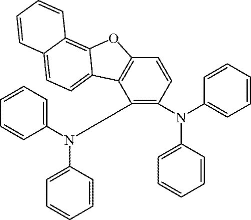 [SMILES: c1ccc(N(c2ccccc2)c2ccc3oc4c5ccccc5ccc4c3c2N(c2ccccc2)c2ccccc2)cc1]

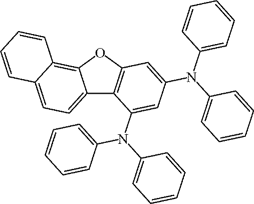 [SMILES: c1ccc(N(c2ccccc2)c2cc(N(c3ccccc3)c3ccccc3)c3c(c2)oc2c4ccccc4ccc23)cc1]

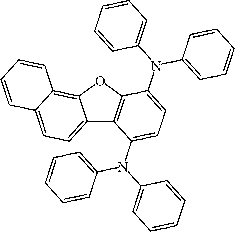 [SMILES: c1ccc(N(c2ccccc2)c2ccc(N(c3ccccc3)c3ccccc3)c3c2oc2c4ccccc4ccc23)cc1]

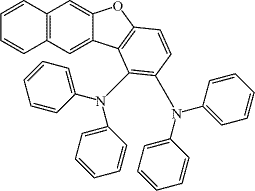 [SMILES: c1ccc(N(c2ccccc2)c2ccc3oc4cc5ccccc5cc4c3c2N(c2ccccc2)c2ccccc2)cc1]

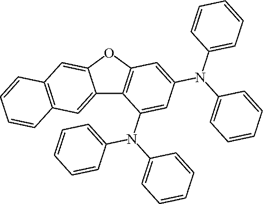 [SMILES: c1ccc(N(c2ccccc2)c2cc(N(c3ccccc3)c3ccccc3)c3c(c2)oc2cc4ccccc4cc23)cc1]

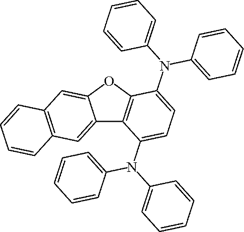 [SMILES: c1ccc(N(c2ccccc2)c2ccc(N(c3ccccc3)c3ccccc3)c3c2oc2cc4ccccc4cc23)cc1]

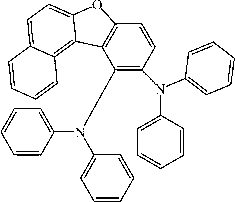 [SMILES: c1ccc(N(c2ccccc2)c2ccc3oc4ccc5ccccc5c4c3c2N(c2ccccc2)c2ccccc2)cc1]

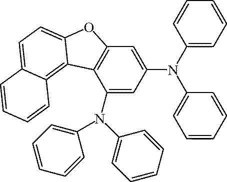 [SMILES: c1ccc(N(c2ccccc2)c2cc(N(c3ccccc3)c3ccccc3)c3c(c2)oc2ccc4ccccc4c23)cc1]

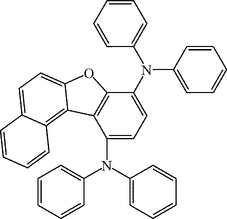 [SMILES: c1ccc(N(c2ccccc2)c2ccc(N(c3ccccc3)c3ccccc3)c3c2oc2ccc4ccccc4c23)cc1]

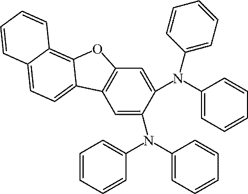 [SMILES: c1ccc(N(c2ccccc2)c2cc3oc4c5ccccc5ccc4c3cc2N(c2ccccc2)c2ccccc2)cc1]

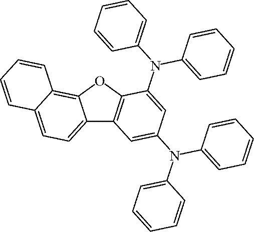 [SMILES: c1ccc(N(c2ccccc2)c2cc(N(c3ccccc3)c3ccccc3)c3oc4c5ccccc5ccc4c3c2)cc1]

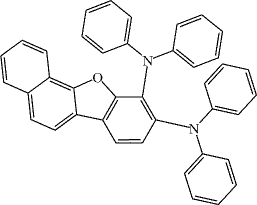 [SMILES: c1ccc(N(c2ccccc2)c2ccc3c(oc4c5ccccc5ccc34)c2N(c2ccccc2)c2ccccc2)cc1]

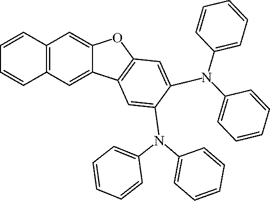 [SMILES: c1ccc(N(c2ccccc2)c2cc3oc4cc5ccccc5cc4c3cc2N(c2ccccc2)c2ccccc2)cc1]

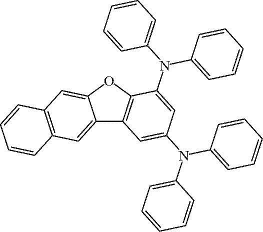 [SMILES: c1ccc(N(c2ccccc2)c2cc(N(c3ccccc3)c3ccccc3)c3oc4cc5ccccc5cc4c3c2)cc1]

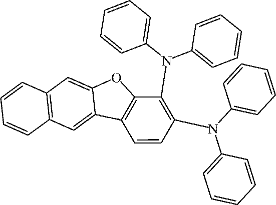 [SMILES: c1ccc(N(c2ccccc2)c2ccc3c(oc4cc5ccccc5cc43)c2N(c2ccccc2)c2ccccc2)cc1]

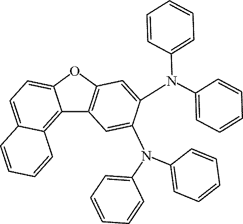 [SMILES: c1ccc(N(c2ccccc2)c2cc3oc4ccc5ccccc5c4c3cc2N(c2ccccc2)c2ccccc2)cc1]

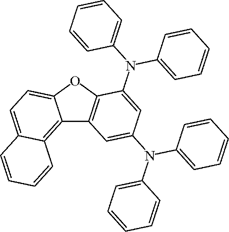 [SMILES: c1ccc(N(c2ccccc2)c2cc(N(c3ccccc3)c3ccccc3)c3oc4ccc5ccccc5c4c3c2)cc1]

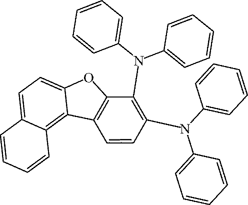 [SMILES: c1ccc(N(c2ccccc2)c2ccc3c(oc4ccc5ccccc5c43)c2N(c2ccccc2)c2ccccc2)cc1]

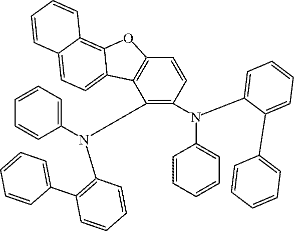 [SMILES: c1ccc(-c2ccccc2N(c2ccccc2)c2ccc3oc4c5ccccc5ccc4c3c2N(c2ccccc2)c2ccccc2-c2ccccc2)cc1]

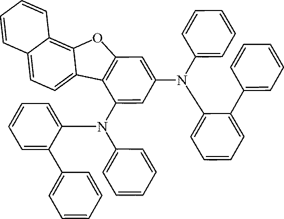 [SMILES: c1ccc(-c2ccccc2N(c2ccccc2)c2cc(N(c3ccccc3)c3ccccc3-c3ccccc3)c3c(c2)oc2c4ccccc4ccc23)cc1]

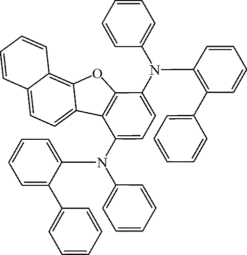 [SMILES: c1ccc(-c2ccccc2N(c2ccccc2)c2ccc(N(c3ccccc3)c3ccccc3-c3ccccc3)c3c2oc2c4ccccc4ccc23)cc1]

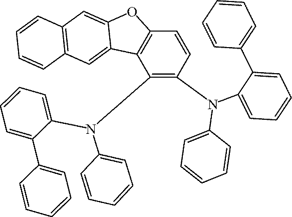 [SMILES: c1ccc(-c2ccccc2N(c2ccccc2)c2ccc3oc4cc5ccccc5cc4c3c2N(c2ccccc2)c2ccccc2-c2ccccc2)cc1]

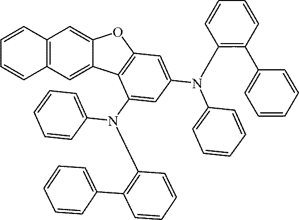 [SMILES: c1ccc(-c2ccccc2N(c2ccccc2)c2cc(N(c3ccccc3)c3ccccc3-c3ccccc3)c3c(c2)oc2cc4ccccc4cc23)cc1]

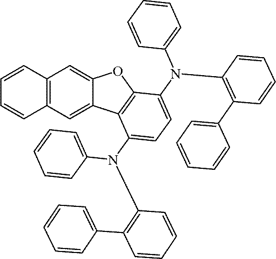 [SMILES: c1ccc(-c2ccccc2N(c2ccccc2)c2ccc(N(c3ccccc3)c3ccccc3-c3ccccc3)c3c2oc2cc4ccccc4cc23)cc1]

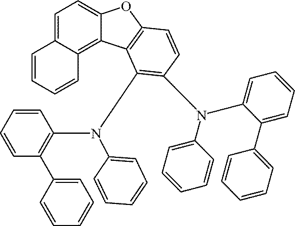 [SMILES: c1ccc(-c2ccccc2N(c2ccccc2)c2ccc3oc4ccc5ccccc5c4c3c2N(c2ccccc2)c2ccccc2-c2ccccc2)cc1]

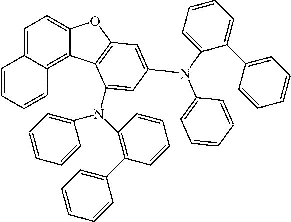 [SMILES: c1ccc(-c2ccccc2N(c2ccccc2)c2cc(N(c3ccccc3)c3ccccc3-c3ccccc3)c3c(c2)oc2ccc4ccccc4c23)cc1]

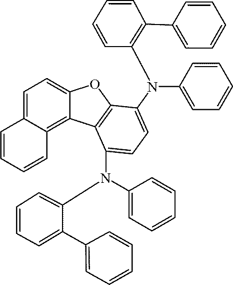 [SMILES: c1ccc(-c2ccccc2N(c2ccccc2)c2ccc(N(c3ccccc3)c3ccccc3-c3ccccc3)c3c2oc2ccc4ccccc4c23)cc1]

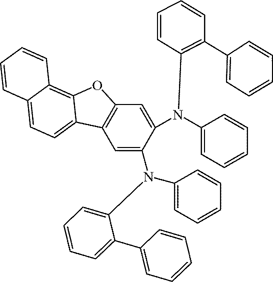 [SMILES: c1ccc(-c2ccccc2N(c2ccccc2)c2cc3oc4c5ccccc5ccc4c3cc2N(c2ccccc2)c2ccccc2-c2ccccc2)cc1]

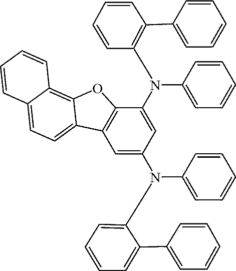 [SMILES: c1ccc(-c2ccccc2N(c2ccccc2)c2cc(N(c3ccccc3)c3ccccc3-c3ccccc3)c3oc4c5ccccc5ccc4c3c2)cc1]

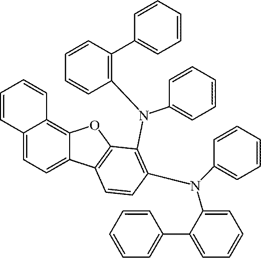 [SMILES: c1ccc(-c2ccccc2N(c2ccccc2)c2ccc3c(oc4c5ccccc5ccc34)c2N(c2ccccc2)c2ccccc2-c2ccccc2)cc1]

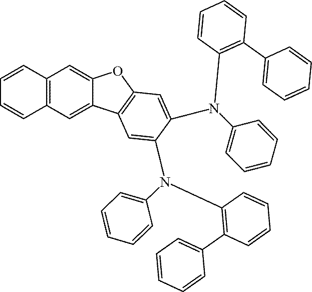 [SMILES: c1ccc(-c2ccccc2N(c2ccccc2)c2cc3oc4cc5ccccc5cc4c3cc2N(c2ccccc2)c2ccccc2-c2ccccc2)cc1]

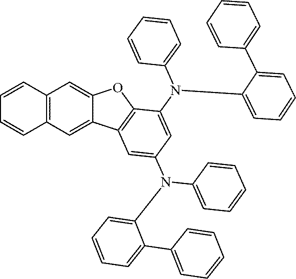 [SMILES: c1ccc(-c2ccccc2N(c2ccccc2)c2cc(N(c3ccccc3)c3ccccc3-c3ccccc3)c3oc4cc5ccccc5cc4c3c2)cc1]

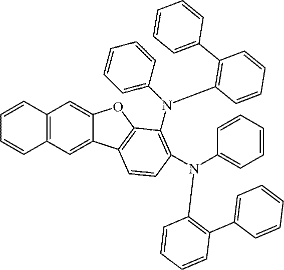 [SMILES: c1ccc(-c2ccccc2N(c2ccccc2)c2ccc3c(oc4cc5ccccc5cc43)c2N(c2ccccc2)c2ccccc2-c2ccccc2)cc1]

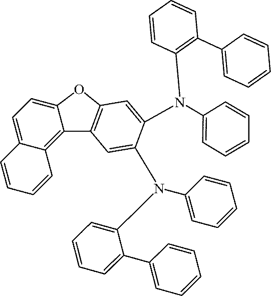 [SMILES: c1ccc(-c2ccccc2N(c2ccccc2)c2cc3oc4ccc5ccccc5c4c3cc2N(c2ccccc2)c2ccccc2-c2ccccc2)cc1]

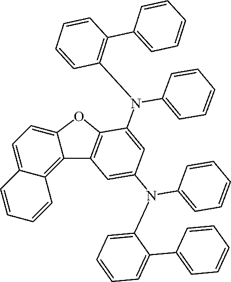 [SMILES: c1ccc(-c2ccccc2N(c2ccccc2)c2cc(N(c3ccccc3)c3ccccc3-c3ccccc3)c3oc4ccc5ccccc5c4c3c2)cc1]

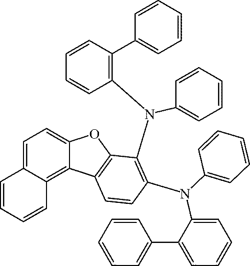 [SMILES: c1ccc(-c2ccccc2N(c2ccccc2)c2ccc3c(oc4ccc5ccccc5c43)c2N(c2ccccc2)c2ccccc2-c2ccccc2)cc1]

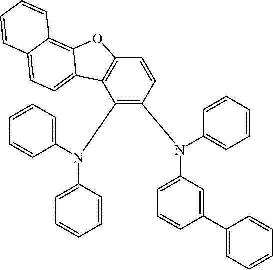 [SMILES: c1ccc(-c2cccc(N(c3ccccc3)c3ccc4oc5c6ccccc6ccc5c4c3N(c3ccccc3)c3ccccc3)c2)cc1]

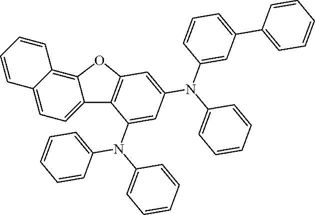 [SMILES: c1ccc(-c2cccc(N(c3ccccc3)c3cc(N(c4ccccc4)c4ccccc4)c4c(c3)oc3c5ccccc5ccc34)c2)cc1]

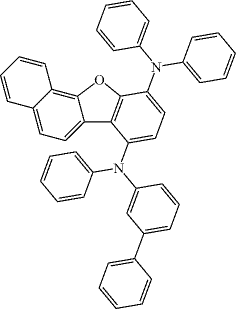 [SMILES: c1ccc(-c2cccc(N(c3ccccc3)c3ccc(N(c4ccccc4)c4ccccc4)c4oc5c6ccccc6ccc5c34)c2)cc1]

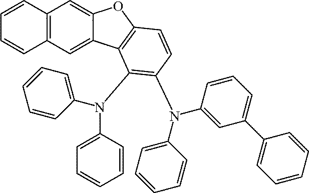 [SMILES: c1ccc(-c2cccc(N(c3ccccc3)c3ccc4oc5cc6ccccc6cc5c4c3N(c3ccccc3)c3ccccc3)c2)cc1]

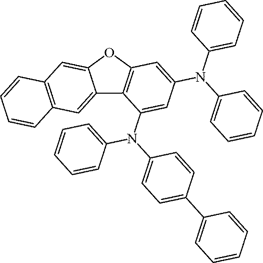 [SMILES: c1ccc(-c2ccc(N(c3ccccc3)c3cc(N(c4ccccc4)c4ccccc4)cc4oc5cc6ccccc6cc5c34)cc2)cc1]

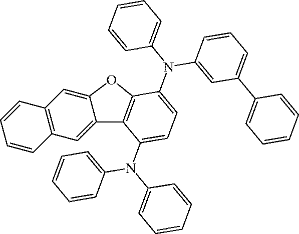 [SMILES: c1ccc(-c2cccc(N(c3ccccc3)c3ccc(N(c4ccccc4)c4ccccc4)c4c3oc3cc5ccccc5cc34)c2)cc1]

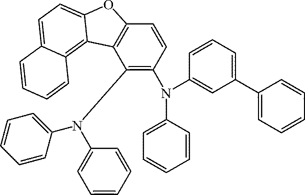 [SMILES: c1ccc(-c2cccc(N(c3ccccc3)c3ccc4oc5ccc6ccccc6c5c4c3N(c3ccccc3)c3ccccc3)c2)cc1]

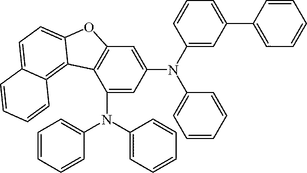 [SMILES: c1ccc(-c2cccc(N(c3ccccc3)c3cc(N(c4ccccc4)c4ccccc4)c4c(c3)oc3ccc5ccccc5c34)c2)cc1]

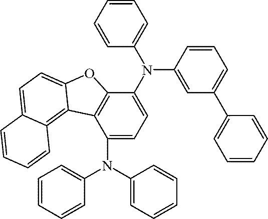 [SMILES: c1ccc(-c2cccc(N(c3ccccc3)c3ccc(N(c4ccccc4)c4ccccc4)c4c3oc3ccc5ccccc5c34)c2)cc1]

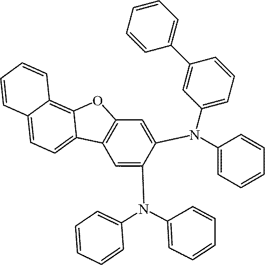 [SMILES: c1ccc(-c2cccc(N(c3ccccc3)c3cc4oc5c6ccccc6ccc5c4cc3N(c3ccccc3)c3ccccc3)c2)cc1]

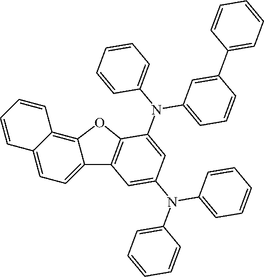 [SMILES: c1ccc(-c2cccc(N(c3ccccc3)c3cc(N(c4ccccc4)c4ccccc4)cc4c3oc3c5ccccc5ccc43)c2)cc1]

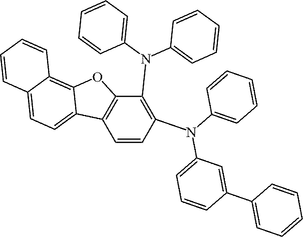 [SMILES: c1ccc(-c2cccc(N(c3ccccc3)c3ccc4c(oc5c6ccccc6ccc45)c3N(c3ccccc3)c3ccccc3)c2)cc1]

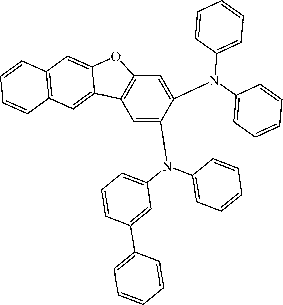 [SMILES: c1ccc(-c2cccc(N(c3ccccc3)c3cc4c(cc3N(c3ccccc3)c3ccccc3)oc3cc5ccccc5cc34)c2)cc1]

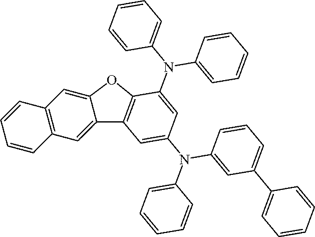 [SMILES: c1ccc(-c2cccc(N(c3ccccc3)c3cc(N(c4ccccc4)c4ccccc4)c4oc5cc6ccccc6cc5c4c3)c2)cc1]

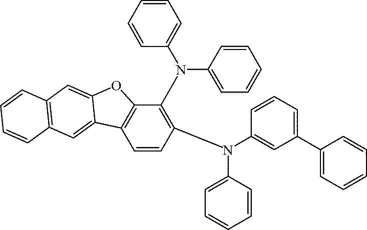 [SMILES: c1ccc(-c2cccc(N(c3ccccc3)c3ccc4c(oc5cc6ccccc6cc54)c3N(c3ccccc3)c3ccccc3)c2)cc1]

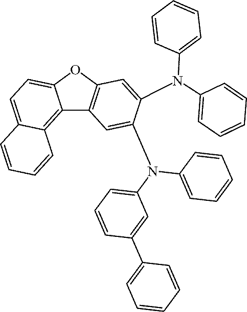 [SMILES: c1ccc(-c2cccc(N(c3ccccc3)c3cc4c(cc3N(c3ccccc3)c3ccccc3)oc3ccc5ccccc5c34)c2)cc1]

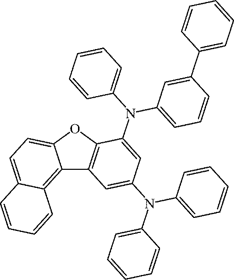 [SMILES: c1ccc(-c2cccc(N(c3ccccc3)c3cc(N(c4ccccc4)c4ccccc4)cc4c3oc3ccc5ccccc5c34)c2)cc1]

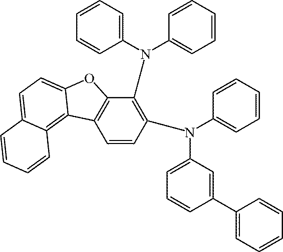 [SMILES: c1ccc(-c2cccc(N(c3ccccc3)c3ccc4c(oc5ccc6ccccc6c54)c3N(c3ccccc3)c3ccccc3)c2)cc1]

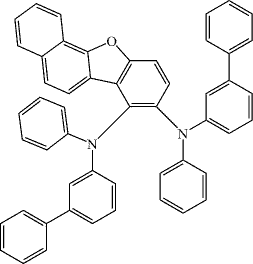 [SMILES: c1ccc(-c2cccc(N(c3ccccc3)c3ccc4oc5c6ccccc6ccc5c4c3N(c3ccccc3)c3cccc(-c4ccccc4)c3)c2)cc1]

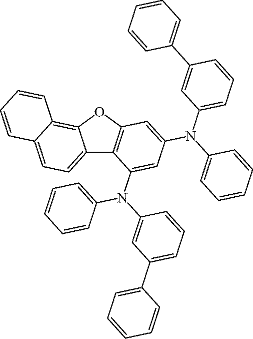 [SMILES: c1ccc(-c2cccc(N(c3ccccc3)c3cc(N(c4ccccc4)c4cccc(-c5ccccc5)c4)c4c(c3)oc3c5ccccc5ccc34)c2)cc1]

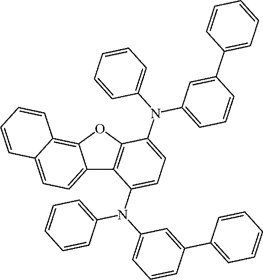 [SMILES: c1ccc(-c2cccc(N(c3ccccc3)c3ccc(N(c4ccccc4)c4cccc(-c5ccccc5)c4)c4c3oc3c5ccccc5ccc34)c2)cc1]

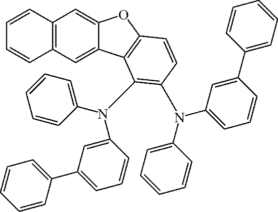 [SMILES: c1ccc(-c2cccc(N(c3ccccc3)c3ccc4oc5cc6ccccc6cc5c4c3N(c3ccccc3)c3cccc(-c4ccccc4)c3)c2)cc1]

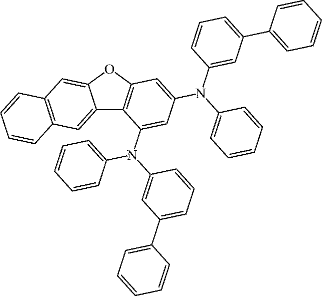 [SMILES: c1ccc(-c2cccc(N(c3ccccc3)c3cc(N(c4ccccc4)c4cccc(-c5ccccc5)c4)c4c(c3)oc3cc5ccccc5cc34)c2)cc1]

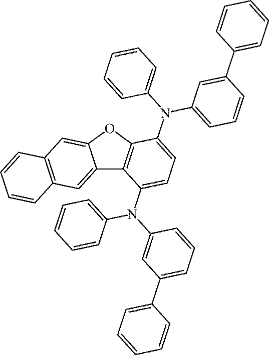 [SMILES: c1ccc(-c2cccc(N(c3ccccc3)c3ccc(N(c4ccccc4)c4cccc(-c5ccccc5)c4)c4c3oc3cc5ccccc5cc34)c2)cc1]

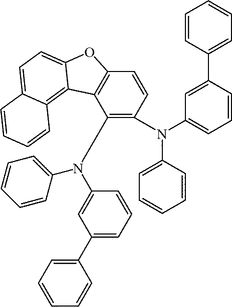 [SMILES: c1ccc(-c2cccc(N(c3ccccc3)c3ccc4oc5ccc6ccccc6c5c4c3N(c3ccccc3)c3cccc(-c4ccccc4)c3)c2)cc1]

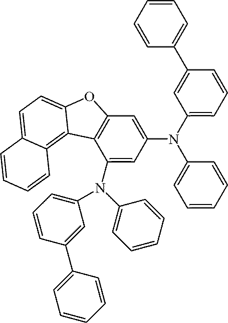 [SMILES: c1ccc(-c2cccc(N(c3ccccc3)c3cc(N(c4ccccc4)c4cccc(-c5ccccc5)c4)c4c(c3)oc3ccc5ccccc5c34)c2)cc1]

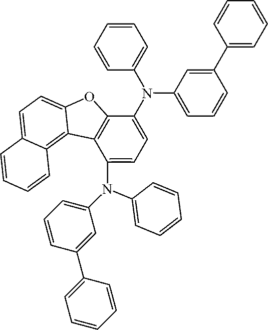 [SMILES: c1ccc(-c2cccc(N(c3ccccc3)c3ccc(N(c4ccccc4)c4cccc(-c5ccccc5)c4)c4c3oc3ccc5ccccc5c34)c2)cc1]

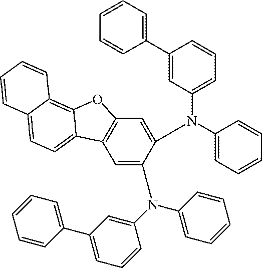 [SMILES: c1ccc(-c2cccc(N(c3ccccc3)c3cc4oc5c6ccccc6ccc5c4cc3N(c3ccccc3)c3cccc(-c4ccccc4)c3)c2)cc1]

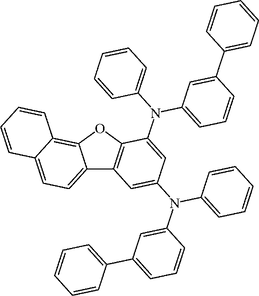 [SMILES: c1ccc(-c2cccc(N(c3ccccc3)c3cc(N(c4ccccc4)c4cccc(-c5ccccc5)c4)c4oc5c6ccccc6ccc5c4c3)c2)cc1]

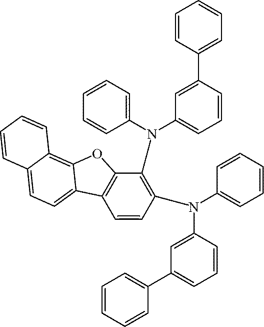 [SMILES: c1ccc(-c2cccc(N(c3ccccc3)c3ccc4c(oc5c6ccccc6ccc45)c3N(c3ccccc3)c3cccc(-c4ccccc4)c3)c2)cc1]

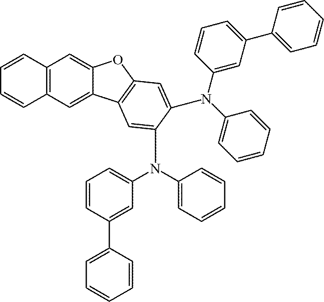 [SMILES: c1ccc(-c2cccc(N(c3ccccc3)c3cc4oc5cc6ccccc6cc5c4cc3N(c3ccccc3)c3cccc(-c4ccccc4)c3)c2)cc1]

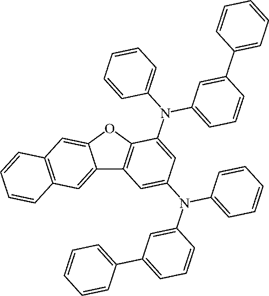 [SMILES: c1ccc(-c2cccc(N(c3ccccc3)c3cc(N(c4ccccc4)c4cccc(-c5ccccc5)c4)c4oc5cc6ccccc6cc5c4c3)c2)cc1]

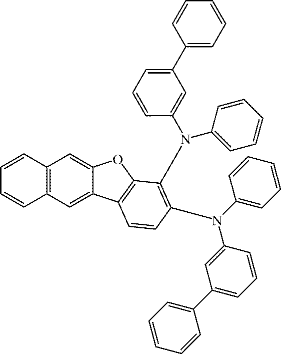 [SMILES: c1ccc(-c2cccc(N(c3ccccc3)c3ccc4c(oc5cc6ccccc6cc54)c3N(c3ccccc3)c3cccc(-c4ccccc4)c3)c2)cc1]

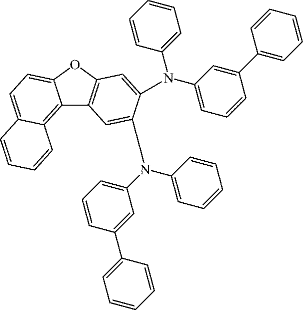 [SMILES: c1ccc(-c2cccc(N(c3ccccc3)c3cc4oc5ccc6ccccc6c5c4cc3N(c3ccccc3)c3cccc(-c4ccccc4)c3)c2)cc1]

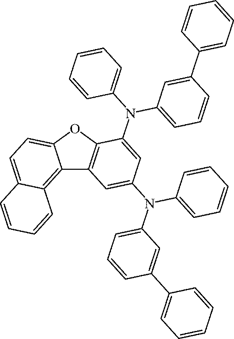 [SMILES: c1ccc(-c2cccc(N(c3ccccc3)c3cc(N(c4ccccc4)c4cccc(-c5ccccc5)c4)c4oc5ccc6ccccc6c5c4c3)c2)cc1]

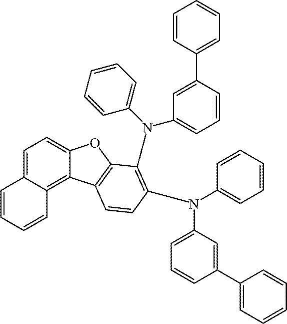 [SMILES: c1ccc(-c2cccc(N(c3ccccc3)c3ccc4c(oc5ccc6ccccc6c54)c3N(c3ccccc3)c3cccc(-c4ccccc4)c3)c2)cc1]

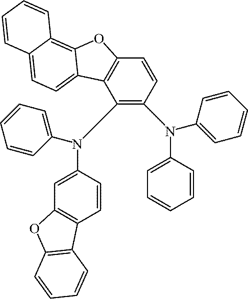 [SMILES: c1ccc(N(c2ccccc2)c2ccc3oc4c5ccccc5ccc4c3c2N(c2ccccc2)c2ccc3c(c2)oc2ccccc23)cc1]

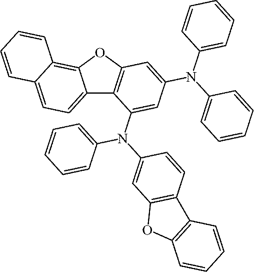 [SMILES: c1ccc(N(c2ccccc2)c2cc(N(c3ccccc3)c3ccc4c(c3)oc3ccccc34)c3c(c2)oc2c4ccccc4ccc23)cc1]

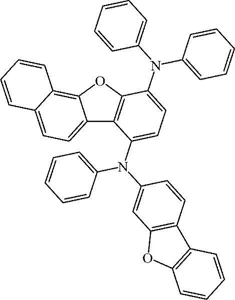 [SMILES: c1ccc(N(c2ccccc2)c2ccc(N(c3ccccc3)c3ccc4c(c3)oc3ccccc34)c3c2oc2c4ccccc4ccc23)cc1]

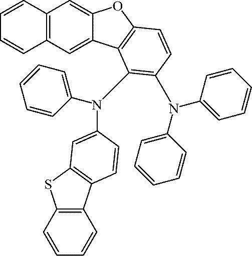 [SMILES: c1ccc(N(c2ccccc2)c2ccc3oc4cc5ccccc5cc4c3c2N(c2ccccc2)c2ccc3c(c2)sc2ccccc23)cc1]

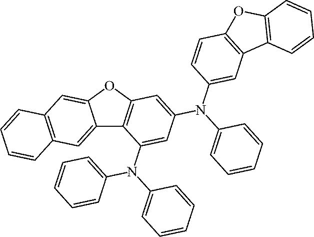 [SMILES: c1ccc(N(c2cc(N(c3ccccc3)c3ccccc3)c3c(c2)oc2cc4ccccc4cc23)c2ccc3oc4ccccc4c3c2)cc1]

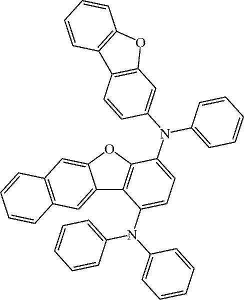 [SMILES: c1ccc(N(c2ccc3c(c2)oc2ccccc23)c2ccc(N(c3ccccc3)c3ccccc3)c3c2oc2cc4ccccc4cc23)cc1]

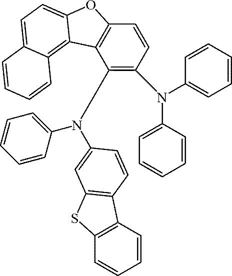 [SMILES: c1ccc(N(c2ccccc2)c2ccc3oc4ccc5ccccc5c4c3c2N(c2ccccc2)c2ccc3c(c2)sc2ccccc23)cc1]

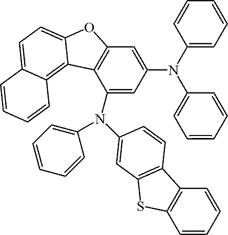 [SMILES: c1ccc(N(c2ccccc2)c2cc(N(c3ccccc3)c3ccc4c(c3)sc3ccccc34)c3c(c2)oc2ccc4ccccc4c23)cc1]

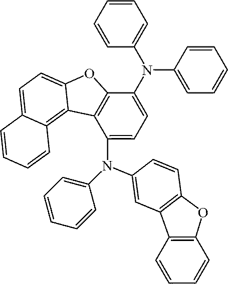 [SMILES: c1ccc(N(c2ccccc2)c2ccc(N(c3ccccc3)c3ccc4oc5ccccc5c4c3)c3c2oc2ccc4ccccc4c23)cc1]

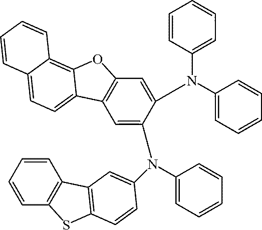 [SMILES: c1ccc(N(c2ccccc2)c2cc3oc4c5ccccc5ccc4c3cc2N(c2ccccc2)c2ccc3sc4ccccc4c3c2)cc1]

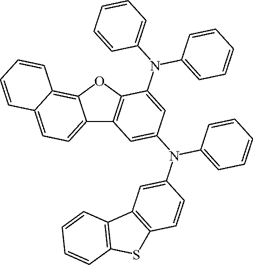 [SMILES: c1ccc(N(c2ccc3sc4ccccc4c3c2)c2cc(N(c3ccccc3)c3ccccc3)c3oc4c5ccccc5ccc4c3c2)cc1]

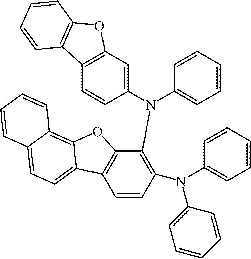 [SMILES: c1ccc(N(c2ccccc2)c2ccc3c(oc4c5ccccc5ccc34)c2N(c2ccccc2)c2ccc3c(c2)oc2ccccc23)cc1]

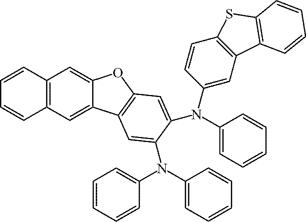 [SMILES: c1ccc(N(c2ccc3sc4ccccc4c3c2)c2cc3oc4cc5ccccc5cc4c3cc2N(c2ccccc2)c2ccccc2)cc1]

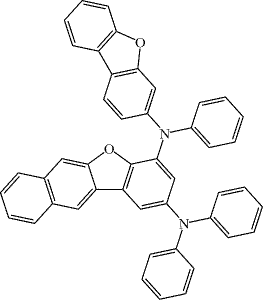 [SMILES: c1ccc(N(c2ccccc2)c2cc(N(c3ccccc3)c3ccc4c(c3)oc3ccccc34)c3oc4cc5ccccc5cc4c3c2)cc1]

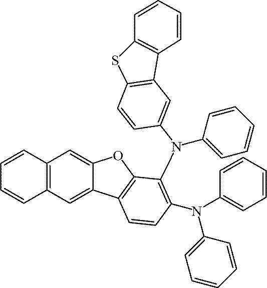 [SMILES: c1ccc(N(c2ccccc2)c2ccc3c(oc4cc5ccccc5cc43)c2N(c2ccccc2)c2ccc3sc4ccccc4c3c2)cc1]

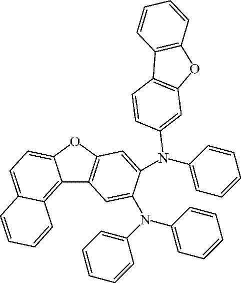 [SMILES: c1ccc(N(c2ccc3c(c2)oc2ccccc23)c2cc3oc4ccc5ccccc5c4c3cc2N(c2ccccc2)c2ccccc2)cc1]

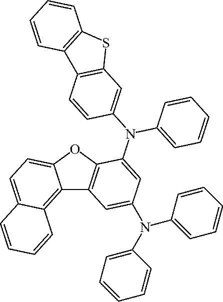 [SMILES: c1ccc(N(c2ccccc2)c2cc(N(c3ccccc3)c3ccc4c(c3)sc3ccccc34)c3oc4ccc5ccccc5c4c3c2)cc1]

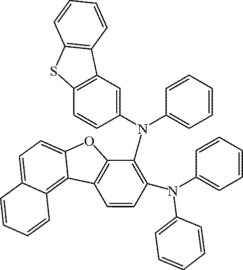 [SMILES: c1ccc(N(c2ccccc2)c2ccc3c(oc4ccc5ccccc5c43)c2N(c2ccccc2)c2ccc3sc4ccccc4c3c2)cc1]

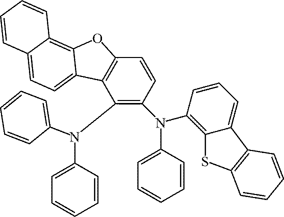 [SMILES: c1ccc(N(c2ccc3oc4c5ccccc5ccc4c3c2N(c2ccccc2)c2ccccc2)c2cccc3c2sc2ccccc23)cc1]

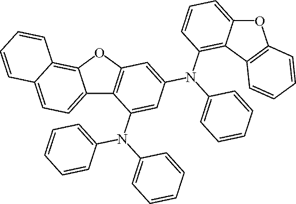 [SMILES: c1ccc(N(c2cc(N(c3ccccc3)c3ccccc3)c3c(c2)oc2c4ccccc4ccc23)c2cccc3oc4ccccc4c23)cc1]

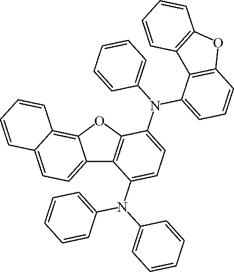 [SMILES: c1ccc(N(c2ccc(N(c3ccccc3)c3ccccc3)c3c2oc2c4ccccc4ccc23)c2cccc3oc4ccccc4c23)cc1]

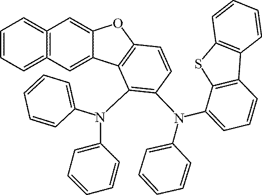

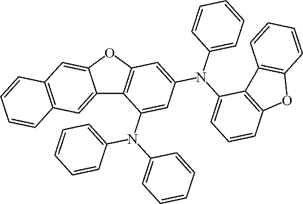 [SMILES: c1ccc(N(c2cc(N(c3ccccc3)c3ccccc3)c3c(c2)oc2cc4ccccc4cc23)c2cccc3oc4ccccc4c23)cc1]

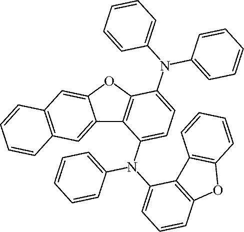 [SMILES: c1ccc(N(c2ccccc2)c2ccc(N(c3ccccc3)c3cccc4oc5ccccc5c34)c3c2oc2cc4ccccc4cc23)cc1]

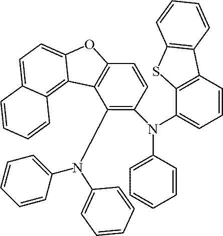 [SMILES: c1ccc(N(c2ccc3oc4ccc5ccccc5c4c3c2N(c2ccccc2)c2ccccc2)c2cccc3c2sc2ccccc23)cc1]

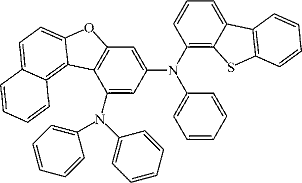 [SMILES: c1ccc(N(c2cc(N(c3ccccc3)c3ccccc3)c3c(c2)oc2ccc4ccccc4c23)c2cccc3c2sc2ccccc23)cc1]

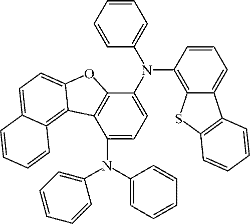 [SMILES: c1ccc(N(c2ccc(N(c3ccccc3)c3ccccc3)c3c2oc2ccc4ccccc4c23)c2cccc3c2sc2ccccc23)cc1]

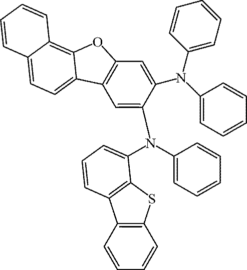 [SMILES: c1ccc(N(c2ccccc2)c2cc3oc4c5ccccc5ccc4c3cc2N(c2ccccc2)c2cccc3c2sc2ccccc23)cc1]

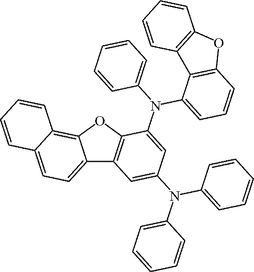 [SMILES: c1ccc(N(c2ccccc2)c2cc(N(c3ccccc3)c3cccc4oc5ccccc5c34)c3oc4c5ccccc5ccc4c3c2)cc1]

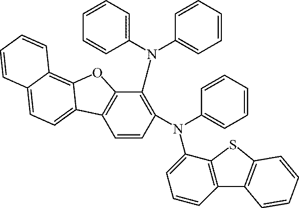 [SMILES: c1ccc(N(c2ccccc2)c2c(N(c3ccccc3)c3cccc4c3sc3ccccc34)ccc3c2oc2c4ccccc4ccc32)cc1]

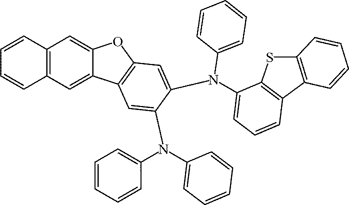 [SMILES: c1ccc(N(c2ccccc2)c2cc3c(cc2N(c2ccccc2)c2cccc4c2sc2ccccc24)oc2cc4ccccc4cc23)cc1]

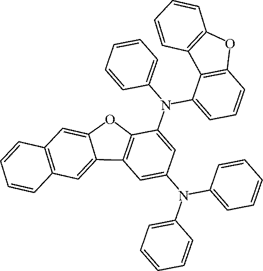 [SMILES: c1ccc(N(c2ccccc2)c2cc(N(c3ccccc3)c3cccc4oc5ccccc5c34)c3oc4cc5ccccc5cc4c3c2)cc1]

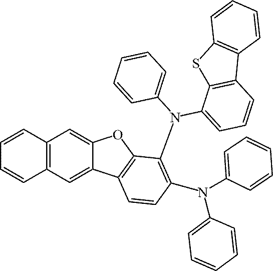 [SMILES: c1ccc(N(c2ccccc2)c2ccc3c(oc4cc5ccccc5cc43)c2N(c2ccccc2)c2cccc3c2sc2ccccc23)cc1]

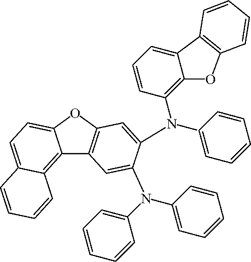

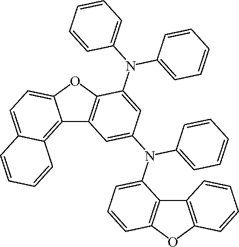 [SMILES: c1ccc(N(c2ccccc2)c2cc(N(c3ccccc3)c3cccc4oc5ccccc5c34)cc3c2oc2ccc4ccccc4c23)cc1]

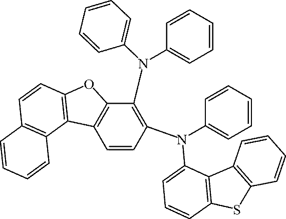 [SMILES: c1ccc(N(c2ccccc2)c2c(N(c3ccccc3)c3cccc4sc5ccccc5c34)ccc3c2oc2ccc4ccccc4c23)cc1]

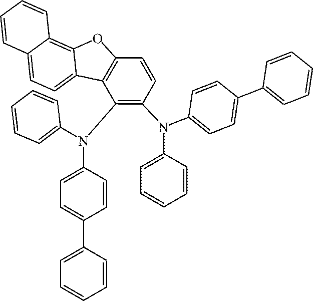 [SMILES: c1ccc(-c2ccc(N(c3ccccc3)c3ccc4oc5c6ccccc6ccc5c4c3N(c3ccccc3)c3ccc(-c4ccccc4)cc3)cc2)cc1]

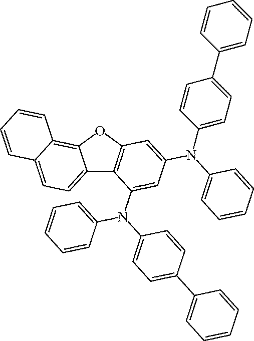 [SMILES: c1ccc(-c2ccc(N(c3ccccc3)c3cc(N(c4ccccc4)c4ccc(-c5ccccc5)cc4)c4c(c3)oc3c5ccccc5ccc34)cc2)cc1]

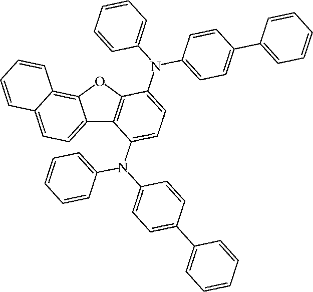 [SMILES: c1ccc(-c2ccc(N(c3ccccc3)c3ccc(N(c4ccccc4)c4ccc(-c5ccccc5)cc4)c4c3oc3c5ccccc5ccc34)cc2)cc1]

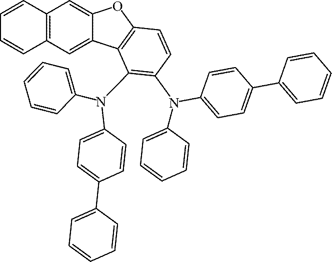 [SMILES: c1ccc(-c2ccc(N(c3ccccc3)c3ccc4oc5cc6ccccc6cc5c4c3N(c3ccccc3)c3ccc(-c4ccccc4)cc3)cc2)cc1]

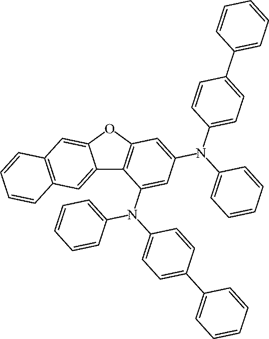 [SMILES: c1ccc(-c2ccc(N(c3ccccc3)c3cc(N(c4ccccc4)c4ccc(-c5ccccc5)cc4)c4c(c3)oc3cc5ccccc5cc34)cc2)cc1]

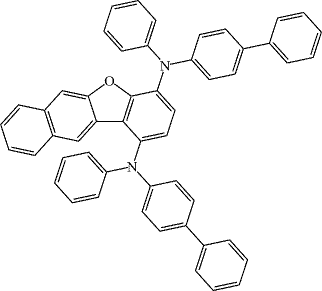 [SMILES: c1ccc(-c2ccc(N(c3ccccc3)c3ccc(N(c4ccccc4)c4ccc(-c5ccccc5)cc4)c4c3oc3cc5ccccc5cc34)cc2)cc1]

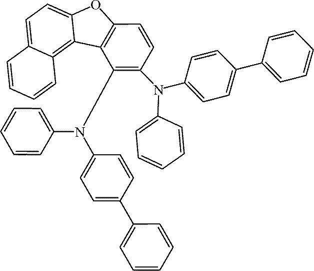 [SMILES: c1ccc(-c2ccc(N(c3ccccc3)c3ccc4oc5ccc6ccccc6c5c4c3N(c3ccccc3)c3ccc(-c4ccccc4)cc3)cc2)cc1]

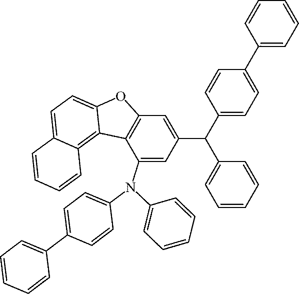 [SMILES: c1ccc(-c2ccc(C(c3ccccc3)c3cc(N(c4ccccc4)c4ccc(-c5ccccc5)cc4)c4c(c3)oc3ccc5ccccc5c34)cc2)cc1]

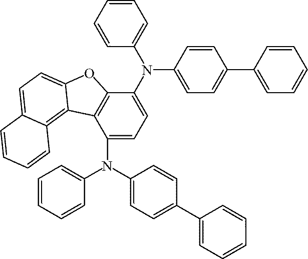 [SMILES: c1ccc(-c2ccc(N(c3ccccc3)c3ccc(N(c4ccccc4)c4ccc(-c5ccccc5)cc4)c4c3oc3ccc5ccccc5c34)cc2)cc1]

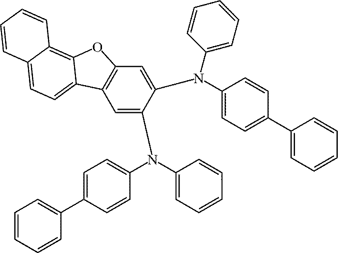 [SMILES: c1ccc(-c2ccc(N(c3ccccc3)c3cc4oc5c6ccccc6ccc5c4cc3N(c3ccccc3)c3ccc(-c4ccccc4)cc3)cc2)cc1]

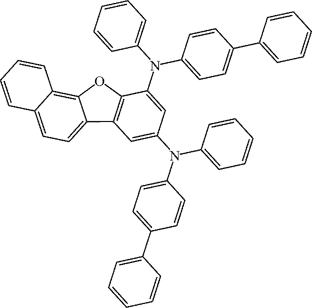 [SMILES: c1ccc(-c2ccc(N(c3ccccc3)c3cc(N(c4ccccc4)c4ccc(-c5ccccc5)cc4)c4oc5c6ccccc6ccc5c4c3)cc2)cc1]

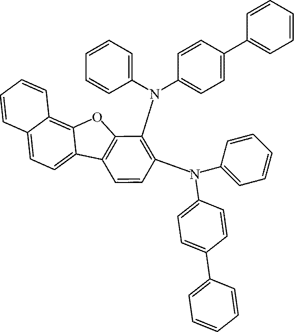 [SMILES: c1ccc(-c2ccc(N(c3ccccc3)c3ccc4c(oc5c6ccccc6ccc45)c3N(c3ccccc3)c3ccc(-c4ccccc4)cc3)cc2)cc1]

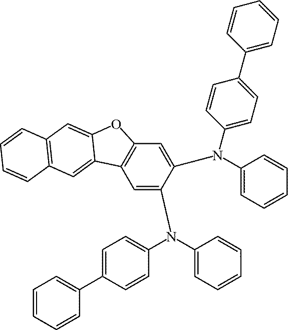 [SMILES: c1ccc(-c2ccc(N(c3ccccc3)c3cc4oc5cc6ccccc6cc5c4cc3N(c3ccccc3)c3ccc(-c4ccccc4)cc3)cc2)cc1]

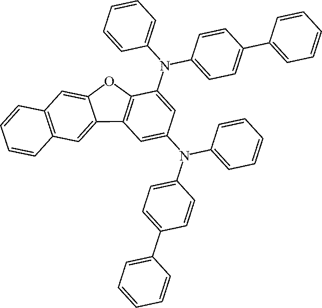 [SMILES: c1ccc(-c2ccc(N(c3ccccc3)c3cc(N(c4ccccc4)c4ccc(-c5ccccc5)cc4)c4oc5cc6ccccc6cc5c4c3)cc2)cc1]

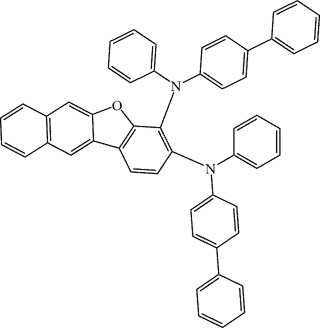 [SMILES: c1ccc(-c2ccc(N(c3ccccc3)c3ccc4c(oc5cc6ccccc6cc54)c3N(c3ccccc3)c3ccc(-c4ccccc4)cc3)cc2)cc1]

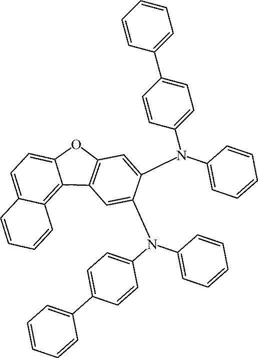 [SMILES: c1ccc(-c2ccc(N(c3ccccc3)c3cc4oc5ccc6ccccc6c5c4cc3N(c3ccccc3)c3ccc(-c4ccccc4)cc3)cc2)cc1]

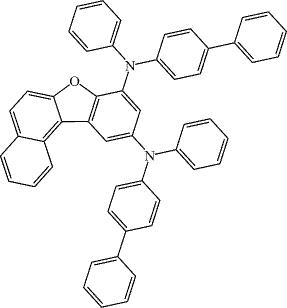 [SMILES: c1ccc(-c2ccc(N(c3ccccc3)c3cc(N(c4ccccc4)c4ccc(-c5ccccc5)cc4)c4oc5ccc6ccccc6c5c4c3)cc2)cc1]

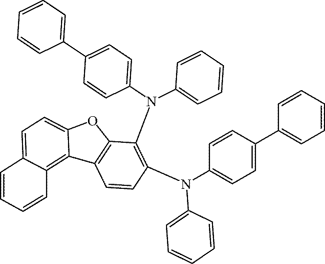 [SMILES: c1ccc(-c2ccc(N(c3ccccc3)c3ccc4c(oc5ccc6ccccc6c54)c3N(c3ccccc3)c3ccc(-c4ccccc4)cc3)cc2)cc1]

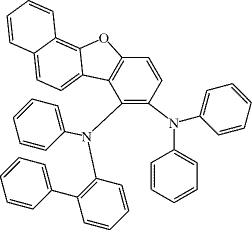 [SMILES: c1ccc(-c2ccccc2N(c2ccccc2)c2c(N(c3ccccc3)c3ccccc3)ccc3oc4c5ccccc5ccc4c23)cc1]

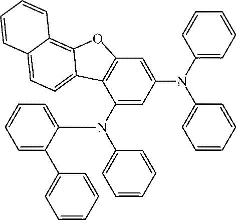 [SMILES: c1ccc(-c2ccccc2N(c2ccccc2)c2cc(N(c3ccccc3)c3ccccc3)cc3oc4c5ccccc5ccc4c23)cc1]

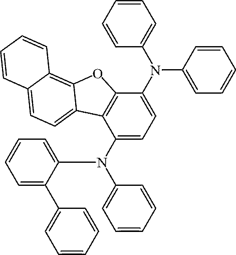 [SMILES: c1ccc(-c2ccccc2N(c2ccccc2)c2ccc(N(c3ccccc3)c3ccccc3)c3oc4c5ccccc5ccc4c23)cc1]

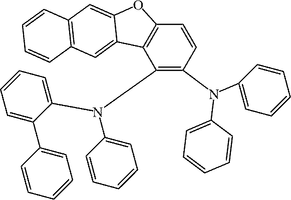 [SMILES: c1ccc(-c2ccccc2N(c2ccccc2)c2c(N(c3ccccc3)c3ccccc3)ccc3oc4cc5ccccc5cc4c23)cc1]

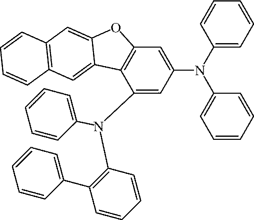 [SMILES: c1ccc(-c2ccccc2N(c2ccccc2)c2cc(N(c3ccccc3)c3ccccc3)cc3oc4cc5ccccc5cc4c23)cc1]

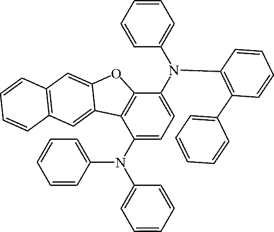 [SMILES: c1ccc(-c2ccccc2N(c2ccccc2)c2ccc(N(c3ccccc3)c3ccccc3)c3c2oc2cc4ccccc4cc23)cc1]

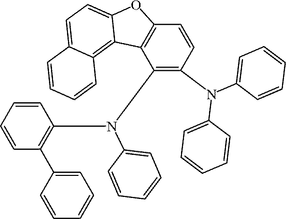 [SMILES: c1ccc(-c2ccccc2N(c2ccccc2)c2c(N(c3ccccc3)c3ccccc3)ccc3oc4ccc5ccccc5c4c23)cc1]

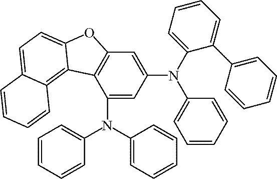 [SMILES: c1ccc(-c2ccccc2N(c2ccccc2)c2cc(N(c3ccccc3)c3ccccc3)c3c(c2)oc2ccc4ccccc4c23)cc1]

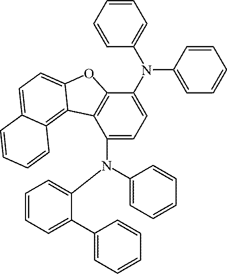 [SMILES: c1ccc(-c2ccccc2N(c2ccccc2)c2ccc(N(c3ccccc3)c3ccccc3)c3oc4ccc5ccccc5c4c23)cc1]

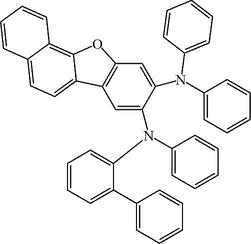 [SMILES: c1ccc(-c2ccccc2N(c2ccccc2)c2cc3c(cc2N(c2ccccc2)c2ccccc2)oc2c4ccccc4ccc32)cc1]

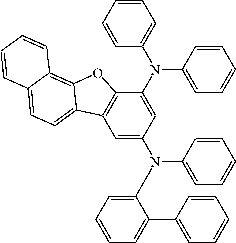 [SMILES: c1ccc(-c2ccccc2N(c2ccccc2)c2cc(N(c3ccccc3)c3ccccc3)c3oc4c5ccccc5ccc4c3c2)cc1]

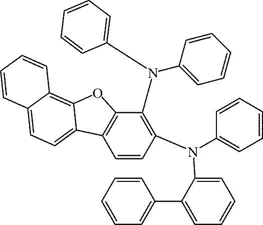 [SMILES: c1ccc(-c2ccccc2N(c2ccccc2)c2ccc3c(oc4c5ccccc5ccc34)c2N(c2ccccc2)c2ccccc2)cc1]

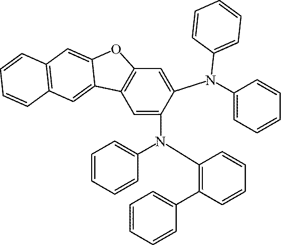 [SMILES: c1ccc(-c2ccccc2N(c2ccccc2)c2cc3c(cc2N(c2ccccc2)c2ccccc2)oc2cc4ccccc4cc23)cc1]

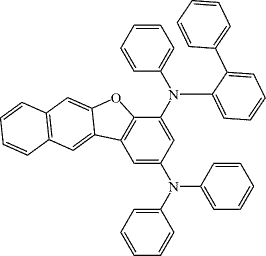 [SMILES: c1ccc(-c2ccccc2N(c2ccccc2)c2cc(N(c3ccccc3)c3ccccc3)cc3c2oc2cc4ccccc4cc23)cc1]

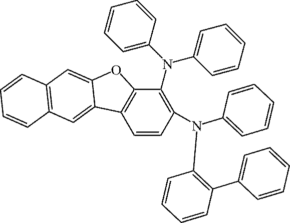 [SMILES: c1ccc(-c2ccccc2N(c2ccccc2)c2ccc3c(oc4cc5ccccc5cc43)c2N(c2ccccc2)c2ccccc2)cc1]

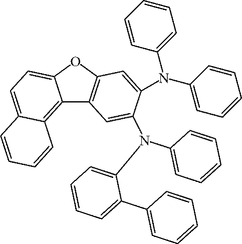 [SMILES: c1ccc(-c2ccccc2N(c2ccccc2)c2cc3c(cc2N(c2ccccc2)c2ccccc2)oc2ccc4ccccc4c23)cc1]

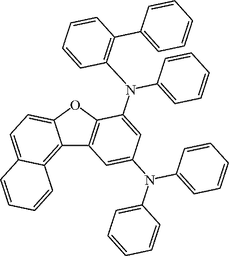 [SMILES: c1ccc(-c2ccccc2N(c2ccccc2)c2cc(N(c3ccccc3)c3ccccc3)cc3c2oc2ccc4ccccc4c23)cc1]

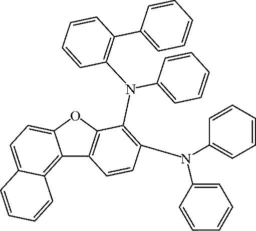 [SMILES: c1ccc(-c2ccccc2N(c2ccccc2)c2c(N(c3ccccc3)c3ccccc3)ccc3c2oc2ccc4ccccc4c23)cc1]

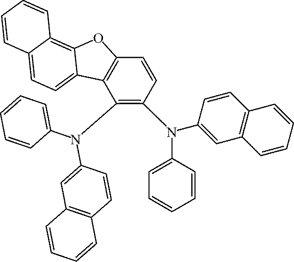 [SMILES: c1ccc(N(c2ccc3ccccc3c2)c2ccc3oc4c5ccccc5ccc4c3c2N(c2ccccc2)c2ccc3ccccc3c2)cc1]

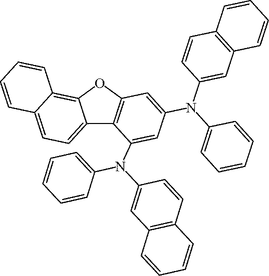 [SMILES: c1ccc(N(c2ccc3ccccc3c2)c2cc(N(c3ccccc3)c3ccc4ccccc4c3)c3c(c2)oc2c4ccccc4ccc23)cc1]

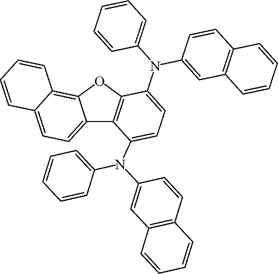 [SMILES: c1ccc(N(c2ccc3ccccc3c2)c2ccc(N(c3ccccc3)c3ccc4ccccc4c3)c3c2oc2c4ccccc4ccc23)cc1]

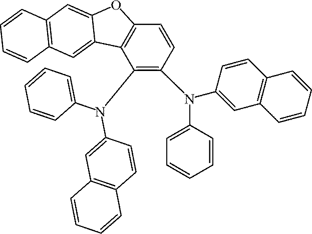 [SMILES: c1ccc(N(c2ccc3ccccc3c2)c2ccc3oc4cc5ccccc5cc4c3c2N(c2ccccc2)c2ccc3ccccc3c2)cc1]

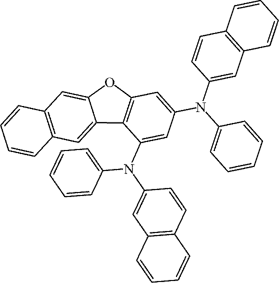 [SMILES: c1ccc(N(c2ccc3ccccc3c2)c2cc(N(c3ccccc3)c3ccc4ccccc4c3)c3c(c2)oc2cc4ccccc4cc23)cc1]

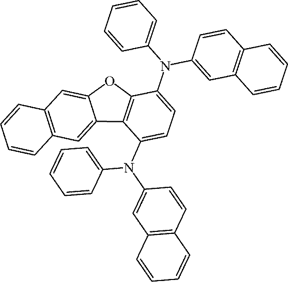 [SMILES: c1ccc(N(c2ccc3ccccc3c2)c2ccc(N(c3ccccc3)c3ccc4ccccc4c3)c3c2oc2cc4ccccc4cc23)cc1]

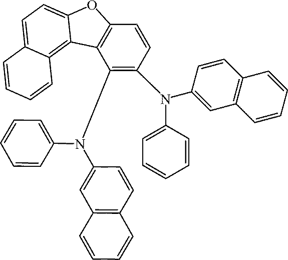 [SMILES: c1ccc(N(c2ccc3ccccc3c2)c2ccc3oc4ccc5ccccc5c4c3c2N(c2ccccc2)c2ccc3ccccc3c2)cc1]

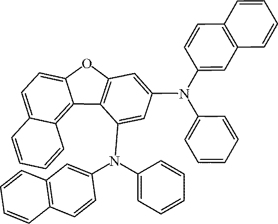 [SMILES: c1ccc(N(c2ccc3ccccc3c2)c2cc(N(c3ccccc3)c3ccc4ccccc4c3)c3c(c2)oc2ccc4ccccc4c23)cc1]

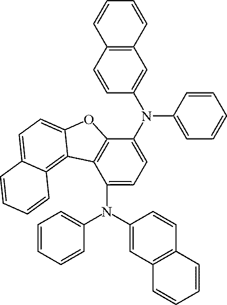 [SMILES: c1ccc(N(c2ccc3ccccc3c2)c2ccc(N(c3ccccc3)c3ccc4ccccc4c3)c3c2oc2ccc4ccccc4c23)cc1]

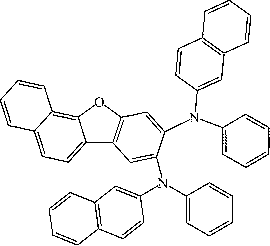 [SMILES: c1ccc(N(c2ccc3ccccc3c2)c2cc3oc4c5ccccc5ccc4c3cc2N(c2ccccc2)c2ccc3ccccc3c2)cc1]

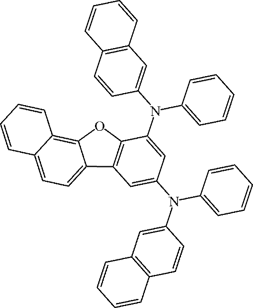 [SMILES: c1ccc(N(c2ccc3ccccc3c2)c2cc(N(c3ccccc3)c3ccc4ccccc4c3)c3oc4c5ccccc5ccc4c3c2)cc1]

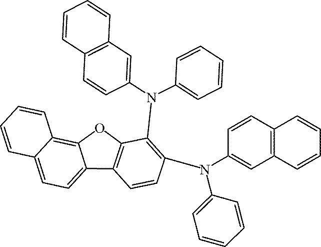 [SMILES: c1ccc(N(c2ccc3ccccc3c2)c2ccc3c(oc4c5ccccc5ccc34)c2N(c2ccccc2)c2ccc3ccccc3c2)cc1]

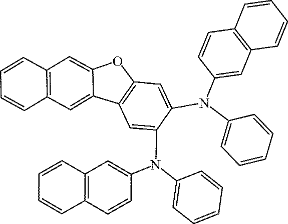 [SMILES: c1ccc(N(c2ccc3ccccc3c2)c2cc3oc4cc5ccccc5cc4c3cc2N(c2ccccc2)c2ccc3ccccc3c2)cc1]

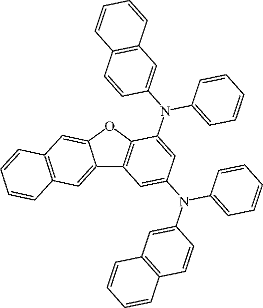 [SMILES: c1ccc(N(c2ccc3ccccc3c2)c2cc(N(c3ccccc3)c3ccc4ccccc4c3)c3oc4cc5ccccc5cc4c3c2)cc1]

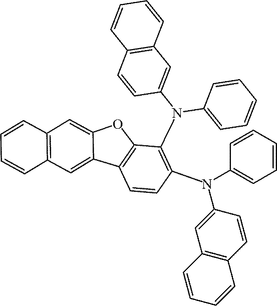 [SMILES: c1ccc(N(c2ccc3ccccc3c2)c2ccc3c(oc4cc5ccccc5cc43)c2N(c2ccccc2)c2ccc3ccccc3c2)cc1]

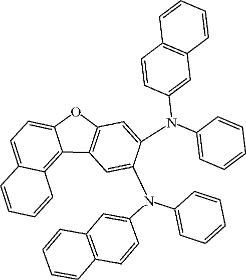 [SMILES: c1ccc(N(c2ccc3ccccc3c2)c2cc3oc4ccc5ccccc5c4c3cc2N(c2ccccc2)c2ccc3ccccc3c2)cc1]

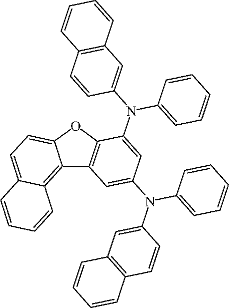 [SMILES: c1ccc(N(c2ccc3ccccc3c2)c2cc(N(c3ccccc3)c3ccc4ccccc4c3)c3oc4ccc5ccccc5c4c3c2)cc1]

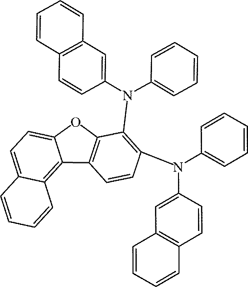 [SMILES: c1ccc(N(c2ccc3ccccc3c2)c2ccc3c(oc4ccc5ccccc5c43)c2N(c2ccccc2)c2ccc3ccccc3c2)cc1]

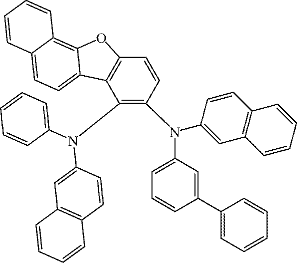 [SMILES: c1ccc(-c2cccc(N(c3ccc4ccccc4c3)c3ccc4oc5c6ccccc6ccc5c4c3N(c3ccccc3)c3ccc4ccccc4c3)c2)cc1]

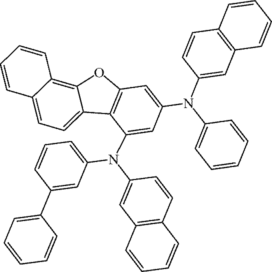 [SMILES: c1ccc(-c2cccc(N(c3ccc4ccccc4c3)c3cc(N(c4ccccc4)c4ccc5ccccc5c4)cc4oc5c6ccccc6ccc5c34)c2)cc1]

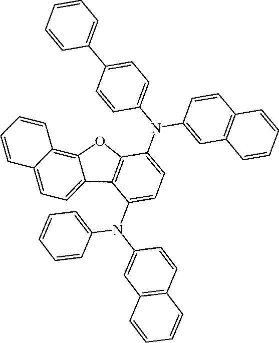 [SMILES: c1ccc(-c2ccc(N(c3ccc4ccccc4c3)c3ccc(N(c4ccccc4)c4ccc5ccccc5c4)c4c3oc3c5ccccc5ccc34)cc2)cc1]

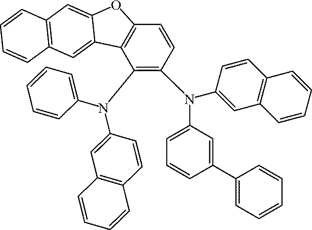 [SMILES: c1ccc(-c2cccc(N(c3ccc4ccccc4c3)c3ccc4oc5cc6ccccc6cc5c4c3N(c3ccccc3)c3ccc4ccccc4c3)c2)cc1]

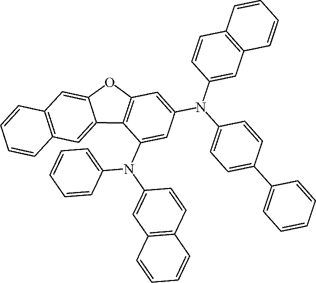 [SMILES: c1ccc(-c2ccc(N(c3ccc4ccccc4c3)c3cc(N(c4ccccc4)c4ccc5ccccc5c4)c4c(c3)oc3cc5ccccc5cc34)cc2)cc1]

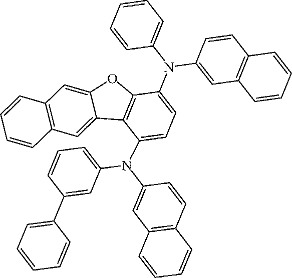 [SMILES: c1ccc(-c2cccc(N(c3ccc4ccccc4c3)c3ccc(N(c4ccccc4)c4ccc5ccccc5c4)c4oc5cc6ccccc6cc5c34)c2)cc1]

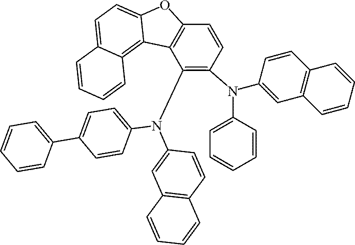 [SMILES: c1ccc(-c2ccc(N(c3ccc4ccccc4c3)c3c(N(c4ccccc4)c4ccc5ccccc5c4)ccc4oc5ccc6ccccc6c5c34)cc2)cc1]

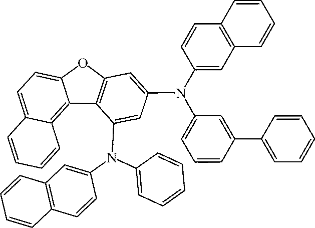 [SMILES: c1ccc(-c2cccc(N(c3ccc4ccccc4c3)c3cc(N(c4ccccc4)c4ccc5ccccc5c4)c4c(c3)oc3ccc5ccccc5c34)c2)cc1]

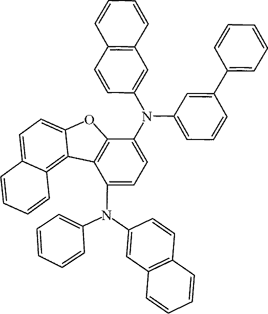 [SMILES: c1ccc(-c2cccc(N(c3ccc4ccccc4c3)c3ccc(N(c4ccccc4)c4ccc5ccccc5c4)c4c3oc3ccc5ccccc5c34)c2)cc1]

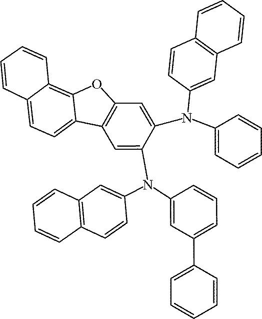 [SMILES: c1ccc(-c2cccc(N(c3ccc4ccccc4c3)c3cc4c(cc3N(c3ccccc3)c3ccc5ccccc5c3)oc3c5ccccc5ccc43)c2)cc1]

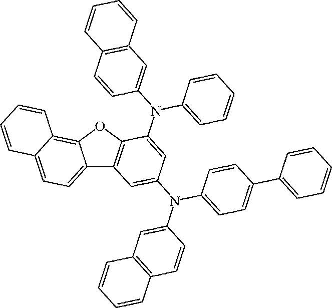 [SMILES: c1ccc(-c2ccc(N(c3ccc4ccccc4c3)c3cc(N(c4ccccc4)c4ccc5ccccc5c4)c4oc5c6ccccc6ccc5c4c3)cc2)cc1]

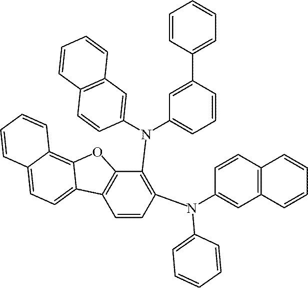 [SMILES: c1ccc(-c2cccc(N(c3ccc4ccccc4c3)c3c(N(c4ccccc4)c4ccc5ccccc5c4)ccc4c3oc3c5ccccc5ccc43)c2)cc1]

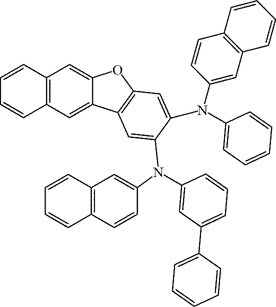 [SMILES: c1ccc(-c2cccc(N(c3ccc4ccccc4c3)c3cc4c(cc3N(c3ccccc3)c3ccc5ccccc5c3)oc3cc5ccccc5cc34)c2)cc1]

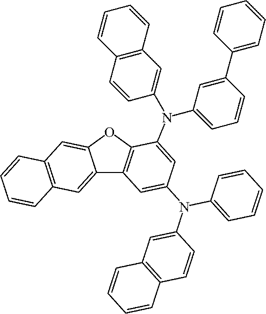 [SMILES: c1ccc(-c2cccc(N(c3ccc4ccccc4c3)c3cc(N(c4ccccc4)c4ccc5ccccc5c4)cc4c3oc3cc5ccccc5cc34)c2)cc1]

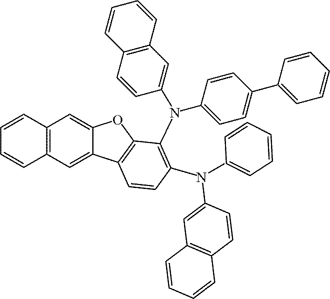

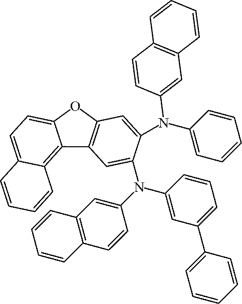 [SMILES: c1ccc(-c2cccc(N(c3ccc4ccccc4c3)c3cc4c(cc3N(c3ccccc3)c3ccc5ccccc5c3)oc3ccc5ccccc5c34)c2)cc1]

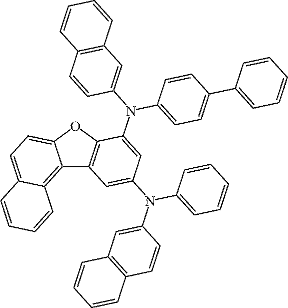 [SMILES: c1ccc(-c2ccc(N(c3ccc4ccccc4c3)c3cc(N(c4ccccc4)c4ccc5ccccc5c4)cc4c3oc3ccc5ccccc5c34)cc2)cc1]

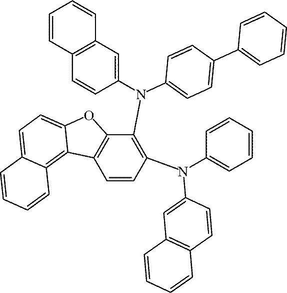 [SMILES: c1ccc(-c2ccc(N(c3ccc4ccccc4c3)c3c(N(c4ccccc4)c4ccc5ccccc5c4)ccc4c3oc3ccc5ccccc5c34)cc2)cc1]

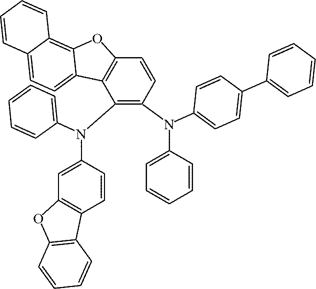 [SMILES: c1ccc(-c2ccc(N(c3ccccc3)c3ccc4oc5c6ccccc6ccc5c4c3N(c3ccccc3)c3ccc4c(c3)oc3ccccc34)cc2)cc1]

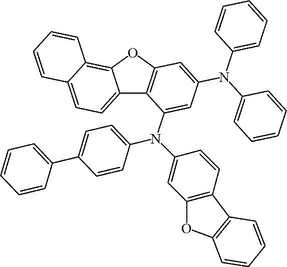 [SMILES: c1ccc(-c2ccc(N(c3ccc4c(c3)oc3ccccc34)c3cc(N(c4ccccc4)c4ccccc4)cc4oc5c6ccccc6ccc5c34)cc2)cc1]

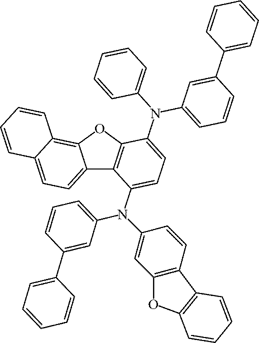 [SMILES: c1ccc(-c2cccc(N(c3ccccc3)c3ccc(N(c4cccc(-c5ccccc5)c4)c4ccc5c(c4)oc4ccccc45)c4c3oc3c5ccccc5ccc34)c2)cc1]

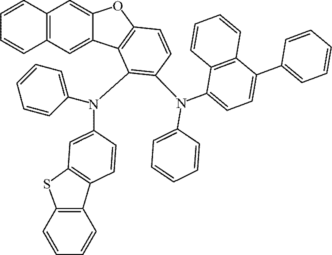 [SMILES: c1ccc(-c2ccc(N(c3ccccc3)c3ccc4oc5cc6ccccc6cc5c4c3N(c3ccccc3)c3ccc4c(c3)sc3ccccc34)c3ccccc23)cc1]

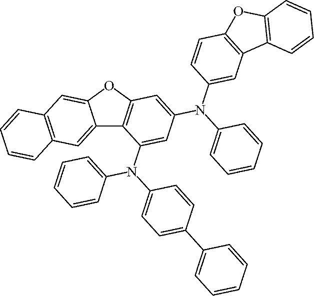 [SMILES: c1ccc(-c2ccc(N(c3ccccc3)c3cc(N(c4ccccc4)c4ccc5oc6ccccc6c5c4)cc4oc5cc6ccccc6cc5c34)cc2)cc1]

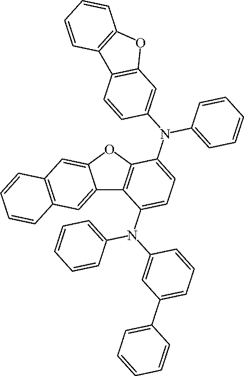 [SMILES: c1ccc(-c2cccc(N(c3ccccc3)c3ccc(N(c4ccccc4)c4ccc5c(c4)oc4ccccc45)c4oc5cc6ccccc6cc5c34)c2)cc1]

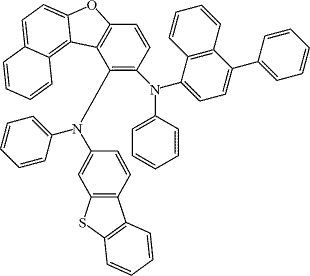 [SMILES: c1ccc(-c2ccc(N(c3ccccc3)c3ccc4oc5ccc6ccccc6c5c4c3N(c3ccccc3)c3ccc4c(c3)sc3ccccc34)c3ccccc23)cc1]

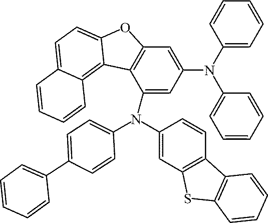 [SMILES: c1ccc(-c2ccc(N(c3ccc4c(c3)sc3ccccc34)c3cc(N(c4ccccc4)c4ccccc4)cc4oc5ccc6ccccc6c5c34)cc2)cc1]

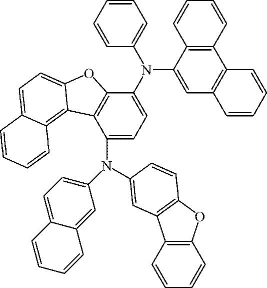 [SMILES: c1ccc(N(c2cc3ccccc3c3ccccc23)c2ccc(N(c3ccc4ccccc4c3)c3ccc4oc5ccccc5c4c3)c3c2oc2ccc4ccccc4c23)cc1]

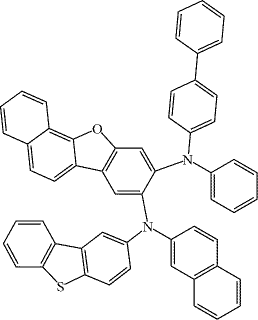 [SMILES: c1ccc(-c2ccc(N(c3ccccc3)c3cc4oc5c6ccccc6ccc5c4cc3N(c3ccc4ccccc4c3)c3ccc4sc5ccccc5c4c3)cc2)cc1]

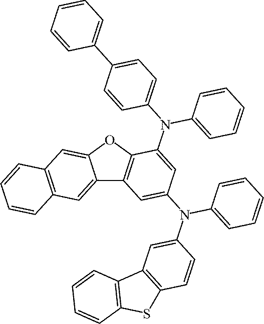 [SMILES: c1ccc(-c2ccc(N(c3ccccc3)c3cc(N(c4ccccc4)c4ccc5sc6ccccc6c5c4)cc4c3oc3cc5ccccc5cc34)cc2)cc1]

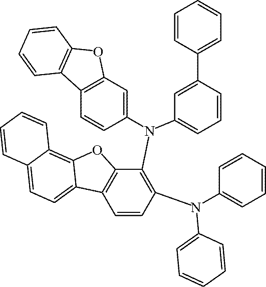 [SMILES: c1ccc(-c2cccc(N(c3ccc4c(c3)oc3ccccc34)c3c(N(c4ccccc4)c4ccccc4)ccc4c3oc3c5ccccc5ccc43)c2)cc1]

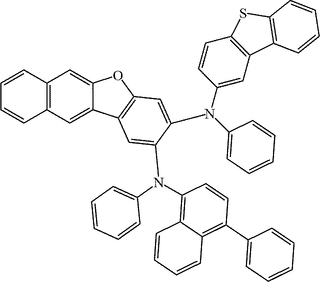 [SMILES: c1ccc(-c2ccc(N(c3ccccc3)c3cc4c(cc3N(c3ccccc3)c3ccc5sc6ccccc6c5c3)oc3cc5ccccc5cc34)c3ccccc23)cc1]

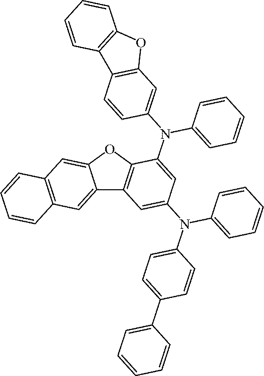 [SMILES: c1ccc(-c2ccc(N(c3ccccc3)c3cc(N(c4ccccc4)c4ccc5c(c4)oc4ccccc45)c4oc5cc6ccccc6cc5c4c3)cc2)cc1]

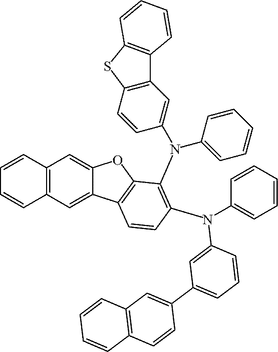 [SMILES: c1ccc(N(c2cccc(-c3ccc4ccccc4c3)c2)c2ccc3c(oc4cc5ccccc5cc43)c2N(c2ccccc2)c2ccc3sc4ccccc4c3c2)cc1]

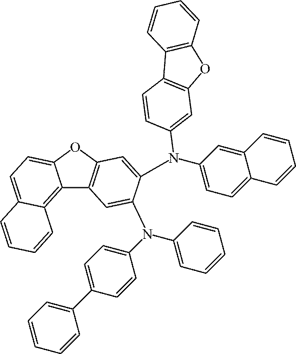 [SMILES: c1ccc(-c2ccc(N(c3ccccc3)c3cc4c(cc3N(c3ccc5ccccc5c3)c3ccc5c(c3)oc3ccccc35)oc3ccc5ccccc5c34)cc2)cc1]

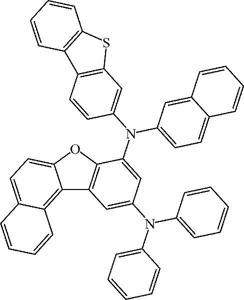 [SMILES: c1ccc(N(c2ccccc2)c2cc(N(c3ccc4ccccc4c3)c3ccc4c(c3)sc3ccccc34)c3oc4ccc5ccccc5c4c3c2)cc1]

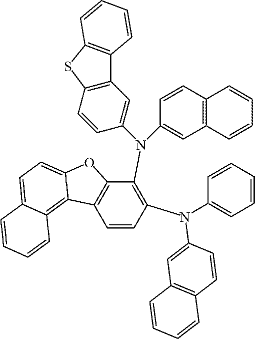 [SMILES: c1ccc(N(c2ccc3ccccc3c2)c2ccc3c(oc4ccc5ccccc5c43)c2N(c2ccc3ccccc3c2)c2ccc3sc4ccccc4c3c2)cc1]

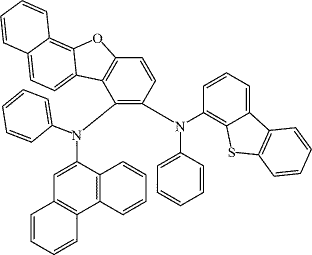 [SMILES: c1ccc(N(c2ccc3oc4c5ccccc5ccc4c3c2N(c2ccccc2)c2cc3ccccc3c3ccccc23)c2cccc3c2sc2ccccc23)cc1]

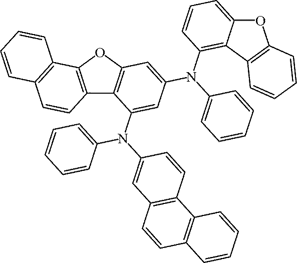 [SMILES: c1ccc(N(c2cc(N(c3ccccc3)c3ccc4c(ccc5ccccc54)c3)c3c(c2)oc2c4ccccc4ccc23)c2cccc3oc4ccccc4c23)cc1]

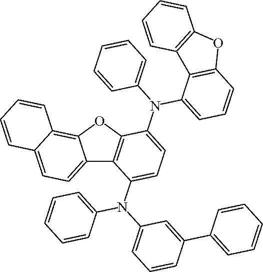 [SMILES: c1ccc(-c2cccc(N(c3ccccc3)c3ccc(N(c4ccccc4)c4cccc5oc6ccccc6c45)c4oc5c6ccccc6ccc5c34)c2)cc1]

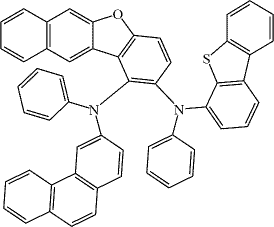 [SMILES: c1ccc(N(c2ccc3oc4cc5ccccc5cc4c3c2N(c2ccccc2)c2ccc3ccc4ccccc4c3c2)c2cccc3c2sc2ccccc23)cc1]

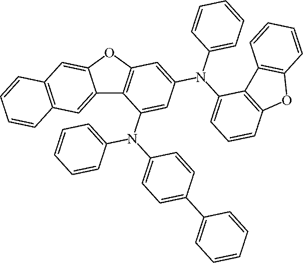 [SMILES: c1ccc(-c2ccc(N(c3ccccc3)c3cc(N(c4ccccc4)c4cccc5oc6ccccc6c45)cc4oc5cc6ccccc6cc5c34)cc2)cc1]

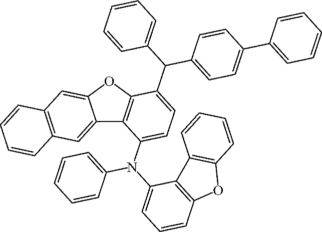 [SMILES: c1ccc(-c2ccc(C(c3ccccc3)c3ccc(N(c4ccccc4)c4cccc5oc6ccccc6c45)c4c3oc3cc5ccccc5cc34)cc2)cc1]

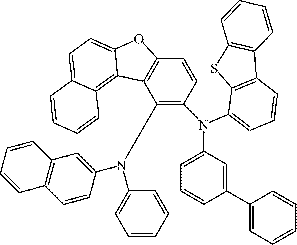 [SMILES: c1ccc(-c2cccc(N(c3ccc4oc5ccc6ccccc6c5c4c3N(c3ccccc3)c3ccc4ccccc4c3)c3cccc4c3sc3ccccc34)c2)cc1]

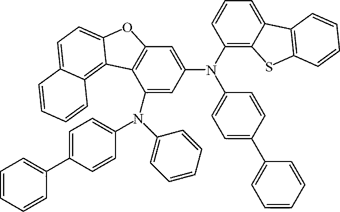 [SMILES: c1ccc(-c2ccc(N(c3cc(N(c4ccccc4)c4ccc(-c5ccccc5)cc4)c4c(c3)oc3ccc5ccccc5c34)c3cccc4c3sc3ccccc34)cc2)cc1]

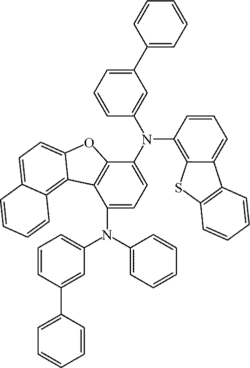 [SMILES: c1ccc(-c2cccc(N(c3ccc(N(c4ccccc4)c4cccc(-c5ccccc5)c4)c4c3oc3ccc5ccccc5c34)c3cccc4c3sc3ccccc34)c2)cc1]

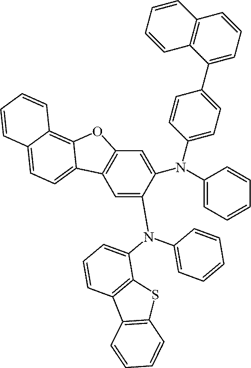 [SMILES: c1ccc(N(c2ccc(-c3cccc4ccccc34)cc2)c2cc3oc4c5ccccc5ccc4c3cc2N(c2ccccc2)c2cccc3c2sc2ccccc23)cc1]

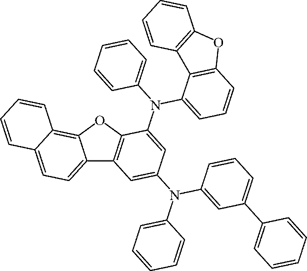 [SMILES: c1ccc(-c2cccc(N(c3ccccc3)c3cc(N(c4ccccc4)c4cccc5oc6ccccc6c45)c4oc5c6ccccc6ccc5c4c3)c2)cc1]

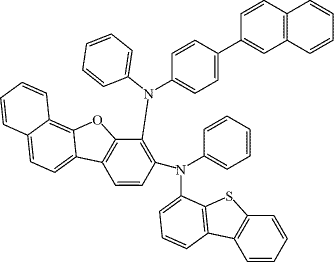 [SMILES: c1ccc(N(c2ccc(-c3ccc4ccccc4c3)cc2)c2c(N(c3ccccc3)c3cccc4c3sc3ccccc34)ccc3c2oc2c4ccccc4ccc32)cc1]

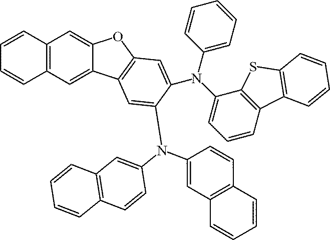 [SMILES: c1ccc(N(c2cc3oc4cc5ccccc5cc4c3cc2N(c2ccc3ccccc3c2)c2ccc3ccccc3c2)c2cccc3c2sc2ccccc23)cc1]

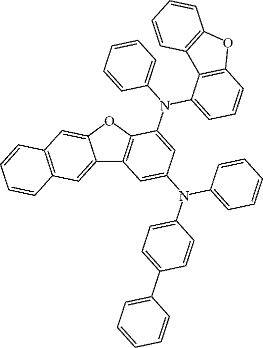 [SMILES: c1ccc(-c2ccc(N(c3ccccc3)c3cc(N(c4ccccc4)c4cccc5oc6ccccc6c45)c4oc5cc6ccccc6cc5c4c3)cc2)cc1]

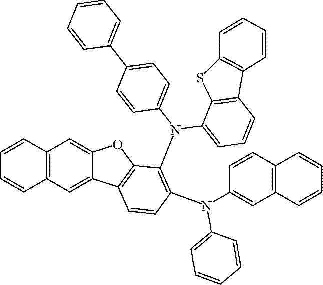 [SMILES: c1ccc(-c2ccc(N(c3c(N(c4ccccc4)c4ccc5ccccc5c4)ccc4c3oc3cc5ccccc5cc34)c3cccc4c3sc3ccccc34)cc2)cc1]

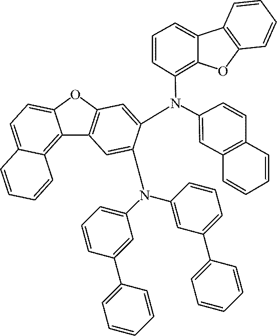 [SMILES: c1ccc(-c2cccc(N(c3cccc(-c4ccccc4)c3)c3cc4c(cc3N(c3ccc5ccccc5c3)c3cccc5c3oc3ccccc35)oc3ccc5ccccc5c34)c2)cc1]

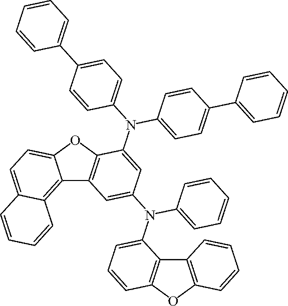 [SMILES: c1ccc(-c2ccc(N(c3ccc(-c4ccccc4)cc3)c3cc(N(c4ccccc4)c4cccc5oc6ccccc6c45)cc4c3oc3ccc5ccccc5c34)cc2)cc1]

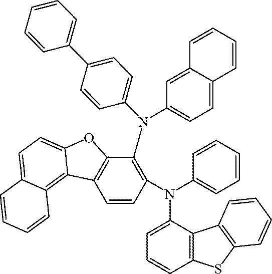 [SMILES: c1ccc(-c2ccc(N(c3ccc4ccccc4c3)c3c(N(c4ccccc4)c4cccc5sc6ccccc6c45)ccc4c3oc3ccc5ccccc5c34)cc2)cc1]

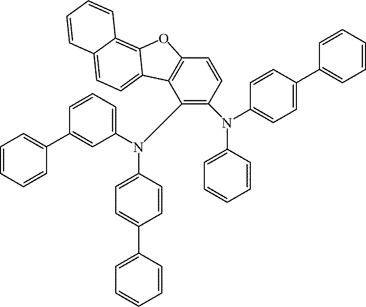 [SMILES: c1ccc(-c2ccc(N(c3ccccc3)c3ccc4oc5c6ccccc6ccc5c4c3N(c3ccc(-c4ccccc4)cc3)c3cccc(-c4ccccc4)c3)cc2)cc1]

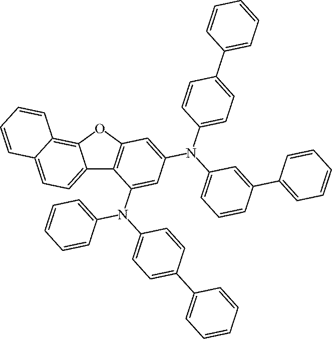 [SMILES: c1ccc(-c2ccc(N(c3cccc(-c4ccccc4)c3)c3cc(N(c4ccccc4)c4ccc(-c5ccccc5)cc4)c4c(c3)oc3c5ccccc5ccc34)cc2)cc1]

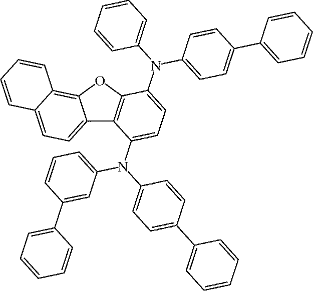 [SMILES: c1ccc(-c2ccc(N(c3ccccc3)c3ccc(N(c4ccc(-c5ccccc5)cc4)c4cccc(-c5ccccc5)c4)c4c3oc3c5ccccc5ccc34)cc2)cc1]

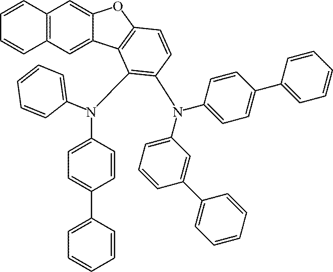 [SMILES: c1ccc(-c2ccc(N(c3cccc(-c4ccccc4)c3)c3ccc4oc5cc6ccccc6cc5c4c3N(c3ccccc3)c3ccc(-c4ccccc4)cc3)cc2)cc1]

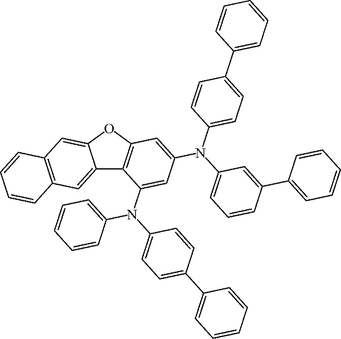 [SMILES: c1ccc(-c2ccc(N(c3cccc(-c4ccccc4)c3)c3cc(N(c4ccccc4)c4ccc(-c5ccccc5)cc4)c4c(c3)oc3cc5ccccc5cc34)cc2)cc1]

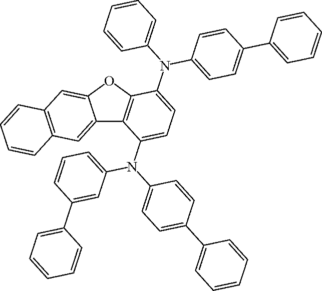 [SMILES: c1ccc(-c2ccc(N(c3ccccc3)c3ccc(N(c4ccc(-c5ccccc5)cc4)c4cccc(-c5ccccc5)c4)c4c3oc3cc5ccccc5cc34)cc2)cc1]

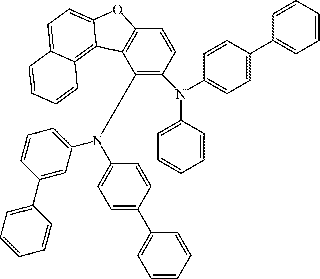 [SMILES: c1ccc(-c2ccc(N(c3ccccc3)c3ccc4oc5ccc6ccccc6c5c4c3N(c3ccc(-c4ccccc4)cc3)c3cccc(-c4ccccc4)c3)cc2)cc1]

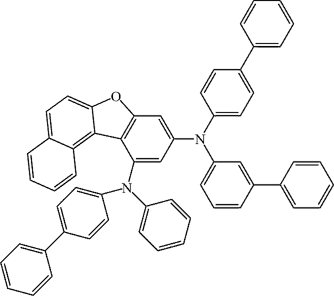 [SMILES: c1ccc(-c2ccc(N(c3cccc(-c4ccccc4)c3)c3cc(N(c4ccccc4)c4ccc(-c5ccccc5)cc4)c4c(c3)oc3ccc5ccccc5c34)cc2)cc1]

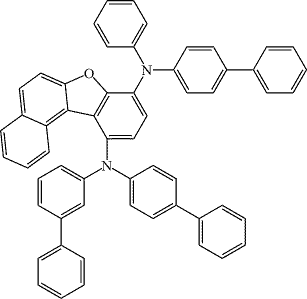 [SMILES: c1ccc(-c2ccc(N(c3ccccc3)c3ccc(N(c4ccc(-c5ccccc5)cc4)c4cccc(-c5ccccc5)c4)c4c3oc3ccc5ccccc5c34)cc2)cc1]

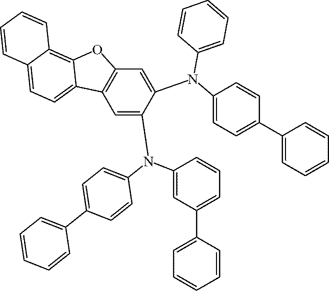 [SMILES: c1ccc(-c2ccc(N(c3ccccc3)c3cc4oc5c6ccccc6ccc5c4cc3N(c3ccc(-c4ccccc4)cc3)c3cccc(-c4ccccc4)c3)cc2)cc1]

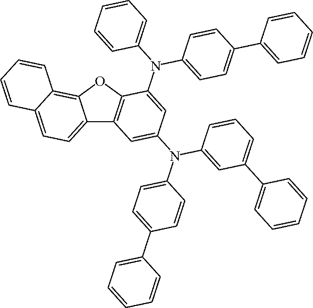 [SMILES: c1ccc(-c2ccc(N(c3cccc(-c4ccccc4)c3)c3cc(N(c4ccccc4)c4ccc(-c5ccccc5)cc4)c4oc5c6ccccc6ccc5c4c3)cc2)cc1]

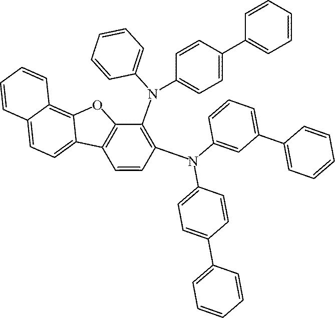 [SMILES: c1ccc(-c2ccc(N(c3cccc(-c4ccccc4)c3)c3ccc4c(oc5c6ccccc6ccc45)c3N(c3ccccc3)c3ccc(-c4ccccc4)cc3)cc2)cc1]

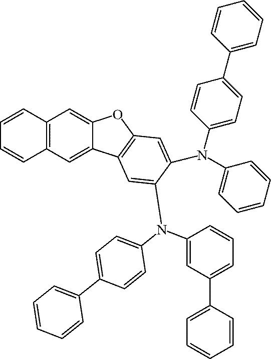 [SMILES: c1ccc(-c2ccc(N(c3ccccc3)c3cc4oc5cc6ccccc6cc5c4cc3N(c3ccc(-c4ccccc4)cc3)c3cccc(-c4ccccc4)c3)cc2)cc1]

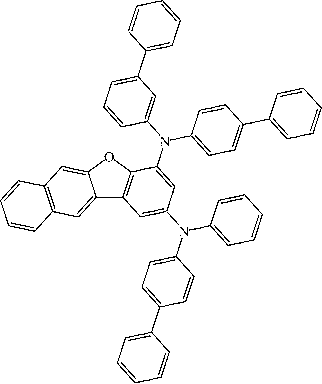 [SMILES: c1ccc(-c2ccc(N(c3ccccc3)c3cc(N(c4ccc(-c5ccccc5)cc4)c4cccc(-c5ccccc5)c4)c4oc5cc6ccccc6cc5c4c3)cc2)cc1]

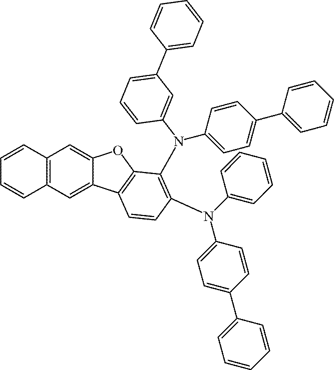 [SMILES: c1ccc(-c2ccc(N(c3ccccc3)c3ccc4c(oc5cc6ccccc6cc54)c3N(c3ccc(-c4ccccc4)cc3)c3cccc(-c4ccccc4)c3)cc2)cc1]

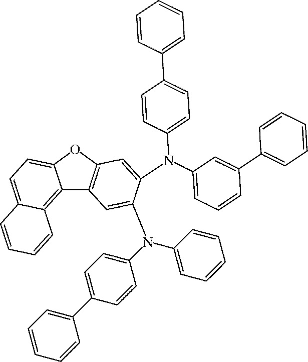 [SMILES: c1ccc(-c2ccc(N(c3cccc(-c4ccccc4)c3)c3cc4oc5ccc6ccccc6c5c4cc3N(c3ccccc3)c3ccc(-c4ccccc4)cc3)cc2)cc1]

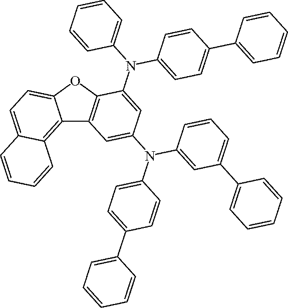 [SMILES: c1ccc(-c2ccc(N(c3cccc(-c4ccccc4)c3)c3cc(N(c4ccccc4)c4ccc(-c5ccccc5)cc4)c4oc5ccc6ccccc6c5c4c3)cc2)cc1]

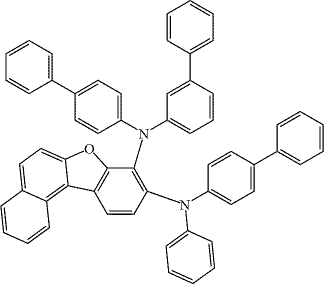 [SMILES: c1ccc(-c2ccc(N(c3ccccc3)c3ccc4c(oc5ccc6ccccc6c54)c3N(c3ccc(-c4ccccc4)cc3)c3cccc(-c4ccccc4)c3)cc2)cc1]

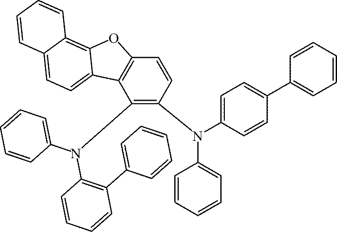 [SMILES: c1ccc(-c2ccc(N(c3ccccc3)c3ccc4oc5c6ccccc6ccc5c4c3N(c3ccccc3)c3ccccc3-c3ccccc3)cc2)cc1]

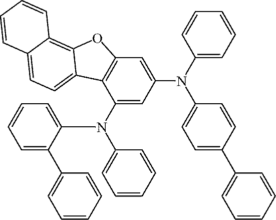 [SMILES: c1ccc(-c2ccc(N(c3ccccc3)c3cc(N(c4ccccc4)c4ccccc4-c4ccccc4)c4c(c3)oc3c5ccccc5ccc34)cc2)cc1]

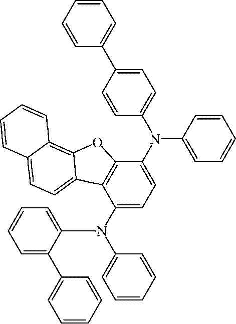 [SMILES: c1ccc(-c2ccc(N(c3ccccc3)c3ccc(N(c4ccccc4)c4ccccc4-c4ccccc4)c4c3oc3c5ccccc5ccc34)cc2)cc1]

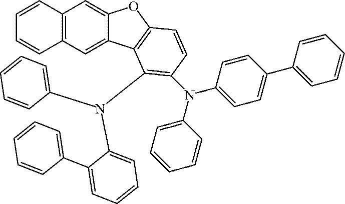 [SMILES: c1ccc(-c2ccc(N(c3ccccc3)c3ccc4oc5cc6ccccc6cc5c4c3N(c3ccccc3)c3ccccc3-c3ccccc3)cc2)cc1]

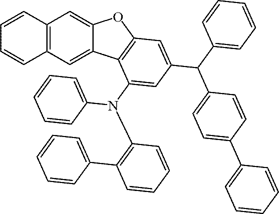 [SMILES: c1ccc(-c2ccc(C(c3ccccc3)c3cc(N(c4ccccc4)c4ccccc4-c4ccccc4)c4c(c3)oc3cc5ccccc5cc34)cc2)cc1]

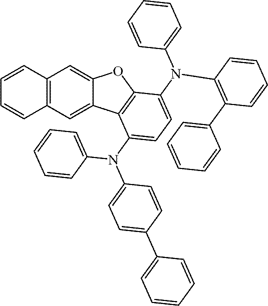 [SMILES: c1ccc(-c2ccc(N(c3ccccc3)c3ccc(N(c4ccccc4)c4ccccc4-c4ccccc4)c4oc5cc6ccccc6cc5c34)cc2)cc1]

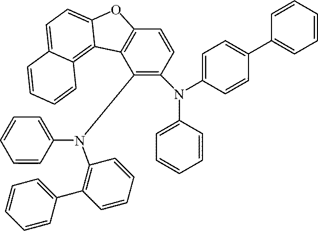 [SMILES: c1ccc(-c2ccc(N(c3ccccc3)c3ccc4oc5ccc6ccccc6c5c4c3N(c3ccccc3)c3ccccc3-c3ccccc3)cc2)cc1]

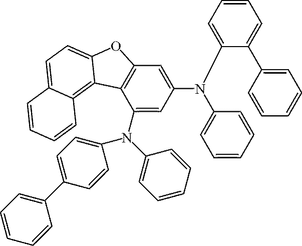 [SMILES: c1ccc(-c2ccc(N(c3ccccc3)c3cc(N(c4ccccc4)c4ccccc4-c4ccccc4)cc4oc5ccc6ccccc6c5c34)cc2)cc1]

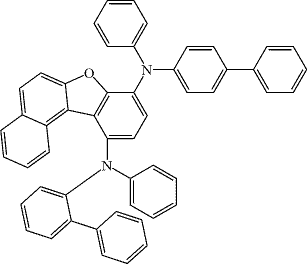 [SMILES: c1ccc(-c2ccc(N(c3ccccc3)c3ccc(N(c4ccccc4)c4ccccc4-c4ccccc4)c4c3oc3ccc5ccccc5c34)cc2)cc1]

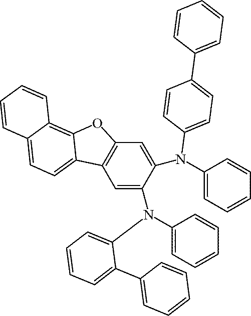 [SMILES: c1ccc(-c2ccc(N(c3ccccc3)c3cc4oc5c6ccccc6ccc5c4cc3N(c3ccccc3)c3ccccc3-c3ccccc3)cc2)cc1]

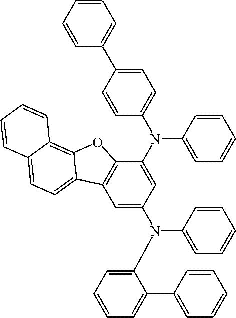 [SMILES: c1ccc(-c2ccc(N(c3ccccc3)c3cc(N(c4ccccc4)c4ccccc4-c4ccccc4)cc4c3oc3c5ccccc5ccc43)cc2)cc1]

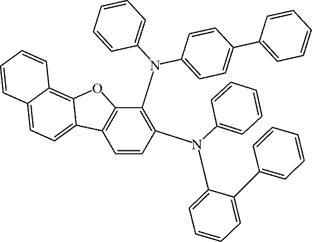 [SMILES: c1ccc(-c2ccc(N(c3ccccc3)c3c(N(c4ccccc4)c4ccccc4-c4ccccc4)ccc4c3oc3c5ccccc5ccc43)cc2)cc1]

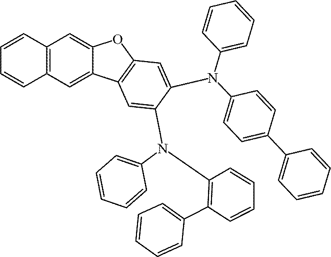 [SMILES: c1ccc(-c2ccc(N(c3ccccc3)c3cc4oc5cc6ccccc6cc5c4cc3N(c3ccccc3)c3ccccc3-c3ccccc3)cc2)cc1]

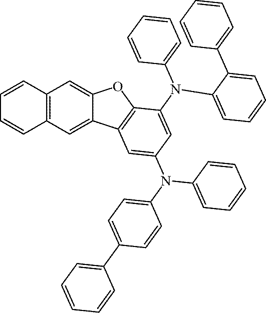 [SMILES: c1ccc(-c2ccc(N(c3ccccc3)c3cc(N(c4ccccc4)c4ccccc4-c4ccccc4)c4oc5cc6ccccc6cc5c4c3)cc2)cc1]

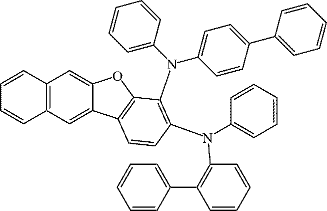 [SMILES: c1ccc(-c2ccc(N(c3ccccc3)c3c(N(c4ccccc4)c4ccccc4-c4ccccc4)ccc4c3oc3cc5ccccc5cc34)cc2)cc1]

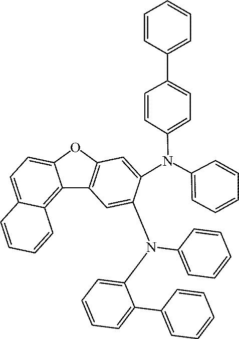 [SMILES: c1ccc(-c2ccc(N(c3ccccc3)c3cc4oc5ccc6ccccc6c5c4cc3N(c3ccccc3)c3ccccc3-c3ccccc3)cc2)cc1]

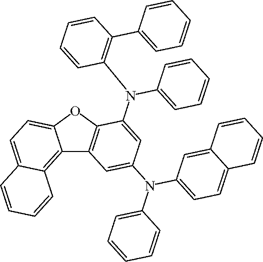 [SMILES: c1ccc(-c2ccccc2N(c2ccccc2)c2cc(N(c3ccccc3)c3ccc4ccccc4c3)cc3c2oc2ccc4ccccc4c23)cc1]

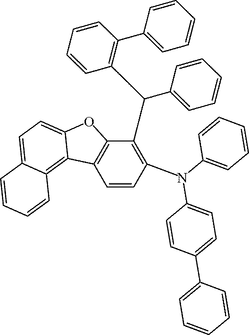 [SMILES: c1ccc(-c2ccc(N(c3ccccc3)c3ccc4c(oc5ccc6ccccc6c54)c3C(c3ccccc3)c3ccccc3-c3ccccc3)cc2)cc1]

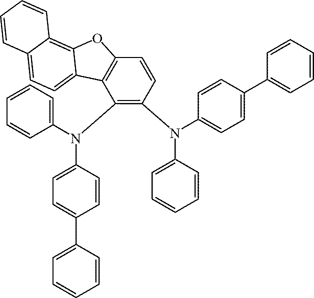 [SMILES: c1ccc(-c2ccc(N(c3ccccc3)c3ccc4oc5c6ccccc6ccc5c4c3N(c3ccccc3)c3ccc(-c4ccccc4)cc3)cc2)cc1]

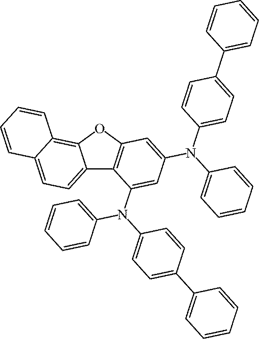 [SMILES: c1ccc(-c2ccc(N(c3ccccc3)c3cc(N(c4ccccc4)c4ccc(-c5ccccc5)cc4)c4c(c3)oc3c5ccccc5ccc34)cc2)cc1]

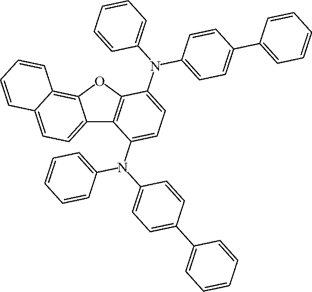 [SMILES: c1ccc(-c2ccc(N(c3ccccc3)c3ccc(N(c4ccccc4)c4ccc(-c5ccccc5)cc4)c4c3oc3c5ccccc5ccc34)cc2)cc1]

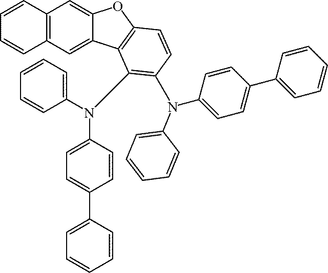 [SMILES: c1ccc(-c2ccc(N(c3ccccc3)c3ccc4oc5cc6ccccc6cc5c4c3N(c3ccccc3)c3ccc(-c4ccccc4)cc3)cc2)cc1]

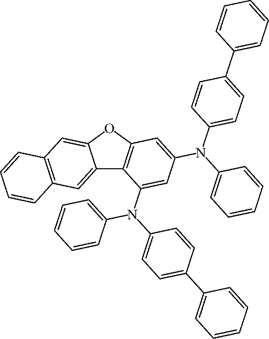 [SMILES: c1ccc(-c2ccc(N(c3ccccc3)c3cc(N(c4ccccc4)c4ccc(-c5ccccc5)cc4)c4c(c3)oc3cc5ccccc5cc34)cc2)cc1]

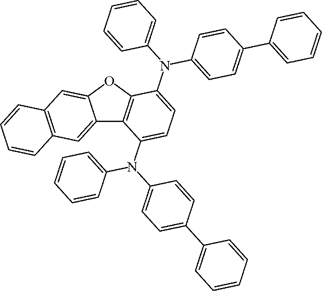 [SMILES: c1ccc(-c2ccc(N(c3ccccc3)c3ccc(N(c4ccccc4)c4ccc(-c5ccccc5)cc4)c4c3oc3cc5ccccc5cc34)cc2)cc1]

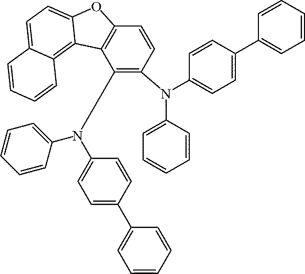 [SMILES: c1ccc(-c2ccc(N(c3ccccc3)c3ccc4oc5ccc6ccccc6c5c4c3N(c3ccccc3)c3ccc(-c4ccccc4)cc3)cc2)cc1]

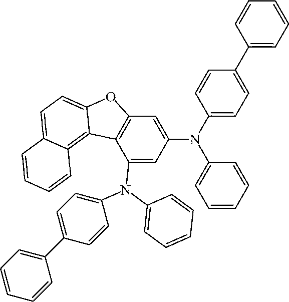 [SMILES: c1ccc(-c2ccc(N(c3ccccc3)c3cc(N(c4ccccc4)c4ccc(-c5ccccc5)cc4)c4c(c3)oc3ccc5ccccc5c34)cc2)cc1]

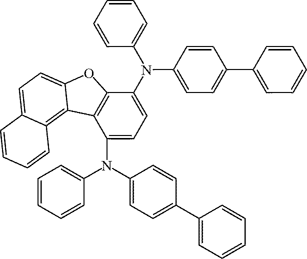 [SMILES: c1ccc(-c2ccc(N(c3ccccc3)c3ccc(N(c4ccccc4)c4ccc(-c5ccccc5)cc4)c4c3oc3ccc5ccccc5c34)cc2)cc1]

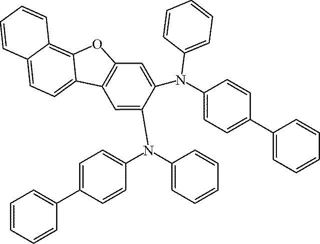 [SMILES: c1ccc(-c2ccc(N(c3ccccc3)c3cc4oc5c6ccccc6ccc5c4cc3N(c3ccccc3)c3ccc(-c4ccccc4)cc3)cc2)cc1]

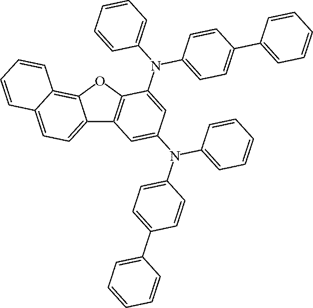 [SMILES: c1ccc(-c2ccc(N(c3ccccc3)c3cc(N(c4ccccc4)c4ccc(-c5ccccc5)cc4)c4oc5c6ccccc6ccc5c4c3)cc2)cc1]

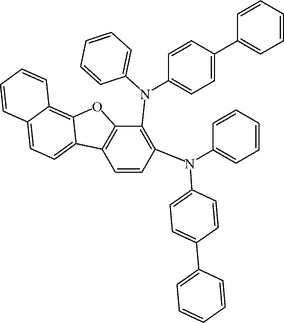 [SMILES: c1ccc(-c2ccc(N(c3ccccc3)c3ccc4c(oc5c6ccccc6ccc45)c3N(c3ccccc3)c3ccc(-c4ccccc4)cc3)cc2)cc1]

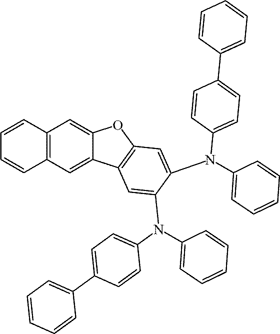 [SMILES: c1ccc(-c2ccc(N(c3ccccc3)c3cc4oc5cc6ccccc6cc5c4cc3N(c3ccccc3)c3ccc(-c4ccccc4)cc3)cc2)cc1]

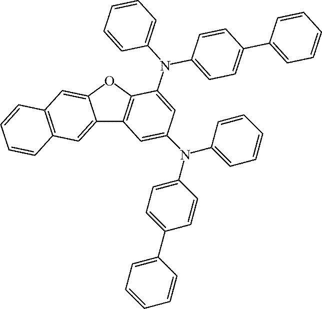 [SMILES: c1ccc(-c2ccc(N(c3ccccc3)c3cc(N(c4ccccc4)c4ccc(-c5ccccc5)cc4)c4oc5cc6ccccc6cc5c4c3)cc2)cc1]

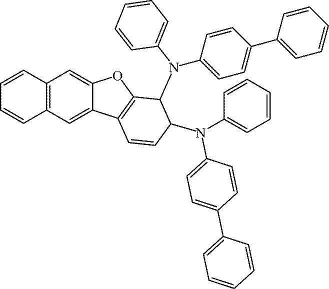 [SMILES: C1=CC(N(c2ccccc2)c2ccc(-c3ccccc3)cc2)C(N(c2ccccc2)c2ccc(-c3ccccc3)cc2)c2oc3cc4ccccc4cc3c21]

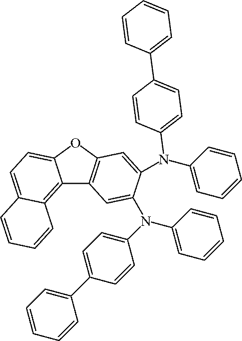 [SMILES: c1ccc(-c2ccc(N(c3ccccc3)c3cc4oc5ccc6ccccc6c5c4cc3N(c3ccccc3)c3ccc(-c4ccccc4)cc3)cc2)cc1]

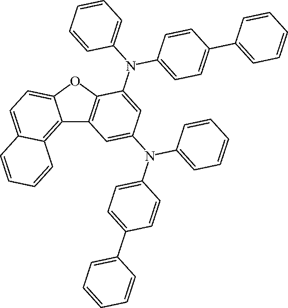 [SMILES: c1ccc(-c2ccc(N(c3ccccc3)c3cc(N(c4ccccc4)c4ccc(-c5ccccc5)cc4)c4oc5ccc6ccccc6c5c4c3)cc2)cc1]

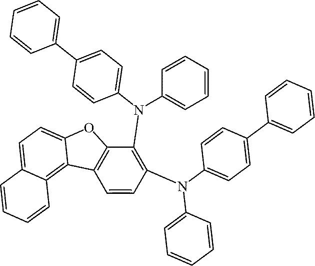 [SMILES: c1ccc(-c2ccc(N(c3ccccc3)c3ccc4c(oc5ccc6ccccc6c54)c3N(c3ccccc3)c3ccc(-c4ccccc4)cc3)cc2)cc1]

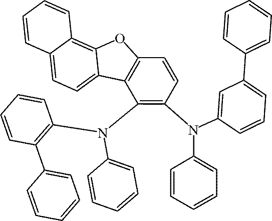 [SMILES: c1ccc(-c2cccc(N(c3ccccc3)c3ccc4oc5c6ccccc6ccc5c4c3N(c3ccccc3)c3ccccc3-c3ccccc3)c2)cc1]

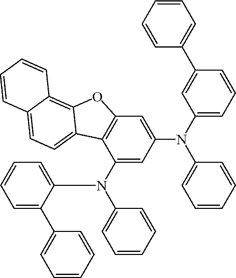 [SMILES: c1ccc(-c2cccc(N(c3ccccc3)c3cc(N(c4ccccc4)c4ccccc4-c4ccccc4)c4c(c3)oc3c5ccccc5ccc34)c2)cc1]

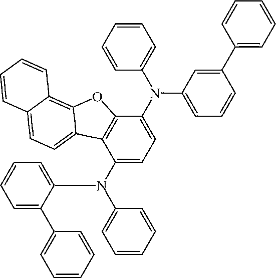 [SMILES: c1ccc(-c2cccc(N(c3ccccc3)c3ccc(N(c4ccccc4)c4ccccc4-c4ccccc4)c4c3oc3c5ccccc5ccc34)c2)cc1]

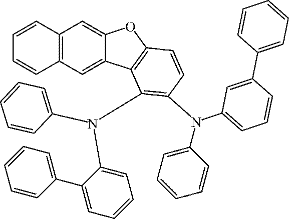 [SMILES: c1ccc(-c2cccc(N(c3ccccc3)c3ccc4oc5cc6ccccc6cc5c4c3N(c3ccccc3)c3ccccc3-c3ccccc3)c2)cc1]

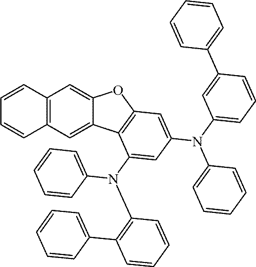 [SMILES: c1ccc(-c2cccc(N(c3ccccc3)c3cc(N(c4ccccc4)c4ccccc4-c4ccccc4)c4c(c3)oc3cc5ccccc5cc34)c2)cc1]

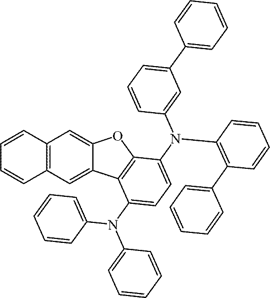 [SMILES: c1ccc(-c2cccc(N(c3ccccc3-c3ccccc3)c3ccc(N(c4ccccc4)c4ccccc4)c4c3oc3cc5ccccc5cc34)c2)cc1]

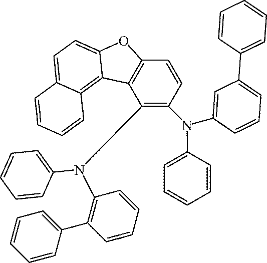 [SMILES: c1ccc(-c2cccc(N(c3ccccc3)c3ccc4oc5ccc6ccccc6c5c4c3N(c3ccccc3)c3ccccc3-c3ccccc3)c2)cc1]

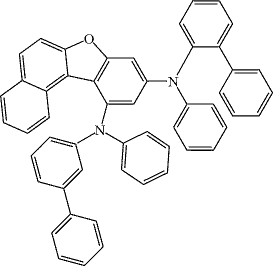

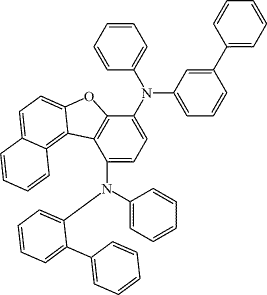 [SMILES: c1ccc(-c2cccc(N(c3ccccc3)c3ccc(N(c4ccccc4)c4ccccc4-c4ccccc4)c4c3oc3ccc5ccccc5c34)c2)cc1]

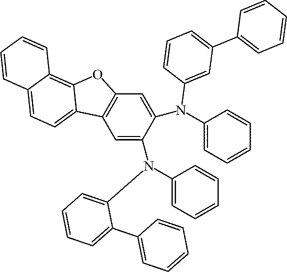 [SMILES: c1ccc(-c2cccc(N(c3ccccc3)c3cc4oc5c6ccccc6ccc5c4cc3N(c3ccccc3)c3ccccc3-c3ccccc3)c2)cc1]

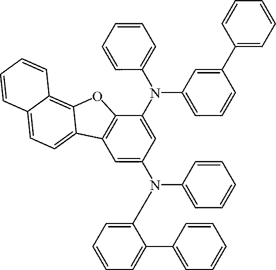 [SMILES: c1ccc(-c2cccc(N(c3ccccc3)c3cc(N(c4ccccc4)c4ccccc4-c4ccccc4)cc4c3oc3c5ccccc5ccc43)c2)cc1]

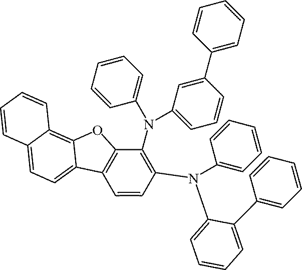 [SMILES: c1ccc(-c2cccc(N(c3ccccc3)c3c(N(c4ccccc4)c4ccccc4-c4ccccc4)ccc4c3oc3c5ccccc5ccc43)c2)cc1]

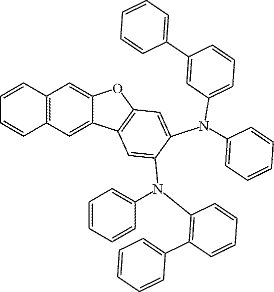 [SMILES: c1ccc(-c2cccc(N(c3ccccc3)c3cc4oc5cc6ccccc6cc5c4cc3N(c3ccccc3)c3ccccc3-c3ccccc3)c2)cc1]

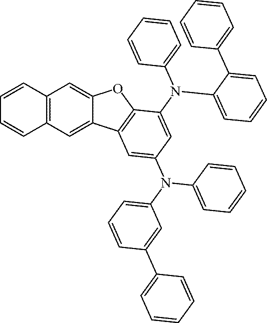 [SMILES: c1ccc(-c2cccc(N(c3ccccc3)c3cc(N(c4ccccc4)c4ccccc4-c4ccccc4)c4oc5cc6ccccc6cc5c4c3)c2)cc1]

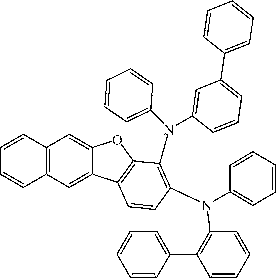 [SMILES: c1ccc(-c2cccc(N(c3ccccc3)c3c(N(c4ccccc4)c4ccccc4-c4ccccc4)ccc4c3oc3cc5ccccc5cc34)c2)cc1]

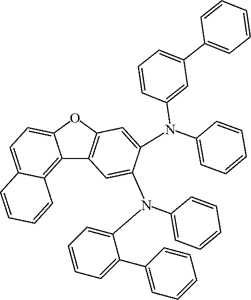 [SMILES: c1ccc(-c2cccc(N(c3ccccc3)c3cc4oc5ccc6ccccc6c5c4cc3N(c3ccccc3)c3ccccc3-c3ccccc3)c2)cc1]

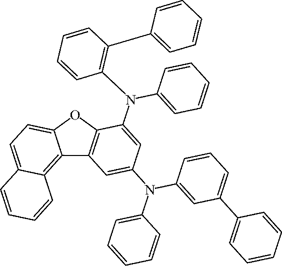 [SMILES: c1ccc(-c2cccc(N(c3ccccc3)c3cc(N(c4ccccc4)c4ccccc4-c4ccccc4)c4oc5ccc6ccccc6c5c4c3)c2)cc1]

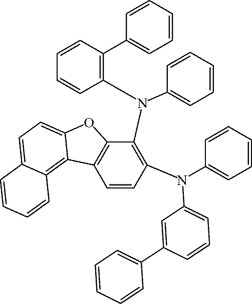 [SMILES: c1ccc(-c2cccc(N(c3ccccc3)c3ccc4c(oc5ccc6ccccc6c54)c3N(c3ccccc3)c3ccccc3-c3ccccc3)c2)cc1]

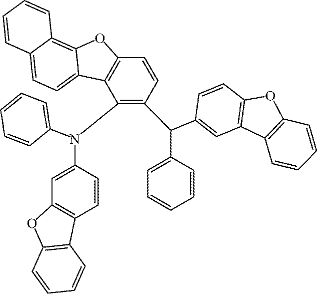 [SMILES: c1ccc(C(c2ccc3oc4ccccc4c3c2)c2ccc3oc4c5ccccc5ccc4c3c2N(c2ccccc2)c2ccc3c(c2)oc2ccccc23)cc1]

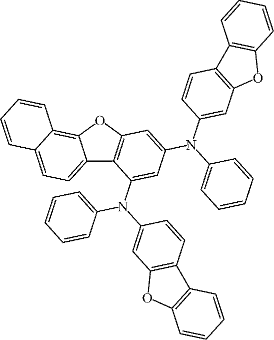 [SMILES: c1ccc(N(c2ccc3c(c2)oc2ccccc23)c2cc(N(c3ccccc3)c3ccc4c(c3)oc3ccccc34)c3c(c2)oc2c4ccccc4ccc23)cc1]

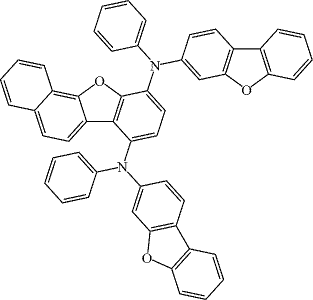 [SMILES: c1ccc(N(c2ccc3c(c2)oc2ccccc23)c2ccc(N(c3ccccc3)c3ccc4c(c3)oc3ccccc34)c3c2oc2c4ccccc4ccc23)cc1]

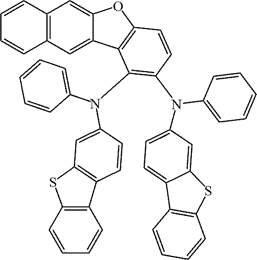 [SMILES: c1ccc(N(c2ccc3c(c2)sc2ccccc23)c2ccc3oc4cc5ccccc5cc4c3c2N(c2ccccc2)c2ccc3c(c2)sc2ccccc23)cc1]

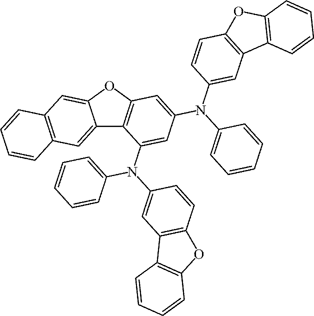 [SMILES: c1ccc(N(c2cc(N(c3ccccc3)c3ccc4oc5ccccc5c4c3)c3c(c2)oc2cc4ccccc4cc23)c2ccc3oc4ccccc4c3c2)cc1]

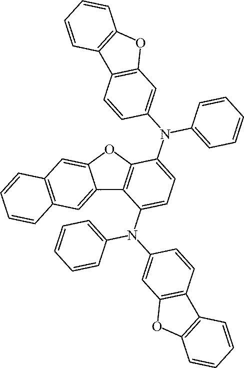 [SMILES: c1ccc(N(c2ccc3c(c2)oc2ccccc23)c2ccc(N(c3ccccc3)c3ccc4c(c3)oc3ccccc34)c3c2oc2cc4ccccc4cc23)cc1]

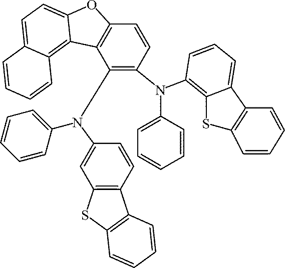 [SMILES: c1ccc(N(c2ccc3oc4ccc5ccccc5c4c3c2N(c2ccccc2)c2ccc3c(c2)sc2ccccc23)c2cccc3c2sc2ccccc23)cc1]

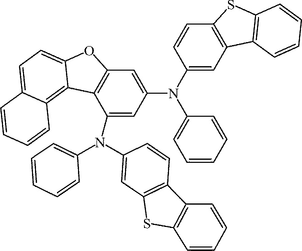 [SMILES: c1ccc(N(c2cc(N(c3ccccc3)c3ccc4c(c3)sc3ccccc34)c3c(c2)oc2ccc4ccccc4c23)c2ccc3sc4ccccc4c3c2)cc1]

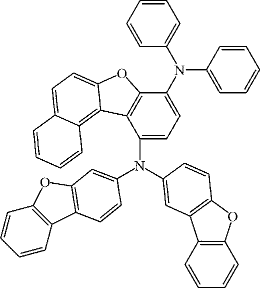 [SMILES: c1ccc(N(c2ccccc2)c2ccc(N(c3ccc4c(c3)oc3ccccc34)c3ccc4oc5ccccc5c4c3)c3c2oc2ccc4ccccc4c23)cc1]

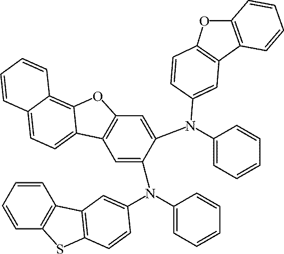 [SMILES: c1ccc(N(c2ccc3oc4ccccc4c3c2)c2cc3oc4c5ccccc5ccc4c3cc2N(c2ccccc2)c2ccc3sc4ccccc4c3c2)cc1]

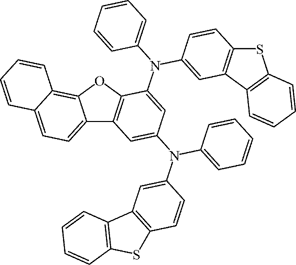 [SMILES: c1ccc(N(c2ccc3sc4ccccc4c3c2)c2cc(N(c3ccccc3)c3ccc4sc5ccccc5c4c3)c3oc4c5ccccc5ccc4c3c2)cc1]

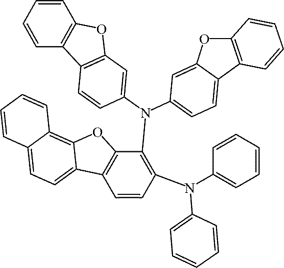 [SMILES: c1ccc(N(c2ccccc2)c2ccc3c(oc4c5ccccc5ccc34)c2N(c2ccc3c(c2)oc2ccccc23)c2ccc3c(c2)oc2ccccc23)cc1]

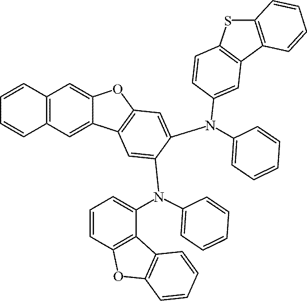 [SMILES: c1ccc(N(c2ccc3sc4ccccc4c3c2)c2cc3oc4cc5ccccc5cc4c3cc2N(c2ccccc2)c2cccc3oc4ccccc4c23)cc1]

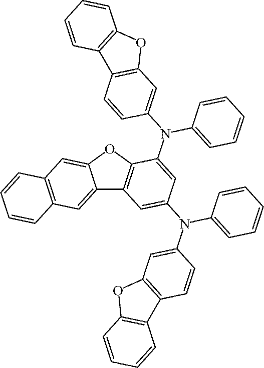 [SMILES: c1ccc(N(c2ccc3c(c2)oc2ccccc23)c2cc(N(c3ccccc3)c3ccc4c(c3)oc3ccccc34)c3oc4cc5ccccc5cc4c3c2)cc1]

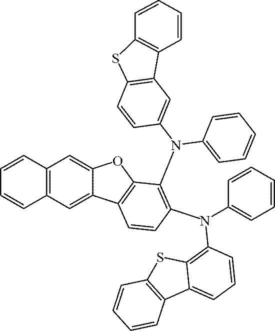 [SMILES: c1ccc(N(c2ccc3sc4ccccc4c3c2)c2c(N(c3ccccc3)c3cccc4c3sc3ccccc34)ccc3c2oc2cc4ccccc4cc23)cc1]

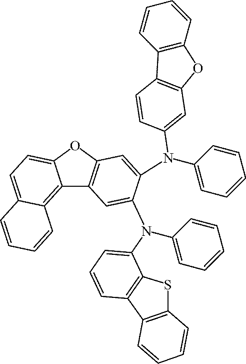 [SMILES: c1ccc(N(c2ccc3c(c2)oc2ccccc23)c2cc3oc4ccc5ccccc5c4c3cc2N(c2ccccc2)c2cccc3c2sc2ccccc23)cc1]

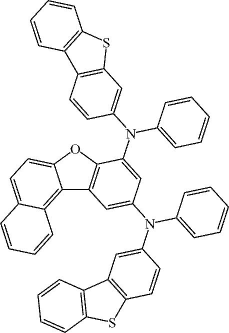 [SMILES: c1ccc(N(c2ccc3sc4ccccc4c3c2)c2cc(N(c3ccccc3)c3ccc4c(c3)sc3ccccc34)c3oc4ccc5ccccc5c4c3c2)cc1]

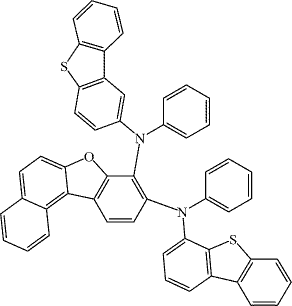 [SMILES: c1ccc(N(c2ccc3sc4ccccc4c3c2)c2c(N(c3ccccc3)c3cccc4c3sc3ccccc34)ccc3c2oc2ccc4ccccc4c23)cc1]

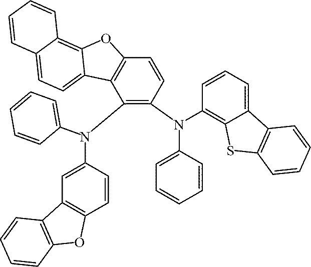 [SMILES: c1ccc(N(c2ccc3oc4c5ccccc5ccc4c3c2N(c2ccccc2)c2ccc3oc4ccccc4c3c2)c2cccc3c2sc2ccccc23)cc1]

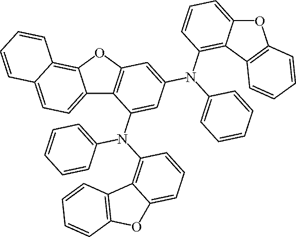 [SMILES: c1ccc(N(c2cc(N(c3ccccc3)c3cccc4oc5ccccc5c34)c3c(c2)oc2c4ccccc4ccc23)c2cccc3oc4ccccc4c23)cc1]

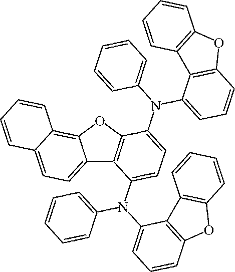 [SMILES: c1ccc(N(c2ccc(N(c3ccccc3)c3cccc4oc5ccccc5c34)c3c2oc2c4ccccc4ccc23)c2cccc3oc4ccccc4c23)cc1]

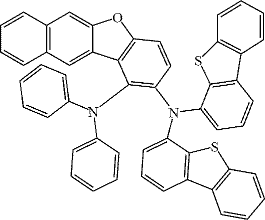 [SMILES: c1ccc(N(c2ccccc2)c2c(N(c3cccc4c3sc3ccccc34)c3cccc4c3sc3ccccc34)ccc3oc4cc5ccccc5cc4c23)cc1]

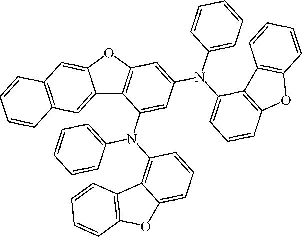 [SMILES: c1ccc(N(c2cc(N(c3ccccc3)c3cccc4oc5ccccc5c34)c3c(c2)oc2cc4ccccc4cc23)c2cccc3oc4ccccc4c23)cc1]

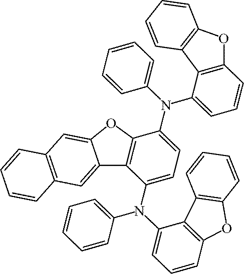 [SMILES: c1ccc(N(c2ccc(N(c3ccccc3)c3cccc4oc5ccccc5c34)c3c2oc2cc4ccccc4cc23)c2cccc3oc4ccccc4c23)cc1]

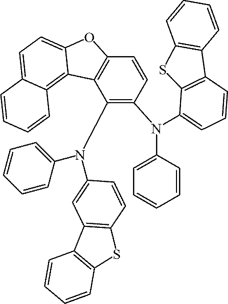 [SMILES: c1ccc(N(c2ccc3oc4ccc5ccccc5c4c3c2N(c2ccccc2)c2ccc3sc4ccccc4c3c2)c2cccc3c2sc2ccccc23)cc1]

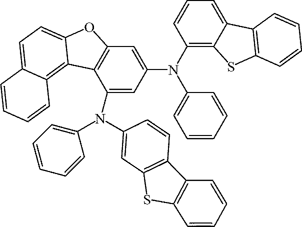 [SMILES: c1ccc(N(c2cc(N(c3ccccc3)c3ccc4c(c3)sc3ccccc34)c3c(c2)oc2ccc4ccccc4c23)c2cccc3c2sc2ccccc23)cc1]

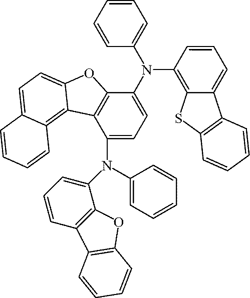 [SMILES: c1ccc(N(c2ccc(N(c3ccccc3)c3cccc4c3oc3ccccc34)c3c2oc2ccc4ccccc4c23)c2cccc3c2sc2ccccc23)cc1]

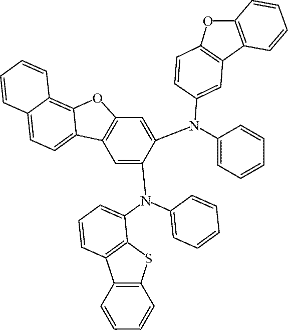 [SMILES: c1ccc(N(c2ccc3oc4ccccc4c3c2)c2cc3oc4c5ccccc5ccc4c3cc2N(c2ccccc2)c2cccc3c2sc2ccccc23)cc1]

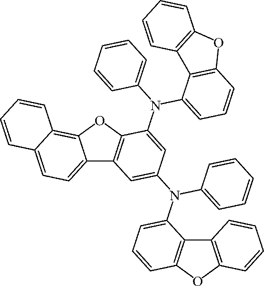 [SMILES: c1ccc(N(c2cc(N(c3ccccc3)c3cccc4oc5ccccc5c34)c3oc4c5ccccc5ccc4c3c2)c2cccc3oc4ccccc4c23)cc1]

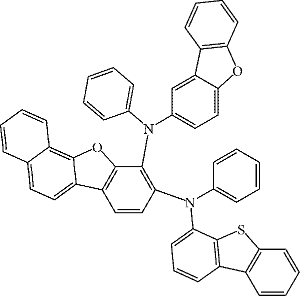 [SMILES: c1ccc(N(c2ccc3oc4ccccc4c3c2)c2c(N(c3ccccc3)c3cccc4c3sc3ccccc34)ccc3c2oc2c4ccccc4ccc32)cc1]

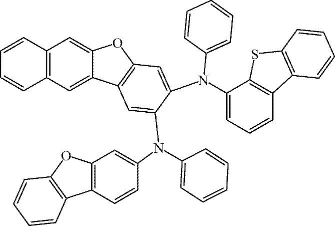 [SMILES: c1ccc(N(c2ccc3c(c2)oc2ccccc23)c2cc3c(cc2N(c2ccccc2)c2cccc4c2sc2ccccc24)oc2cc4ccccc4cc23)cc1]

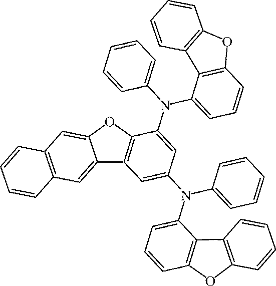 [SMILES: c1ccc(N(c2cc(N(c3ccccc3)c3cccc4oc5ccccc5c34)c3oc4cc5ccccc5cc4c3c2)c2cccc3oc4ccccc4c23)cc1]

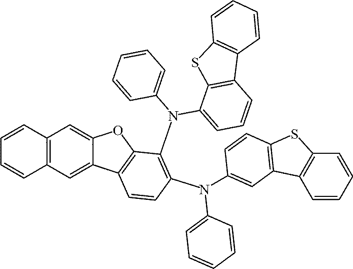 [SMILES: c1ccc(N(c2ccc3sc4ccccc4c3c2)c2ccc3c(oc4cc5ccccc5cc43)c2N(c2ccccc2)c2cccc3c2sc2ccccc23)cc1]

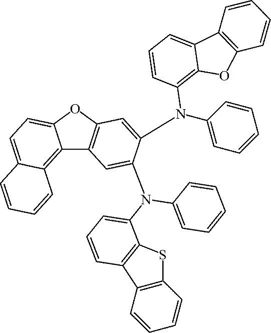 [SMILES: c1ccc(N(c2cc3oc4ccc5ccccc5c4c3cc2N(c2ccccc2)c2cccc3c2sc2ccccc23)c2cccc3c2oc2ccccc23)cc1]

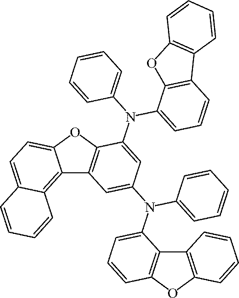 [SMILES: c1ccc(N(c2cccc3c2oc2ccccc23)c2cc(N(c3ccccc3)c3cccc4oc5ccccc5c34)cc3c2oc2ccc4ccccc4c23)cc1]

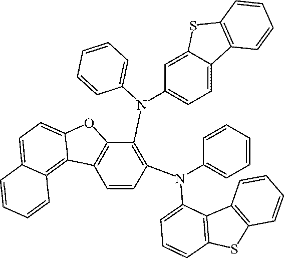 [SMILES: c1ccc(N(c2ccc3c(c2)sc2ccccc23)c2c(N(c3ccccc3)c3cccc4sc5ccccc5c34)ccc3c2oc2ccc4ccccc4c23)cc1]

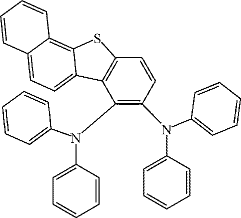 [SMILES: c1ccc(N(c2ccccc2)c2ccc3sc4c5ccccc5ccc4c3c2N(c2ccccc2)c2ccccc2)cc1]

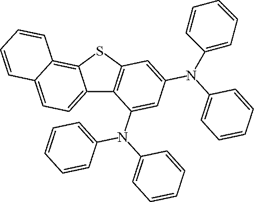 [SMILES: c1ccc(N(c2ccccc2)c2cc(N(c3ccccc3)c3ccccc3)c3c(c2)sc2c4ccccc4ccc23)cc1]

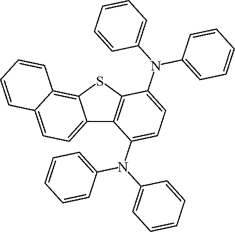 [SMILES: c1ccc(N(c2ccccc2)c2ccc(N(c3ccccc3)c3ccccc3)c3c2sc2c4ccccc4ccc23)cc1]

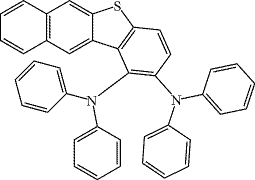 [SMILES: c1ccc(N(c2ccccc2)c2ccc3sc4cc5ccccc5cc4c3c2N(c2ccccc2)c2ccccc2)cc1]

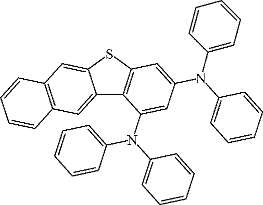 [SMILES: c1ccc(N(c2ccccc2)c2cc(N(c3ccccc3)c3ccccc3)c3c(c2)sc2cc4ccccc4cc23)cc1]

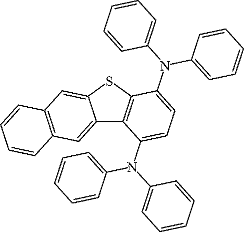 [SMILES: c1ccc(N(c2ccccc2)c2ccc(N(c3ccccc3)c3ccccc3)c3c2sc2cc4ccccc4cc23)cc1]

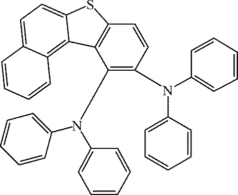 [SMILES: c1ccc(N(c2ccccc2)c2ccc3sc4ccc5ccccc5c4c3c2N(c2ccccc2)c2ccccc2)cc1]

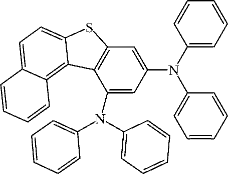 [SMILES: c1ccc(N(c2ccccc2)c2cc(N(c3ccccc3)c3ccccc3)c3c(c2)sc2ccc4ccccc4c23)cc1]

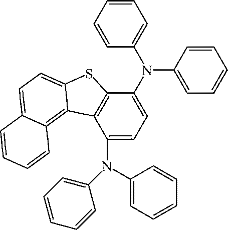 [SMILES: c1ccc(N(c2ccccc2)c2ccc(N(c3ccccc3)c3ccccc3)c3c2sc2ccc4ccccc4c23)cc1]

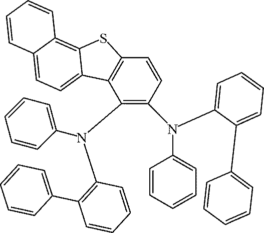 [SMILES: c1ccc(-c2ccccc2N(c2ccccc2)c2ccc3sc4c5ccccc5ccc4c3c2N(c2ccccc2)c2ccccc2-c2ccccc2)cc1]

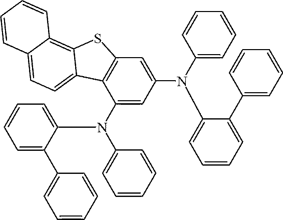 [SMILES: c1ccc(-c2ccccc2N(c2ccccc2)c2cc(N(c3ccccc3)c3ccccc3-c3ccccc3)c3c(c2)sc2c4ccccc4ccc23)cc1]

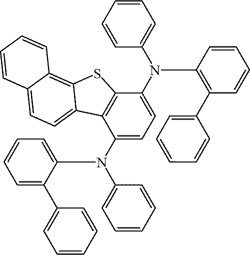 [SMILES: c1ccc(-c2ccccc2N(c2ccccc2)c2ccc(N(c3ccccc3)c3ccccc3-c3ccccc3)c3c2sc2c4ccccc4ccc23)cc1]

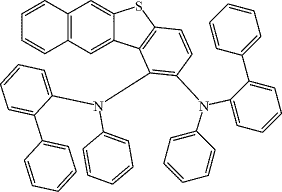 [SMILES: c1ccc(-c2ccccc2N(c2ccccc2)c2ccc3sc4cc5ccccc5cc4c3c2N(c2ccccc2)c2ccccc2-c2ccccc2)cc1]

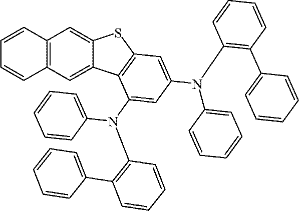 [SMILES: c1ccc(-c2ccccc2N(c2ccccc2)c2cc(N(c3ccccc3)c3ccccc3-c3ccccc3)c3c(c2)sc2cc4ccccc4cc23)cc1]

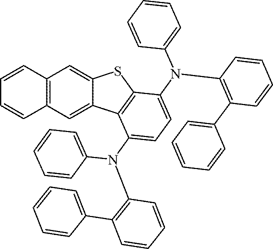 [SMILES: c1ccc(-c2ccccc2N(c2ccccc2)c2ccc(N(c3ccccc3)c3ccccc3-c3ccccc3)c3c2sc2cc4ccccc4cc23)cc1]

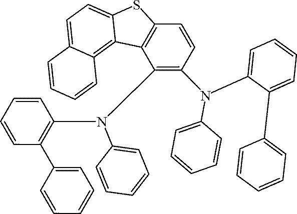 [SMILES: c1ccc(-c2ccccc2N(c2ccccc2)c2ccc3sc4ccc5ccccc5c4c3c2N(c2ccccc2)c2ccccc2-c2ccccc2)cc1]

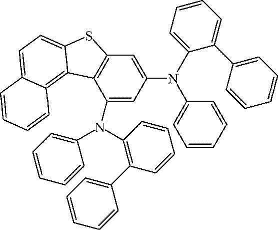 [SMILES: c1ccc(-c2ccccc2N(c2ccccc2)c2cc(N(c3ccccc3)c3ccccc3-c3ccccc3)c3c(c2)sc2ccc4ccccc4c23)cc1]

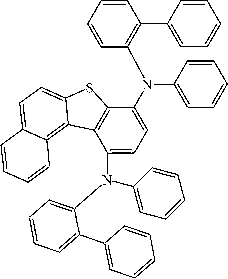 [SMILES: c1ccc(-c2ccccc2N(c2ccccc2)c2ccc(N(c3ccccc3)c3ccccc3-c3ccccc3)c3c2sc2ccc4ccccc4c23)cc1]

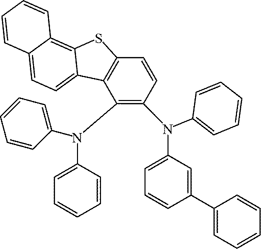 [SMILES: c1ccc(-c2cccc(N(c3ccccc3)c3ccc4sc5c6ccccc6ccc5c4c3N(c3ccccc3)c3ccccc3)c2)cc1]

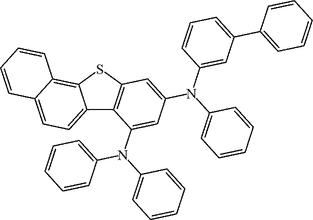 [SMILES: c1ccc(-c2cccc(N(c3ccccc3)c3cc(N(c4ccccc4)c4ccccc4)c4c(c3)sc3c5ccccc5ccc34)c2)cc1]

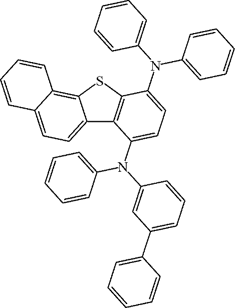 [SMILES: c1ccc(-c2cccc(N(c3ccccc3)c3ccc(N(c4ccccc4)c4ccccc4)c4sc5c6ccccc6ccc5c34)c2)cc1]

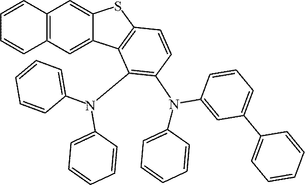 [SMILES: c1ccc(-c2cccc(N(c3ccccc3)c3ccc4sc5cc6ccccc6cc5c4c3N(c3ccccc3)c3ccccc3)c2)cc1]

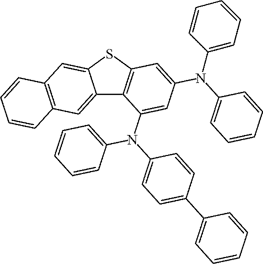 [SMILES: c1ccc(-c2ccc(N(c3ccccc3)c3cc(N(c4ccccc4)c4ccccc4)cc4sc5cc6ccccc6cc5c34)cc2)cc1]

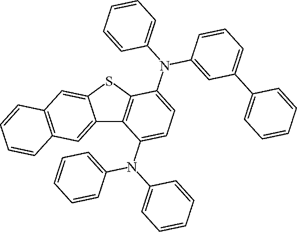 [SMILES: c1ccc(-c2cccc(N(c3ccccc3)c3ccc(N(c4ccccc4)c4ccccc4)c4c3sc3cc5ccccc5cc34)c2)cc1]

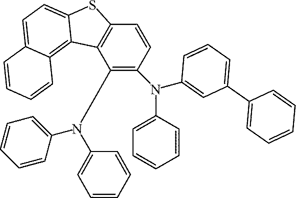 [SMILES: c1ccc(-c2cccc(N(c3ccccc3)c3ccc4sc5ccc6ccccc6c5c4c3N(c3ccccc3)c3ccccc3)c2)cc1]

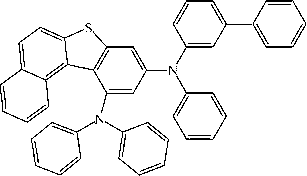 [SMILES: c1ccc(-c2cccc(N(c3ccccc3)c3cc(N(c4ccccc4)c4ccccc4)c4c(c3)sc3ccc5ccccc5c34)c2)cc1]

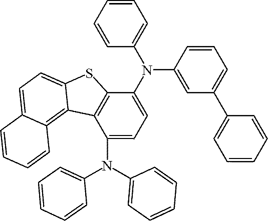 [SMILES: c1ccc(-c2cccc(N(c3ccccc3)c3ccc(N(c4ccccc4)c4ccccc4)c4c3sc3ccc5ccccc5c34)c2)cc1]

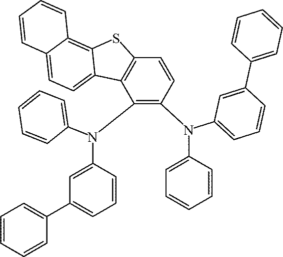 [SMILES: c1ccc(-c2cccc(N(c3ccccc3)c3ccc4sc5c6ccccc6ccc5c4c3N(c3ccccc3)c3cccc(-c4ccccc4)c3)c2)cc1]

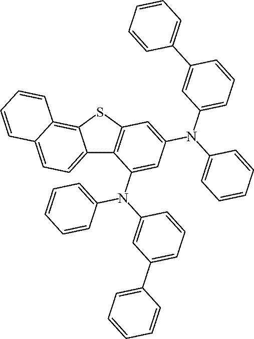 [SMILES: c1ccc(-c2cccc(N(c3ccccc3)c3cc(N(c4ccccc4)c4cccc(-c5ccccc5)c4)c4c(c3)sc3c5ccccc5ccc34)c2)cc1]

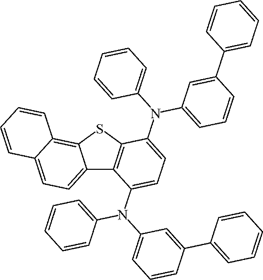 [SMILES: c1ccc(-c2cccc(N(c3ccccc3)c3ccc(N(c4ccccc4)c4cccc(-c5ccccc5)c4)c4c3sc3c5ccccc5ccc34)c2)cc1]

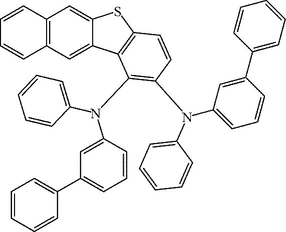 [SMILES: c1ccc(-c2cccc(N(c3ccccc3)c3ccc4sc5cc6ccccc6cc5c4c3N(c3ccccc3)c3cccc(-c4ccccc4)c3)c2)cc1]

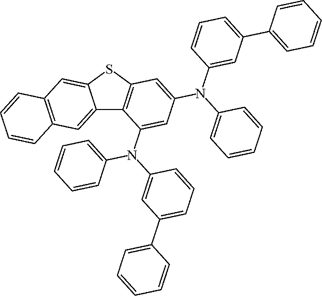 [SMILES: c1ccc(-c2cccc(N(c3ccccc3)c3cc(N(c4ccccc4)c4cccc(-c5ccccc5)c4)c4c(c3)sc3cc5ccccc5cc34)c2)cc1]

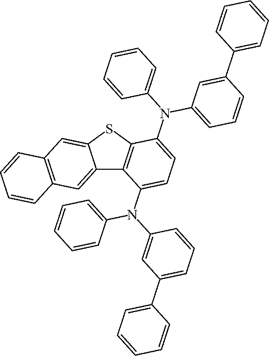 [SMILES: c1ccc(-c2cccc(N(c3ccccc3)c3ccc(N(c4ccccc4)c4cccc(-c5ccccc5)c4)c4c3sc3cc5ccccc5cc34)c2)cc1]

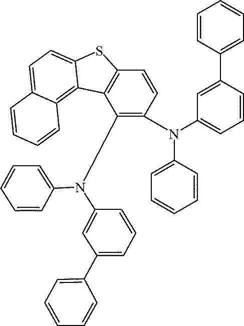 [SMILES: c1ccc(-c2cccc(N(c3ccccc3)c3ccc4sc5ccc6ccccc6c5c4c3N(c3ccccc3)c3cccc(-c4ccccc4)c3)c2)cc1]

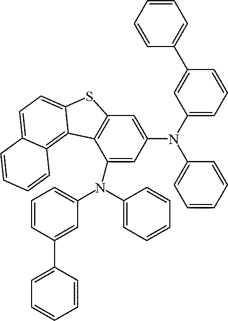 [SMILES: c1ccc(-c2cccc(N(c3ccccc3)c3cc(N(c4ccccc4)c4cccc(-c5ccccc5)c4)c4c(c3)sc3ccc5ccccc5c34)c2)cc1]

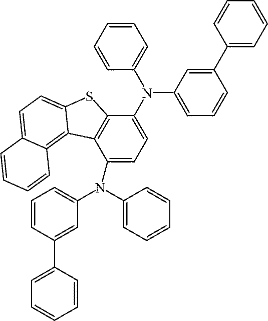 [SMILES: c1ccc(-c2cccc(N(c3ccccc3)c3ccc(N(c4ccccc4)c4cccc(-c5ccccc5)c4)c4c3sc3ccc5ccccc5c34)c2)cc1]

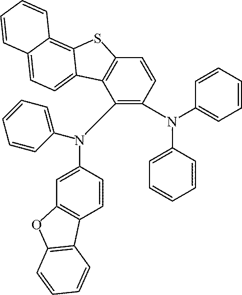 [SMILES: c1ccc(N(c2ccccc2)c2ccc3sc4c5ccccc5ccc4c3c2N(c2ccccc2)c2ccc3c(c2)oc2ccccc23)cc1]

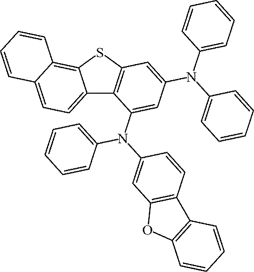 [SMILES: c1ccc(N(c2ccccc2)c2cc(N(c3ccccc3)c3ccc4c(c3)oc3ccccc34)c3c(c2)sc2c4ccccc4ccc23)cc1]

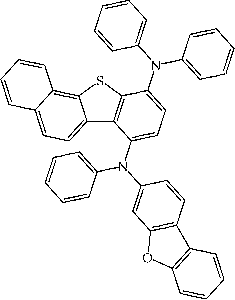 [SMILES: c1ccc(N(c2ccccc2)c2ccc(N(c3ccccc3)c3ccc4c(c3)oc3ccccc34)c3c2sc2c4ccccc4ccc23)cc1]

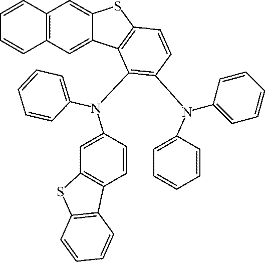 [SMILES: c1ccc(N(c2ccccc2)c2ccc3sc4cc5ccccc5cc4c3c2N(c2ccccc2)c2ccc3c(c2)sc2ccccc23)cc1]

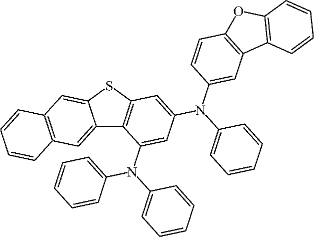 [SMILES: c1ccc(N(c2cc(N(c3ccccc3)c3ccccc3)c3c(c2)sc2cc4ccccc4cc23)c2ccc3oc4ccccc4c3c2)cc1]

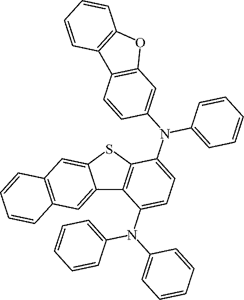 [SMILES: c1ccc(N(c2ccc3c(c2)oc2ccccc23)c2ccc(N(c3ccccc3)c3ccccc3)c3c2sc2cc4ccccc4cc23)cc1]

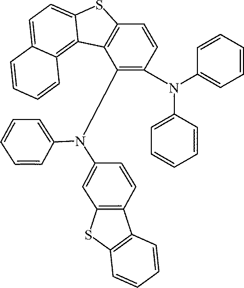 [SMILES: c1ccc(N(c2ccccc2)c2ccc3sc4ccc5ccccc5c4c3c2N(c2ccccc2)c2ccc3c(c2)sc2ccccc23)cc1]

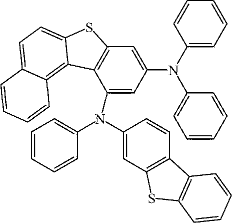 [SMILES: c1ccc(N(c2ccccc2)c2cc(N(c3ccccc3)c3ccc4c(c3)sc3ccccc34)c3c(c2)sc2ccc4ccccc4c23)cc1]

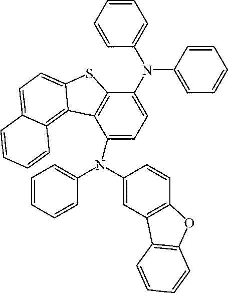 [SMILES: c1ccc(N(c2ccccc2)c2ccc(N(c3ccccc3)c3ccc4oc5ccccc5c4c3)c3c2sc2ccc4ccccc4c23)cc1]

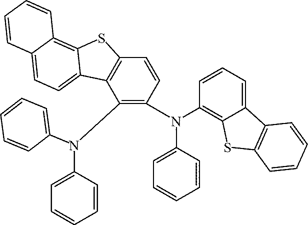 [SMILES: c1ccc(N(c2ccc3sc4c5ccccc5ccc4c3c2N(c2ccccc2)c2ccccc2)c2cccc3c2sc2ccccc23)cc1]

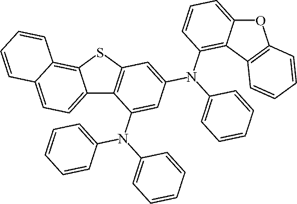 [SMILES: c1ccc(N(c2cc(N(c3ccccc3)c3ccccc3)c3c(c2)sc2c4ccccc4ccc23)c2cccc3oc4ccccc4c23)cc1]

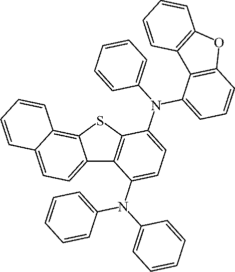 [SMILES: c1ccc(N(c2ccc(N(c3ccccc3)c3ccccc3)c3c2sc2c4ccccc4ccc23)c2cccc3oc4ccccc4c23)cc1]

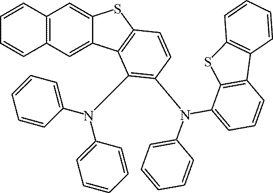 [SMILES: c1ccc(N(c2ccc3sc4cc5ccccc5cc4c3c2N(c2ccccc2)c2ccccc2)c2cccc3c2sc2ccccc23)cc1]

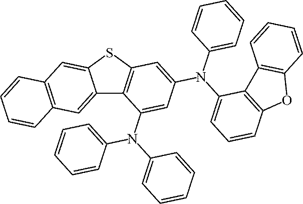 [SMILES: c1ccc(N(c2cc(N(c3ccccc3)c3ccccc3)c3c(c2)sc2cc4ccccc4cc23)c2cccc3oc4ccccc4c23)cc1]

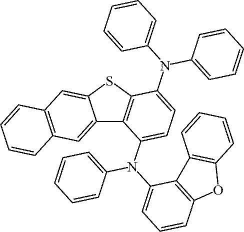 [SMILES: c1ccc(N(c2ccccc2)c2ccc(N(c3ccccc3)c3cccc4oc5ccccc5c34)c3c2sc2cc4ccccc4cc23)cc1]

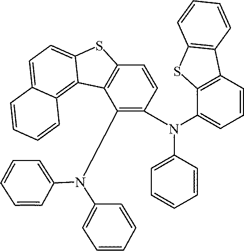 [SMILES: c1ccc(N(c2ccc3sc4ccc5ccccc5c4c3c2N(c2ccccc2)c2ccccc2)c2cccc3c2sc2ccccc23)cc1]

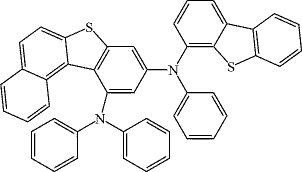 [SMILES: c1ccc(N(c2cc(N(c3ccccc3)c3ccccc3)c3c(c2)sc2ccc4ccccc4c23)c2cccc3c2sc2ccccc23)cc1]

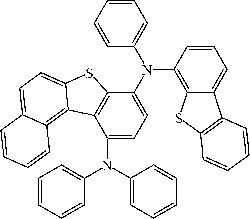 [SMILES: c1ccc(N(c2cccc3c2sc2ccccc23)c2ccc(N(c3ccccc3)c3ccccc3)c3c2sc2ccc4ccccc4c23)cc1]

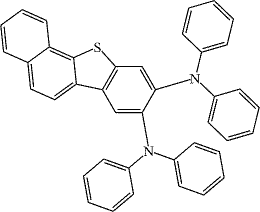 [SMILES: c1ccc(N(c2ccccc2)c2cc3sc4c5ccccc5ccc4c3cc2N(c2ccccc2)c2ccccc2)cc1]

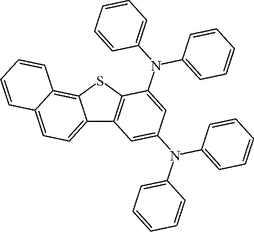 [SMILES: c1ccc(N(c2ccccc2)c2cc(N(c3ccccc3)c3ccccc3)c3sc4c5ccccc5ccc4c3c2)cc1]

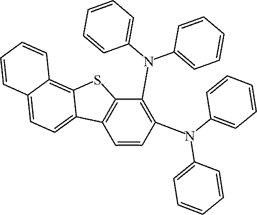 [SMILES: c1ccc(N(c2ccccc2)c2ccc3c(sc4c5ccccc5ccc34)c2N(c2ccccc2)c2ccccc2)cc1]

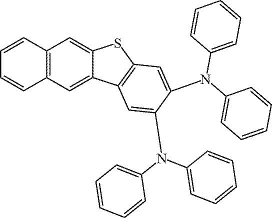 [SMILES: c1ccc(N(c2ccccc2)c2cc3sc4cc5ccccc5cc4c3cc2N(c2ccccc2)c2ccccc2)cc1]

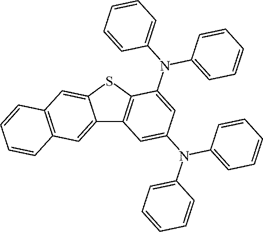 [SMILES: c1ccc(N(c2ccccc2)c2cc(N(c3ccccc3)c3ccccc3)c3sc4cc5ccccc5cc4c3c2)cc1]

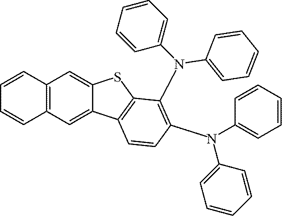 [SMILES: c1ccc(N(c2ccccc2)c2ccc3c(sc4cc5ccccc5cc43)c2N(c2ccccc2)c2ccccc2)cc1]

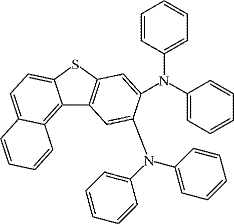 [SMILES: c1ccc(N(c2ccccc2)c2cc3sc4ccc5ccccc5c4c3cc2N(c2ccccc2)c2ccccc2)cc1]

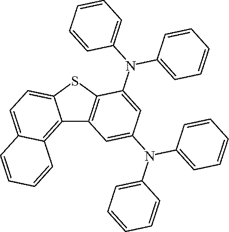 [SMILES: c1ccc(N(c2ccccc2)c2cc(N(c3ccccc3)c3ccccc3)c3sc4ccc5ccccc5c4c3c2)cc1]

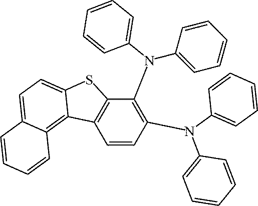 [SMILES: c1ccc(N(c2ccccc2)c2ccc3c(sc4ccc5ccccc5c43)c2N(c2ccccc2)c2ccccc2)cc1]

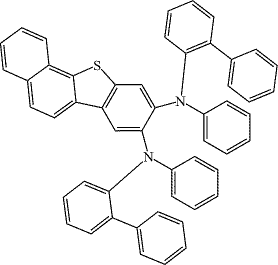 [SMILES: c1ccc(-c2ccccc2N(c2ccccc2)c2cc3sc4c5ccccc5ccc4c3cc2N(c2ccccc2)c2ccccc2-c2ccccc2)cc1]

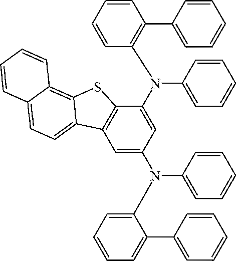 [SMILES: c1ccc(-c2ccccc2N(c2ccccc2)c2cc(N(c3ccccc3)c3ccccc3-c3ccccc3)c3sc4c5ccccc5ccc4c3c2)cc1]

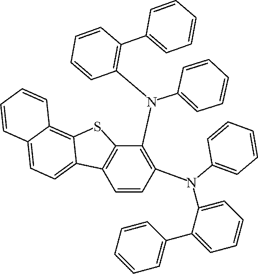 [SMILES: c1ccc(-c2ccccc2N(c2ccccc2)c2ccc3c(sc4c5ccccc5ccc34)c2N(c2ccccc2)c2ccccc2-c2ccccc2)cc1]

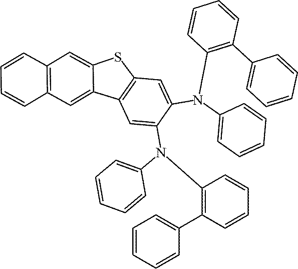 [SMILES: c1ccc(-c2ccccc2N(c2ccccc2)c2cc3sc4cc5ccccc5cc4c3cc2N(c2ccccc2)c2ccccc2-c2ccccc2)cc1]

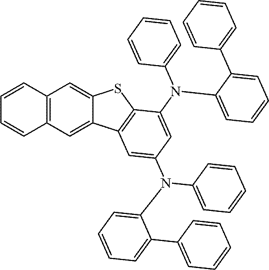 [SMILES: c1ccc(-c2ccccc2N(c2ccccc2)c2cc(N(c3ccccc3)c3ccccc3-c3ccccc3)c3sc4cc5ccccc5cc4c3c2)cc1]

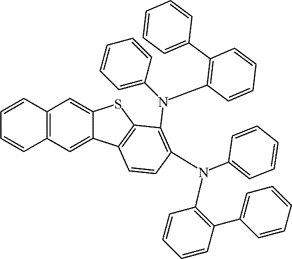 [SMILES: c1ccc(-c2ccccc2N(c2ccccc2)c2ccc3c(sc4cc5ccccc5cc43)c2N(c2ccccc2)c2ccccc2-c2ccccc2)cc1]

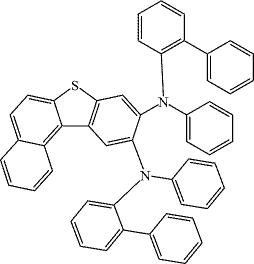 [SMILES: c1ccc(-c2ccccc2N(c2ccccc2)c2cc3sc4ccc5ccccc5c4c3cc2N(c2ccccc2)c2ccccc2-c2ccccc2)cc1]

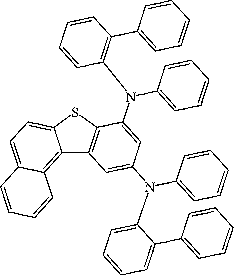 [SMILES: c1ccc(-c2ccccc2N(c2ccccc2)c2cc(N(c3ccccc3)c3ccccc3-c3ccccc3)c3sc4ccc5ccccc5c4c3c2)cc1]

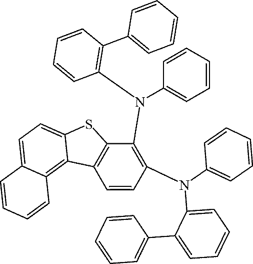 [SMILES: c1ccc(-c2ccccc2N(c2ccccc2)c2ccc3c(sc4ccc5ccccc5c43)c2N(c2ccccc2)c2ccccc2-c2ccccc2)cc1]

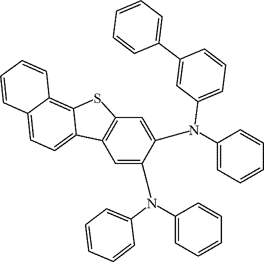 [SMILES: c1ccc(-c2cccc(N(c3ccccc3)c3cc4sc5c6ccccc6ccc5c4cc3N(c3ccccc3)c3ccccc3)c2)cc1]

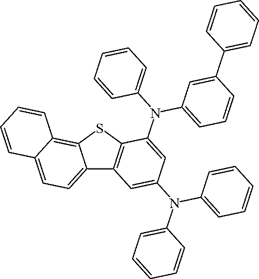 [SMILES: c1ccc(-c2cccc(N(c3ccccc3)c3cc(N(c4ccccc4)c4ccccc4)cc4c3sc3c5ccccc5ccc43)c2)cc1]

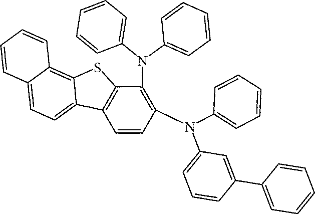 [SMILES: c1ccc(-c2cccc(N(c3ccccc3)c3ccc4c(sc5c6ccccc6ccc45)c3N(c3ccccc3)c3ccccc3)c2)cc1]

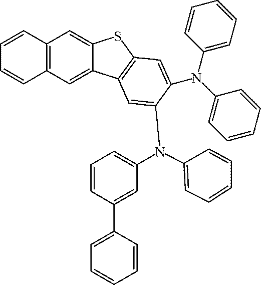 [SMILES: c1ccc(-c2cccc(N(c3ccccc3)c3cc4c(cc3N(c3ccccc3)c3ccccc3)sc3cc5ccccc5cc34)c2)cc1]

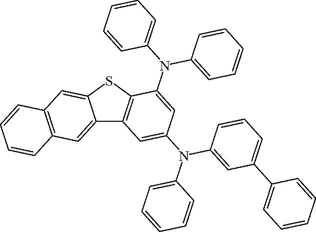 [SMILES: c1ccc(-c2cccc(N(c3ccccc3)c3cc(N(c4ccccc4)c4ccccc4)c4sc5cc6ccccc6cc5c4c3)c2)cc1]

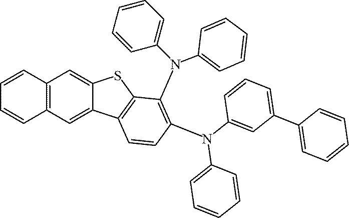 [SMILES: c1ccc(-c2cccc(N(c3ccccc3)c3ccc4c(sc5cc6ccccc6cc54)c3N(c3ccccc3)c3ccccc3)c2)cc1]

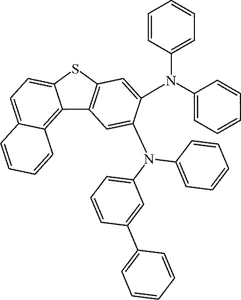 [SMILES: c1ccc(-c2cccc(N(c3ccccc3)c3cc4c(cc3N(c3ccccc3)c3ccccc3)sc3ccc5ccccc5c34)c2)cc1]

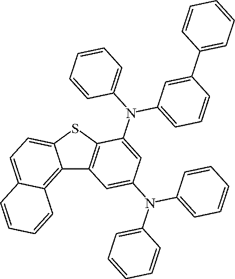 [SMILES: c1ccc(-c2cccc(N(c3ccccc3)c3cc(N(c4ccccc4)c4ccccc4)cc4c3sc3ccc5ccccc5c34)c2)cc1]

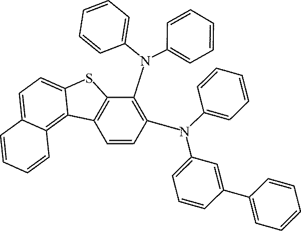 [SMILES: c1ccc(-c2cccc(N(c3ccccc3)c3ccc4c(sc5ccc6ccccc6c54)c3N(c3ccccc3)c3ccccc3)c2)cc1]

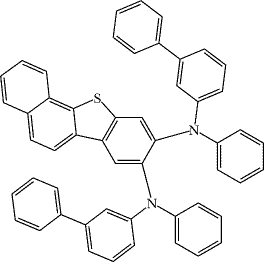 [SMILES: c1ccc(-c2cccc(N(c3ccccc3)c3cc4sc5c6ccccc6ccc5c4cc3N(c3ccccc3)c3cccc(-c4ccccc4)c3)c2)cc1]

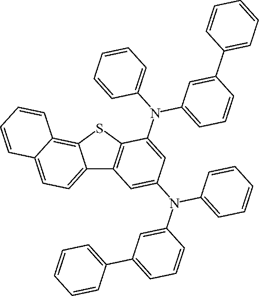 [SMILES: c1ccc(-c2cccc(N(c3ccccc3)c3cc(N(c4ccccc4)c4cccc(-c5ccccc5)c4)c4sc5c6ccccc6ccc5c4c3)c2)cc1]

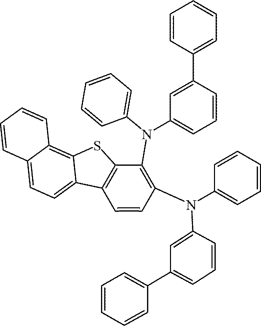 [SMILES: c1ccc(-c2cccc(N(c3ccccc3)c3ccc4c(sc5c6ccccc6ccc45)c3N(c3ccccc3)c3cccc(-c4ccccc4)c3)c2)cc1]

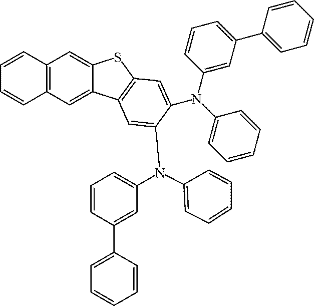 [SMILES: c1ccc(-c2cccc(N(c3ccccc3)c3cc4sc5cc6ccccc6cc5c4cc3N(c3ccccc3)c3cccc(-c4ccccc4)c3)c2)cc1]

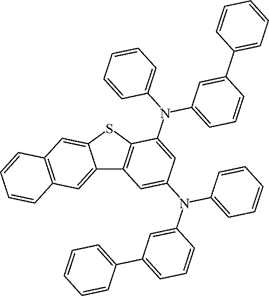 [SMILES: c1ccc(-c2cccc(N(c3ccccc3)c3cc(N(c4ccccc4)c4cccc(-c5ccccc5)c4)c4sc5cc6ccccc6cc5c4c3)c2)cc1]

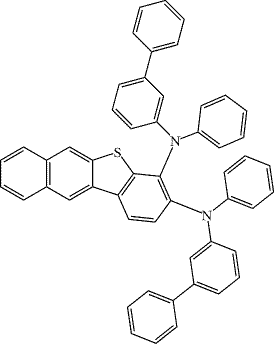 [SMILES: c1ccc(-c2cccc(N(c3ccccc3)c3ccc4c(sc5cc6ccccc6cc54)c3N(c3ccccc3)c3cccc(-c4ccccc4)c3)c2)cc1]

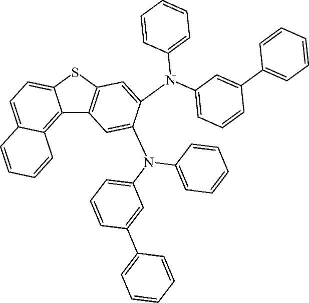 [SMILES: c1ccc(-c2cccc(N(c3ccccc3)c3cc4sc5ccc6ccccc6c5c4cc3N(c3ccccc3)c3cccc(-c4ccccc4)c3)c2)cc1]

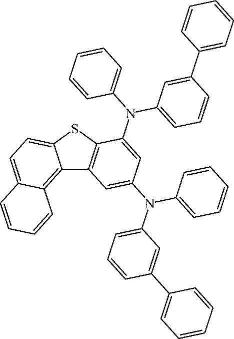 [SMILES: c1ccc(-c2cccc(N(c3ccccc3)c3cc(N(c4ccccc4)c4cccc(-c5ccccc5)c4)c4sc5ccc6ccccc6c5c4c3)c2)cc1]

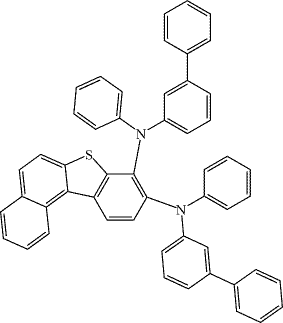 [SMILES: c1ccc(-c2cccc(N(c3ccccc3)c3ccc4c(sc5ccc6ccccc6c54)c3N(c3ccccc3)c3cccc(-c4ccccc4)c3)c2)cc1]

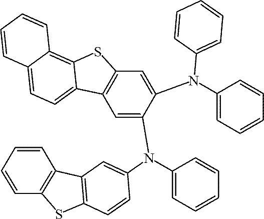 [SMILES: c1ccc(N(c2ccccc2)c2cc3sc4c5ccccc5ccc4c3cc2N(c2ccccc2)c2ccc3sc4ccccc4c3c2)cc1]

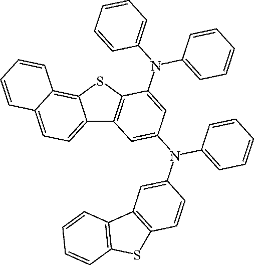 [SMILES: c1ccc(N(c2ccc3sc4ccccc4c3c2)c2cc(N(c3ccccc3)c3ccccc3)c3sc4c5ccccc5ccc4c3c2)cc1]

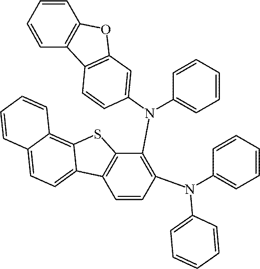 [SMILES: c1ccc(N(c2ccccc2)c2ccc3c(sc4c5ccccc5ccc34)c2N(c2ccccc2)c2ccc3c(c2)oc2ccccc23)cc1]

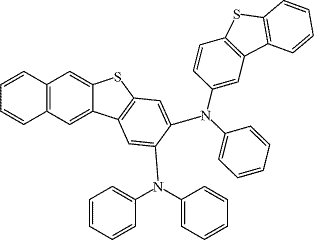 [SMILES: c1ccc(N(c2ccc3sc4ccccc4c3c2)c2cc3sc4cc5ccccc5cc4c3cc2N(c2ccccc2)c2ccccc2)cc1]

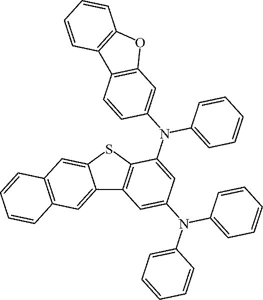 [SMILES: c1ccc(N(c2ccccc2)c2cc(N(c3ccccc3)c3ccc4c(c3)oc3ccccc34)c3sc4cc5ccccc5cc4c3c2)cc1]

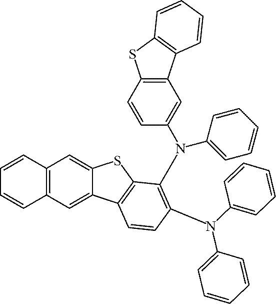 [SMILES: c1ccc(N(c2ccccc2)c2ccc3c(sc4cc5ccccc5cc43)c2N(c2ccccc2)c2ccc3sc4ccccc4c3c2)cc1]

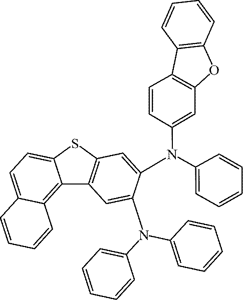 [SMILES: c1ccc(N(c2ccc3c(c2)oc2ccccc23)c2cc3sc4ccc5ccccc5c4c3cc2N(c2ccccc2)c2ccccc2)cc1]

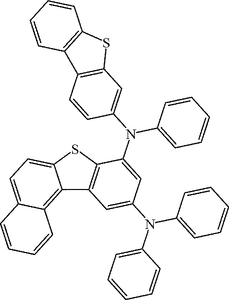 [SMILES: c1ccc(N(c2ccccc2)c2cc(N(c3ccccc3)c3ccc4c(c3)sc3ccccc34)c3sc4ccc5ccccc5c4c3c2)cc1]

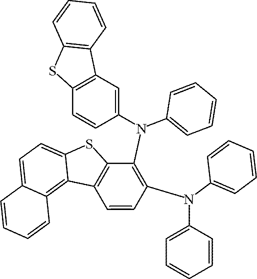 [SMILES: c1ccc(N(c2ccccc2)c2ccc3c(sc4ccc5ccccc5c43)c2N(c2ccccc2)c2ccc3sc4ccccc4c3c2)cc1]

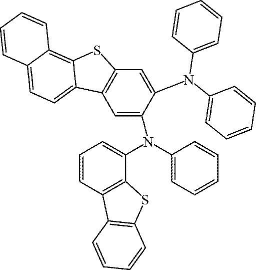 [SMILES: c1ccc(N(c2ccccc2)c2cc3sc4c5ccccc5ccc4c3cc2N(c2ccccc2)c2cccc3c2sc2ccccc23)cc1]

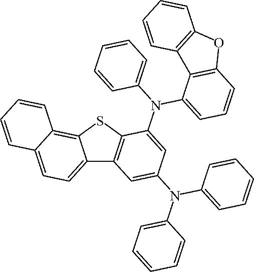 [SMILES: c1ccc(N(c2ccccc2)c2cc(N(c3ccccc3)c3cccc4oc5ccccc5c34)c3sc4c5ccccc5ccc4c3c2)cc1]

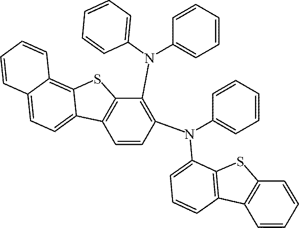 [SMILES: c1ccc(N(c2ccc3c(sc4c5ccccc5ccc34)c2N(c2ccccc2)c2ccccc2)c2cccc3c2sc2ccccc23)cc1]

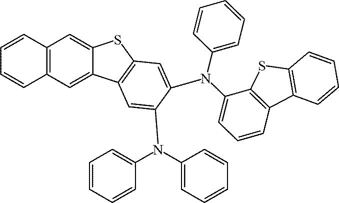 [SMILES: c1ccc(N(c2ccccc2)c2cc3c(cc2N(c2ccccc2)c2cccc4c2sc2ccccc24)sc2cc4ccccc4cc23)cc1]

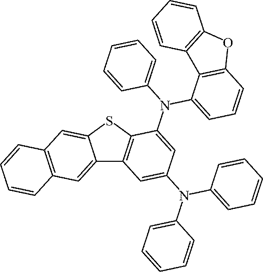 [SMILES: c1ccc(N(c2ccccc2)c2cc(N(c3ccccc3)c3cccc4oc5ccccc5c34)c3sc4cc5ccccc5cc4c3c2)cc1]

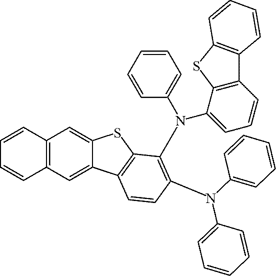 [SMILES: c1ccc(N(c2ccccc2)c2ccc3c(sc4cc5ccccc5cc43)c2N(c2ccccc2)c2cccc3c2sc2ccccc23)cc1]

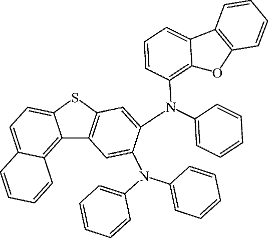 [SMILES: c1ccc(N(c2ccccc2)c2cc3c(cc2N(c2ccccc2)c2cccc4c2oc2ccccc24)sc2ccc4ccccc4c23)cc1]

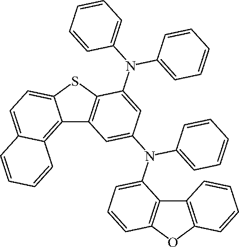 [SMILES: c1ccc(N(c2ccccc2)c2cc(N(c3ccccc3)c3cccc4oc5ccccc5c34)cc3c2sc2ccc4ccccc4c23)cc1]

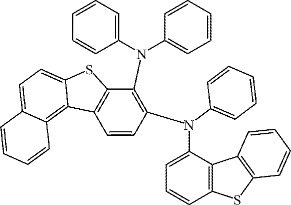 [SMILES: c1ccc(N(c2ccccc2)c2c(N(c3ccccc3)c3cccc4sc5ccccc5c34)ccc3c2sc2ccc4ccccc4c23)cc1]

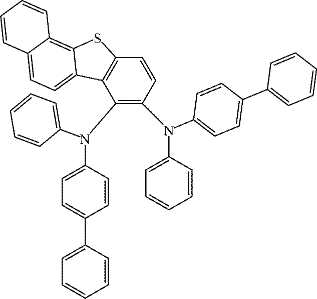 [SMILES: c1ccc(-c2ccc(N(c3ccccc3)c3ccc4sc5c6ccccc6ccc5c4c3N(c3ccccc3)c3ccc(-c4ccccc4)cc3)cc2)cc1]

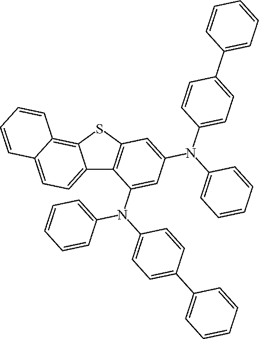 [SMILES: c1ccc(-c2ccc(N(c3ccccc3)c3cc(N(c4ccccc4)c4ccc(-c5ccccc5)cc4)c4c(c3)sc3c5ccccc5ccc34)cc2)cc1]

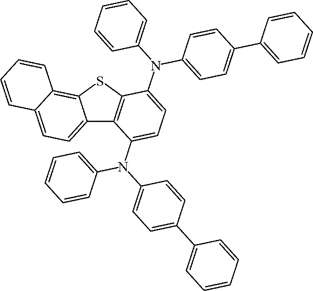 [SMILES: c1ccc(-c2ccc(N(c3ccccc3)c3ccc(N(c4ccccc4)c4ccc(-c5ccccc5)cc4)c4c3sc3c5ccccc5ccc34)cc2)cc1]

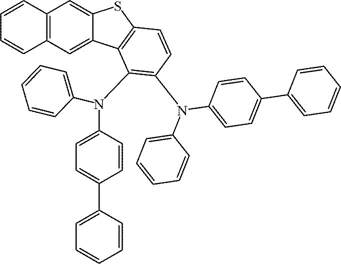 [SMILES: c1ccc(-c2ccc(N(c3ccccc3)c3ccc4sc5cc6ccccc6cc5c4c3N(c3ccccc3)c3ccc(-c4ccccc4)cc3)cc2)cc1]

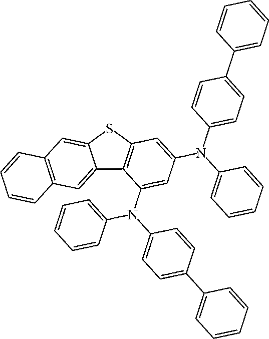 [SMILES: c1ccc(-c2ccc(N(c3ccccc3)c3cc(N(c4ccccc4)c4ccc(-c5ccccc5)cc4)c4c(c3)sc3cc5ccccc5cc34)cc2)cc1]

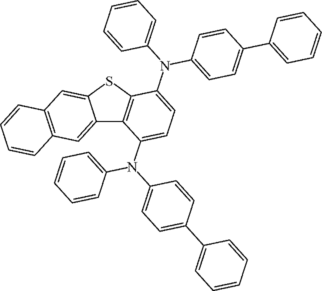 [SMILES: c1ccc(-c2ccc(N(c3ccccc3)c3ccc(N(c4ccccc4)c4ccc(-c5ccccc5)cc4)c4c3sc3cc5ccccc5cc34)cc2)cc1]

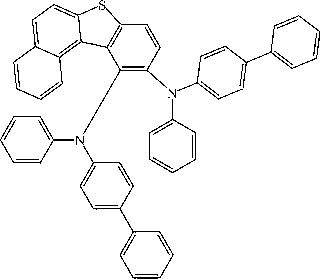 [SMILES: c1ccc(-c2ccc(N(c3ccccc3)c3ccc4sc5ccc6ccccc6c5c4c3N(c3ccccc3)c3ccc(-c4ccccc4)cc3)cc2)cc1]

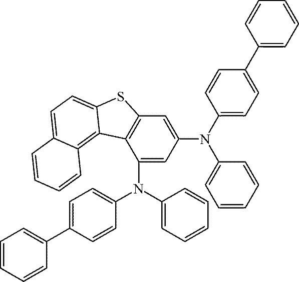 [SMILES: c1ccc(-c2ccc(N(c3ccccc3)c3cc(N(c4ccccc4)c4ccc(-c5ccccc5)cc4)c4c(c3)sc3ccc5ccccc5c34)cc2)cc1]

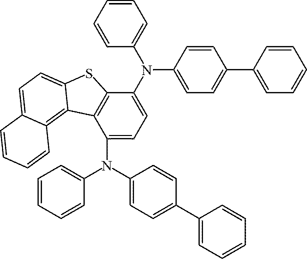 [SMILES: c1ccc(-c2ccc(N(c3ccccc3)c3ccc(N(c4ccccc4)c4ccc(-c5ccccc5)cc4)c4c3sc3ccc5ccccc5c34)cc2)cc1]

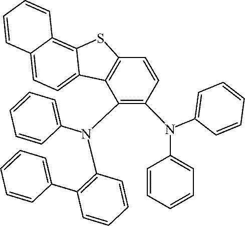 [SMILES: c1ccc(-c2ccccc2N(c2ccccc2)c2c(N(c3ccccc3)c3ccccc3)ccc3sc4c5ccccc5ccc4c23)cc1]

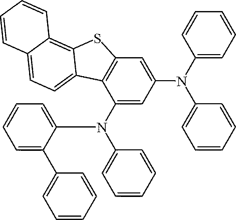 [SMILES: c1ccc(-c2ccccc2N(c2ccccc2)c2cc(N(c3ccccc3)c3ccccc3)cc3sc4c5ccccc5ccc4c23)cc1]

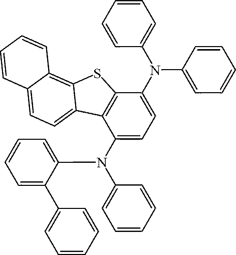 [SMILES: c1ccc(-c2ccccc2N(c2ccccc2)c2ccc(N(c3ccccc3)c3ccccc3)c3sc4c5ccccc5ccc4c23)cc1]

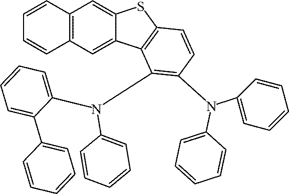 [SMILES: c1ccc(-c2ccccc2N(c2ccccc2)c2c(N(c3ccccc3)c3ccccc3)ccc3sc4cc5ccccc5cc4c23)cc1]

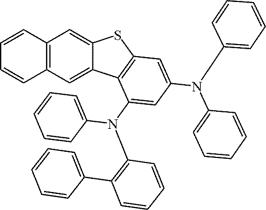 [SMILES: c1ccc(-c2ccccc2N(c2ccccc2)c2cc(N(c3ccccc3)c3ccccc3)cc3sc4cc5ccccc5cc4c23)cc1]

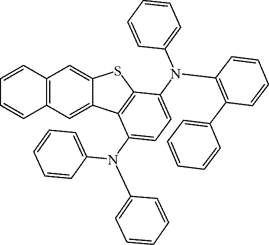 [SMILES: c1ccc(-c2ccccc2N(c2ccccc2)c2ccc(N(c3ccccc3)c3ccccc3)c3c2sc2cc4ccccc4cc23)cc1]

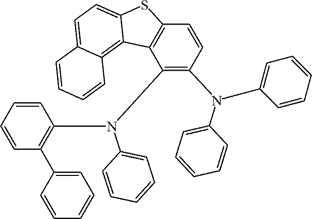

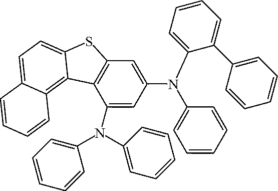 [SMILES: c1ccc(-c2ccccc2N(c2ccccc2)c2cc(N(c3ccccc3)c3ccccc3)c3c(c2)sc2ccc4ccccc4c23)cc1]

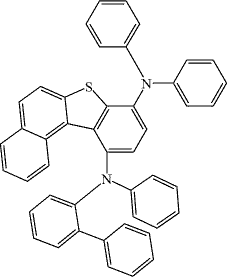 [SMILES: c1ccc(-c2ccccc2N(c2ccccc2)c2ccc(N(c3ccccc3)c3ccccc3)c3sc4ccc5ccccc5c4c23)cc1]

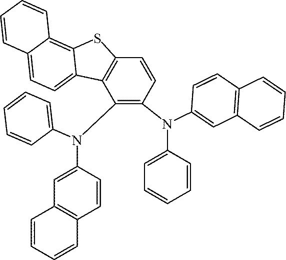 [SMILES: c1ccc(N(c2ccc3ccccc3c2)c2ccc3sc4c5ccccc5ccc4c3c2N(c2ccccc2)c2ccc3ccccc3c2)cc1]

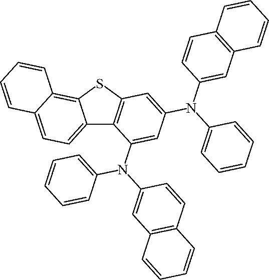 [SMILES: c1ccc(N(c2ccc3ccccc3c2)c2cc(N(c3ccccc3)c3ccc4ccccc4c3)c3c(c2)sc2c4ccccc4ccc23)cc1]

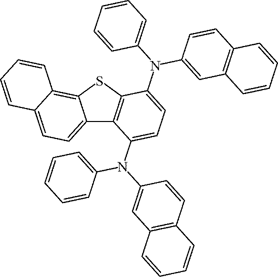 [SMILES: c1ccc(N(c2ccc3ccccc3c2)c2ccc(N(c3ccccc3)c3ccc4ccccc4c3)c3c2sc2c4ccccc4ccc23)cc1]

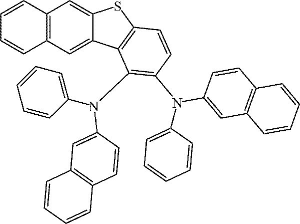 [SMILES: c1ccc(N(c2ccc3ccccc3c2)c2ccc3sc4cc5ccccc5cc4c3c2N(c2ccccc2)c2ccc3ccccc3c2)cc1]

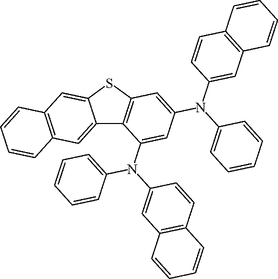 [SMILES: c1ccc(N(c2ccc3ccccc3c2)c2cc(N(c3ccccc3)c3ccc4ccccc4c3)c3c(c2)sc2cc4ccccc4cc23)cc1]

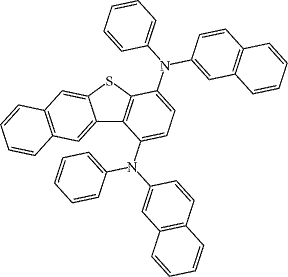 [SMILES: c1ccc(N(c2ccc3ccccc3c2)c2ccc(N(c3ccccc3)c3ccc4ccccc4c3)c3c2sc2cc4ccccc4cc23)cc1]

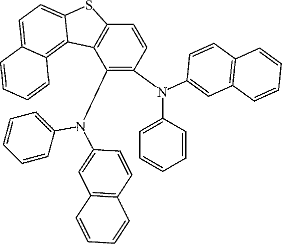 [SMILES: c1ccc(N(c2ccc3ccccc3c2)c2ccc3sc4ccc5ccccc5c4c3c2N(c2ccccc2)c2ccc3ccccc3c2)cc1]

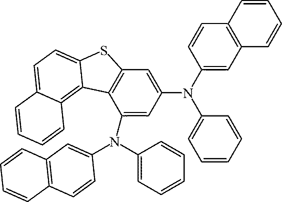 [SMILES: c1ccc(N(c2ccc3ccccc3c2)c2cc(N(c3ccccc3)c3ccc4ccccc4c3)c3c(c2)sc2ccc4ccccc4c23)cc1]

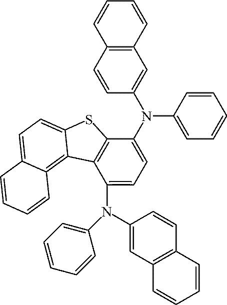 [SMILES: c1ccc(N(c2ccc3ccccc3c2)c2ccc(N(c3ccccc3)c3ccc4ccccc4c3)c3c2sc2ccc4ccccc4c23)cc1]

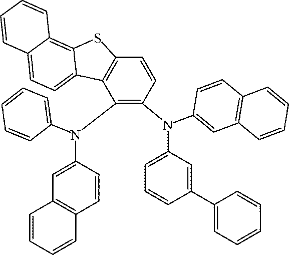 [SMILES: c1ccc(-c2cccc(N(c3ccc4ccccc4c3)c3ccc4sc5c6ccccc6ccc5c4c3N(c3ccccc3)c3ccc4ccccc4c3)c2)cc1]

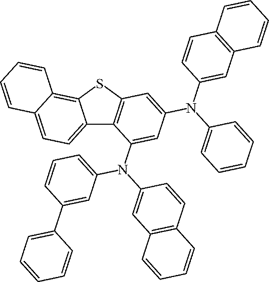 [SMILES: c1ccc(-c2cccc(N(c3ccc4ccccc4c3)c3cc(N(c4ccccc4)c4ccc5ccccc5c4)cc4sc5c6ccccc6ccc5c34)c2)cc1]

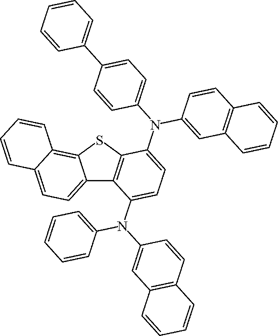 [SMILES: c1ccc(-c2ccc(N(c3ccc4ccccc4c3)c3ccc(N(c4ccccc4)c4ccc5ccccc5c4)c4c3sc3c5ccccc5ccc34)cc2)cc1]

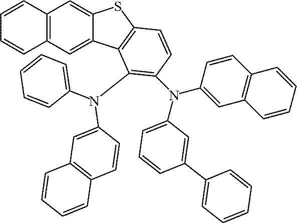 [SMILES: c1ccc(-c2cccc(N(c3ccc4ccccc4c3)c3ccc4sc5cc6ccccc6cc5c4c3N(c3ccccc3)c3ccc4ccccc4c3)c2)cc1]

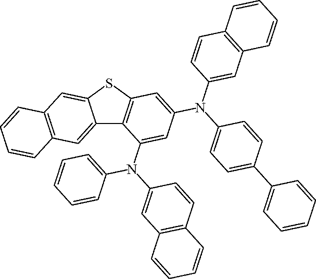 [SMILES: c1ccc(-c2ccc(N(c3ccc4ccccc4c3)c3cc(N(c4ccccc4)c4ccc5ccccc5c4)c4c(c3)sc3cc5ccccc5cc34)cc2)cc1]

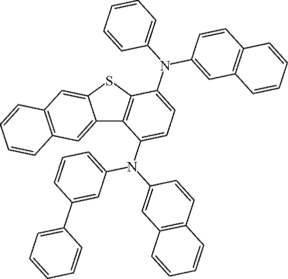 [SMILES: c1ccc(-c2cccc(N(c3ccc4ccccc4c3)c3ccc(N(c4ccccc4)c4ccc5ccccc5c4)c4sc5cc6ccccc6cc5c34)c2)cc1]

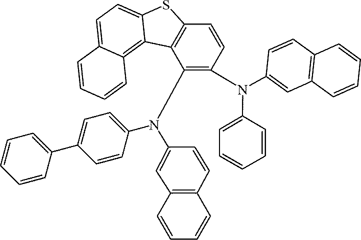 [SMILES: c1ccc(-c2ccc(N(c3ccc4ccccc4c3)c3c(N(c4ccccc4)c4ccc5ccccc5c4)ccc4sc5ccc6ccccc6c5c34)cc2)cc1]

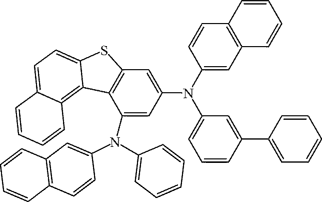 [SMILES: c1ccc(-c2cccc(N(c3ccc4ccccc4c3)c3cc(N(c4ccccc4)c4ccc5ccccc5c4)c4c(c3)sc3ccc5ccccc5c34)c2)cc1]

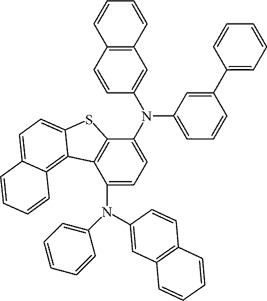 [SMILES: c1ccc(-c2cccc(N(c3ccc4ccccc4c3)c3ccc(N(c4ccccc4)c4ccc5ccccc5c4)c4c3sc3ccc5ccccc5c34)c2)cc1]

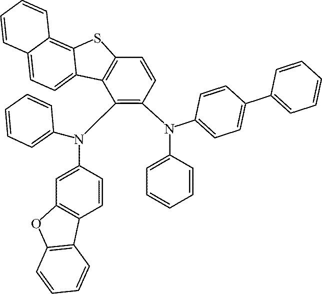 [SMILES: c1ccc(-c2ccc(N(c3ccccc3)c3ccc4sc5c6ccccc6ccc5c4c3N(c3ccccc3)c3ccc4c(c3)oc3ccccc34)cc2)cc1]

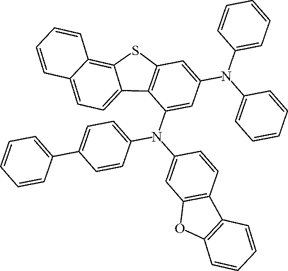 [SMILES: c1ccc(-c2ccc(N(c3ccc4c(c3)oc3ccccc34)c3cc(N(c4ccccc4)c4ccccc4)cc4sc5c6ccccc6ccc5c34)cc2)cc1]

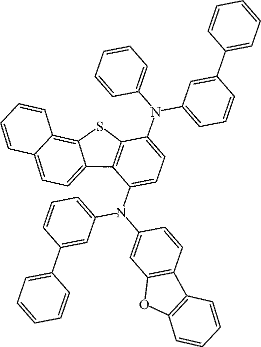

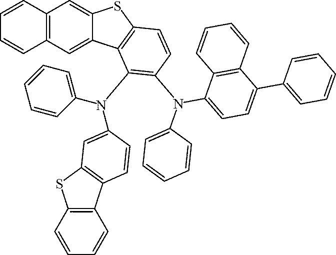 [SMILES: c1ccc(-c2ccc(N(c3ccccc3)c3ccc4sc5cc6ccccc6cc5c4c3N(c3ccccc3)c3ccc4c(c3)sc3ccccc34)c3ccccc23)cc1]

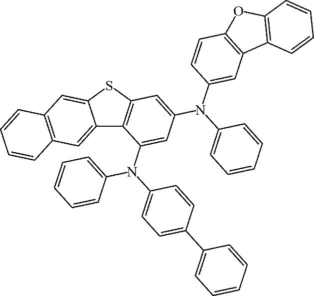 [SMILES: c1ccc(-c2ccc(N(c3ccccc3)c3cc(N(c4ccccc4)c4ccc5oc6ccccc6c5c4)cc4sc5cc6ccccc6cc5c34)cc2)cc1]

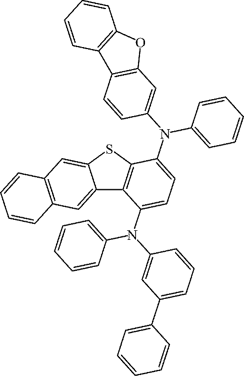 [SMILES: c1ccc(-c2cccc(N(c3ccccc3)c3ccc(N(c4ccccc4)c4ccc5c(c4)oc4ccccc45)c4sc5cc6ccccc6cc5c34)c2)cc1]

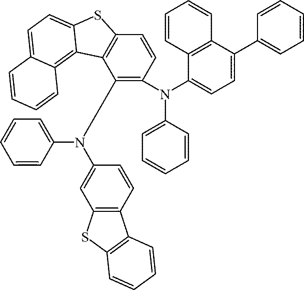 [SMILES: c1ccc(-c2ccc(N(c3ccccc3)c3ccc4sc5ccc6ccccc6c5c4c3N(c3ccccc3)c3ccc4c(c3)sc3ccccc34)c3ccccc23)cc1]

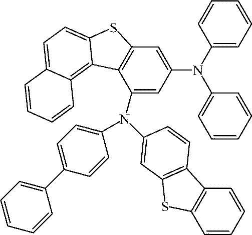 [SMILES: c1ccc(-c2ccc(N(c3ccc4c(c3)sc3ccccc34)c3cc(N(c4ccccc4)c4ccccc4)cc4sc5ccc6ccccc6c5c34)cc2)cc1]

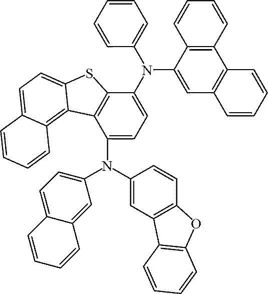 [SMILES: c1ccc(N(c2cc3ccccc3c3ccccc23)c2ccc(N(c3ccc4ccccc4c3)c3ccc4oc5ccccc5c4c3)c3c2sc2ccc4ccccc4c23)cc1]

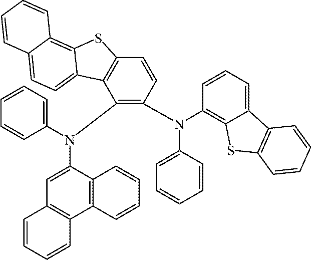 [SMILES: c1ccc(N(c2ccc3sc4c5ccccc5ccc4c3c2N(c2ccccc2)c2cc3ccccc3c3ccccc23)c2cccc3c2sc2ccccc23)cc1]

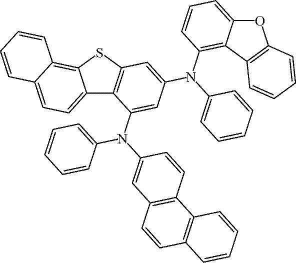 [SMILES: c1ccc(N(c2cc(N(c3ccccc3)c3ccc4c(ccc5ccccc54)c3)c3c(c2)sc2c4ccccc4ccc23)c2cccc3oc4ccccc4c23)cc1]

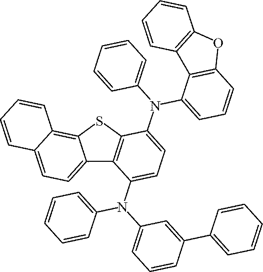 [SMILES: c1ccc(-c2cccc(N(c3ccccc3)c3ccc(N(c4ccccc4)c4cccc5oc6ccccc6c45)c4sc5c6ccccc6ccc5c34)c2)cc1]

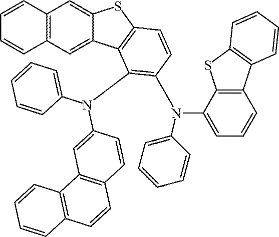 [SMILES: c1ccc(N(c2ccc3sc4cc5ccccc5cc4c3c2N(c2ccccc2)c2ccc3ccc4ccccc4c3c2)c2cccc3c2sc2ccccc23)cc1]

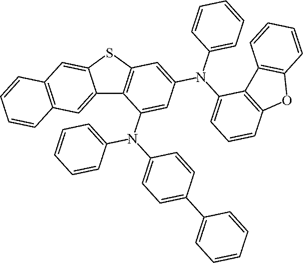 [SMILES: c1ccc(-c2ccc(N(c3ccccc3)c3cc(N(c4ccccc4)c4cccc5oc6ccccc6c45)cc4sc5cc6ccccc6cc5c34)cc2)cc1]

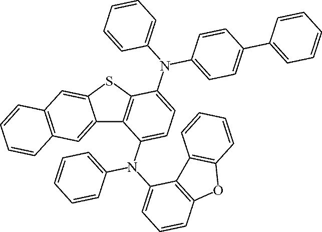 [SMILES: c1ccc(-c2ccc(N(c3ccccc3)c3ccc(N(c4ccccc4)c4cccc5oc6ccccc6c45)c4c3sc3cc5ccccc5cc34)cc2)cc1]

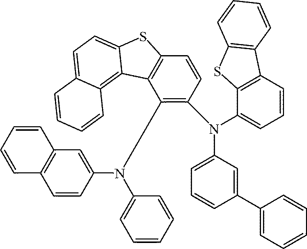 [SMILES: c1ccc(-c2cccc(N(c3ccc4sc5ccc6ccccc6c5c4c3N(c3ccccc3)c3ccc4ccccc4c3)c3cccc4c3sc3ccccc34)c2)cc1]

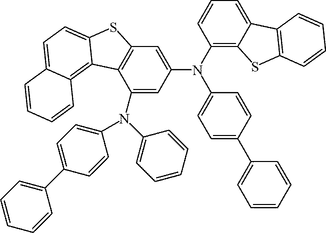 [SMILES: c1ccc(-c2ccc(N(c3cc(N(c4ccccc4)c4ccc(-c5ccccc5)cc4)c4c(c3)sc3ccc5ccccc5c34)c3cccc4c3sc3ccccc34)cc2)cc1]

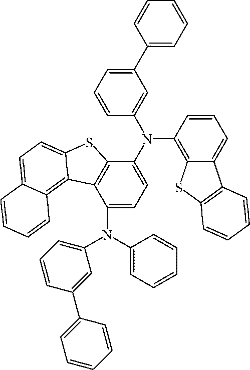 [SMILES: c1ccc(-c2cccc(N(c3cccc4c3sc3ccccc34)c3ccc(N(c4ccccc4)c4cccc(-c5ccccc5)c4)c4c3sc3ccc5ccccc5c34)c2)cc1]

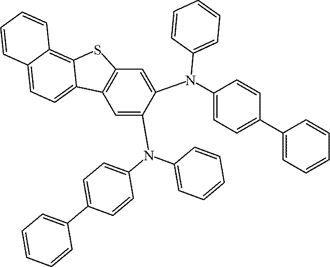 [SMILES: c1ccc(-c2ccc(N(c3ccccc3)c3cc4sc5c6ccccc6ccc5c4cc3N(c3ccccc3)c3ccc(-c4ccccc4)cc3)cc2)cc1]

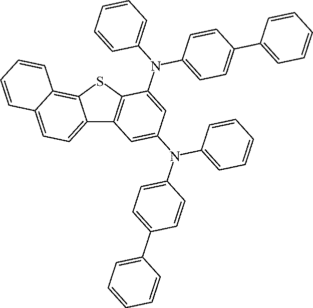 [SMILES: c1ccc(-c2ccc(N(c3ccccc3)c3cc(N(c4ccccc4)c4ccc(-c5ccccc5)cc4)c4sc5c6ccccc6ccc5c4c3)cc2)cc1]

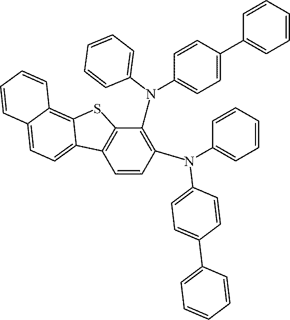 [SMILES: c1ccc(-c2ccc(N(c3ccccc3)c3ccc4c(sc5c6ccccc6ccc45)c3N(c3ccccc3)c3ccc(-c4ccccc4)cc3)cc2)cc1]

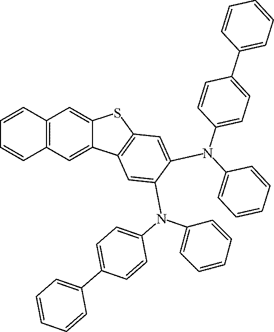 [SMILES: c1ccc(-c2ccc(N(c3ccccc3)c3cc4sc5cc6ccccc6cc5c4cc3N(c3ccccc3)c3ccc(-c4ccccc4)cc3)cc2)cc1]

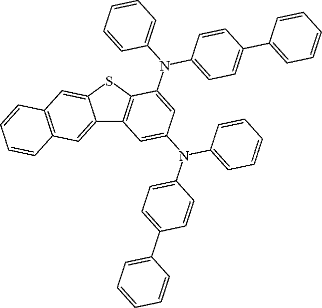 [SMILES: c1ccc(-c2ccc(N(c3ccccc3)c3cc(N(c4ccccc4)c4ccc(-c5ccccc5)cc4)c4sc5cc6ccccc6cc5c4c3)cc2)cc1]

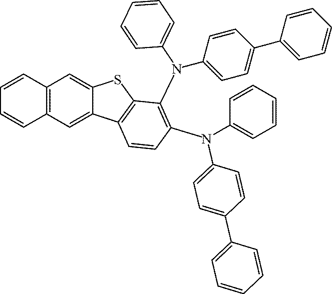 [SMILES: c1ccc(-c2ccc(N(c3ccccc3)c3ccc4c(sc5cc6ccccc6cc54)c3N(c3ccccc3)c3ccc(-c4ccccc4)cc3)cc2)cc1]

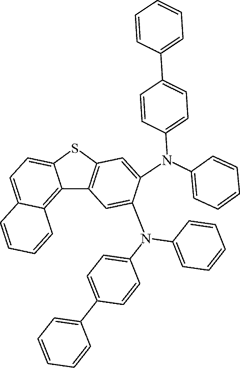 [SMILES: c1ccc(-c2ccc(N(c3ccccc3)c3cc4sc5ccc6ccccc6c5c4cc3N(c3ccccc3)c3ccc(-c4ccccc4)cc3)cc2)cc1]

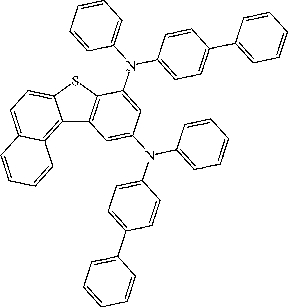 [SMILES: c1ccc(-c2ccc(N(c3ccccc3)c3cc(N(c4ccccc4)c4ccc(-c5ccccc5)cc4)c4sc5ccc6ccccc6c5c4c3)cc2)cc1]

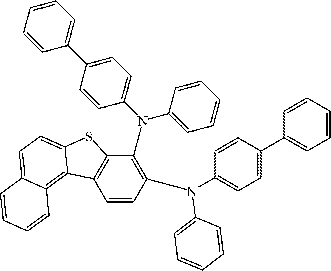 [SMILES: c1ccc(-c2ccc(N(c3ccccc3)c3ccc4c(sc5ccc6ccccc6c54)c3N(c3ccccc3)c3ccc(-c4ccccc4)cc3)cc2)cc1]

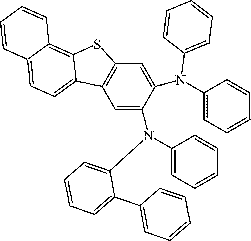 [SMILES: c1ccc(-c2ccccc2N(c2ccccc2)c2cc3c(cc2N(c2ccccc2)c2ccccc2)sc2c4ccccc4ccc32)cc1]

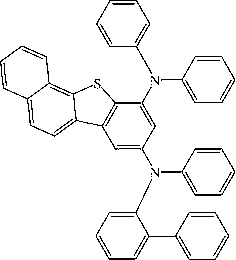 [SMILES: c1ccc(-c2ccccc2N(c2ccccc2)c2cc(N(c3ccccc3)c3ccccc3)c3sc4c5ccccc5ccc4c3c2)cc1]

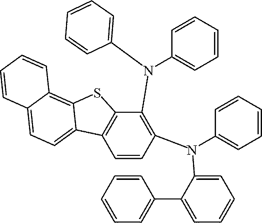 [SMILES: c1ccc(-c2ccccc2N(c2ccccc2)c2ccc3c(sc4c5ccccc5ccc34)c2N(c2ccccc2)c2ccccc2)cc1]

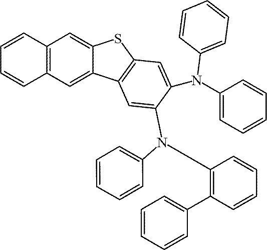 [SMILES: c1ccc(-c2ccccc2N(c2ccccc2)c2cc3c(cc2N(c2ccccc2)c2ccccc2)sc2cc4ccccc4cc23)cc1]

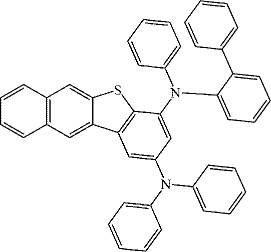 [SMILES: c1ccc(-c2ccccc2N(c2ccccc2)c2cc(N(c3ccccc3)c3ccccc3)cc3c2sc2cc4ccccc4cc23)cc1]

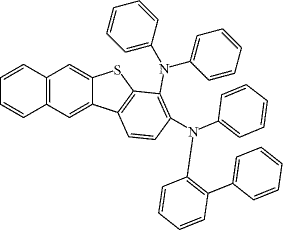 [SMILES: c1ccc(-c2ccccc2N(c2ccccc2)c2ccc3c(sc4cc5ccccc5cc43)c2N(c2ccccc2)c2ccccc2)cc1]

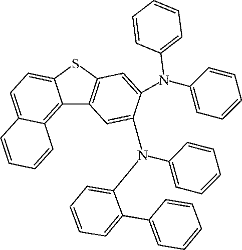 [SMILES: c1ccc(-c2ccccc2N(c2ccccc2)c2cc3c(cc2N(c2ccccc2)c2ccccc2)sc2ccc4ccccc4c23)cc1]

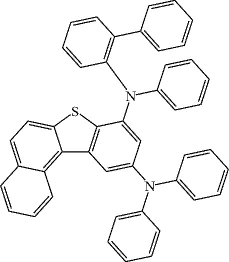 [SMILES: c1ccc(-c2ccccc2N(c2ccccc2)c2cc(N(c3ccccc3)c3ccccc3)cc3c2sc2ccc4ccccc4c23)cc1]

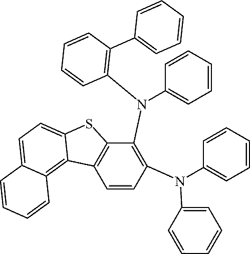 [SMILES: c1ccc(-c2ccccc2N(c2ccccc2)c2c(N(c3ccccc3)c3ccccc3)ccc3c2sc2ccc4ccccc4c23)cc1]

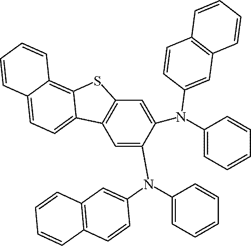 [SMILES: c1ccc(N(c2ccc3ccccc3c2)c2cc3sc4c5ccccc5ccc4c3cc2N(c2ccccc2)c2ccc3ccccc3c2)cc1]

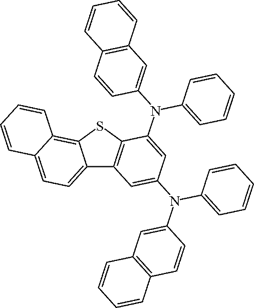 [SMILES: c1ccc(N(c2ccc3ccccc3c2)c2cc(N(c3ccccc3)c3ccc4ccccc4c3)c3sc4c5ccccc5ccc4c3c2)cc1]

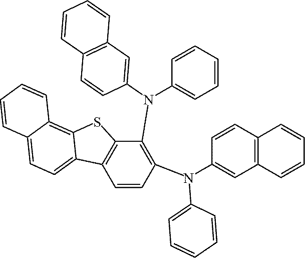 [SMILES: c1ccc(N(c2ccc3ccccc3c2)c2ccc3c(sc4c5ccccc5ccc34)c2N(c2ccccc2)c2ccc3ccccc3c2)cc1]

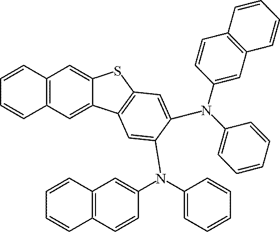 [SMILES: c1ccc(N(c2ccc3ccccc3c2)c2cc3sc4cc5ccccc5cc4c3cc2N(c2ccccc2)c2ccc3ccccc3c2)cc1]

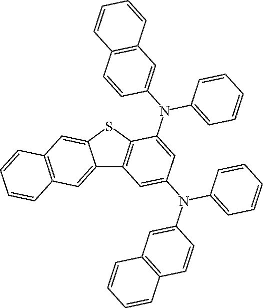 [SMILES: c1ccc(N(c2ccc3ccccc3c2)c2cc(N(c3ccccc3)c3ccc4ccccc4c3)c3sc4cc5ccccc5cc4c3c2)cc1]

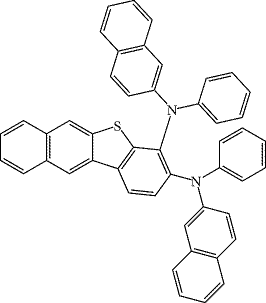 [SMILES: c1ccc(N(c2ccc3ccccc3c2)c2ccc3c(sc4cc5ccccc5cc43)c2N(c2ccccc2)c2ccc3ccccc3c2)cc1]

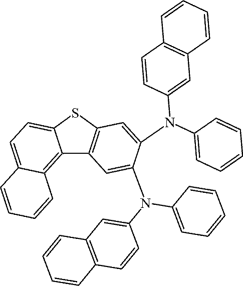 [SMILES: c1ccc(N(c2ccc3ccccc3c2)c2cc3sc4ccc5ccccc5c4c3cc2N(c2ccccc2)c2ccc3ccccc3c2)cc1]

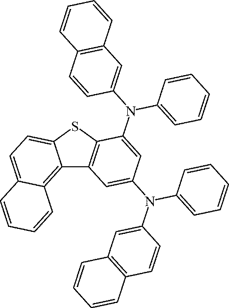 [SMILES: c1ccc(N(c2ccc3ccccc3c2)c2cc(N(c3ccccc3)c3ccc4ccccc4c3)c3sc4ccc5ccccc5c4c3c2)cc1]

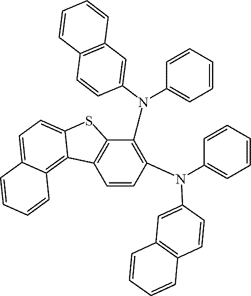 [SMILES: c1ccc(N(c2ccc3ccccc3c2)c2ccc3c(sc4ccc5ccccc5c43)c2N(c2ccccc2)c2ccc3ccccc3c2)cc1]

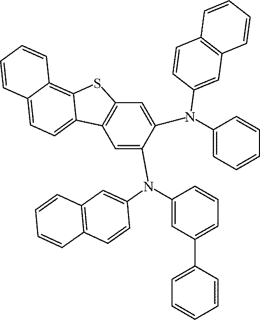 [SMILES: c1ccc(-c2cccc(N(c3ccc4ccccc4c3)c3cc4c(cc3N(c3ccccc3)c3ccc5ccccc5c3)sc3c5ccccc5ccc43)c2)cc1]

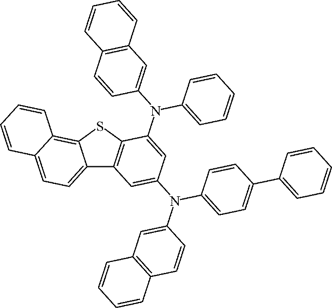 [SMILES: c1ccc(-c2ccc(N(c3ccc4ccccc4c3)c3cc(N(c4ccccc4)c4ccc5ccccc5c4)c4sc5c6ccccc6ccc5c4c3)cc2)cc1]

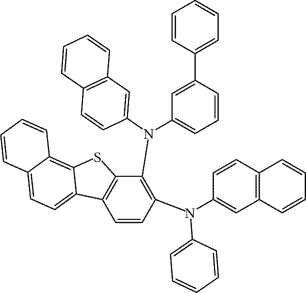 [SMILES: c1ccc(-c2cccc(N(c3ccc4ccccc4c3)c3c(N(c4ccccc4)c4ccc5ccccc5c4)ccc4c3sc3c5ccccc5ccc43)c2)cc1]

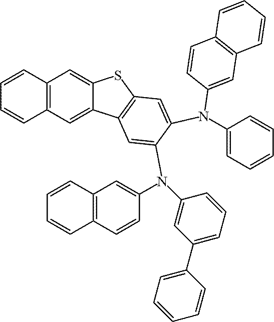 [SMILES: c1ccc(-c2cccc(N(c3ccc4ccccc4c3)c3cc4c(cc3N(c3ccccc3)c3ccc5ccccc5c3)sc3cc5ccccc5cc34)c2)cc1]

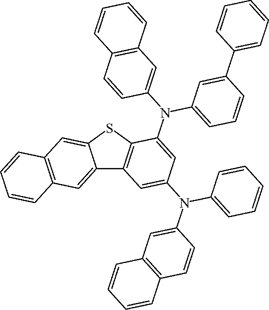 [SMILES: c1ccc(-c2cccc(N(c3ccc4ccccc4c3)c3cc(N(c4ccccc4)c4ccc5ccccc5c4)cc4c3sc3cc5ccccc5cc34)c2)cc1]

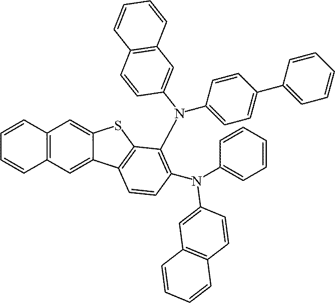 [SMILES: c1ccc(-c2ccc(N(c3ccc4ccccc4c3)c3c(N(c4ccccc4)c4ccc5ccccc5c4)ccc4c3sc3cc5ccccc5cc34)cc2)cc1]

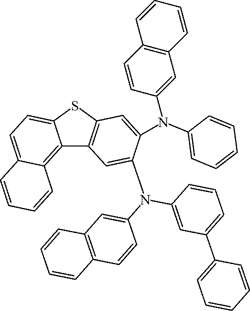 [SMILES: c1ccc(-c2cccc(N(c3ccc4ccccc4c3)c3cc4c(cc3N(c3ccccc3)c3ccc5ccccc5c3)sc3ccc5ccccc5c34)c2)cc1]

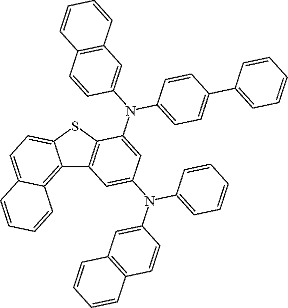 [SMILES: c1ccc(-c2ccc(N(c3ccc4ccccc4c3)c3cc(N(c4ccccc4)c4ccc5ccccc5c4)cc4c3sc3ccc5ccccc5c34)cc2)cc1]

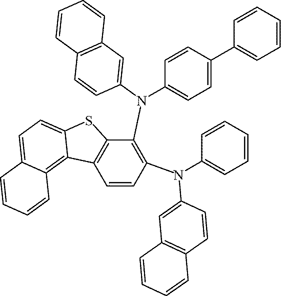 [SMILES: c1ccc(-c2ccc(N(c3ccc4ccccc4c3)c3c(N(c4ccccc4)c4ccc5ccccc5c4)ccc4c3sc3ccc5ccccc5c34)cc2)cc1]

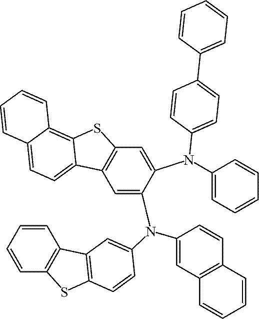 [SMILES: c1ccc(-c2ccc(N(c3ccccc3)c3cc4sc5c6ccccc6ccc5c4cc3N(c3ccc4ccccc4c3)c3ccc4sc5ccccc5c4c3)cc2)cc1]

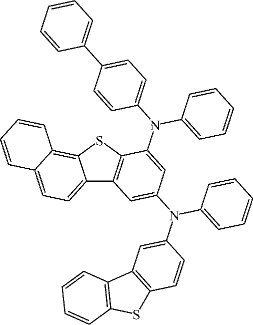 [SMILES: c1ccc(-c2ccc(N(c3ccccc3)c3cc(N(c4ccccc4)c4ccc5sc6ccccc6c5c4)cc4c3sc3c5ccccc5ccc43)cc2)cc1]

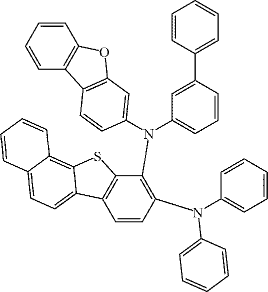 [SMILES: c1ccc(-c2cccc(N(c3ccc4c(c3)oc3ccccc34)c3c(N(c4ccccc4)c4ccccc4)ccc4c3sc3c5ccccc5ccc43)c2)cc1]

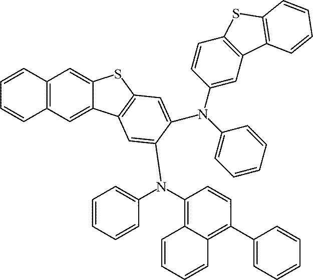 [SMILES: c1ccc(-c2ccc(N(c3ccccc3)c3cc4c(cc3N(c3ccccc3)c3ccc5sc6ccccc6c5c3)sc3cc5ccccc5cc34)c3ccccc23)cc1]

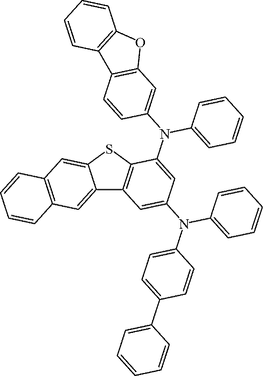 [SMILES: c1ccc(-c2ccc(N(c3ccccc3)c3cc(N(c4ccccc4)c4ccc5c(c4)oc4ccccc45)c4sc5cc6ccccc6cc5c4c3)cc2)cc1]

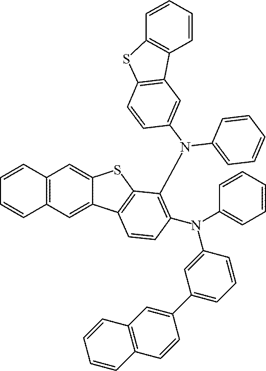 [SMILES: c1ccc(N(c2cccc(-c3ccc4ccccc4c3)c2)c2ccc3c(sc4cc5ccccc5cc43)c2N(c2ccccc2)c2ccc3sc4ccccc4c3c2)cc1]

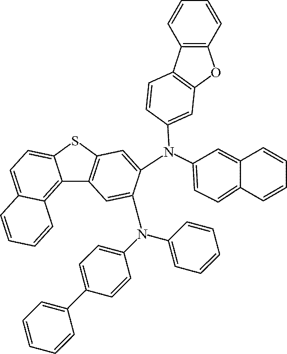 [SMILES: c1ccc(-c2ccc(N(c3ccccc3)c3cc4c(cc3N(c3ccc5ccccc5c3)c3ccc5c(c3)oc3ccccc35)sc3ccc5ccccc5c34)cc2)cc1]

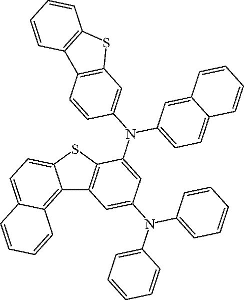 [SMILES: c1ccc(N(c2ccccc2)c2cc(N(c3ccc4ccccc4c3)c3ccc4c(c3)sc3ccccc34)c3sc4ccc5ccccc5c4c3c2)cc1]

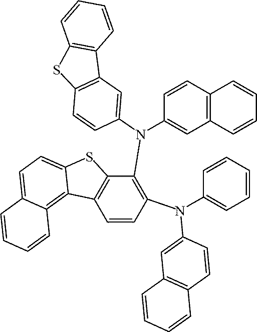 [SMILES: c1ccc(N(c2ccc3ccccc3c2)c2ccc3c(sc4ccc5ccccc5c43)c2N(c2ccc3ccccc3c2)c2ccc3sc4ccccc4c3c2)cc1]

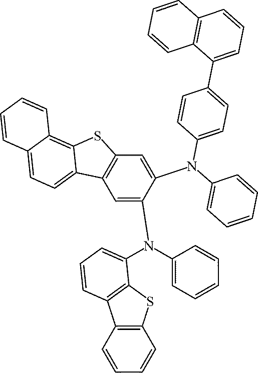 [SMILES: c1ccc(N(c2ccc(-c3cccc4ccccc34)cc2)c2cc3sc4c5ccccc5ccc4c3cc2N(c2ccccc2)c2cccc3c2sc2ccccc23)cc1]

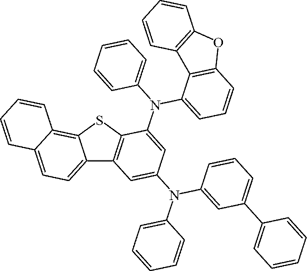 [SMILES: c1ccc(-c2cccc(N(c3ccccc3)c3cc(N(c4ccccc4)c4cccc5oc6ccccc6c45)c4sc5c6ccccc6ccc5c4c3)c2)cc1]

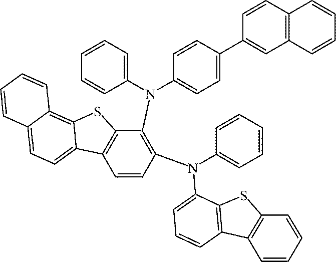 [SMILES: c1ccc(N(c2ccc3c(sc4c5ccccc5ccc34)c2N(c2ccccc2)c2ccc(-c3ccc4ccccc4c3)cc2)c2cccc3c2sc2ccccc23)cc1]

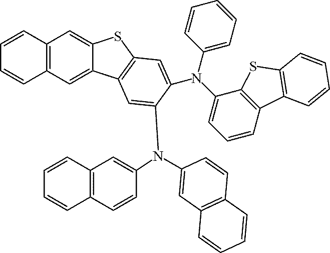 [SMILES: c1ccc(N(c2cc3sc4cc5ccccc5cc4c3cc2N(c2ccc3ccccc3c2)c2ccc3ccccc3c2)c2cccc3c2sc2ccccc23)cc1]

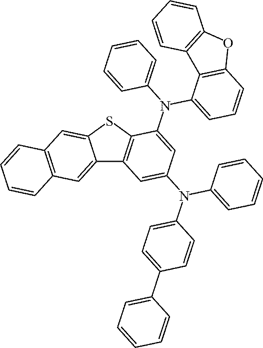 [SMILES: c1ccc(-c2ccc(N(c3ccccc3)c3cc(N(c4ccccc4)c4cccc5oc6ccccc6c45)c4sc5cc6ccccc6cc5c4c3)cc2)cc1]

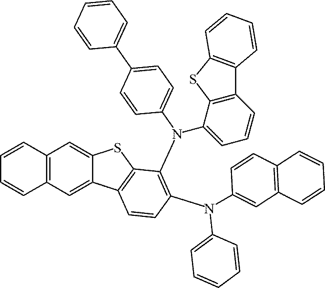 [SMILES: c1ccc(-c2ccc(N(c3cccc4c3sc3ccccc34)c3c(N(c4ccccc4)c4ccc5ccccc5c4)ccc4c3sc3cc5ccccc5cc34)cc2)cc1]

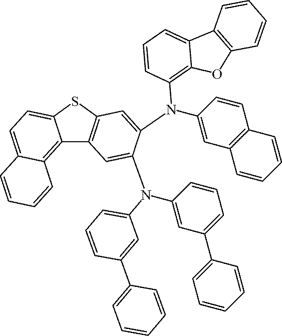 [SMILES: c1ccc(-c2cccc(N(c3cccc(-c4ccccc4)c3)c3cc4c(cc3N(c3ccc5ccccc5c3)c3cccc5c3oc3ccccc35)sc3ccc5ccccc5c34)c2)cc1]

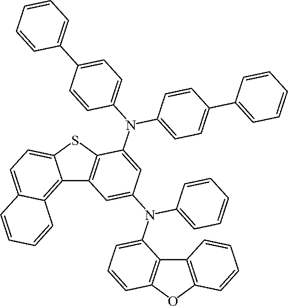 [SMILES: c1ccc(-c2ccc(N(c3ccc(-c4ccccc4)cc3)c3cc(N(c4ccccc4)c4cccc5oc6ccccc6c45)cc4c3sc3ccc5ccccc5c34)cc2)cc1]

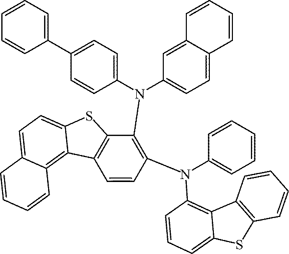 [SMILES: c1ccc(-c2ccc(N(c3ccc4ccccc4c3)c3c(N(c4ccccc4)c4cccc5sc6ccccc6c45)ccc4c3sc3ccc5ccccc5c34)cc2)cc1]

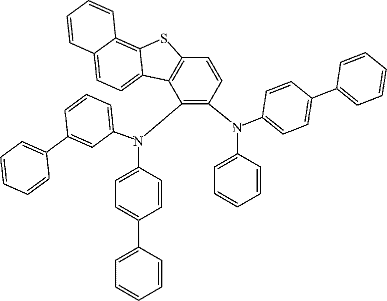 [SMILES: c1ccc(-c2ccc(N(c3ccccc3)c3ccc4sc5c6ccccc6ccc5c4c3N(c3ccc(-c4ccccc4)cc3)c3cccc(-c4ccccc4)c3)cc2)cc1]

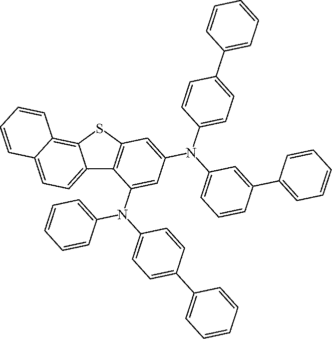 [SMILES: c1ccc(-c2ccc(N(c3cccc(-c4ccccc4)c3)c3cc(N(c4ccccc4)c4ccc(-c5ccccc5)cc4)c4c(c3)sc3c5ccccc5ccc34)cc2)cc1]

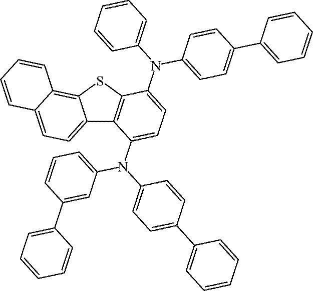 [SMILES: c1ccc(-c2ccc(N(c3ccccc3)c3ccc(N(c4ccc(-c5ccccc5)cc4)c4cccc(-c5ccccc5)c4)c4c3sc3c5ccccc5ccc34)cc2)cc1]

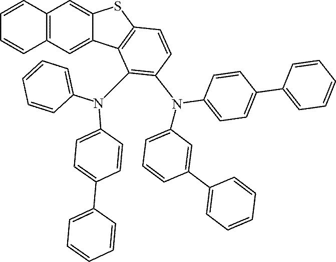 [SMILES: c1ccc(-c2ccc(N(c3cccc(-c4ccccc4)c3)c3ccc4sc5cc6ccccc6cc5c4c3N(c3ccccc3)c3ccc(-c4ccccc4)cc3)cc2)cc1]

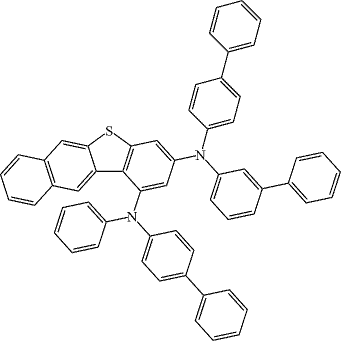 [SMILES: c1ccc(-c2ccc(N(c3cccc(-c4ccccc4)c3)c3cc(N(c4ccccc4)c4ccc(-c5ccccc5)cc4)c4c(c3)sc3cc5ccccc5cc34)cc2)cc1]

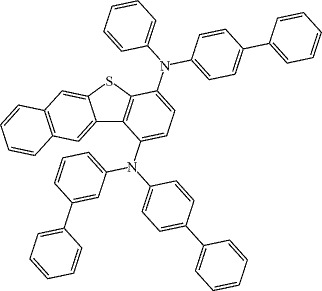 [SMILES: c1ccc(-c2ccc(N(c3ccccc3)c3ccc(N(c4ccc(-c5ccccc5)cc4)c4cccc(-c5ccccc5)c4)c4c3sc3cc5ccccc5cc34)cc2)cc1]

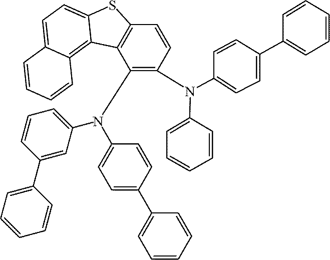 [SMILES: c1ccc(-c2ccc(N(c3ccccc3)c3ccc4sc5ccc6ccccc6c5c4c3N(c3ccc(-c4ccccc4)cc3)c3cccc(-c4ccccc4)c3)cc2)cc1]

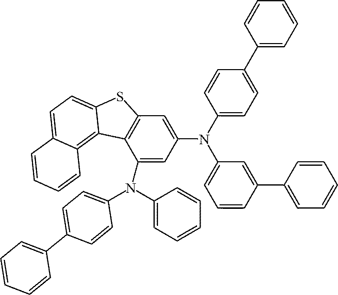 [SMILES: c1ccc(-c2ccc(N(c3cccc(-c4ccccc4)c3)c3cc(N(c4ccccc4)c4ccc(-c5ccccc5)cc4)c4c(c3)sc3ccc5ccccc5c34)cc2)cc1]

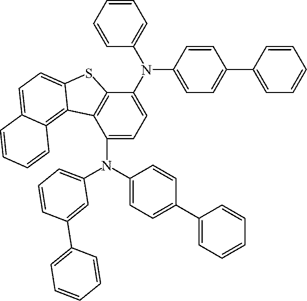 [SMILES: c1ccc(-c2ccc(N(c3ccccc3)c3ccc(N(c4ccc(-c5ccccc5)cc4)c4cccc(-c5ccccc5)c4)c4c3sc3ccc5ccccc5c34)cc2)cc1]

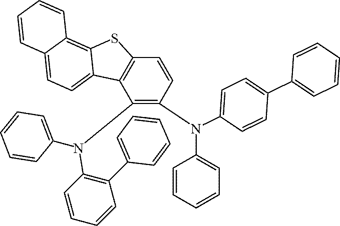 [SMILES: c1ccc(-c2ccc(N(c3ccccc3)c3ccc4sc5c6ccccc6ccc5c4c3N(c3ccccc3)c3ccccc3-c3ccccc3)cc2)cc1]

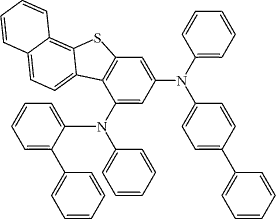 [SMILES: c1ccc(-c2ccc(N(c3ccccc3)c3cc(N(c4ccccc4)c4ccccc4-c4ccccc4)c4c(c3)sc3c5ccccc5ccc34)cc2)cc1]

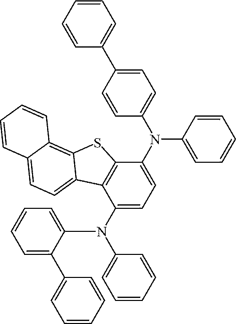 [SMILES: c1ccc(-c2ccc(N(c3ccccc3)c3ccc(N(c4ccccc4)c4ccccc4-c4ccccc4)c4c3sc3c5ccccc5ccc34)cc2)cc1]

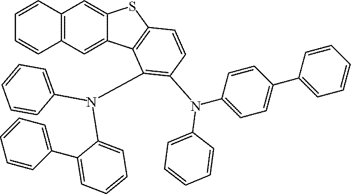 [SMILES: c1ccc(-c2ccc(N(c3ccccc3)c3ccc4sc5cc6ccccc6cc5c4c3N(c3ccccc3)c3ccccc3-c3ccccc3)cc2)cc1]

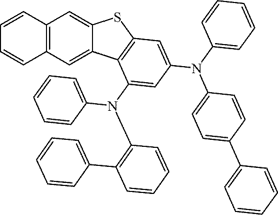 [SMILES: c1ccc(-c2ccc(N(c3ccccc3)c3cc(N(c4ccccc4)c4ccccc4-c4ccccc4)c4c(c3)sc3cc5ccccc5cc34)cc2)cc1]

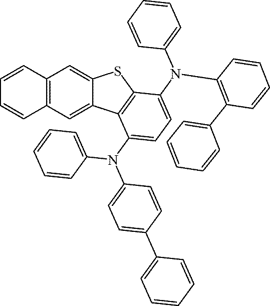 [SMILES: c1ccc(-c2ccc(N(c3ccccc3)c3ccc(N(c4ccccc4)c4ccccc4-c4ccccc4)c4sc5cc6ccccc6cc5c34)cc2)cc1]

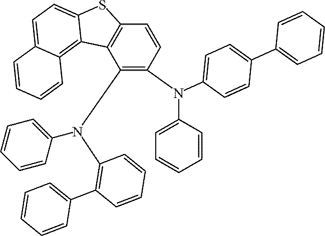 [SMILES: c1ccc(-c2ccc(N(c3ccccc3)c3ccc4sc5ccc6ccccc6c5c4c3N(c3ccccc3)c3ccccc3-c3ccccc3)cc2)cc1]

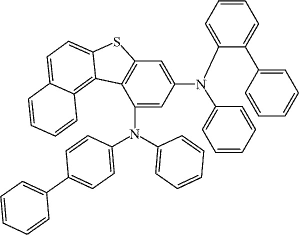 [SMILES: c1ccc(-c2ccc(N(c3ccccc3)c3cc(N(c4ccccc4)c4ccccc4-c4ccccc4)cc4sc5ccc6ccccc6c5c34)cc2)cc1]

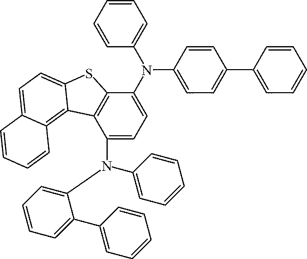 [SMILES: c1ccc(-c2ccc(N(c3ccccc3)c3ccc(N(c4ccccc4)c4ccccc4-c4ccccc4)c4c3sc3ccc5ccccc5c34)cc2)cc1]

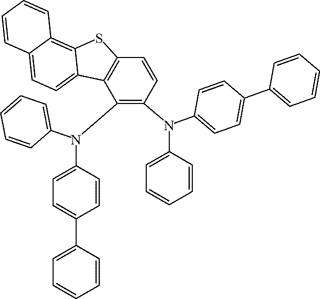 [SMILES: c1ccc(-c2ccc(N(c3ccccc3)c3ccc4sc5c6ccccc6ccc5c4c3N(c3ccccc3)c3ccc(-c4ccccc4)cc3)cc2)cc1]

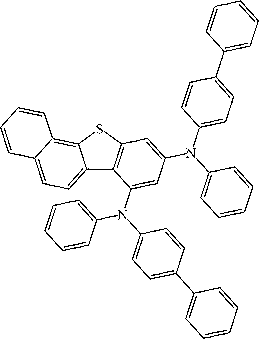 [SMILES: c1ccc(-c2ccc(N(c3ccccc3)c3cc(N(c4ccccc4)c4ccc(-c5ccccc5)cc4)c4c(c3)sc3c5ccccc5ccc34)cc2)cc1]

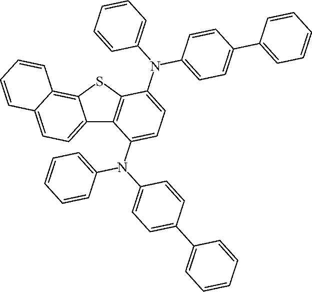 [SMILES: c1ccc(-c2ccc(N(c3ccccc3)c3ccc(N(c4ccccc4)c4ccc(-c5ccccc5)cc4)c4c3sc3c5ccccc5ccc34)cc2)cc1]

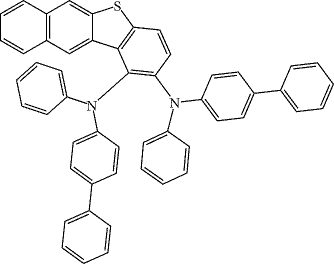 [SMILES: c1ccc(-c2ccc(N(c3ccccc3)c3ccc4sc5cc6ccccc6cc5c4c3N(c3ccccc3)c3ccc(-c4ccccc4)cc3)cc2)cc1]

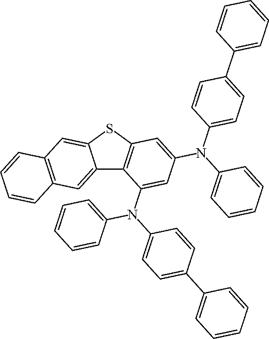 [SMILES: c1ccc(-c2ccc(N(c3ccccc3)c3cc(N(c4ccccc4)c4ccc(-c5ccccc5)cc4)c4c(c3)sc3cc5ccccc5cc34)cc2)cc1]

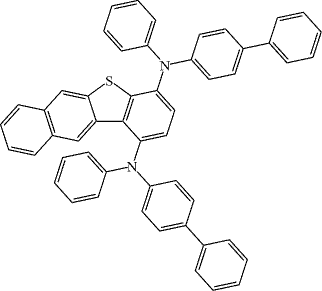 [SMILES: c1ccc(-c2ccc(N(c3ccccc3)c3ccc(N(c4ccccc4)c4ccc(-c5ccccc5)cc4)c4c3sc3cc5ccccc5cc34)cc2)cc1]

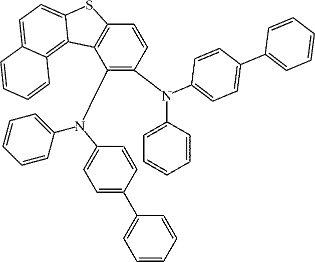 [SMILES: c1ccc(-c2ccc(N(c3ccccc3)c3ccc4sc5ccc6ccccc6c5c4c3N(c3ccccc3)c3ccc(-c4ccccc4)cc3)cc2)cc1]

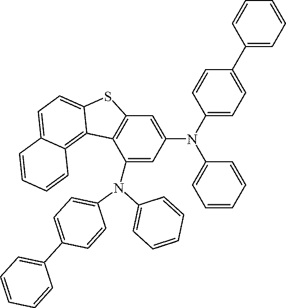 [SMILES: c1ccc(-c2ccc(N(c3ccccc3)c3cc(N(c4ccccc4)c4ccc(-c5ccccc5)cc4)c4c(c3)sc3ccc5ccccc5c34)cc2)cc1]

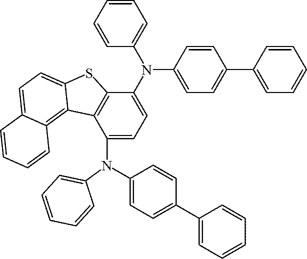 [SMILES: c1ccc(-c2ccc(N(c3ccccc3)c3ccc(N(c4ccccc4)c4ccc(-c5ccccc5)cc4)c4c3sc3ccc5ccccc5c34)cc2)cc1]

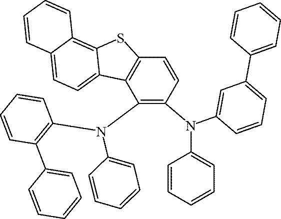 [SMILES: c1ccc(-c2cccc(N(c3ccccc3)c3ccc4sc5c6ccccc6ccc5c4c3N(c3ccccc3)c3ccccc3-c3ccccc3)c2)cc1]

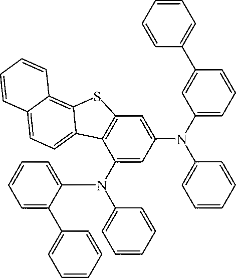 [SMILES: c1ccc(-c2cccc(N(c3ccccc3)c3cc(N(c4ccccc4)c4ccccc4-c4ccccc4)c4c(c3)sc3c5ccccc5ccc34)c2)cc1]

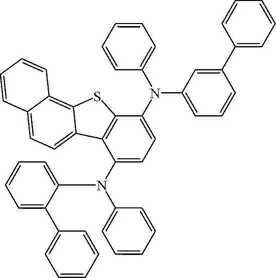 [SMILES: c1ccc(-c2cccc(N(c3ccccc3)c3ccc(N(c4ccccc4)c4ccccc4-c4ccccc4)c4c3sc3c5ccccc5ccc34)c2)cc1]

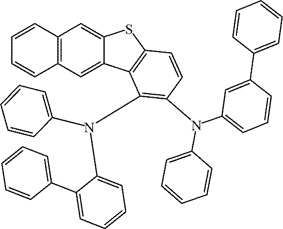 [SMILES: c1ccc(-c2cccc(N(c3ccccc3)c3ccc4sc5cc6ccccc6cc5c4c3N(c3ccccc3)c3ccccc3-c3ccccc3)c2)cc1]

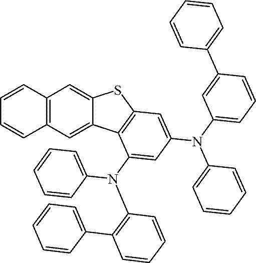 [SMILES: c1ccc(-c2cccc(N(c3ccccc3)c3cc(N(c4ccccc4)c4ccccc4-c4ccccc4)c4c(c3)sc3cc5ccccc5cc34)c2)cc1]

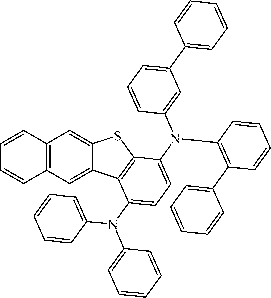 [SMILES: c1ccc(-c2cccc(N(c3ccccc3-c3ccccc3)c3ccc(N(c4ccccc4)c4ccccc4)c4c3sc3cc5ccccc5cc34)c2)cc1]

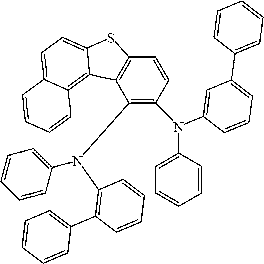 [SMILES: c1ccc(-c2cccc(N(c3ccccc3)c3ccc4sc5ccc6ccccc6c5c4c3N(c3ccccc3)c3ccccc3-c3ccccc3)c2)cc1]

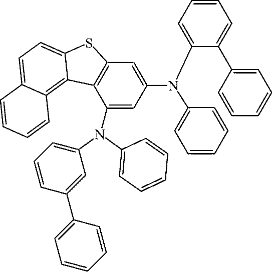 [SMILES: c1ccc(-c2cccc(N(c3ccccc3)c3cc(N(c4ccccc4)c4ccccc4-c4ccccc4)cc4sc5ccc6ccccc6c5c34)c2)cc1]

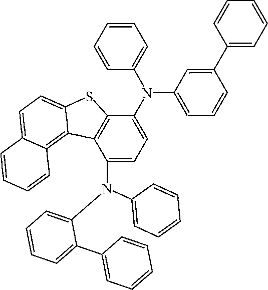 [SMILES: c1ccc(-c2cccc(N(c3ccccc3)c3ccc(N(c4ccccc4)c4ccccc4-c4ccccc4)c4c3sc3ccc5ccccc5c34)c2)cc1]

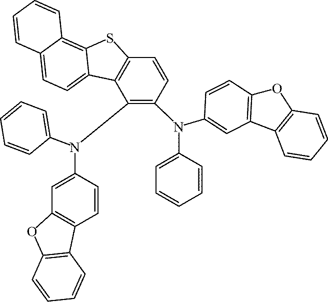 [SMILES: c1ccc(N(c2ccc3oc4ccccc4c3c2)c2ccc3sc4c5ccccc5ccc4c3c2N(c2ccccc2)c2ccc3c(c2)oc2ccccc23)cc1]

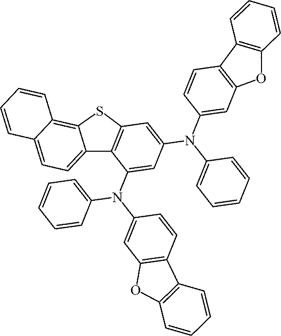 [SMILES: c1ccc(N(c2ccc3c(c2)oc2ccccc23)c2cc(N(c3ccccc3)c3ccc4c(c3)oc3ccccc34)c3c(c2)sc2c4ccccc4ccc23)cc1]

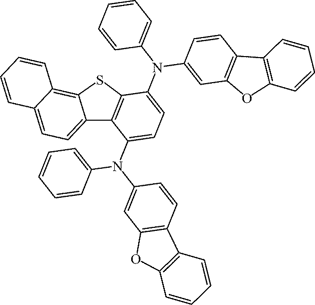 [SMILES: c1ccc(N(c2ccc3c(c2)oc2ccccc23)c2ccc(N(c3ccccc3)c3ccc4c(c3)oc3ccccc34)c3c2sc2c4ccccc4ccc23)cc1]

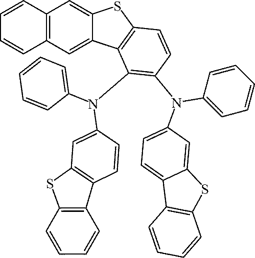 [SMILES: c1ccc(N(c2ccc3c(c2)sc2ccccc23)c2ccc3sc4cc5ccccc5cc4c3c2N(c2ccccc2)c2ccc3c(c2)sc2ccccc23)cc1]

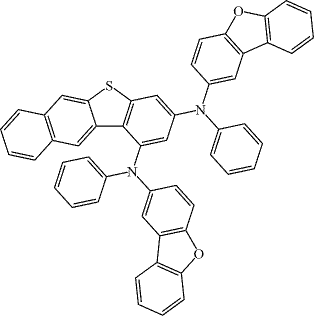 [SMILES: c1ccc(N(c2cc(N(c3ccccc3)c3ccc4oc5ccccc5c4c3)c3c(c2)sc2cc4ccccc4cc23)c2ccc3oc4ccccc4c3c2)cc1]

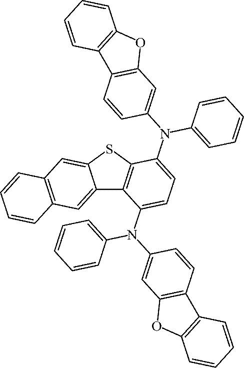 [SMILES: c1ccc(N(c2ccc3c(c2)oc2ccccc23)c2ccc(N(c3ccccc3)c3ccc4c(c3)oc3ccccc34)c3c2sc2cc4ccccc4cc23)cc1]

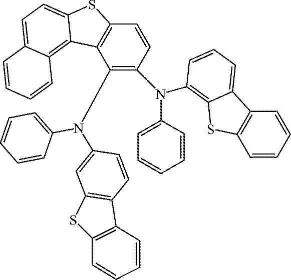 [SMILES: c1ccc(N(c2ccc3sc4ccc5ccccc5c4c3c2N(c2ccccc2)c2ccc3c(c2)sc2ccccc23)c2cccc3c2sc2ccccc23)cc1]

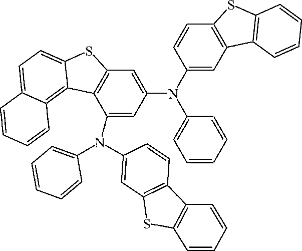 [SMILES: c1ccc(N(c2cc(N(c3ccccc3)c3ccc4c(c3)sc3ccccc34)c3c(c2)sc2ccc4ccccc4c23)c2ccc3sc4ccccc4c3c2)cc1]

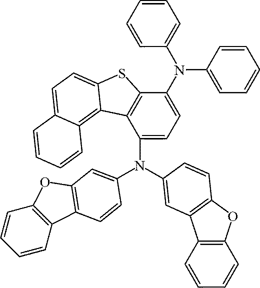 [SMILES: c1ccc(N(c2ccccc2)c2ccc(N(c3ccc4c(c3)oc3ccccc34)c3ccc4oc5ccccc5c4c3)c3c2sc2ccc4ccccc4c23)cc1]

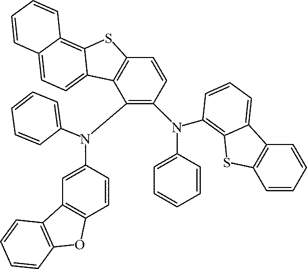 [SMILES: c1ccc(N(c2ccc3sc4c5ccccc5ccc4c3c2N(c2ccccc2)c2ccc3oc4ccccc4c3c2)c2cccc3c2sc2ccccc23)cc1]

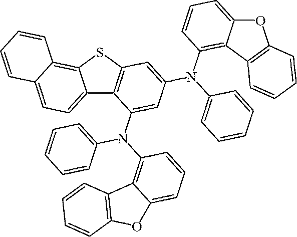 [SMILES: c1ccc(N(c2cc(N(c3ccccc3)c3cccc4oc5ccccc5c34)c3c(c2)sc2c4ccccc4ccc23)c2cccc3oc4ccccc4c23)cc1]

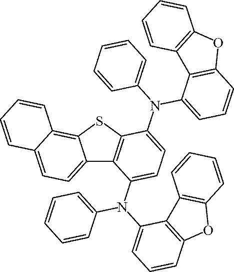 [SMILES: c1ccc(N(c2ccc(N(c3ccccc3)c3cccc4oc5ccccc5c34)c3c2sc2c4ccccc4ccc23)c2cccc3oc4ccccc4c23)cc1]

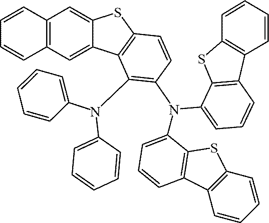 [SMILES: c1ccc(N(c2ccccc2)c2c(N(c3cccc4c3sc3ccccc34)c3cccc4c3sc3ccccc34)ccc3sc4cc5ccccc5cc4c23)cc1]

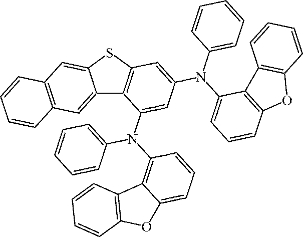 [SMILES: c1ccc(N(c2cc(N(c3ccccc3)c3cccc4oc5ccccc5c34)c3c(c2)sc2cc4ccccc4cc23)c2cccc3oc4ccccc4c23)cc1]

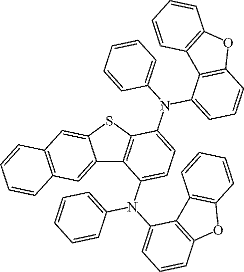 [SMILES: c1ccc(N(c2ccc(N(c3ccccc3)c3cccc4oc5ccccc5c34)c3c2sc2cc4ccccc4cc23)c2cccc3oc4ccccc4c23)cc1]

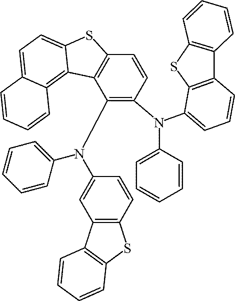 [SMILES: c1ccc(N(c2ccc3sc4ccc5ccccc5c4c3c2N(c2ccccc2)c2ccc3sc4ccccc4c3c2)c2cccc3c2sc2ccccc23)cc1]

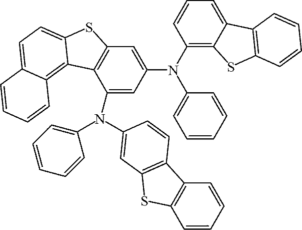 [SMILES: c1ccc(N(c2cc(N(c3ccccc3)c3ccc4c(c3)sc3ccccc34)c3c(c2)sc2ccc4ccccc4c23)c2cccc3c2sc2ccccc23)cc1]

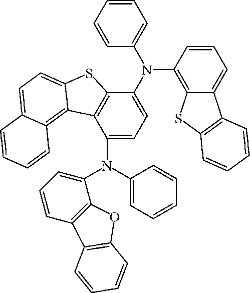 [SMILES: c1ccc(N(c2cccc3c2sc2ccccc23)c2ccc(N(c3ccccc3)c3cccc4c3oc3ccccc34)c3c2sc2ccc4ccccc4c23)cc1]

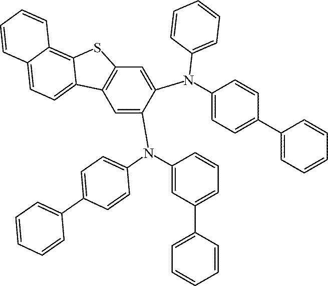 [SMILES: c1ccc(-c2ccc(N(c3ccccc3)c3cc4sc5c6ccccc6ccc5c4cc3N(c3ccc(-c4ccccc4)cc3)c3cccc(-c4ccccc4)c3)cc2)cc1]

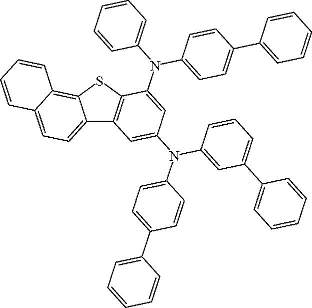 [SMILES: c1ccc(-c2ccc(N(c3cccc(-c4ccccc4)c3)c3cc(N(c4ccccc4)c4ccc(-c5ccccc5)cc4)c4sc5c6ccccc6ccc5c4c3)cc2)cc1]

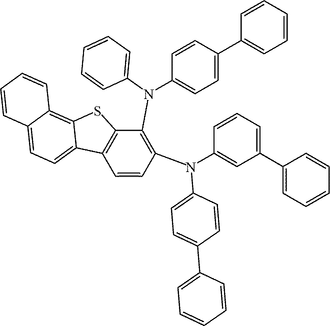 [SMILES: c1ccc(-c2ccc(N(c3cccc(-c4ccccc4)c3)c3ccc4c(sc5c6ccccc6ccc45)c3N(c3ccccc3)c3ccc(-c4ccccc4)cc3)cc2)cc1]

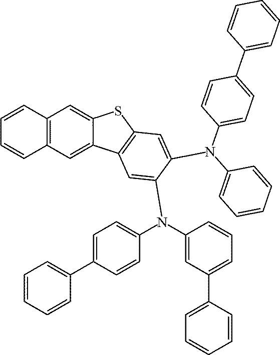 [SMILES: c1ccc(-c2ccc(N(c3ccccc3)c3cc4sc5cc6ccccc6cc5c4cc3N(c3ccc(-c4ccccc4)cc3)c3cccc(-c4ccccc4)c3)cc2)cc1]

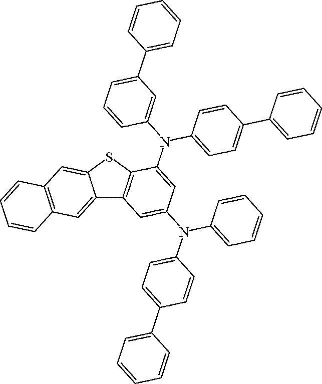 [SMILES: c1ccc(-c2ccc(N(c3ccccc3)c3cc(N(c4ccc(-c5ccccc5)cc4)c4cccc(-c5ccccc5)c4)c4sc5cc6ccccc6cc5c4c3)cc2)cc1]

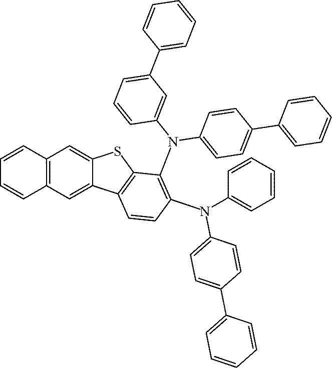 [SMILES: c1ccc(-c2ccc(N(c3ccccc3)c3ccc4c(sc5cc6ccccc6cc54)c3N(c3ccc(-c4ccccc4)cc3)c3cccc(-c4ccccc4)c3)cc2)cc1]

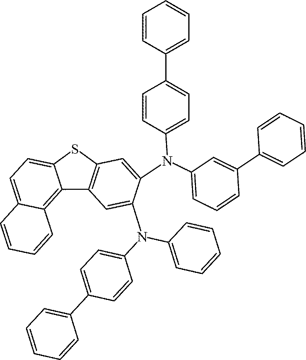 [SMILES: c1ccc(-c2ccc(N(c3cccc(-c4ccccc4)c3)c3cc4sc5ccc6ccccc6c5c4cc3N(c3ccccc3)c3ccc(-c4ccccc4)cc3)cc2)cc1]

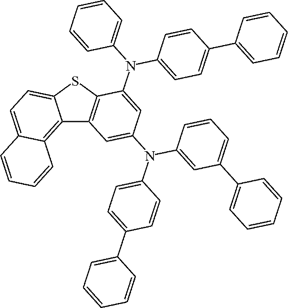 [SMILES: c1ccc(-c2ccc(N(c3cccc(-c4ccccc4)c3)c3cc(N(c4ccccc4)c4ccc(-c5ccccc5)cc4)c4sc5ccc6ccccc6c5c4c3)cc2)cc1]

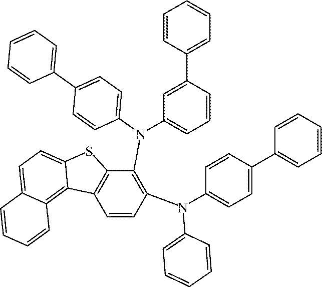 [SMILES: c1ccc(-c2ccc(N(c3ccccc3)c3ccc4c(sc5ccc6ccccc6c54)c3N(c3ccc(-c4ccccc4)cc3)c3cccc(-c4ccccc4)c3)cc2)cc1]

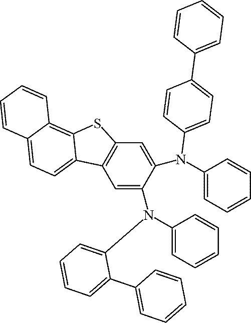 [SMILES: c1ccc(-c2ccc(N(c3ccccc3)c3cc4sc5c6ccccc6ccc5c4cc3N(c3ccccc3)c3ccccc3-c3ccccc3)cc2)cc1]

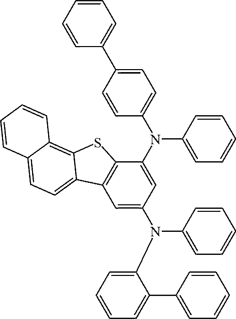 [SMILES: c1ccc(-c2ccc(N(c3ccccc3)c3cc(N(c4ccccc4)c4ccccc4-c4ccccc4)cc4c3sc3c5ccccc5ccc43)cc2)cc1]

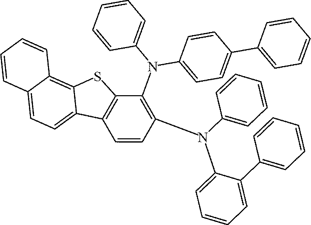 [SMILES: c1ccc(-c2ccc(N(c3ccccc3)c3c(N(c4ccccc4)c4ccccc4-c4ccccc4)ccc4c3sc3c5ccccc5ccc43)cc2)cc1]

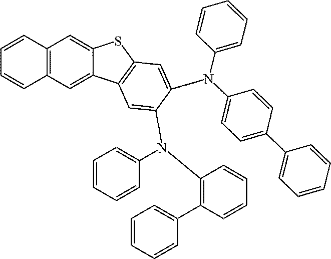 [SMILES: c1ccc(-c2ccc(N(c3ccccc3)c3cc4sc5cc6ccccc6cc5c4cc3N(c3ccccc3)c3ccccc3-c3ccccc3)cc2)cc1]

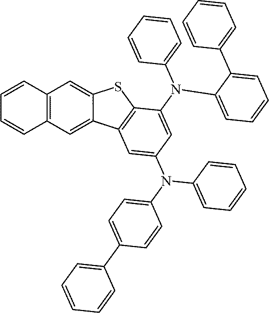 [SMILES: c1ccc(-c2ccc(N(c3ccccc3)c3cc(N(c4ccccc4)c4ccccc4-c4ccccc4)c4sc5cc6ccccc6cc5c4c3)cc2)cc1]

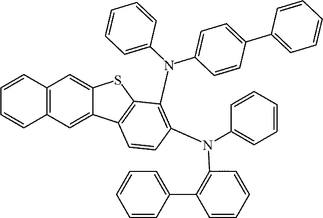 [SMILES: c1ccc(-c2ccc(N(c3ccccc3)c3c(N(c4ccccc4)c4ccccc4-c4ccccc4)ccc4c3sc3cc5ccccc5cc34)cc2)cc1]

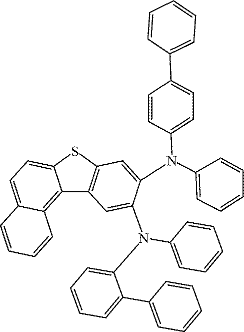 [SMILES: c1ccc(-c2ccc(N(c3ccccc3)c3cc4sc5ccc6ccccc6c5c4cc3N(c3ccccc3)c3ccccc3-c3ccccc3)cc2)cc1]

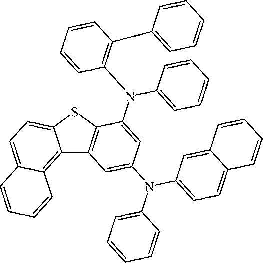 [SMILES: c1ccc(-c2ccccc2N(c2ccccc2)c2cc(N(c3ccccc3)c3ccc4ccccc4c3)cc3c2sc2ccc4ccccc4c23)cc1]

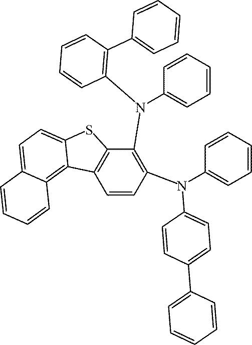 [SMILES: c1ccc(-c2ccc(N(c3ccccc3)c3ccc4c(sc5ccc6ccccc6c54)c3N(c3ccccc3)c3ccccc3-c3ccccc3)cc2)cc1]

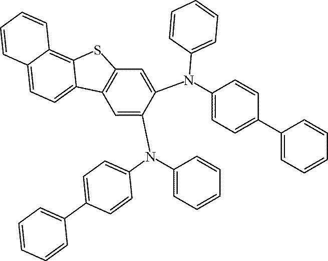 [SMILES: c1ccc(-c2ccc(N(c3ccccc3)c3cc4sc5c6ccccc6ccc5c4cc3N(c3ccccc3)c3ccc(-c4ccccc4)cc3)cc2)cc1]

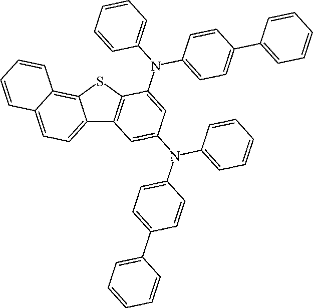 [SMILES: c1ccc(-c2ccc(N(c3ccccc3)c3cc(N(c4ccccc4)c4ccc(-c5ccccc5)cc4)c4sc5c6ccccc6ccc5c4c3)cc2)cc1]

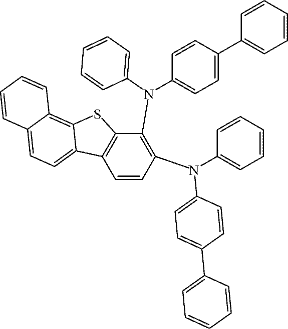 [SMILES: c1ccc(-c2ccc(N(c3ccccc3)c3ccc4c(sc5c6ccccc6ccc45)c3N(c3ccccc3)c3ccc(-c4ccccc4)cc3)cc2)cc1]

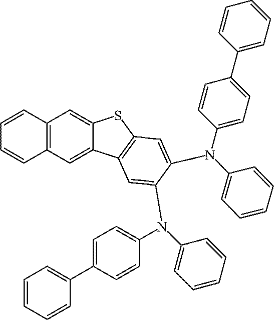 [SMILES: c1ccc(-c2ccc(N(c3ccccc3)c3cc4sc5cc6ccccc6cc5c4cc3N(c3ccccc3)c3ccc(-c4ccccc4)cc3)cc2)cc1]

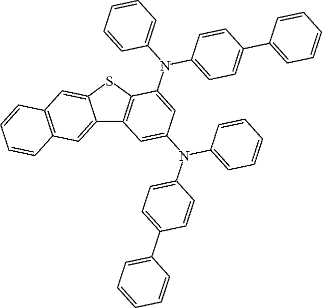 [SMILES: c1ccc(-c2ccc(N(c3ccccc3)c3cc(N(c4ccccc4)c4ccc(-c5ccccc5)cc4)c4sc5cc6ccccc6cc5c4c3)cc2)cc1]

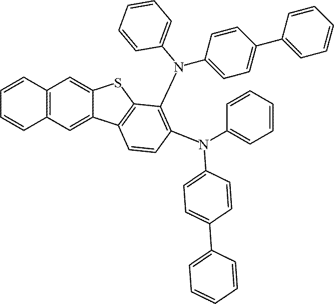 [SMILES: c1ccc(-c2ccc(N(c3ccccc3)c3ccc4c(sc5cc6ccccc6cc54)c3N(c3ccccc3)c3ccc(-c4ccccc4)cc3)cc2)cc1]

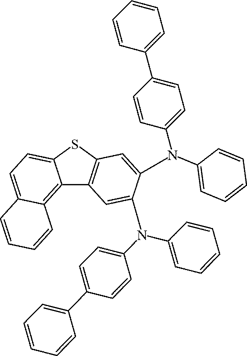 [SMILES: c1ccc(-c2ccc(N(c3ccccc3)c3cc4sc5ccc6ccccc6c5c4cc3N(c3ccccc3)c3ccc(-c4ccccc4)cc3)cc2)cc1]

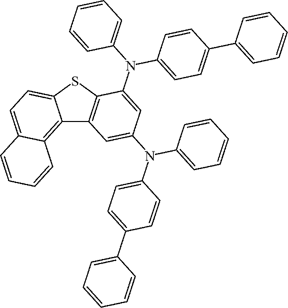 [SMILES: c1ccc(-c2ccc(N(c3ccccc3)c3cc(N(c4ccccc4)c4ccc(-c5ccccc5)cc4)c4sc5ccc6ccccc6c5c4c3)cc2)cc1]

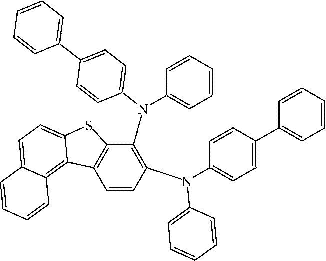 [SMILES: c1ccc(-c2ccc(N(c3ccccc3)c3ccc4c(sc5ccc6ccccc6c54)c3N(c3ccccc3)c3ccc(-c4ccccc4)cc3)cc2)cc1]

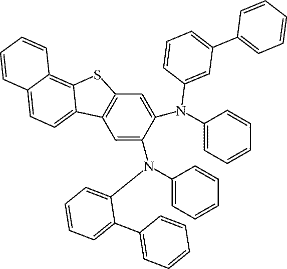 [SMILES: c1ccc(-c2cccc(N(c3ccccc3)c3cc4sc5c6ccccc6ccc5c4cc3N(c3ccccc3)c3ccccc3-c3ccccc3)c2)cc1]

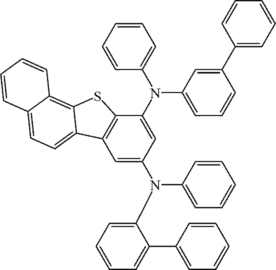 [SMILES: c1ccc(-c2cccc(N(c3ccccc3)c3cc(N(c4ccccc4)c4ccccc4-c4ccccc4)cc4c3sc3c5ccccc5ccc43)c2)cc1]

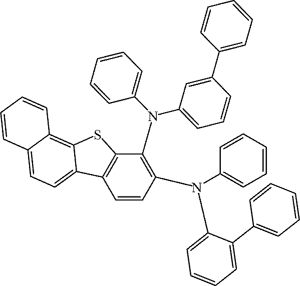 [SMILES: c1ccc(-c2cccc(N(c3ccccc3)c3c(N(c4ccccc4)c4ccccc4-c4ccccc4)ccc4c3sc3c5ccccc5ccc43)c2)cc1]

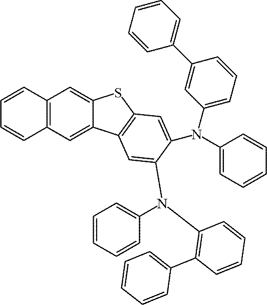 [SMILES: c1ccc(-c2cccc(N(c3ccccc3)c3cc4sc5cc6ccccc6cc5c4cc3N(c3ccccc3)c3ccccc3-c3ccccc3)c2)cc1]

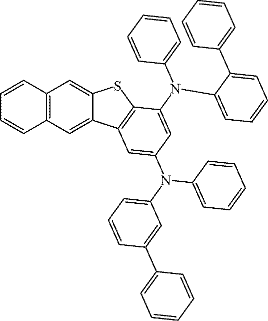 [SMILES: c1ccc(-c2cccc(N(c3ccccc3)c3cc(N(c4ccccc4)c4ccccc4-c4ccccc4)c4sc5cc6ccccc6cc5c4c3)c2)cc1]

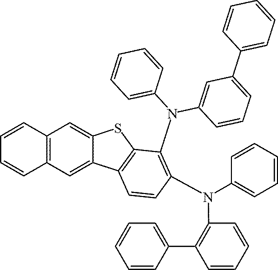 [SMILES: c1ccc(-c2cccc(N(c3ccccc3)c3c(N(c4ccccc4)c4ccccc4-c4ccccc4)ccc4c3sc3cc5ccccc5cc34)c2)cc1]

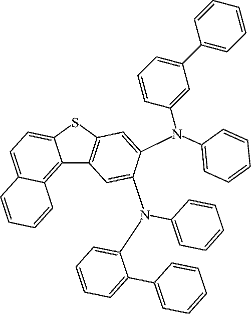 [SMILES: c1ccc(-c2cccc(N(c3ccccc3)c3cc4sc5ccc6ccccc6c5c4cc3N(c3ccccc3)c3ccccc3-c3ccccc3)c2)cc1]

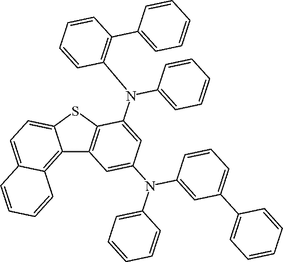 [SMILES: c1ccc(-c2cccc(N(c3ccccc3)c3cc(N(c4ccccc4)c4ccccc4-c4ccccc4)c4sc5ccc6ccccc6c5c4c3)c2)cc1]

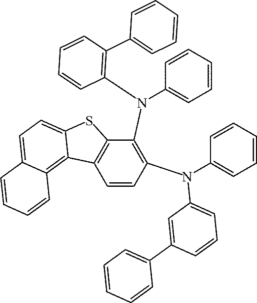 [SMILES: c1ccc(-c2cccc(N(c3ccccc3)c3ccc4c(sc5ccc6ccccc6c54)c3N(c3ccccc3)c3ccccc3-c3ccccc3)c2)cc1]

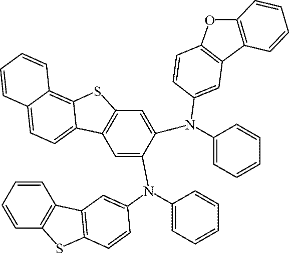 [SMILES: c1ccc(N(c2ccc3oc4ccccc4c3c2)c2cc3sc4c5ccccc5ccc4c3cc2N(c2ccccc2)c2ccc3sc4ccccc4c3c2)cc1]

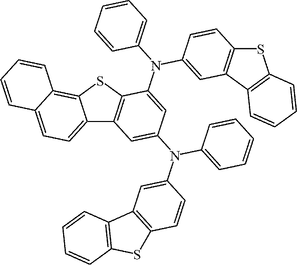 [SMILES: c1ccc(N(c2ccc3sc4ccccc4c3c2)c2cc(N(c3ccccc3)c3ccc4sc5ccccc5c4c3)c3sc4c5ccccc5ccc4c3c2)cc1]

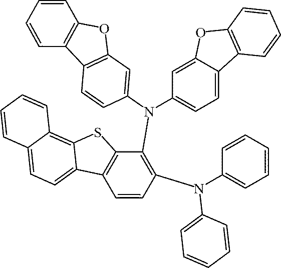 [SMILES: c1ccc(N(c2ccccc2)c2ccc3c(sc4c5ccccc5ccc34)c2N(c2ccc3c(c2)oc2ccccc23)c2ccc3c(c2)oc2ccccc23)cc1]

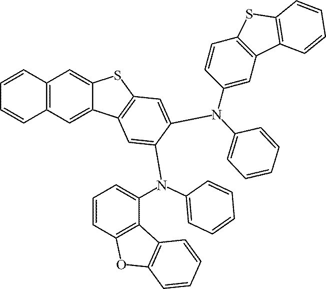 [SMILES: c1ccc(N(c2ccc3sc4ccccc4c3c2)c2cc3sc4cc5ccccc5cc4c3cc2N(c2ccccc2)c2cccc3oc4ccccc4c23)cc1]

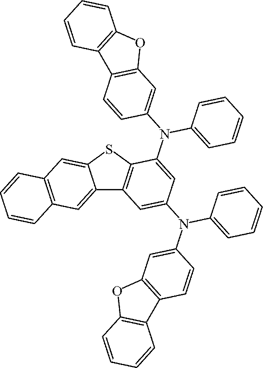 [SMILES: c1ccc(N(c2ccc3c(c2)oc2ccccc23)c2cc(N(c3ccccc3)c3ccc4c(c3)oc3ccccc34)c3sc4cc5ccccc5cc4c3c2)cc1]

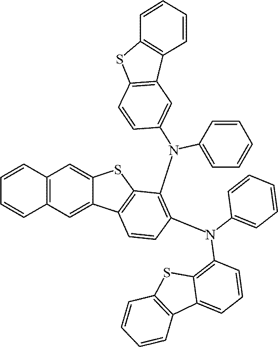 [SMILES: c1ccc(N(c2ccc3c(sc4cc5ccccc5cc43)c2N(c2ccccc2)c2ccc3sc4ccccc4c3c2)c2cccc3c2sc2ccccc23)cc1]

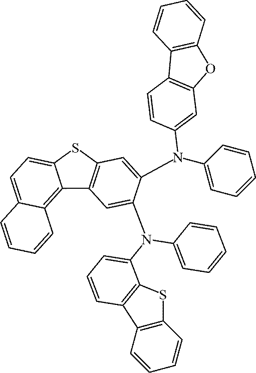 [SMILES: c1ccc(N(c2ccc3c(c2)oc2ccccc23)c2cc3sc4ccc5ccccc5c4c3cc2N(c2ccccc2)c2cccc3c2sc2ccccc23)cc1]

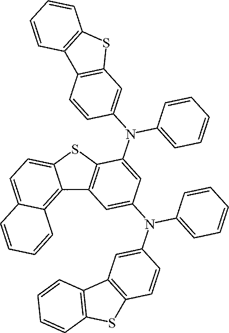 [SMILES: c1ccc(N(c2ccc3sc4ccccc4c3c2)c2cc(N(c3ccccc3)c3ccc4c(c3)sc3ccccc34)c3sc4ccc5ccccc5c4c3c2)cc1]

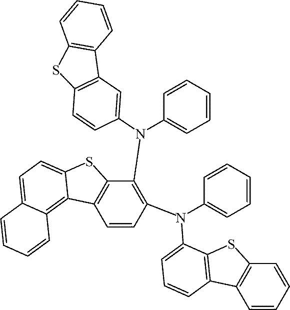 [SMILES: c1ccc(N(c2ccc3c(sc4ccc5ccccc5c43)c2N(c2ccccc2)c2ccc3sc4ccccc4c3c2)c2cccc3c2sc2ccccc23)cc1]

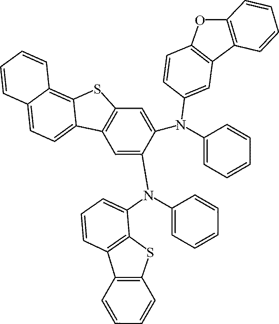 [SMILES: c1ccc(N(c2ccc3oc4ccccc4c3c2)c2cc3sc4c5ccccc5ccc4c3cc2N(c2ccccc2)c2cccc3c2sc2ccccc23)cc1]

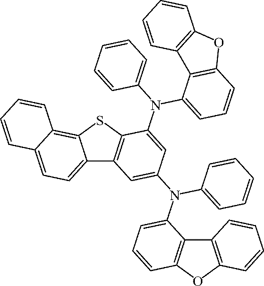 [SMILES: c1ccc(N(c2cc(N(c3ccccc3)c3cccc4oc5ccccc5c34)c3sc4c5ccccc5ccc4c3c2)c2cccc3oc4ccccc4c23)cc1]

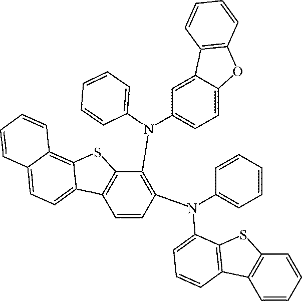 [SMILES: c1ccc(N(c2ccc3c(sc4c5ccccc5ccc34)c2N(c2ccccc2)c2ccc3oc4ccccc4c3c2)c2cccc3c2sc2ccccc23)cc1]

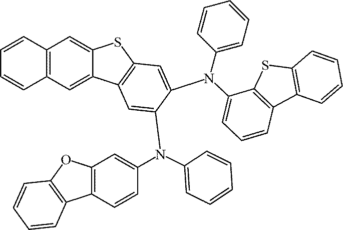 [SMILES: c1ccc(N(c2ccc3c(c2)oc2ccccc23)c2cc3c(cc2N(c2ccccc2)c2cccc4c2sc2ccccc24)sc2cc4ccccc4cc23)cc1]

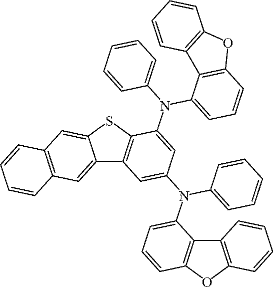 [SMILES: c1ccc(N(c2cc(N(c3ccccc3)c3cccc4oc5ccccc5c34)c3sc4cc5ccccc5cc4c3c2)c2cccc3oc4ccccc4c23)cc1]

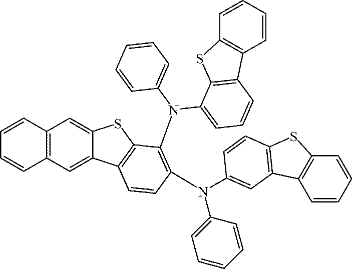 [SMILES: c1ccc(N(c2ccc3sc4ccccc4c3c2)c2ccc3c(sc4cc5ccccc5cc43)c2N(c2ccccc2)c2cccc3c2sc2ccccc23)cc1]

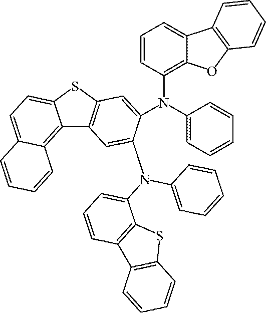 [SMILES: c1ccc(N(c2cc3sc4ccc5ccccc5c4c3cc2N(c2ccccc2)c2cccc3c2sc2ccccc23)c2cccc3c2oc2ccccc23)cc1]

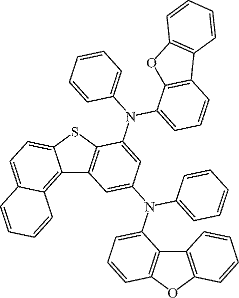 [SMILES: c1ccc(N(c2cccc3c2oc2ccccc23)c2cc(N(c3ccccc3)c3cccc4oc5ccccc5c34)cc3c2sc2ccc4ccccc4c23)cc1]

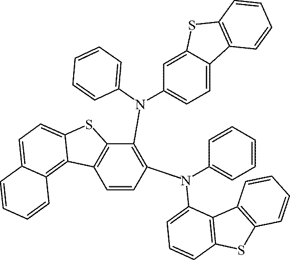 [SMILES: c1ccc(N(c2ccc3c(c2)sc2ccccc23)c2c(N(c3ccccc3)c3cccc4sc5ccccc5c34)ccc3c2sc2ccc4ccccc4c23)cc1]

The compound of Chemical Formula 1 can be prepared according to the preparation method as shown in Reaction Scheme 1 below.

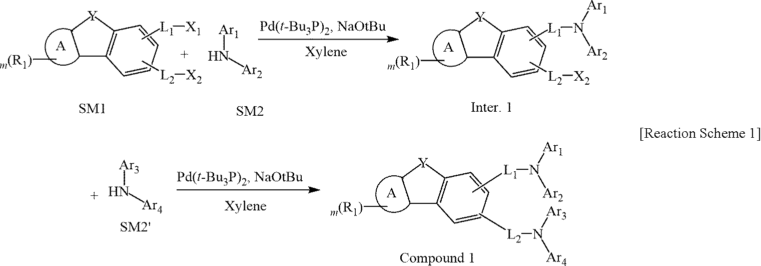

In Reaction Scheme 1, the remaining definitions excluding X₁ and X₂ are as defined above, and X₁ and X₂ are each independently a halogen, for example bromo, or chloro.

Specifically, the compound of Chemical Formula 1 is prepared by combining starting materials SM1, SM2 and SM2′ through an amine substitution reaction. These reactions are preferably carried out in the presence of a palladium catalyst and a base. The type of the reactive group and the catalyst used in the above reaction scheme can be appropriately changed. The above preparation method can be further specified in preparation examples described hereinafter.

### Organic Light Emitting Device

The present invention provides an organic light emitting device including the compound of Formula 1. In one example, the present invention provides an organic light emitting device including: a first electrode; a second electrode provided at a side opposite to the first electrode; and at least one layer of organic material layers provided between the first electrode and the second electrode, wherein the at least one layer of the organic material layers includes a compound of Chemical Formula 1.

The organic material layer of the organic light emitting device of the present invention can have a single layer structure, or it can have a multilayered structure in which two or more organic material layers are stacked. For example, the organic light emitting device of the present invention can have a structure including a hole injection layer, a hole transport layer, an electron inhibition layer, a light emitting layer, a hole blocking injection layer, an electron transport layer, an electron injection layer, and the like as the organic material layer. However, the structure of the organic light emitting device is not limited thereto, and it can include a smaller number of organic layers.

Further, the organic material layer can include a hole injection layer, a hole transport layer, or a layer simultaneously performing hole injection and transport, wherein the hole injection layer, the hole transport layer, and the layer simultaneously performing hole injection and transport include a compound of Chemical Formula 1.

Further, the organic material layer can include an electron inhibition layer, wherein the electron inhibition layer includes a compound of Chemical Formula 1.

Further, the organic material layer can include a light emitting layer, wherein the light emitting layer includes a compound of Chemical Formula 1. In this case, the compound of Chemical Formula 1 can be used as a host compound of the light emitting layer, preferably a red host compound. In this case, other compounds other than the compound of Chemical Formula 1 as the host compound can be used as the cohost compound.

Further, the organic material layer can include an electron transport layer or an electron injection layer, wherein the electron transport layer, and the electron injection layer include a compound of Chemical Formula 1.

Further, the organic material layer can include an electron blocking layer, wherein the electron blocking layer includes a compound of Chemical Formula 1.

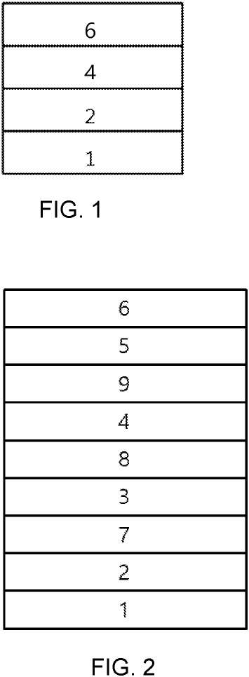

Further, the organic light emitting device according to the present invention can be a normal type of organic light emitting device in which an anode, one or more organic material layers, and a cathode are sequentially stacked on a substrate. Further, the organic light emitting device according to the present invention can be an inverted type of organic light emitting device in which a cathode, one or more organic material layers, and an anode are sequentially stacked on a substrate. For example, the structure of an organic light emitting device according to an embodiment of the present invention is illustrated in FIGS. 1 and 2.

FIG. 1 shows an example of an organic light emitting device including a substrate 1, an anode 2, a light emitting layer 4, and a cathode 6. In such a structure, the compound of Chemical Formula 1 can be included in the light emitting layer 4.

FIG. 2 shows an example of an organic light emitting device including a substrate 1, an anode 2, a hole injection layer 7, a hole transport layer 3, an electron inhibition layer 8, a light emitting layer 4, a hole blocking layer 9, an electron injection and transport layer 5, and a cathode 6. In such a structure, the compound of Chemical Formula 1 can be included in the light emitting layer 4.

The organic light emitting device according to the present invention can be manufactured by materials and methods known in the art, except that at least one layer of the organic material layers includes the compound of Chemical Formula 1. In addition, when the organic light emitting device includes a plurality of organic material layers, the organic material layers can be formed of the same material or different materials.

For example, the organic light emitting device according to the present invention can be manufactured by sequentially stacking a first electrode, an organic material layer and a second electrode on a substrate. In this case, the organic light emitting device can be manufactured by depositing a metal, metal oxides having conductivity, or an alloy thereof on the substrate by using a PVD (physical vapor deposition) method such as a sputtering method or an e-beam evaporation method to form the anode, forming an organic material layer including the hole injection layer, the hole transport layer, the light emitting layer, and the electron transport layer thereon, and then depositing a material that can be used as the cathode thereon. In addition to such a method, the organic light emitting device can be manufactured by sequentially depositing a cathode material, an organic material layer and an anode material on a substrate.

In addition, the compound of Chemical Formula 1 can be formed into an organic layer by a solution coating method as well as a vacuum deposition method at the time of manufacturing an organic light emitting device. Herein, the solution coating method means spin coating, dip coating, doctor blading, inkjet printing, screen printing, a spray method, roll coating, or the like, but is not limited thereto.

In addition to such a method, the organic light emitting device can be manufactured by sequentially depositing a cathode material, an organic material layer, and an anode material on a substrate (International Publication WO 2003/012890). However, the manufacturing method is not limited thereto.

For example, the first electrode is an anode and the second electrode is a cathode, or the first electrode is a cathode and the second electrode is an anode.

As the anode material, generally, a material having a large work function is preferably used so that holes can be smoothly injected into the organic material layer. Specific examples of the anode material include metals such as vanadium, chrome, copper, zinc, and gold, or an alloy thereof; metal oxides such as zinc oxides, indium oxides, indium tin oxides (ITO), and indium zinc oxides (IZO); a combination of metals and oxides, such as ZnO:Al or SnO₂:Sb; conductive polymers such as poly(3-methylthiophene), poly[3,4-(ethylene-1,2-dioxy)thiophene] (PEDOT), polypyrrole, and polyaniline, and the like, but are not limited thereto.

As the cathode material, generally, a material having a small work function is preferably used so that electrons can be easily injected into the organic material layer. Specific examples of the cathode material include metals such as magnesium, calcium, sodium, potassium, titanium, indium, yttrium, lithium, gadolinium, aluminum, silver, tin, and lead, or an alloy thereof; a multilayered structure material such as LiF/Al or LiO₂/Al, and the like, but are not limited thereto.

The hole injection layer is a layer for injecting holes from the electrode, and the hole injection material is preferably a compound which has a capability of transporting the holes, thus has a hole injecting effect in the anode and an excellent hole-injecting effect to the light emitting layer or the light emitting material, prevents excitons produced in the light emitting layer from moving to a hole injection layer or the electron injection material, and is excellent in the ability to form a thin film. It is preferable that a HOMO (highest occupied molecular orbital) of the hole injection material is between the work function of the anode material and a HOMO of a peripheral organic material layer. Specific examples of the hole injection material include metal porphyrin, oligothiophene, an arylamine-based organic material, a hexanitrilehexaazatriphenylene-based organic material, a quinacridone-based organic material, a perylene-based organic material, anthraquinone, a polyaniline and polythiophene-based conductive polymer, and the like, but are not limited thereto.

The hole transport layer is a layer that receives holes from a hole injection layer and transports the holes to the light emitting layer. The hole transport material is suitably a material having large mobility to the holes, which can receive holes from the anode or the hole injection layer and transfer the holes to the light emitting layer. Specific examples thereof include the compound of Chemical Formula 1 or an arylamine-based organic material, a conductive polymer, a block copolymer in which a conjugate portion and a non-conjugate portion are present together, and the like, but are not limited thereto.

The electron inhibition layer (electron blocking layer) refers to a layer which is formed on the hole transport layer, preferably provided in contact with the light emitting layer, and serves to adjust the hole mobility, prevent excessive movement of electrons, and increase the probability of hole-electron coupling, thereby improving the efficiency of the organic light emitting device. The electron blocking layer includes an electron blocking material, and examples of such electron blocking material can include the compound of Chemical Formula 1, and further includes an arylamine-based organic material or the like, but is not limited thereto.

The light emitting material is a material capable of emitting light in the visible light region by combining holes and electrons respectively transported from the hole transport layer and the electron transport layer, and having good quantum efficiency for fluorescence or phosphorescence. Specific examples include an 8-hydroxyquinoline aluminum (Alq₃) complex; carbazo-based compounds; dimerized styryl compounds; BAlq; 10-hydroxybenzoquinoline-metal compounds; benzoxazole, benzothiazole, and benzimidazole-based compounds; poly (p-phenylene vinylene) (PPV) -based polymers; spiro compounds; and polyfluorene, rubrene, and the like, but are not limited thereto.

The light emitting layer can include a host material and a dopant material. The host material can be a fused aromatic ring derivative, a heterocycle-containing compound or the like in addition to the compound of Chemical Formula 1. Specific examples of the fused aromatic ring derivatives include anthracenyl derivatives, pyrene derivatives, naphthalene derivatives, pentacene derivatives, phenanthrene compounds, fluoranthene compounds, and the like. Examples of the heterocyclic-containing compounds include carbazole derivatives, dibenzofuran derivatives, ladder-type furan compounds, pyrimidine derivatives, and the like, but are not limited thereto.

The dopant material includes an aromatic amine derivative, a styrylamine compound, a boron complex, a fluoranthene compound, a metal complex, and the like. Specifically, the aromatic amine derivative is a substituted or unsubstituted fused aromatic ring derivative having an arylamino group, and examples thereof include pyrene, anthracenyl, chrycenyl, periflanthene and the like, which have an arylamino group. The styrylamine compound is a compound where at least one arylvinyl group is substituted in substituted or unsubstituted arylamine, in which one or two or more substituent groups selected from the group consisting of an aryl group, a silyl group, an alkyl group, a cycloalkyl group, and an arylamino group are substituted or unsubstituted. Specific examples thereof include styrylamine, styryldiamine, styryltriamine, styryltetramine, and the like, but are not limited thereto. Further, the metal complex includes an iridium complex, a platinum complex, and the like, but is not limited thereto.

More specifically, the dopant material can include compounds having the following structures, but is not limited thereto.

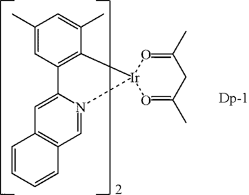

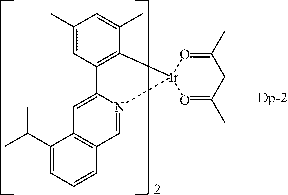

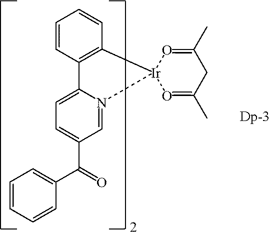

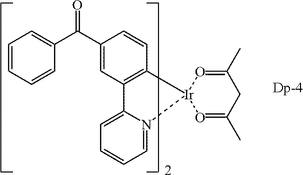

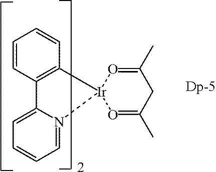

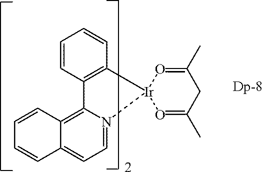

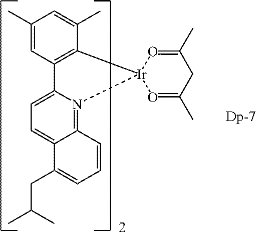

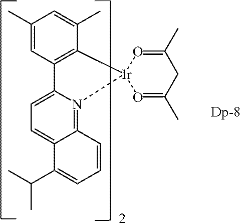

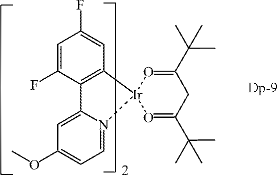

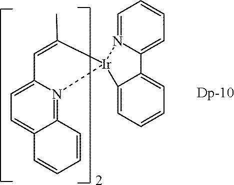

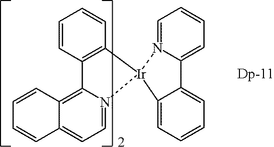

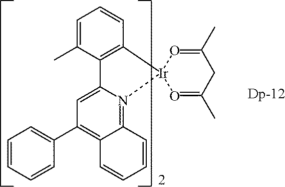

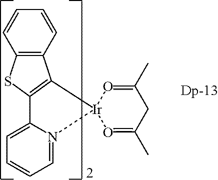

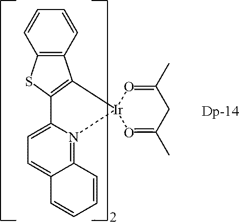

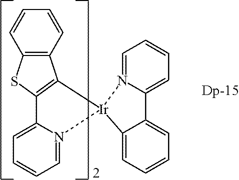

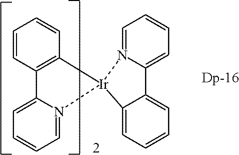

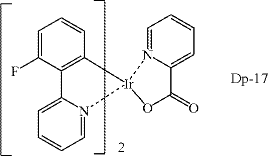

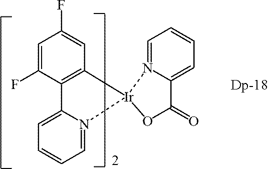

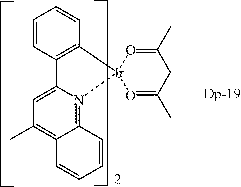

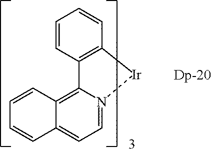

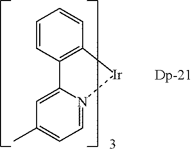

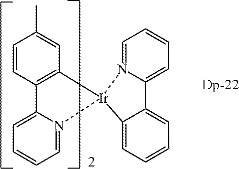

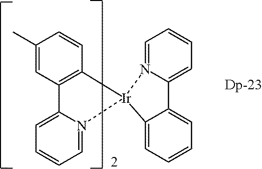

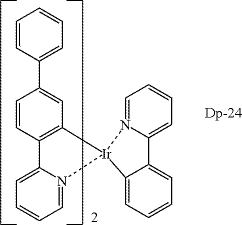

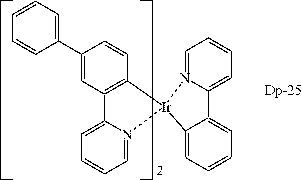

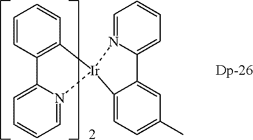

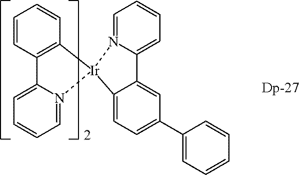

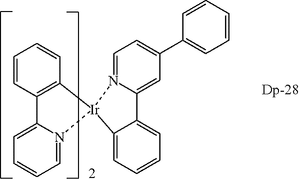

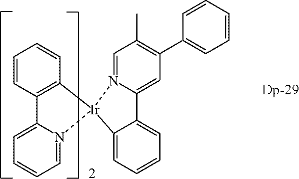

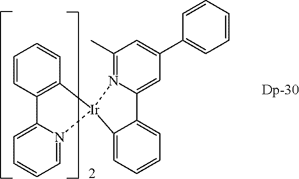

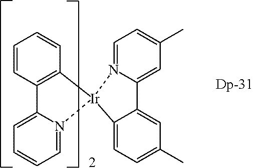

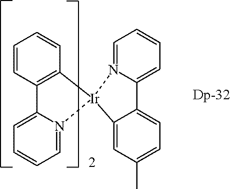

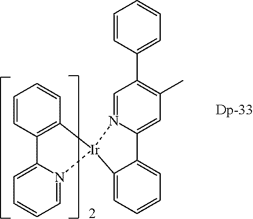

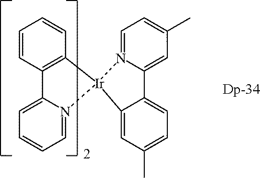

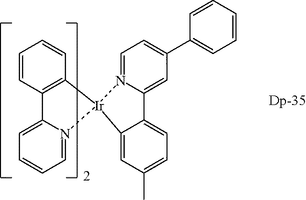

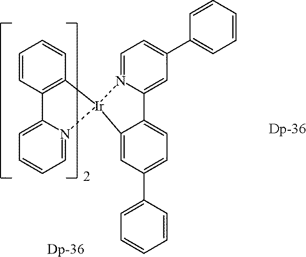

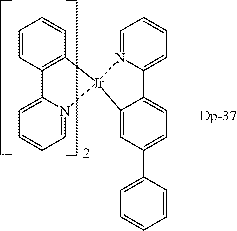

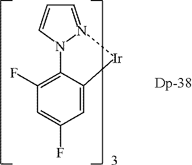

The hole blocking layer refers to a layer which is formed on the light emitting layer, preferably provided in contact with the light emitting layer, and serves to adjust the electron mobility, prevent excessive movement of holes, and increase the probability of hole-electron coupling, thereby improving the efficiency of the organic light emitting device. The hole blocking layer includes a hole blocking material, and examples of such hole blocking material can include a compound having an electron-withdrawing group introduced therein, such as azine derivatives including triazine; triazole derivatives; oxadiazole derivatives; phenanthroline derivatives; phosphine oxide derivatives, but is not limited thereto.

The electron injection and transport layer is a layer for simultaneously performing the roles of an electron transport layer and an electron injection layer that inject electrons from an electrode and transport the received electrons up to the light emitting layer, and is formed on the light emitting layer or the hole blocking layer. The electron injection and transport material is suitably a material which can receive electrons well from a cathode and transfer the electrons to a light emitting layer, and has a large mobility for electrons. Specific examples of the electron injection and transport material include: an Al complex of 8-hydroxyquinoline; a complex including Alq3; an organic radical compound; a hydroxyflavone-metal complex, a triazine derivative, and the like, but are not limited thereto. Alternatively, it can be used together with fluorenone, anthraquinodimethane, diphenoquinone, thiopyran dioxide, oxazole, oxadiazole, triazole, imidazole, perylenetetracarboxylic acid, fluorenylidene methane, anthrone, and the like, and derivatives thereof, a metal complex compound, a nitrogen-containing 5-membered ring derivative, and the like, but are not limited thereto.

The electron injection and transport layer can also be formed as a separate layer such as an electron injection layer and an electron transport layer. In such a case, the electron transport layer is formed on the light emitting layer or the hole blocking layer, and the above-mentioned electron injection and transport material can be used as the electron transport material included in the electron transport layer. In addition, the electron injection layer is formed on the electron transport layer, and examples of the electron injection material included in the electron injection layer include LiF, NaCl, CsF, Li2O, BaO, fluorenone, anthraquinodimethane, diphenoquinone, thiopyran dioxide, oxazole, oxadiazole, triazole, imidazole, perylenetetracarboxylic acid, fluorenylidene methane, anthrone, and the like, and derivatives thereof, a metal complex compound, a nitrogen-containing 5-membered ring derivative, and the like.

The metal complex compound includes 8-hydroxyquinolinato lithium, bis(8-hydroxyquinolinato)-zinc, bis(8-hydroxyquinolinato)copper, bis(8-hydroxy-quinolinato)manganese, tris(8-hydroxyquinolinato)-aluminum, tris(2-methyl-8-hydroxyquinolinato)aluminum, tris(8-hydroxyquinolinato)gallium, bis(10-hydroxy-benzo[h]quinolinato)beryllium, bis(10-hydroxy-benzo[h]quinolinato) zinc, bis(2-methyl-8-quinolinato)chlorogallium, bis(2-methyl-8-quinolinato) (o-cresolato)gallium, bis(2-methyl-8-quinolinato) (1-naphtholato)aluminum, bis(2-methyl-8-quinolinato) (2-naphtholato) gallium, and the like, but are not limited thereto.

The organic light emitting device according to the present disclosure can be a bottom emission device, a top emission device, or a double-sided light emitting device, and in particular, can be a bottom emission device that requires relatively high luminous efficiency.

In addition, the compound of Chemical Formula 1 can be included in an organic solar cell or an organic transistor in addition to an organic light emitting device.

Hereinafter, preferred examples are presented to assist in the understanding of the present disclosure. However, the following examples are for illustrative purposes only, and is not intended to limit the content of the present disclosure.

### Preparation Example - Preparation of an Intermediate Compound

### Preparation Example 1: Preparation of Compound A-a

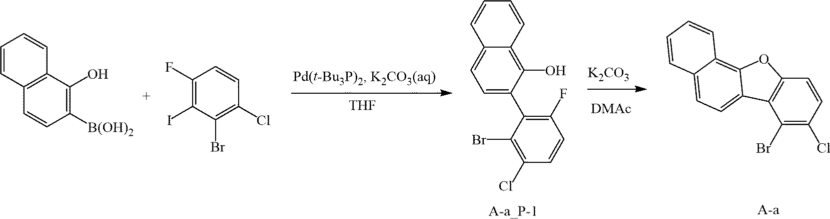

(1-hydroxynaphthalen-2-yl)boronic acid (10 g, 53.2 mmol) and 2-bromo-1-chloro-4-fluoro-3-iodobenzene (35.7 g, 106.4 mmol) were added to 200 mL of tetrahydrofuran, and then stirred and refluxed under a nitrogen atmosphere. Then, potassium carbonate(22.1 g, 159.6 mmol) was dissolved in 66 ml of water, and the aqueous solution was added thereto, and then sufficiently stirred. Then, bis(tri-tert-butylphosphine)palladium(0) (0.3 g, 0.5 mmol) was added thereto. After 9 hours of reaction, the reaction mixture was cooled to room temperature. The reaction mixture was separated into an organic layer and an aqueous layer, and then the organic layer was distilled. This was dissolved in chloroform, and washed two times with water, then the organic layer was separated, removed, and dried over anhydrous magnesium sulfate, and distilled under reduced pressure. The concentrated material was purified by silica gel column chromatography to produce 14.3 g of Compound A-a_P-1. (yield 77%, MS: [M+H] ⁺= 351)

Compound A-a_P-1 (10 g, 28.4 mmol) and potassium carbonate (11.8 g, 85.3 mmol) were added to 200 mL of DMAc, and then stirred and refluxed under a nitrogen atmosphere. After 9 hours of reaction, the reaction mixture was cooled to room temperature, and then the organic solvent was distilled under reduced pressure. This was dissolved in chloroform, and washed two times with water, then the organic layer was separated, removed, and dried over anhydrous magnesium sulfate, and distilled under reduced pressure. The concentrated material was purified by silica gel column chromatography to produce 6.8 g of Compound A-a. (yield 72%, MS: [M+H] ⁺= 331)

### Preparation Example 2: Preparation of Compound A-b

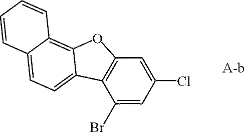 [SMILES: Clc1cc(Br)c2c(c1)oc1c3ccccc3ccc12]

197 Compound A-b was synthesized by the same method as the preparation Example 1, except that 1-bromo-5-chloro-3-fluoro-2-iodobenzene was used instead of 2-bromo-1-chloro-4-fluoro-3-iodobenzene.

### Preparation Example 3: Preparation of Compound A-c

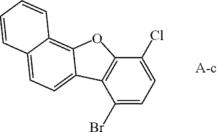 [SMILES: Clc1ccc(Br)c2c1oc1c3ccccc3ccc12]

Compound A-c was synthesized by the same method as the preparation Example 1, except that 1-bromo-4-chloro-3-fluoro-2-iodobenzene was used instead of 2-bromo-1-chloro-4-fluoro-3-iodobenzene.

### Preparation Example 4: Preparation of Compound A-d

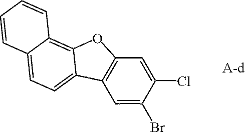 [SMILES: Clc1cc2oc3c4ccccc4ccc3c2cc1Br]

Compound A-e was synthesized by the same method as the preparation Example 1, except that 5-bromo-1-chloro-2-fluoro-3-iodobenzene was used instead of 2-bromo-1-chloro-4-fluoro-3-iodobenzene.

### Preparation Example 5: Preparation of Compound A-e

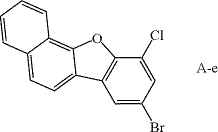 [SMILES: Clc1cc(Br)cc2c1oc1c3ccccc3ccc21]

Compound A-d was synthesized by the same method as the preparation Example 1, except that 1-bromo-2-chloro-4-fluoro-5-iodobenzene was used instead of 2-bromo-1-chloro-4-fluoro-3-iodobenzene.

### Preparation Example 6: Preparation of Compound A-f

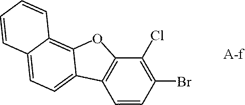 [SMILES: Clc1c(Br)ccc2c1oc1c3ccccc3ccc21]

Compound A-f was synthesized by the same method as the preparation Example 1, except that 1–bromo-2–chloro- 3–fluoro-4–iodobenzene was used instead of 2–bromo-1– chloro-4–fluoro-3–ipdpbemzeme.

### Preparation Example 7: Preparation of Compound A-g

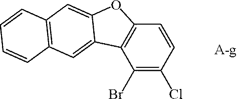 [SMILES: Clc1ccc2oc3cc4ccccc4cc3c2c1Br]

Compound A-g was synthesized by the same method as the preparation Example 1, except that (3–hydroxynphthalen-2–yl) boronic acid was used instead of (1–hydroxynaphthalen-2–yl) boronic acid.

### Preparation Example 8: Preparation of Compound A-h

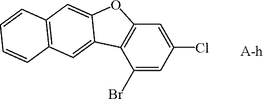 [SMILES: Clc1cc(Br)c2c(c1)oc1cc3ccccc3cc12]

Compound A-h was synthesized by the same method as the preparation Example 1, except that (3–hydroxynaphthalen–2–yl) boronic acid was used instead of (1–hydroxynaphthalen–2–yl)boronic acid and 1–bromo–5–chloro–3–fluoro-2–iodobenzene was used instead of 2–bromo-1–chloro-4–fluoro–3–iodobenzene.

### Preparation Example 9: Preparation of Compound A-i

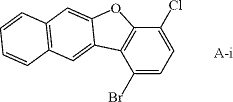 [SMILES: Clc1ccc(Br)c2c1oc1cc3ccccc3cc12]

Compound A–i was synthesized by the same method as the preparation Example 1, except that (3–hydroxynaphthalen-2–yl) boronic acid was used instead of (1–hydroxynaphthalen-2–yl)boronic acid and 1–bromo-4–chloro-3–fluoro-2–iodobenzene was used instead of 2–bromo-1–chloro-4–fluoro–3–iodobenzene.

### Preparation Example 10: Preparation of Compound A-j

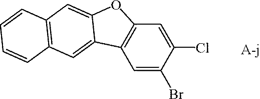 [SMILES: Clc1cc2oc3cc4ccccc4cc3c2cc1Br]

Comound A-j was synthesized by the same method as the preparatin Example 1, except that (3–hydroxynaphthalen-2–yl)boronic acid was used instead of (1–hydroxynaphthalen-2–yl)boronic acid and 1–bromo-2–chloro-4fluoro–5–iodobenzene was used instead of 2–Bromo– 1–chloro-4–fluoro-3–iodenzene.

### Preparation Example 11: Preparation of Compound A-k

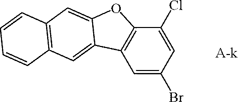 [SMILES: Clc1cc(Br)cc2c1oc1cc3ccccc3cc12]

Compound A-k was synthesized by the same method as the preparation Example 1, except that (3–hydroxynaphthalen-e-yl)boronic acid was used instead of (1–hydroxynaphthalen–2–yl)boronic acid and 5–bromo-1–chloro-2–fluoro–3–iodobenzene was used instead of 2–bromo- 1–chloro-4–fluoro–2–iodobenzene.

### Preparation Example 12: Preparation of Compound A-1

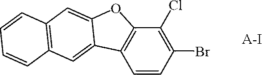 [SMILES: Clc1c(Br)ccc2c1oc1cc3ccccc3cc12]

Compound A-l was synthesized by the same method as the preparation Example 1, except that (3–hydroxynaphthalen-2–yl) boronic acid was used instead of (1–hydroxynaphthalen-2–yl) boronic acid and 1–bromo-2–chloro-3–fluoro-4–iodobenzene was used instead of 2–bromo-1–chloro-4–fluoro-3–iodobenzene.

### Preparation Example 13: Preparation of Compound A-m

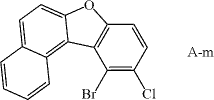 [SMILES: Clc1ccc2oc3ccc4ccccc4c3c2c1Br]

Counpound A-m was synthesized by the same methode as the preparation Example 1, except that (2–hydroxynaphthalen-1–yl) boronic acid was used instead of (1–hydroxynaphthalen-2–yl)boronic acid.

### Preparation Example 14: Preparation of Compound A-n

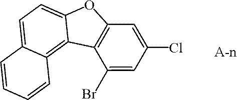 [SMILES: Clc1cc(Br)c2c(c1)oc1ccc3ccccc3c12]

Compound A-n was synthesized by the same method as the preparation Example 1, except that (2–hydroxynaphthalen-1–yl) boronic acid was used instead of (1–hydroxynaphthalen-2–yl) boronic acid and 1–bromo-5–chloro–3–fluoro–2–iodobenzene was used instead of 2–bromo-1–chloro–4–fluoro–3–iodobenzene,

### Preparation Example 15: Preparation of Compound A-o

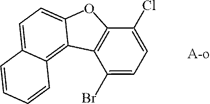 [SMILES: Clc1ccc(Br)c2c1oc1ccc3ccccc3c12]

comound A-o was synthesized by the same method as the preparation Example 1, except that (2–hydroxynaphthalen–1–yl)boronic acid was used instead of (1–hydroxynaphthalen–2–yl)boronic acid and 1–bromo–4–chloro–3–fluoro–2–iodobenzene was used instead of 2– bromo-1–chloro-4–fluoro-3–iodobenzene.

### Preparation Example 16: Preparation of Compound A-p

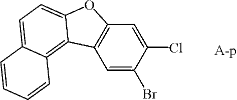 [SMILES: Clc1cc2oc3ccc4ccccc4c3c2cc1Br]

Compound A-p was synthesed by the same method as the preparation Example 1, except that (2–hydroxynaphthalen–1–ly)boronic acid was used instead of (1–hydroxynaphthalen–2–yl)boronix acid and 1–bromo–2–clhoro–4–fluoro–5–iodobenzene was used instead of 2– bromo–1–chloro–4–fluoro-3–iodobenzene.

### Preparation Example 17: Preparation of Compound A-q

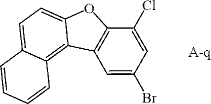 [SMILES: Clc1cc(Br)cc2c1oc1ccc3ccccc3c12]

Compound A-q was synthesed by the same method as the preparation Example 1, except that (2–hydroxynaphthalen–1–ly)boronic acid was used instead of (1–hydroxynaphthalen-2–yl)boronic acid and 5–bromo–1–chloro-2–fluoro–3–iodobenzene was used instead of 2– bromo-1–chloro-4–fluoro-3–idibebzebe.

### Preparation Example 18: Preparation of Compound A-r

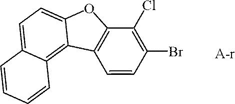 [SMILES: Clc1c(Br)ccc2c1oc1ccc3ccccc3c12]

Compound A-r was synthesed by the same method as the preparation Example 1, except that (2–hydroxynaphthalen–1–ly)boronic acid was used instead of (1–hydroxynaphthalen–2–yl)boronic acid and 1–bromo–2–chloro–3–fluoro–4–iodobenzene was used instead of 2– bromo–1–chloro–4–fluoro–3–iodobenzene.

### Preparation Example 19: Preparation of Compound B-a

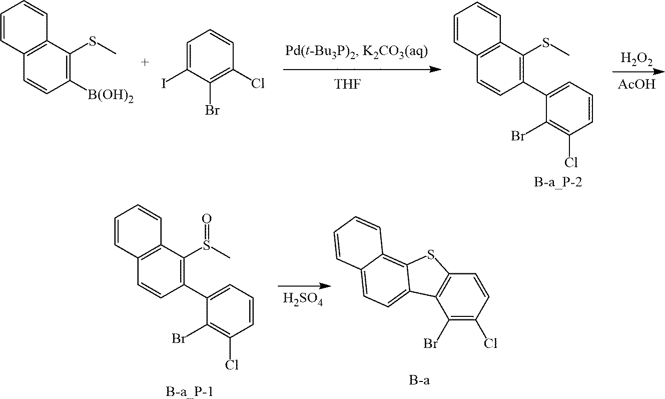

(1- (methylthio)naphthalen-2-yl)boronic acid (10 g, 45.9 mmol) and 2-bromo-1-chloro-3-iodobenzene (16 g, 50.4 mmol) were added to 200 mL of tetrahydrofuran, and then stirred and refluxed under a nitrogen atmosphere. Then, potassium carbonate (19 g, 137.6 mmol) was dissolved in 57 ml of water, and the aqueous solution was added thereto, and then sufficiently stirred. Then, bis(tri-tert-butylphosphine)palladium(0) (0.2 g, 0.5 mmol) was added thereto. After 12 hours of reaction, the reaction mixture was cooled to room temperature. The reaction mixture was separated into an organic layer and an aqueous layer, and then the organic layer was distilled. This was dissolved in chloroform, and washed two times with water, then the organic layer was separated, removed, and dried over anhydrous magnesium sulfate, and distilled under reduced pressure. The concentrated material was purified by silica gel column chromatography to produce Compound B-a _P-2. (yield 73%, MS: [M+H] ⁺= 363)

Compound B-a_P-2 (10 g, 27.5 mmol) and Hydrogen Peroxide (1 g, 30.2 mmol) were added to 200 mL of acetic acid, and then stirred and refluxed under a nitrogen atmosphere. After 3 hours of reaction, the reaction product was poured into water to precipitate crystals and filtered. This filtrated solid was dissolved in chloroform, and washed two times with water, then the organic layer was separated, removed, and dried over anhydrous magnesium sulfate, and distilled under reduced pressure. The concentrated material was purified by silica gel column chromatography to produce 7.4 g of Compound B-a_P-1. (yield 71%, MS: [M+H] ⁺= 379)

Compound B-a_P-1 (10 g, 26.3 mmol) were added to 200 mL of H₂SO₄, and then stirred and refluxed under a nitrogen atmosphere. After 2 hours of reaction, the reaction product was poured into water to precipitate crystals and filtered. This filtrated solid was dissolved in chloroform, and washed two times with water, then the organic layer was separated, removed, and dried over anhydrous magnesium sulfate, and distilled under reduced pressure. The concentrated material was purified by silica gel column chromatography to produce 7.1 g of Compound B-a. (yield 78%, MS: [M+H] ⁺= 347)

### Preparation Example 20: Preparation of Compound B-b

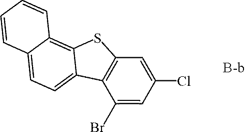 [SMILES: Clc1cc(Br)c2c(c1)sc1c3ccccc3ccc12]

Compound B-b was synthesized by the same method as the preparation Example 19, except that 2-bromo-4-chloro-1-iodobenzene was used instead of 2-bromo-1-chloro-3-iodobenzene.

### Preparation Example 21: Preparation of Compound B-c

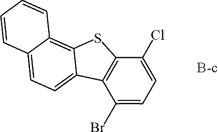 [SMILES: Clc1ccc(Br)c2c1sc1c3ccccc3ccc12]

Compound B-c was synthesized by the same method as the preparation Example 19, except that 1-bromo-4-chloro-2-iodobenzene was used instead of 2-bromo-1-chloro-3-iodobenzene.

### Preparation Example 22: Preparation of Compound B-d

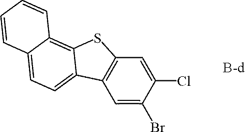 [SMILES: Clc1cc2sc3c4ccccc4ccc3c2cc1Br]

Compound B-d was synthesized by the same method as the preparation Example 19, except that 2-bromo-1-chloro-4-iodobenzene was used instead of 2-bromo-1-chloro-3-iodobenzene.

### Preparation Example 23: Preparation of Compound B-e

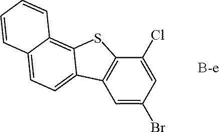 [SMILES: Clc1cc(Br)cc2c1sc1c3ccccc3ccc21]

Compound B-e was synthesized by the same method as the preparation Example 19, except that 1-bromo-3-chloro-5-iodobenzene was used instead of 2-bromo-1-chloro-3-iodobenzene.

### Preparation Example 24: Preparation of Compound B-f

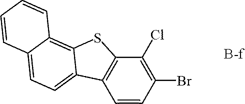 [SMILES: Clc1c(Br)ccc2c1sc1c3ccccc3ccc21]

Compound B-f was synthesized by the same method as the preparation Example 19, except that 1-bromo-2-chloro-4-iodobenzene was used instead of 2-bromo-1-chloro-3-iodobenzene.

### Preparation Example 25: Preparation of Compound B-g

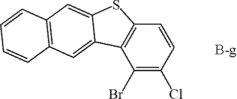 [SMILES: Clc1ccc2sc3cc4ccccc4cc3c2c1Br]

Compound B-g was synthesized by the same method as the preparation Example 19, except that 3-(methylthio)naphthalen-2-yl)boronic acid was used instead of (1-(methylthio)naphthalen-2-yl)boronic acid.

### Preparation Example 26: Preparation of Compound B-h

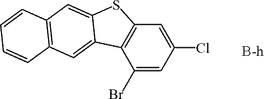 [SMILES: Clc1cc(Br)c2c(c1)sc1cc3ccccc3cc12]

Compound B-h was synthesized by the same method as the preparation Example 19, except that (3-(methylthio)naphthalen-2-yl)boronic acid was used instead of (1-(methylthio)naphthalen-2-yl)boronic acid and 2-bromo-4-chloro-1-iodobenzene was used instead of 2-bromo-1-chloro-3-iodobenzene.

### Preparation Example 27: Preparation of Compound B-i

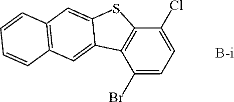 [SMILES: Clc1ccc(Br)c2c1sc1cc3ccccc3cc12]

Compound B-i was synthesized by the same method as the preparation Example 19, except that (3-(methylthio)naphthalen-2-yl)boronic acid was used instead of (1-(methylthio)naphthalen-2-yl)boronic acid and 1-bromo-4-chloro-2-iodobenzene was used instead of 2-bromo-1-chloro-3-iodobenzene.

### Preparation Example 28: Preparation of Compound B-j

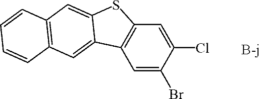 [SMILES: Clc1cc2sc3cc4ccccc4cc3c2cc1Br]

Compound B-j was synthesized by the same method as the preparation Example 19, except that (3-(methylthio)naphthalen-2-yl)boronic acid was used instead of (1-(methylthio)naphthalen-2-yl)boronic acid and 2-bromo-1-chloro-4-iodobenzene was used instead of 2-bromo-1-chloro-3-iodobenzene.

### Preparation Example 29: Preparation of Compound B-k

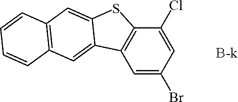 [SMILES: Clc1cc(Br)cc2c1sc1cc3ccccc3cc12]

Compound B-k was synthesized by the same method as the preparation Example 19, except that (3-(methylthio)naphthalen-2-yl)boronic acid was used instead of (1-(methylthio)naphthalen-2-yl)boronic acid and 1-bromo-3-chloro-5-iodobenzene was used instead of 2-bromo-1-chloro-3-iodobenzene.

### Preparation Example 30: Preparation of Compound B-1

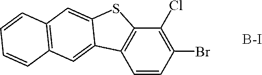 [SMILES: Clc1c(Br)ccc2c1sc1cc3ccccc3cc12]

Compound B-1 was synthesized by the same method as the preparation Example 19, except that (3-(methylthio)naphthalen-2-yl)boronic acid was used instead of (1-(methylthio)naphthalen-2-yl)boronic acid and 1-bromo-2-chloro-4-iodobenzene was used instead of 2-bromo-1-chloro-3-iodobenzene.

### Preparation Example 31: Preparation of Compound B-m

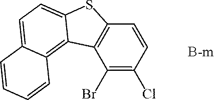 [SMILES: Clc1ccc2sc3ccc4ccccc4c3c2c1Br]

Compound B-m was synthesized by the same method as the preparation Example 19, except that (2-(methylthio)naphthalen-1-yl)boronic acid was used instead of (1-(methylthio)naphthalen-2-yl)boronic acid.

### Preparation Example 32: Preparation of Compound B-n

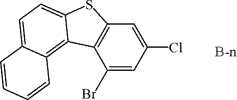 [SMILES: Clc1cc(Br)c2c(c1)sc1ccc3ccccc3c12]

Compound B-1 was synthesized by the same method as the preparation Example 19, except that (2-(methylthio)naphthalen-1-yl)boronic acid was used instead of (1-(methylthio)naphthalen-2-yl)boronic acid and 2-bromo-4-chloro-1-iodobenzene was used instead of 2-bromo-1-chloro-3-iodobenzene.

### Preparation Example 33: Preparation of Compound B-o

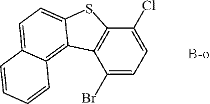 [SMILES: Clc1ccc(Br)c2c1sc1ccc3ccccc3c12]

Compound B-o was synthesized by the same method as the preparation Example 19, except that (2-(methylthio)naphthalen-1-yl)boronic acid was used instead of (1-(methylthio)naphthalen-2-yl)boronic acid and 1-bromo-4-chloro-2-iodobenzene was used instead of 2-bromo-1-chloro-3-iodobenzene.

### Preparation Example 34: Preparation of Compound B-p

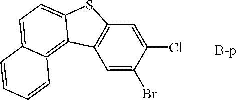 [SMILES: Clc1cc2sc3ccc4ccccc4c3c2cc1Br]

Compound B-p was synthesized by the same method as the preparation Example 19, except that (2-(methylthio)naphthalen-1-yl)boronic acid was used instead of (1-(methylthio)naphthalen-2-yl)boronic acid and 2-bromo-1-chloro-4-iodobenzene was used instead of 2-bromo-1-chloro-3-iodobenzene.

### Preparation Example 35: Preparation of Compound B-q

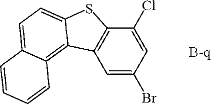 [SMILES: Clc1cc(Br)cc2c1sc1ccc3ccccc3c12]

Compound B-q was synthesized by the same method as the preparation Example 19, except that (2-(methylthio)naphthalen-1-yl)boronic acid was used instead of (1-(methylthio)naphthalen-2-yl)boronic acid and 1-bromo-3-chloro-5-iodobenzene was used instead of 2-bromo-1-chloro-3-iodobenzene.

### Preparation Example 36: Preparation of Compound B-r

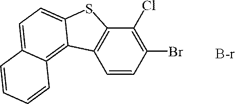 [SMILES: Clc1c(Br)ccc2c1sc1ccc3ccccc3c12]

Compound B-r was synthesized by the same method as the preparation Example 19, except that (2-(methylthio)naphthalen-1-yl)boronic acid was used instead of (1-(methylthio)naphthalen-2-yl)boronic acid and 1-bromo-2-chloro-4-iodobenzene was used instead of 2-bromo-1-chloro-3-iodobenzene.

### Synthesis Example

### Synthesis Example 1: Synthesis of Compound 1

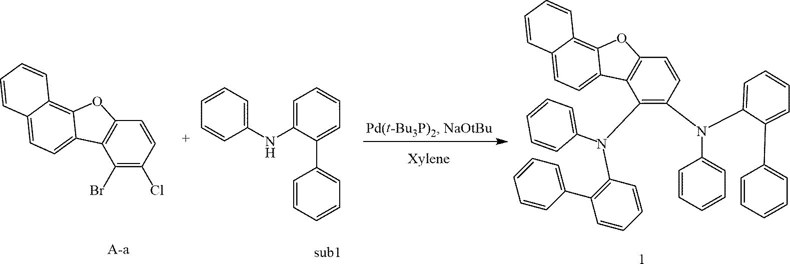

Compound A-a (10 g, 30.2 mmol) and Compound sub 1 (15.2 g, 61.8 mmol) and sodium tert-butoxide (7.2 g, 75.4 mmol) were added to 200 ml of xylene under a nitrogen atmosphere, and the mixture was stirred and refluxed. Then, bis (tri-tert-butylphosphine)palladium (0) (0.3 g, 0.6 mmol) was added thereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material was purified by silica gel column chromatography to produce 14.4 g of Compound 1. (yield 68%, MS: [M+H] ⁺= 705) .

### Synthesis Example 2: Synthesis of Compound 2

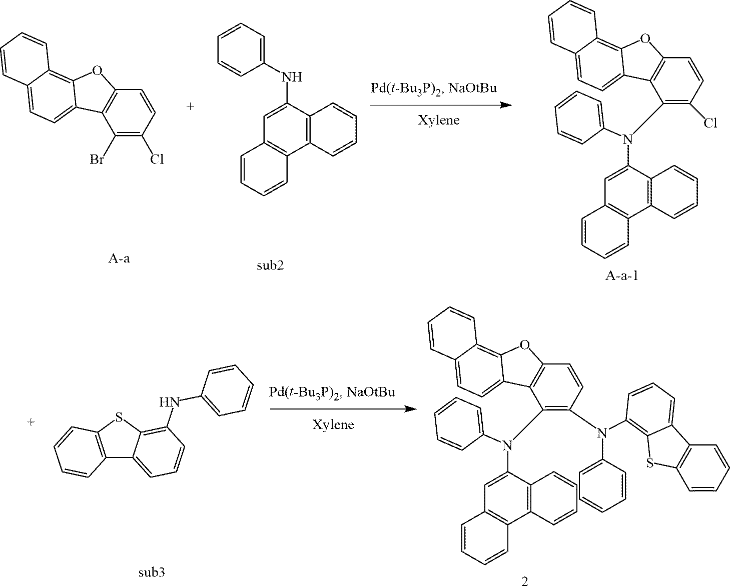

Compound A-a (10 g, 30.2 mmol), sub2 (8.2 g, 30.5 mmol), and sodium tert-butoxide (3.5 g, 36.2 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material was purified by silica gel column chromatography to produce 9.4 g of Compound A-a-1. (yield 60%, MS: [M+H] ⁺= 520).

Compound A-a-1 (10 g, 19.2 mmol), sub3 (5.4 g, 19.6 mmol) and sodium tert-butoxide (2.4 g, 25 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material was purified by silica gel column chromatography to produce 7.6 g of Compound 2. (yield 52%, MS: [M+H] ⁺= 759).

### Synthesis Example 3: Synthesis of Compound 3

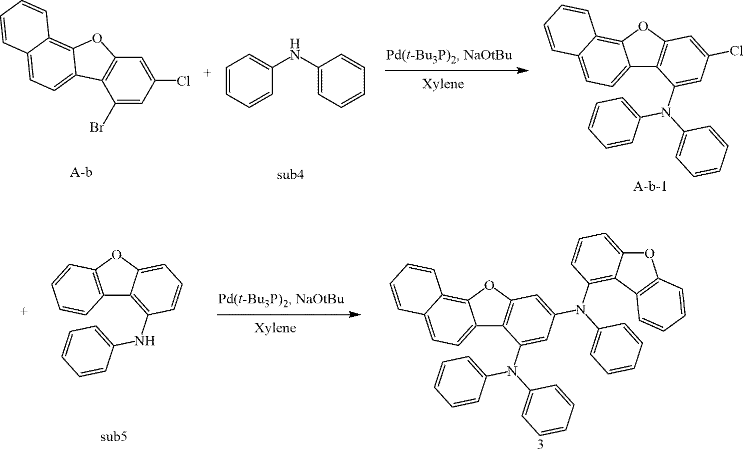

Compound A-b (10 g, 30.2 mmol), sub4 (5.2 g, 30.5 mmol) and sodium tert-butoxide (3.5 g, 36.2 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material was purified by silica gel column chromatography to produce 9.4 g of Compound A-b-1. (yield 56%, MS: [M+H] ⁺= 420).

Compound A-b-1 (10 g, 23.8 mmol), sub5 (6.3 g, 24.3 mmol), and sodium tert-butoxide (3 g, 31 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material was purified by silica gel column chromatography to produce 7.8 g of Compound 3. (yield 51%, MS: [M+H] ⁺= 643).

### Synthesis Example 4: Synthesis of Compound 4

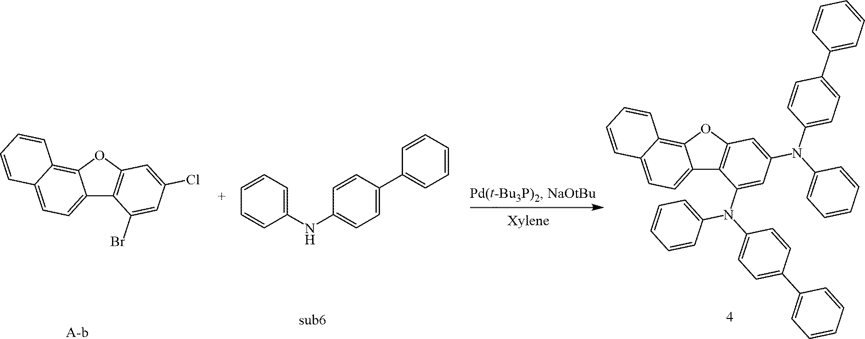

Compound A-b (10 g, 30.2 mmol), sub6 (15.2 g, 61.8 mmol) and sodium tert-butoxide (7.2 g, 75.4 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material was purified by silica gel column chromatography to produce 11 g of Compound 4. (yield 52%, MS: [M+H] ⁺= 705).

### Synthesis Example 5: Synthesis of Compound 5

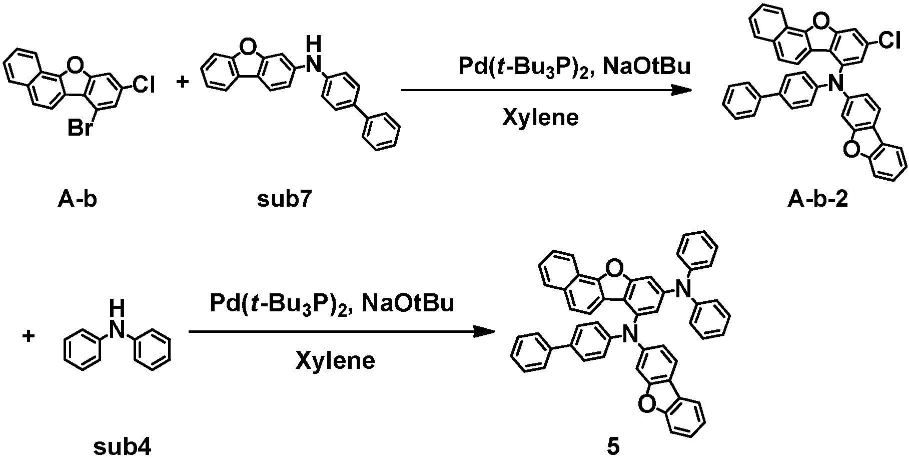 Compound A-b (10 g, 30.2 mmol), sub7 (10.2 g, 30.5 mmol) and sodium tert-butoxide (3.5 g, 36.2 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material was purified by silica gel column chromatography to produce 10.8 g of Compound A-b-2. (yield 61%, MS: [M+H] ⁺= 586).

Compound A-b-2 (10 g, 17.1 mmol), sub4 (2.9 g, 17.4 mmol), and sodium tert-butoxide (2.1 g, 22.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material was purified by silica gel column chromatography to produce 7.4 g of Compound 5. (yield 60%, MS: [M+H] ⁺= 719).

### Synthesis Example 6: Synthesis of Compound 6

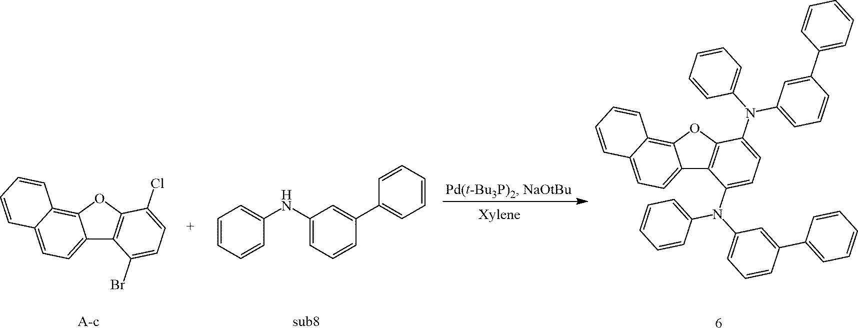

Compound A-c (10 g, 30.2 mmol), sub8 (15.2 g, 61.8 mmol), and sodium tert-butoxide (7.2 g, 75.4 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material waspurified by silica gel column chromatography to produce 14.7 g of Compound 6. (yield 69%, MS: [M+H] ⁺= 705).

### Synthesis Example 7: Synthesis of Compound 7

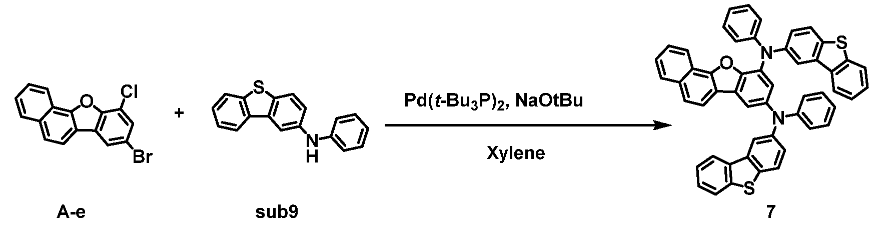 Compound A-e (10 g, 30.2 mmol), sub9 (17 g, 61.8 mmol), and sodium tert-butoxide (7.2 g, 75.4 mmol) were added to 200 ml of xylene under a nitrogen atmosphere, and the mixture was stirred and refluxed. Then, bis(tri-tert-butylphosphine)palladium (0) (0.3 g, 0.6 mmol) was added thereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material was purified by silica gel column chromatography to produce 14.3 g of Compound 7. (yield 62%, MS: [M+H] ⁺= 765).

### Synthesis Example 8: Synthesis of Compound 8

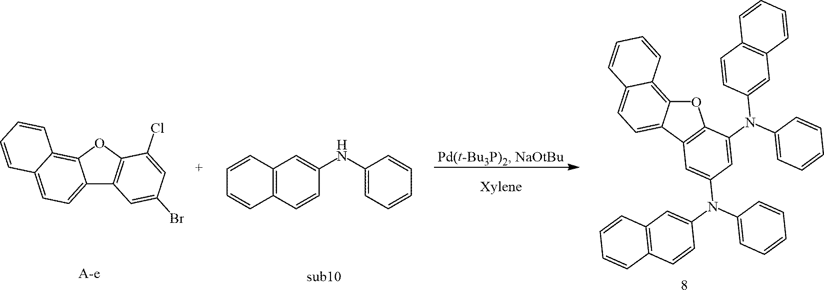

Compound A-e (10 g, 30.2 mmol), sub10 (13.6 g, 61.8 mmol), and sodium tert-butoxide (7.2 g, 75.4 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material was purified by silica gel column chromatography to produce 12.4 g of Compound 8. (yield 63%, MS: [M+H] ⁺= 653).

### Synthesis Example 9: Synthesis of Compound 9

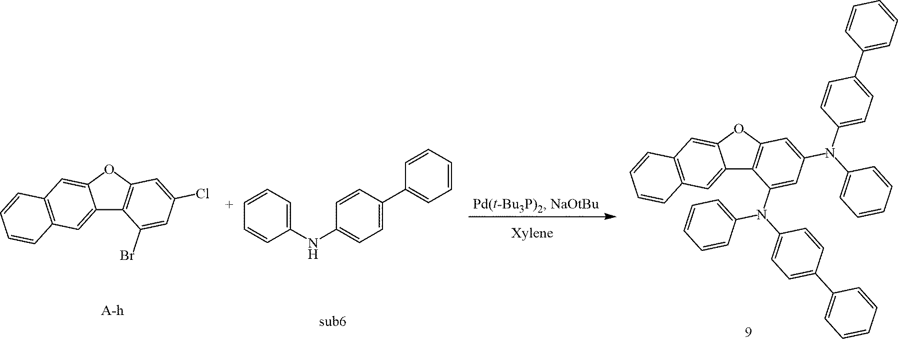

Compound A-h (10 g, 30.2 mmol), sub6 (15.2 g, 61.8 mmol) and sodium tert-butoxide (7.2 g, 75.4 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material was purified by silica gel column chromatography to produce 11 g of Compound 9. (yield 52%, MS: [M+H] ⁺= 705).

### Synthesis Example 10: Synthesis of Compound 10

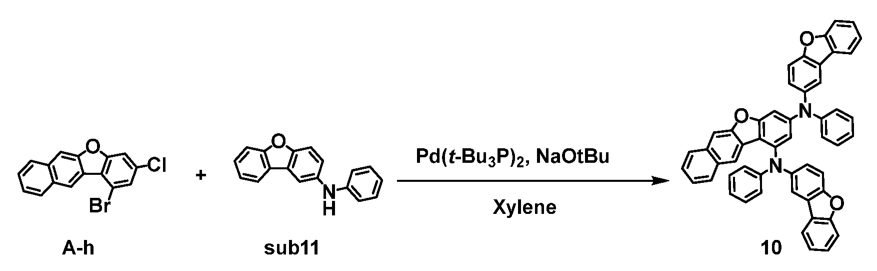 Compound A-h (10 g, 30.2 mmol), sub11 (16 g, 61.8 mmol) and sodium tert-butoxide (7.2 g, 75.4 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material was purified by silica gel column chromatography to produce 14.4 g of Compound 10. (yield 65%, MS: [M+H] ⁺= 733).

### Synthesis Example 11: Synthesis of Compound 11

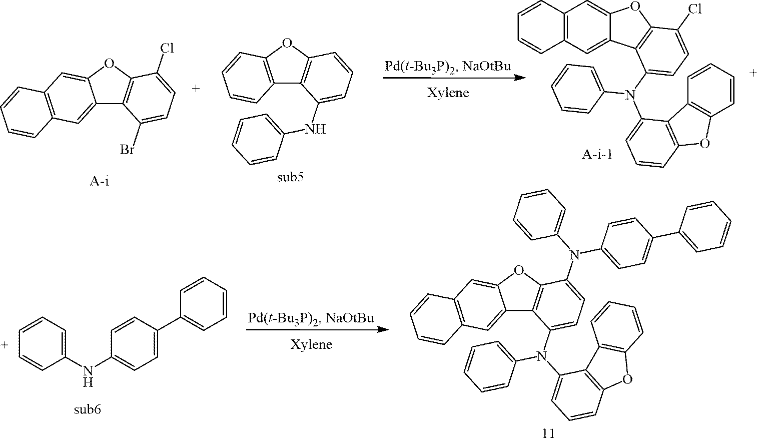

Compound A-i (10 g, 30.2 mmol), sub5 (7.9 g, 30.5 mmol) and sodium tert-butoxide (3.5 g, 36.2 mmol) were added to 200 ml of xylene under a nitrogen atmosphere, and the mixture was stirred and refluxed. Then, bis (tri-tert-butylphosphine)palladium(0) (0.2 g, 0.3 mmol) was added thereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilledunder reduced pressure. The concentrated material was purified by silica gel column chromatography to produce 8.6 g of Compound A-i-1. (yield 56%, MS: [M+H] ⁺= 510) .

Compound A-i-1 (10 g, 19.6 mmol), sub6 (4.9 g, 20 mmol) and sodium tert-butoxide (2.4 g, 25.5 mmol) were added to 200 ml of xylene under a nitrogen atmosphere, and the mixture was stirred and refluxed. Then, bis (tri-tert-butylphosphine)palladium(0) (0.1 g, 0.2 mmol) was added thereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material was purified by silica gel column chromatography to produce 9.2 g of Compound 11. (yield 65%, MS: [M+H] ⁺= 719).

### Synthesis Example 12: Synthesis of Compound 12

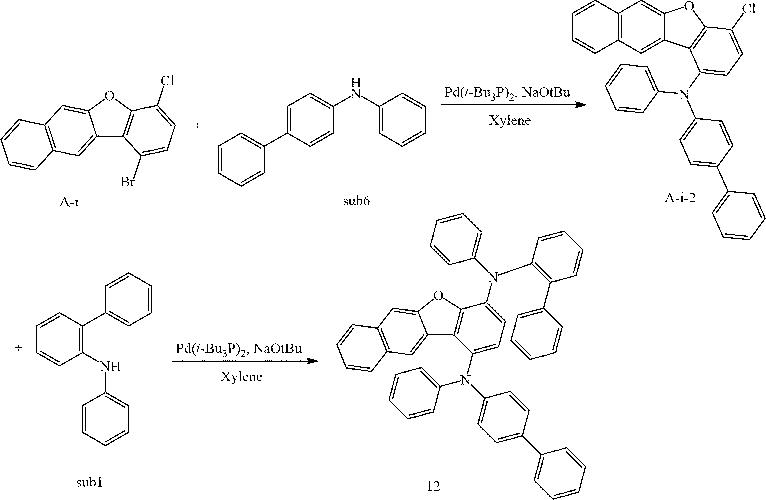

Compound A-i (10 g, 30.2 mmol), sub6 (7.5 g, 30.5 mmol), and sodium tert-butoxide (3.5 g, 36.2 mmol) were added to 200 ml of xylene under a nitrogen atmosphere, and the mixture was stirred and refluxed. Then, bis (tri-tert-butylphosphine)palladium(0) (0.2 g, 0.3 mmol) was added thereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material was purified by silica gel column chromatography to produce 8.8 g of Compound A-i-2. (yield 59%, MS: [M+H] ⁺= 496).

Compound A-i-2 (10 g, 20.2 mmol), sub1 (5 g, 20.6 mmol), and sodium tert-butoxide (2.5 g, 26.2 mmol) were added to 200 ml of xylene under a nitrogen atmosphere, and the mixture was stirred and refluxed. Then, bis (tri-tert-butylphosphine)palladium (0) (0.1 g, 0.2 mmol) was added thereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material was purified by silica gel column chromatography to produce 7.5 g of Compound 12. (yield 53%, MS: [M+H] ⁺= 705).

### Synthesis Example 13: Synthesis of Compound 13

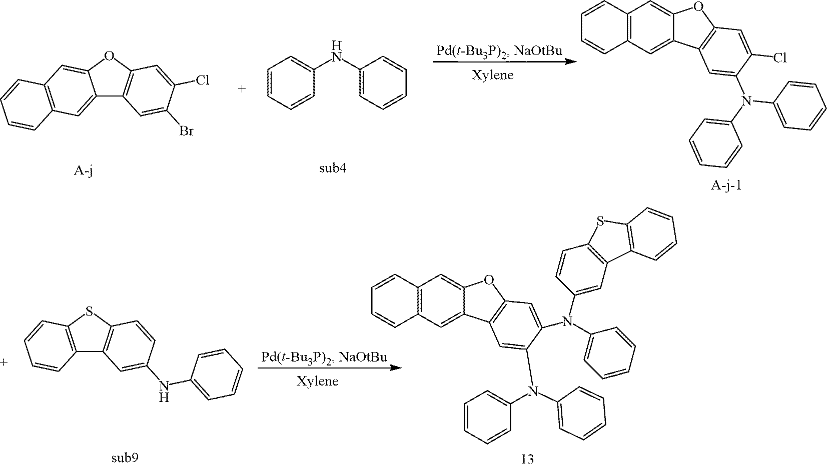

Compound A-j (10 g, 30.2 mmol), sub4 (5.2 g, 30.5 mmol), and sodium tert-butoxide (3.5 g, 36.2 mmol) were added to 200 ml of xylene under a nitrogen atmosphere, and themixture was stirred and refluxed. Then, bis (tri-tert-butylphosphine)palladium (0) (0.2 g, 0.3 mmol) was added thereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material was purified by silica gel column chromatography to produce 8.2 g of Compound A-j-1. (yield 65%, MS: [M+H] ⁺= 420).

Compound A-j-1 (10 g, 23.8 mmol), sub9 (6.7 g, 24.3 mmol), and sodium tert-butoxide (3 g, 31 mmol) were added to 200 ml of xylene under a nitrogen atmosphere, and the mixture was stirred and refluxed. Then, bis (tri-tert-butylphosphine)palladium (0) (0.1 g, 0.2 mmol) was added thereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material was purified by silica gel column chromatography to produce 10.2 g of Compound 13. (yield 53%, MS: [M+H] ⁺= 659).

### Synthesis Example 14: Synthesis of Compound 14

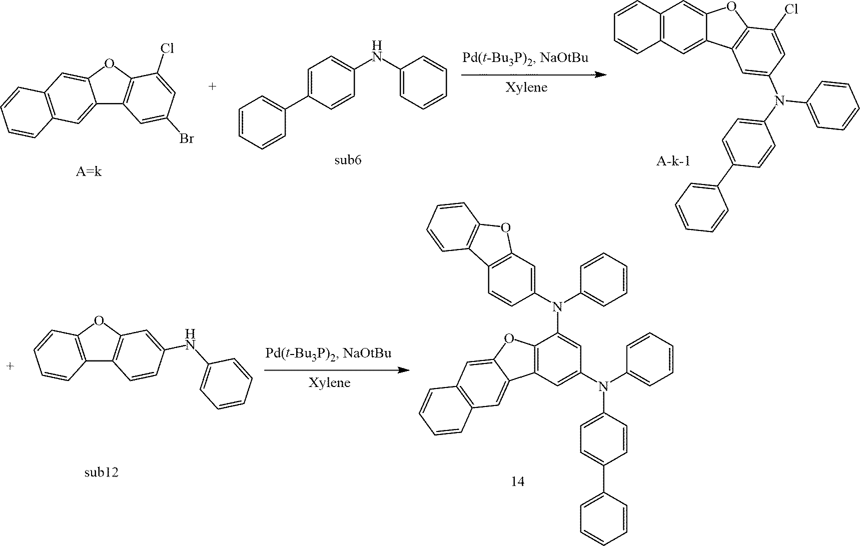

Compound A-k (10 g, 30.2 mmol), sub6 (7.5 g, 30.5 mmol), and sodium tert-butoxide (3.5 g, 36.2 mmol)were added to 200 ml of xylene under a nitrogen atmosphere, and the mixture was stirred and refluxed. Then, bis(tri-tert-butylphosphine)palladium(0) (0.2 g, 0.3 mmol) was added thereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material was purified by silica gel column chromatography to produce 9.7 g of Compound A-k-1. (yield 65%, MS: [M+H] ⁺= 496).

Compound A-k-1 (10 g, 20.2 mmol), sub12 (5.3 g, 20.6 mmol), and sodium tert-butoxide (2.5 g, 26.2 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material was purified by silica gel column chromatography to produce 9.4 g of Compound 14. (yield 53%, MS: [M+H] ⁺= 719).

### Synthesis Example 15: Synthesis of Compound 15

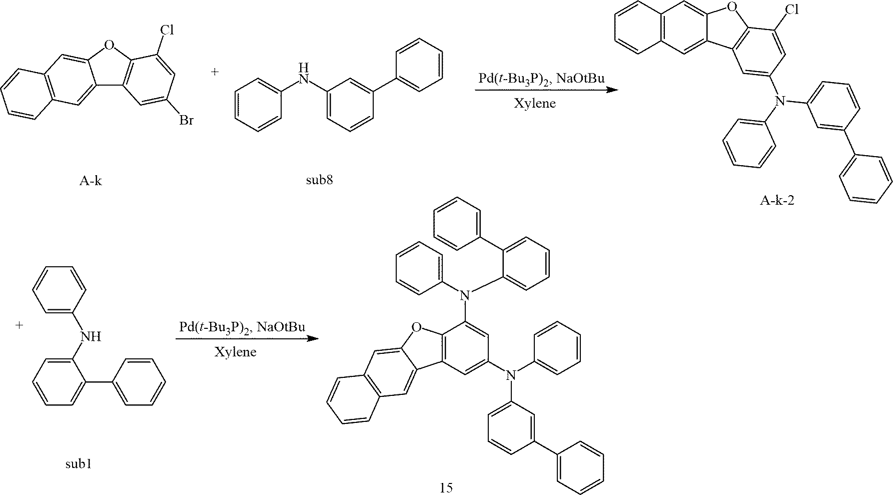

Compound A-k (10 g, 30.2 mmol), sub8 (7.5 g, 30.5 mmol), and sodium tert-butoxide (3.5 g, 36.2 mmol) were added to 200 ml of xylene under a nitrogen atmosphere, and the mixture was stirred and refluxed. Then, bis (tri-tert-butylphosphine)palladium(0) (0.2 g, 0.3 mmol) was addedthereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material was purified by silica gel column chromatography to produce 8.7 g of Compound A-k-2. (yield 58%, MS: [M+H] ⁺= 496) .

Compound A-k-2 (10 g, 20.2 mmol), sub1 (5 g, 20.6 mmol), and sodium tert-butoxide (2.5 g, 26.2 mmol) were added to 200 ml of xylene under a nitrogen atmosphere, and the mixture was stirred and refluxed. Then, bis (tri-tert-butylphosphine)palladium (0) (0.1 g, 0.2 mmol) was added thereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material was purified by silica gel column chromatography to produce 7.7 g of Compound 15. (yield 54%, MS: [M+H] ⁺= 705).

### Synthesis Example 16: Synthesis of Compound 16

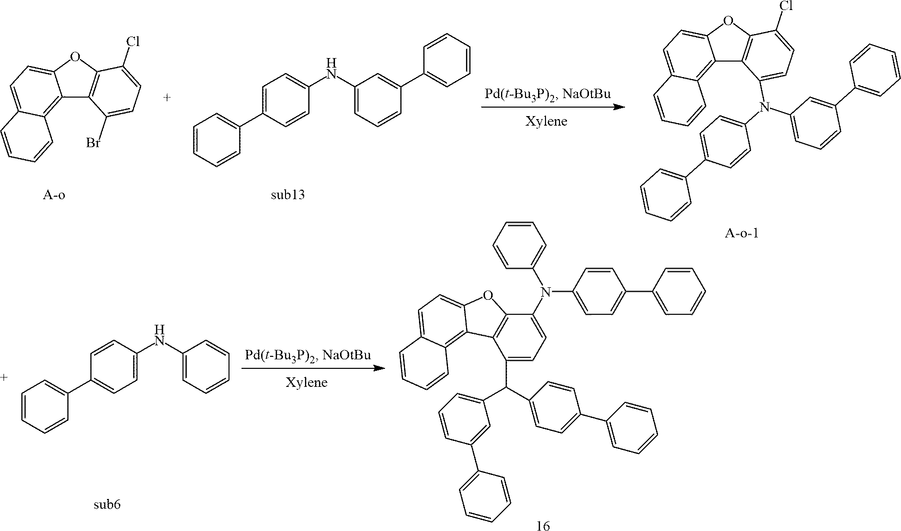

Compound A-o (10 g, 30.2 mmol), sub13 (9.8 g, 30.5 mmol), and sodium tert-butoxide (3.5 g, 36.2 mmol) were added to 200 ml of xylene under a nitrogen atmosphere, and the mixture was stirred and refluxed. Then, bis(tri-tert-butylphosphine)palladium (0) (0.2 g, 0.3 mmol) was added thereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material was purified by silica gel column chromatography to produce 10.5 g of Compound A-o-1. (yield 61%, MS: [M+H] ⁺= 572) .

Compound A-o-1 (10 g, 17.5 mmol), sub6 (4.4 g, 17.8 mmol), and sodium tert-butoxide (2.2 g, 22.7 mmol were added to 200 ml of xylene under a nitrogen atmosphere, and the mixture was stirred and refluxed. Then, bis(tri-tert-butylphosphine)palladium(0) (0.1 g, 0.2 mmol) was added thereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material was purified by silica gel column chromatography to produce 8.6 g of Compound 16. (yield 63%, MS: [M+H] ⁺= 781).

### Synthesis Example 17: Synthesis of Compound 17

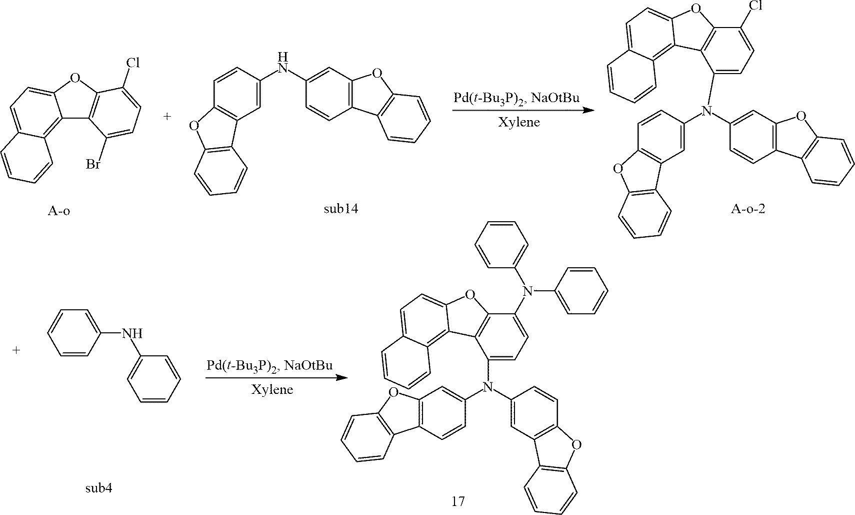

Compound A-o (10 g, 30.2 mmol), sub14 (10.6 g, 30.5 mmol), and sodium tert-butoxide (3.5 g, 36.2 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. After reacting for 5 hours, the reaction mixturewas cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material was purified by silica gel column chromatography to produce 11.4 g of Compound A-o-2. (yield 63%, MS: [M+H] ⁺= 600).

Compound A-o-2 (10 g, 16.7 mmol), sub4 (2.9 g, 17 mmol), and sodium tert-butoxide (2.1 g, 21.7 mmol) were added to 200 ml of xylene under a nitrogen atmosphere, and the mixture was stirred and refluxed. Then, bis(tri-tert-butylphosphine)palladium (0) (0.1 g, 0.2 mmol) was added thereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material was purified by silica gel column chromatography to produce 6.3 g of Compound 17. (yield 52%, MS: [M+H] ⁺= 733).

### Synthesis Example 18: Synthesis of Compound 18

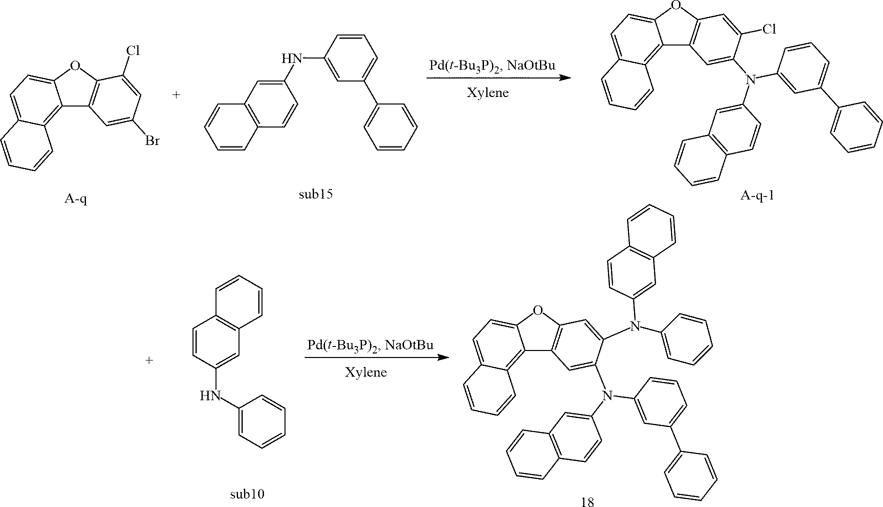

Compound A-q (10 g, 30.2 mmol), sub15 (13.1 g, 33.2 mmol), sodium tert-butoxide (19.2 g, 90.5 mmol) were added to 200 ml of xylene under a nitrogen atmosphere, and the mixture was stirred and refluxed. Then, bis (tri-tert-butylphosphine)palladium (0) (0.3 g, 0.6 mmol) was added thereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material was purified by silica gel column chromatography to produce 8.9 g of Compound A-q-1. (yield 54%, MS: [M+H] ⁺= 546) .

Compound A-q-1 (10 g, 18.3 mmol), sub10 (4.1 g, 18.7 mmol), and sodium tert-butoxide (2.3 g, 23.8 mmol) were added to 200 ml of xylene under a nitrogen atmosphere, and the mixture was stirred and refluxed. Then, bis(tri-tert-butylphosphine)palladium (0) (0.1 g, 0.2 mmol) was added thereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material was purified by silica gel column chromatography to produce 9.3 g of Compound 18. (yield 70%, MS: [M+H] ⁺= 729).

### Synthesis Example 19: Synthesis of Compound 19

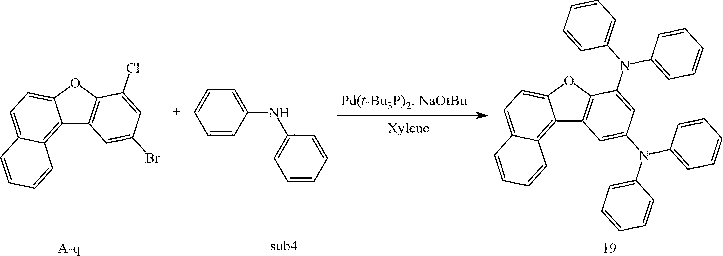

Compound A-q (10 g, 30.2 mmol), sub4 (10.5 g, 61.8 mmol), and sodium tert-butoxide (7.2 g, 75.4 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material was purified by silica gel column chromatography to produce 9.3 g of Compound 19. (yield 56%, MS: [M+H] ⁺= 553).

### Synthesis Example 20: Synthesis of Compound 20

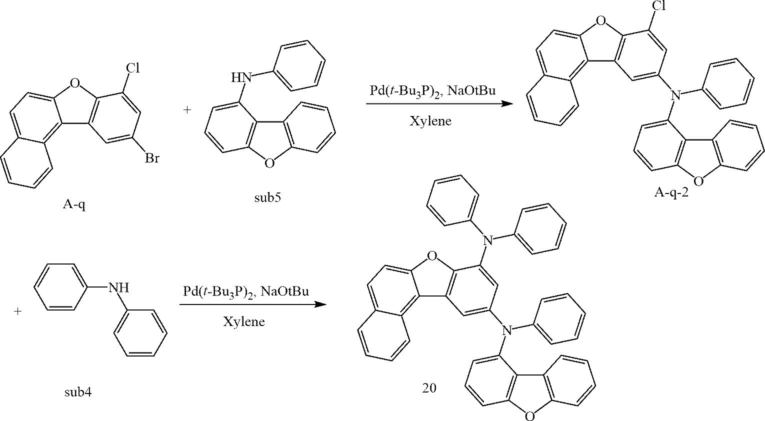

Compound A-q (10 g, 30.2 mmol), sub5 (7.9 g, 30.5 mmol), and sodium tert-butoxide (3.5 g, 36.2 mmol) were added to 200 ml of xylene under a nitrogen atmosphere, and the mixture was stirred and refluxed. Then, bis (tri-tert-butylphosphine)palladium(0) (0.2 g, 0.3 mmol) was added thereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material was purified by silica gel column chromatography to produce 9.8 g of Compound A-q-2. (yield 64%, MS: [M+H] ⁺= 510).

Compound A-q-2 (10 g, 19.6 mmol), sub4 (3.4 g, 20 mmol), and sodium tert-butoxide (2.4 g, 25.5 mmol) were added to 200 ml of xylene under a nitrogen atmosphere, and the mixture was stirred and refluxed. Then, bis (tri-tert-butylphosphine)palladium(0) (0.1 g, 0.2 mmol) was added thereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material was purified by silica gel column chromatography to produce 7.9 g of Compound 20. (yield 63%, MS: [M+H] ⁺= 643).

### Synthesis Example 21: Synthesis of Compound 21

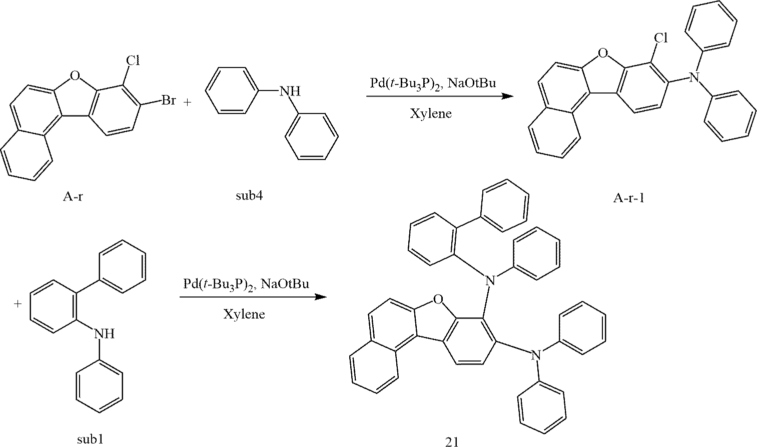

Compound A-r (10 g, 30.2 mmol), sub4 (5.2 g, 30.5 mmol), sodium tert-butoxide (3.5 g, 36.2 mmol) were added to 200 ml of xylene under a nitrogen atmosphere, and the mixture was stirred and refluxed. Then, bis(tri-tert-butylphosphine)palladium(0) (0.2 g, 0.3 mmol) was added thereto. After reacting for 5 hours, the reaction mixture was cooled to room temperature, the solvent removed underreduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material was purified by silica gel column chromatography to produce 6.6 g of Compound A-r-1. (yield 52%, MS: [M+H] ⁺= 420) .

Compound A-r-1 (10 g, 23.8 mmol), sub1 (6 g, 24.3 mmol), and sodium tert-butoxide (3 g, 31 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material was purified by silica gel column chromatography to produce 10 g of Compound 21. (yield 67%, MS: [M+H] ⁺= 629).

### Synthesis Example 22: Synthesis of Compound 22

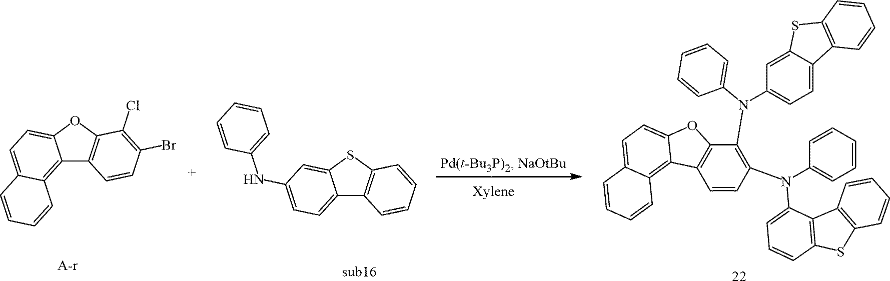

Compound A-r (10 g, 30.2 mmol), sub16 (17 g, 61.8 mmol), and sodium tert-butoxide (7.2 g, 75.4 mmol) were added to200 ml of xylene under a nitrogen atmosphere, and the mixture was stirred and refluxed. Then, bis (tri-tert-butylphosphine)palladium (0) (0.3 g, 0.6 mmol) was added thereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material was purified by silica gel column chromatography to produce 15.9 g of Compound 22. (yield 69%, MS: [M+H] ⁺= 765) .

### Synthesis Example 23: Synthesis of Compound 23

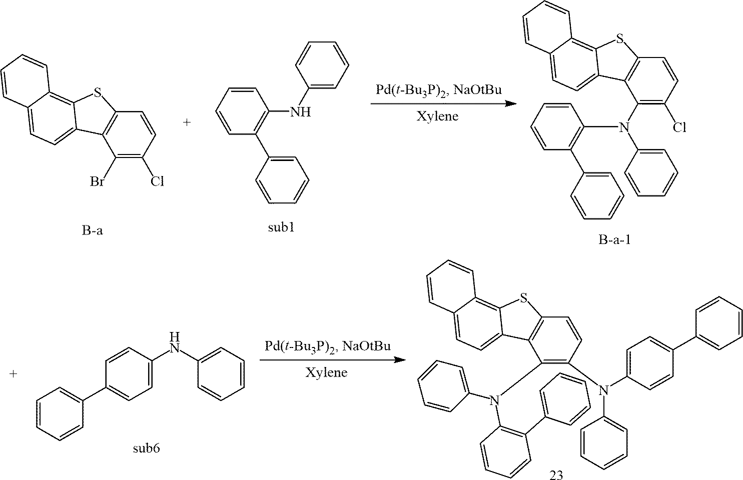

Compound B-a (10 g, 28.8 mmol), sub1 (7.1 g, 29.1 mmol), and sodium tert-butoxide (3.3 g, 34.5 mmol) were added to 200 ml of xylene under a nitrogen atmosphere, and the mixture was stirred and refluxed. Then, bis(tri-tert-butylphosphine)palladium(0) (0.1 g, 0.3 mmol) was addedthereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material was purified by silica gel column chromatography to produce 10.1 g of Compound B-a-1. (yield 69%, MS: [M+H] ⁺= 512).

Compound B-a-1 (10 g, 19.5 mmol), sub6 (4.9 g, 19.9 mmol), and sodium tert-butoxide (2.4 g, 25.4 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material was purified by silica gel column chromatography to produce 8.2 g of Compound 23. (yield 58%, MS: [M+H] ⁺= 721).

### Synthesis Example 24: Synthesis of Compound 24

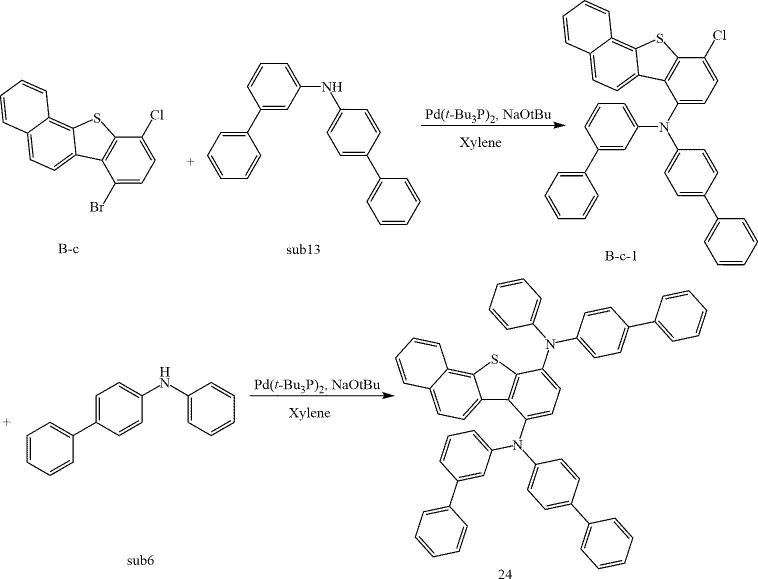

Compound B-c (10 g, 28.8 mmol), sub13 (9.3 g, 29.1 mmol), and sodium tert-butoxide (3.3 g, 34.5 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material was purified by silica gel column chromatography to produce 11.1 g of Compound B-c-1. (yield 66%, MS: [M+H] ⁺= 588).

Compound B-c-1 (10 g, 17 mmol), sub6 (4.3 g, 17.3 mmol), and sodium tert-butoxide (2.1 g, 22.1 mmol) were added to 200 ml of xylene under a nitrogen atmosphere, and the mixture was stirred and refluxed. Then, bis(tri-tert-butylphosphine)palladium (0) (0.1 g, 0.2 mmol) was added thereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material was purified by silica gel column chromatography to produce 9.5 g of Compound 24. (yield 70%, MS: [M+H]⁺= 797).

### Synthesis Example 25: Synthesis of Compound 25

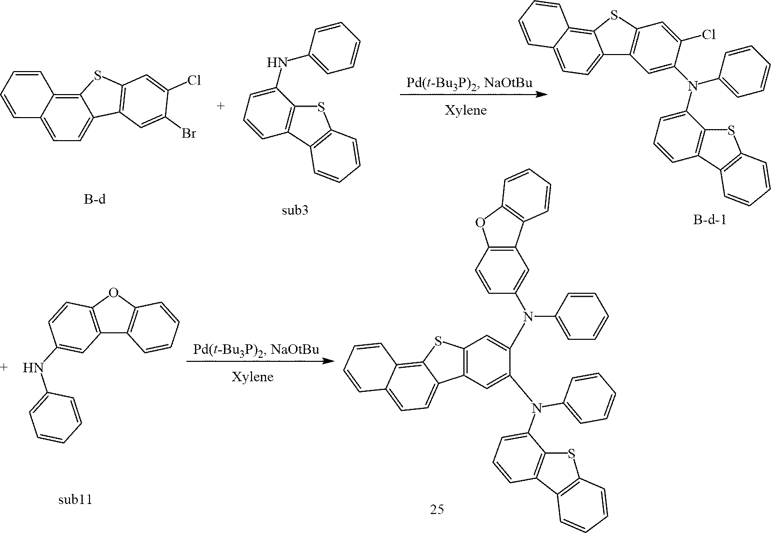

Compound B-d (10 g, 28.8 mmol), sub3 (8 g, 29.1 mmol), and sodium tert-butoxide (3.3 g, 34.5 mmol) were added to 200 ml of xylene under a nitrogen atmosphere, and themixture was stirred and refluxed. Then, bis(tri-tert-butylphosphine)palladium(0) (0.1 g, 0.3 mmol) was added thereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material was purified by silica gel column chromatography to produce 10.7 g of Compound B-d-1. (yield 69%, MS: [M+H]⁺= 542).

Compound B-d-1 (10 g, 18.4 mmol), sub11 (4.9 g, 18.8 mmol), and sodium tert-butoxide (2.3 g, 24 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material was purified by silica gel column chromatography to produce 9.6 g of Compound 25. (yield 68%, MS: [M+H]⁺= 756).

### Synthesis Example 26: Synthesis of Compound 26

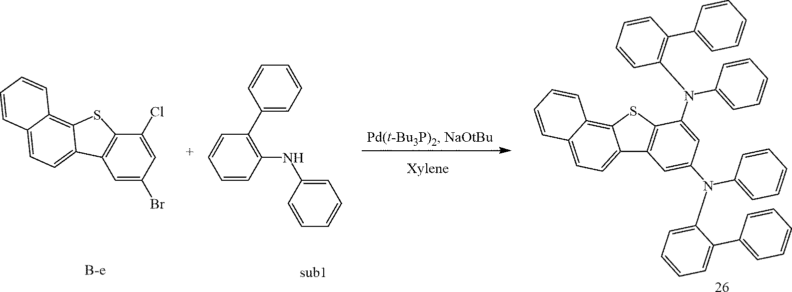

Compound B-e (10 g, 28.8 mmol), sub1 (14.5 g, 59 mmol), and sodium tert-butoxide (6.9 g, 71.9 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material was purified by silica gel column chromatography to produce 11 g of Compound 26. (yield 53%, MS: [M+H]⁺= 721).

### Synthesis Example 27: Synthesis of Compound 27

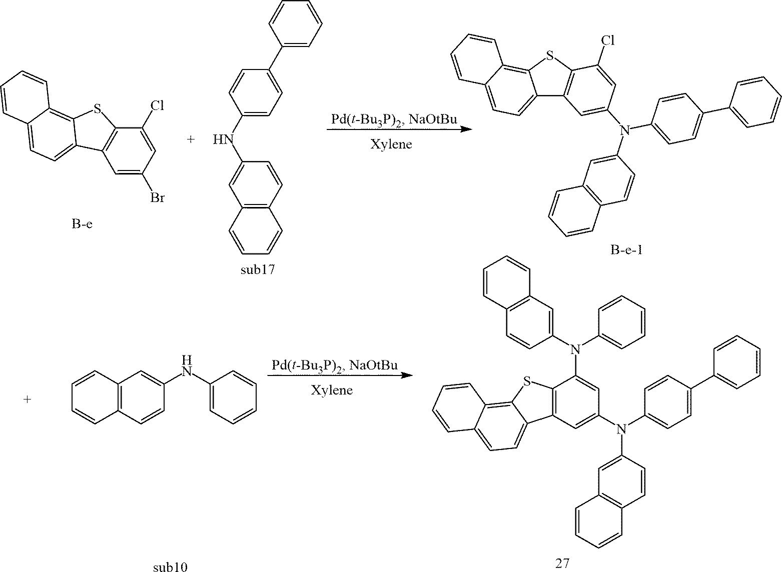

Compound B-e (10 g, 28.8 mmol), sub17 (8.6 g, 29.1 mmol), and sodium tert-butoxide (3.3 g, 34.5 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material was purified by silica gel column chromatography to produce 8.4 g of Compound B-e-1. (yield 52%, MS: [M+H]⁺= 562).

Compound B-e-1 (10 g, 17.8 mmol), sub10 (4 g, 18.1 mmol), and sodium tert-butoxide (2.2 g, 23.1 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material was purified by silica gel column chromatography to produce 7.7 g of Compound 27. (yield 58%, MS: [M+H]⁺= 745) .

### Synthesis Example 28: Synthesis of Compound 28

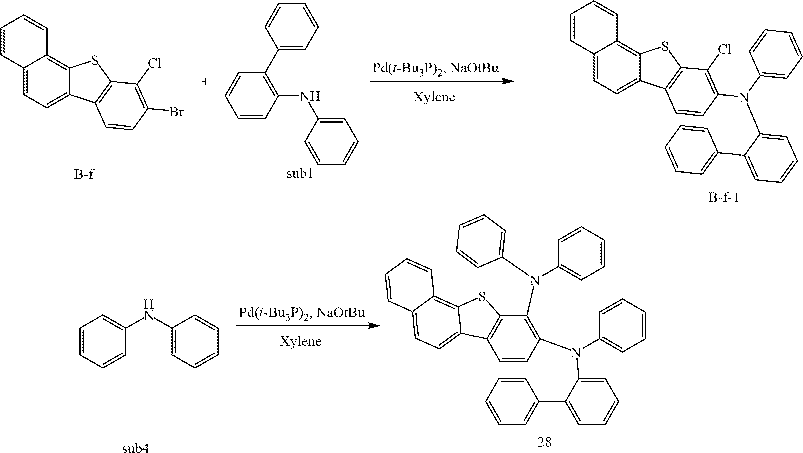

Compound B-f (10 g, 28.8 mmol), sub1 (7.1 g, 29.1 mmol), and sodium tert-butoxide (3.3 g, 34.5 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material waspurified by silica gel column chromatography to produce 9.3 g of Compound B-f-1. (yield 63%, MS: [M+H]⁺= 512).

Compound B-f-1 (10 g, 19.5 mmol), sub4 (3.4 g, 19.9 mmol), and sodium tert-butoxide (2.4 g, 25.4 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material was purified by silica gel column chromatography to produce 7 g of Compound 28. (yield 56%, MS: [M+H]⁺= 645).

### Synthesis Example 29: Synthesis of Compound 29

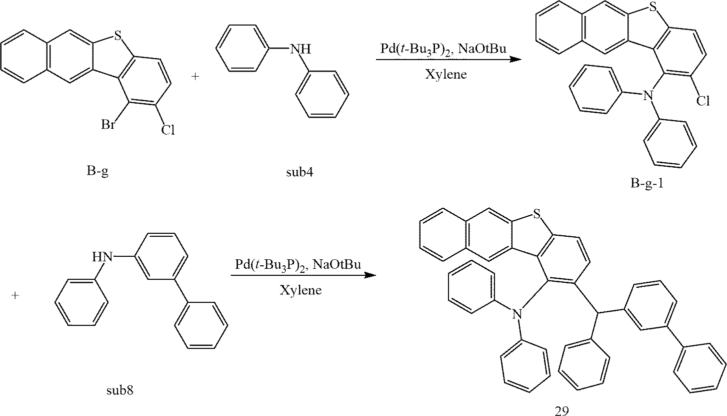

Compound B-g (10 g, 28.8 mmol), sub4 (4.9 g, 29.1 mmol), and sodium tert-butoxide (3.3 g, 34.5 mmol) were added to 200 ml of xylene under a nitrogen atmosphere, and the mixture was stirred and refluxed. Then, bis(tri-tert-butylphosphine)palladium (0) (0.1 g, 0.3 mmol) was added thereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material was purified by silica gel column chromatography to produce 8.3 g of Compound B-g-1. (yield 67%, MS: [M+H]⁺= 433).

Compound B-g-1 (10 g, 23.1 mmol), sub8 (5.8 g, 23.6 mmol), and sodium tert-butoxide (2.9 g, 30 mmol) were added to 200 ml of xylene under a nitrogen atmosphere, and the mixture was stirred and refluxed. Then, bis(tri-tert-butylphosphine)palladium (0) (0.1 g, 0.2 mmol) was added thereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material was purified by silica gel column chromatography to produce 9.4 g of Compound 29. (yield 63%, MS: [M+H]⁺= 645).

### Synthesis Example 30: Synthesis of Compound 30

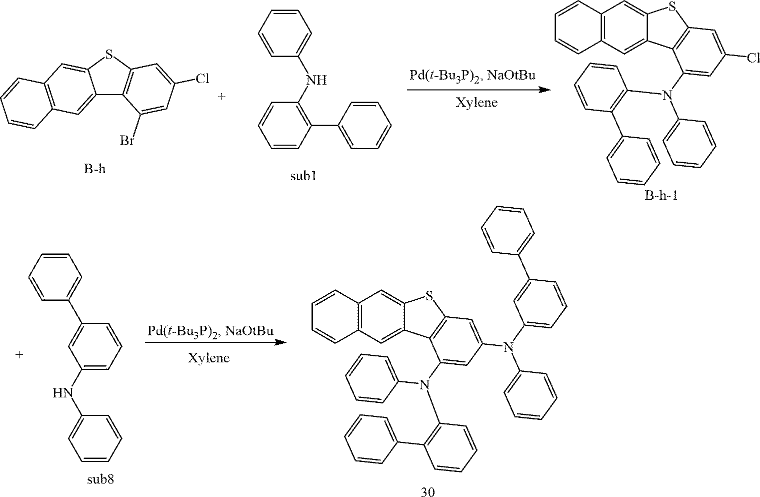

Compound B-h (10 g, 28.8 mmol), sub1 (7.1 g, 29.1 mmol), and sodium tert-butoxide (3.3 g, 34.5 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material was purified by silica gel column chromatography to produce 8.1 g of Compound B-h-1. (yield 55%, MS: [M+H]⁺= 512).

Compound B-h-1 (10 g, 23.1 mmol), sub8 (5.8 g, 23.6 mmol), and sodium tert-butoxide (2.9 g, 30 mmol) were added to 200 ml of xylene under a nitrogen atmosphere, and the mixture was stirred and refluxed. Then, bis(tri-tert-butylphosphine)palladium (0) (0.1 g, 0.2 mmol) was added thereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material was purified by silica gel column chromatography to produce 8.7 g of Compound 30. (yield 52%, MS: [M+H]⁺= 721).

### Synthesis Example 31: Synthesis of Compound 31

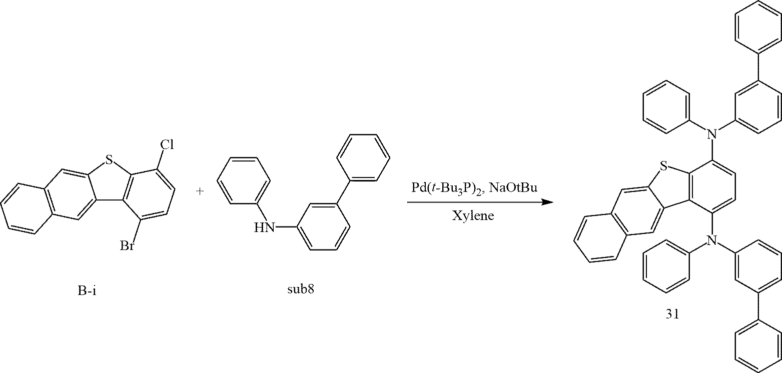

Compound B-i (10 g, 28.8 mmol), sub8 (14.5 g, 59 mmol), and sodium tert-butoxide (6.9 g, 71.9 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. After reacting for 5 hours, the reaction mixture was cooled to room temperature, the solvent removed underreduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material was purified by silica gel column chromatography to produce 12.4 g of Compound 31. (yield 60%, MS: [M+H]⁺= 721).

### Synthesis Example 32: Synthesis of Compound 32

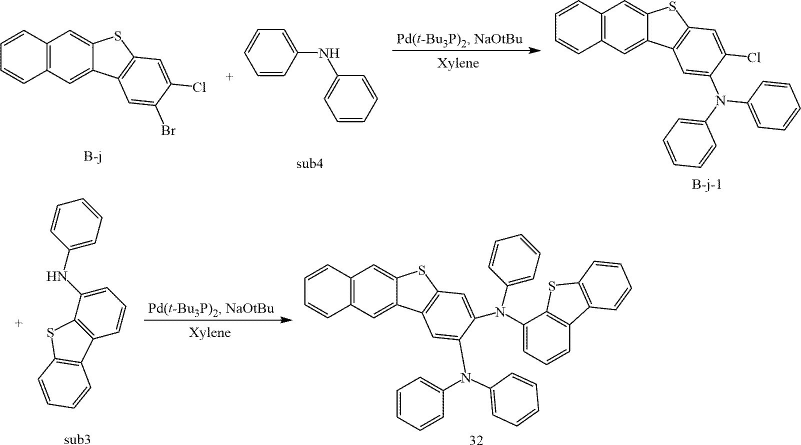

Compound B-j (10 g, 28.8 mmol), sub4 (4.9 g, 29.1 mmol), sodium tert-butoxide (3.3 g, 34.5 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto,stirred, and then filtered, and the filtrate was distilled under reduced pressure. The concentrated material was purified by silica gel column chromatography to produce 6.6 g of Compound B-j-1. (yield 53%, MS: [M+H]⁺= 436) .

Compound B-j-1 (10 g, 22.9 mmol), sub3 (6.4 g, 23.4 mmol), and sodium tert-butoxide (2.9 g, 29.8 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material was purified by silica gel column chromatography to produce 8 g of Compound 32. (yield 52%, MS: [M+H]⁺= 675).

### Synthesis Example 33: Synthesis of Compound 33

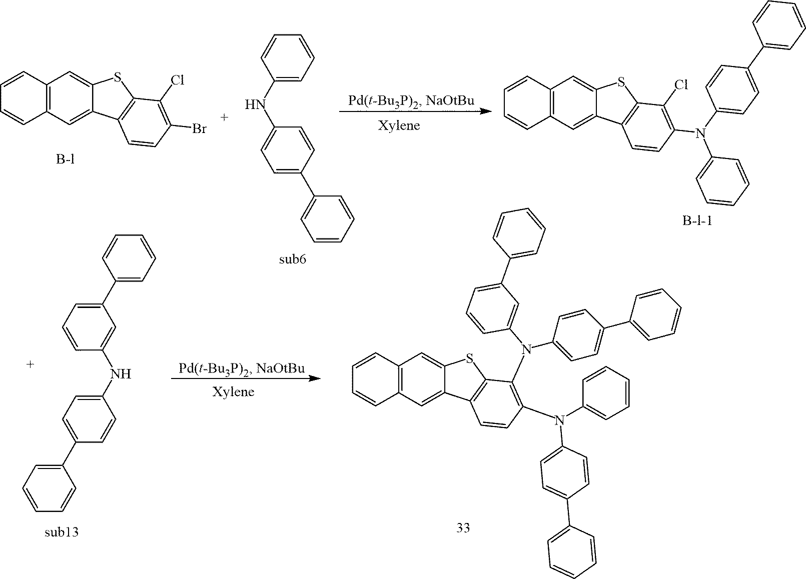

Compound B-1 (10 g, 28.8 mmol), sub6 (7.1 g, 29.1 mmol), and sodium tert-butoxide (3.3 g, 34.5 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material was purified by silica gel column chromatography to produce 8.2 g of Compound B-l-1. (yield 56%, MS: [M+H]⁺= 512).

Compound B-l-1 (10 g, 19.5 mmol), sub13 (6.4 g, 19.9 mmol), and sodium tert-butoxide (2.4 g, 25.4 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material was purified by silica gel column chromatography to produce 10.9 g of Compound 33. (yield 70%, MS: [M+H]⁺= 797).

### Synthesis Example 34: Synthesis of Compound 34

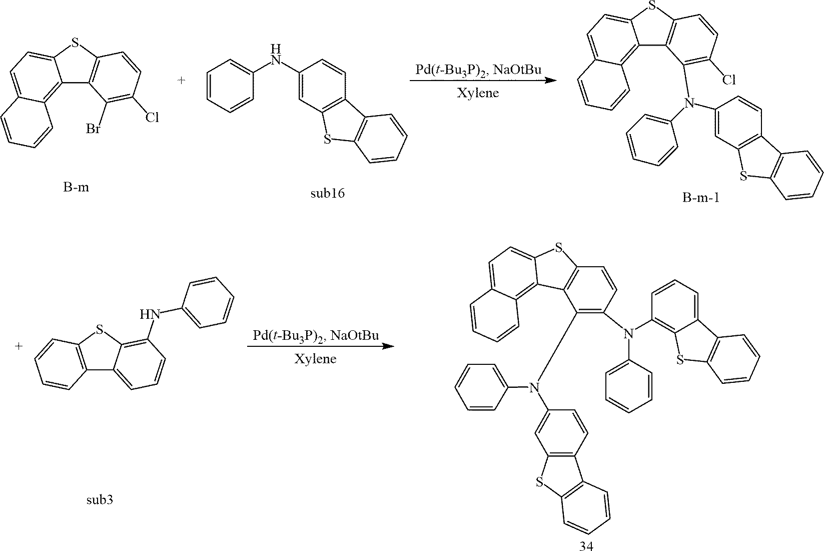

Compound B-m (10 g, 28.8 mmol), sub16 (8 g, 29.1 mmol), and sodium tert-butoxide (3.3 g, 34.5 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilledunder reduced pressure. The concentrated material was purified by silica gel column chromatography to produce 10.3 g of Compound B-m-1. (yield 97%, MS: [M+H]⁺= 542).

Compound B-m-1 (10 g, 18.4 mmol), sub3 (5.2 g, 18.8 mmol), and sodium tert-butoxide (2.3 g, 24 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material was purified by silica gel column chromatography to produce 9.1 g of Compound 34. (yield 63%, MS: [M+H]⁺= 781).

### Synthesis Example 35: Synthesis of Compound 35

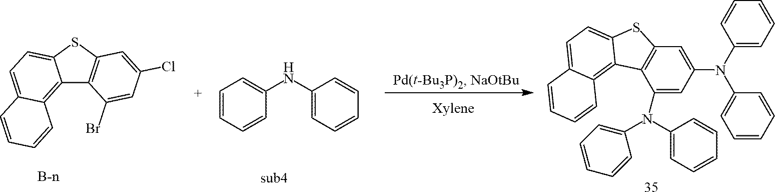

Compound B-n (10 g, 28.8 mmol), sub4 (10 g, 59 mmol), and sodium tert-butoxide (6.9 g, 71.9 mmol) were added to 200 ml of xylene under a nitrogen atmosphere, and the mixture was stirred and refluxed. Then, bis(tri-tert-butylphosphine)palladium(0) (0.3 g, 0.6 mmol) was added thereto. After reacting for 5 hours, the reaction mixturewas cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material was purified by silica gel column chromatography to produce 9.3 g of Compound 35. (yield 57%, MS: [M+H]⁺= 569).

### Synthesis Example 36: Synthesis of Compound 36

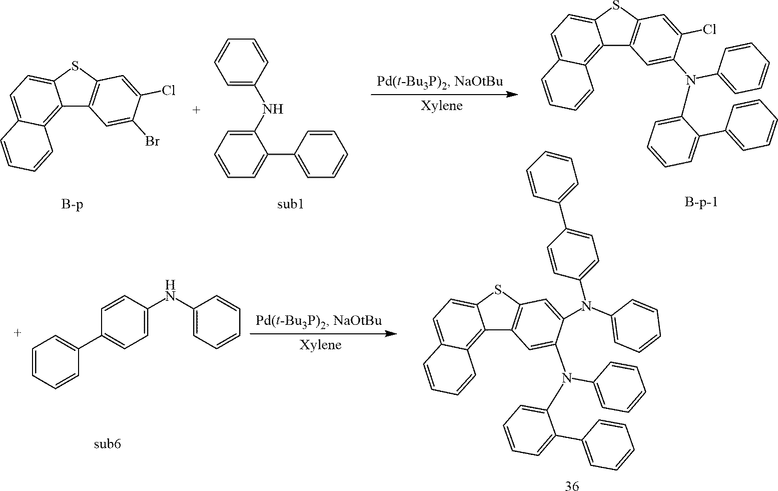

Compound B-p (10 g, 28.8 mmol), sub1 (7.1 g, 29.1 mmol), and sodium tert-butoxide (3.3 g, 34.5 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. After reacting for 5 hours, the reaction mixturewas cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material was purified by silica gel column chromatography to produce 7.9 g of Compound B-p-1. (yield 54%, MS: [M+H]⁺= 512) .

Compound B-p-1 (10 g, 19.5 mmol), sub6 (4.9 g, 19.9 mmol), and sodium tert-butoxide (2.4 g, 25.4 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material was purified by silica gel column chromatography to produce 8.4 g of Compound 36. (yield 60%, MS: [M+H]⁺= 721).

### Synthesis Example 37: Synthesis of Compound 37

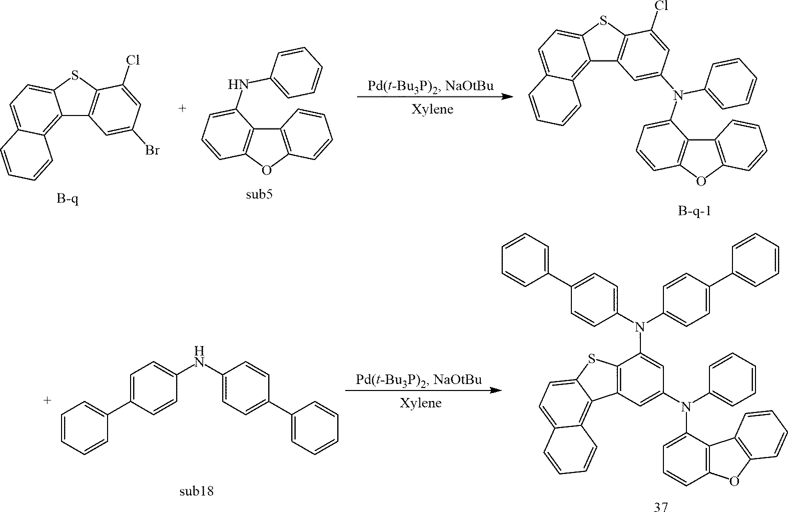 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21]

Compound B-q (10 g, 28.8 mmol), sub5 (7.5 g, 29.1 mmol), and sodium tert-butoxide (3.3 g, 34.5 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material was purified by silica gel column chromatography to produce 8.9 g of Compound B-q-1. (yield 59%, MS: [M+H]⁺= 526).

Compound B-q-1 (10 g, 19 mmol), sub18 (6.2 g, 19.4 mmol), and sodium tert-butoxide (2.4 g, 24.7 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material was purified by silica gel column chromatography to produce 7.7 g of Compound 37. (yield 50%, MS: [M+H]⁺= 811).

### Synthesis Example 38: Synthesis of Compound 38

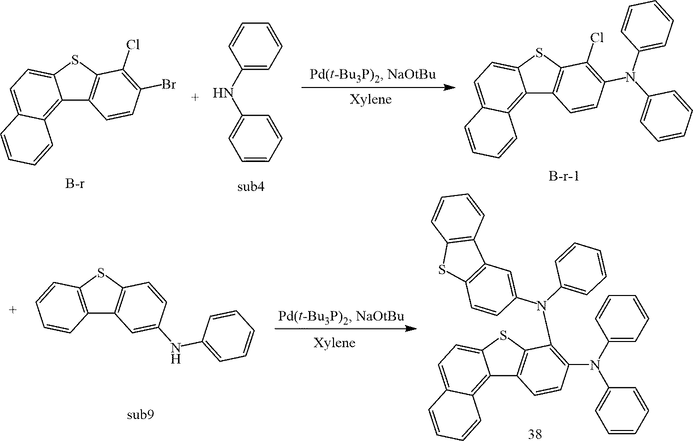 [SMILES: N#CC(=C1C(=C(C#N)c2c(F)c(F)c(C#N)c(F)c2F)C1=C(C#N)c1c(F)c(F)c(C#N)c(F)c1F)c1c(F)c(F)c(C#N)c(F)c1F]

Compound B-r (10 g, 28.8 mmol), sub4 (4.9 g, 29.1 mmol), and sodium tert-butoxide (3.3 g, 34.5 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material waspurified by silica gel column chromatography to produce 7.6 g of Compound B-r-1. (yield 61%, MS: [M+H]⁺= 436) .

Compound B-r-1 (10 g, 22.9 mmol), sub9 (6.4 g, 23.4 mmol), and sodium tert-butoxide (2.9 g, 29.8 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material was purified by silica gel column chromatography to produce 9 g of Compound 38. (yield 58%, MS: [M+H]⁺= 675).

### Synthesis Example 39: Synthesis of Compound 39

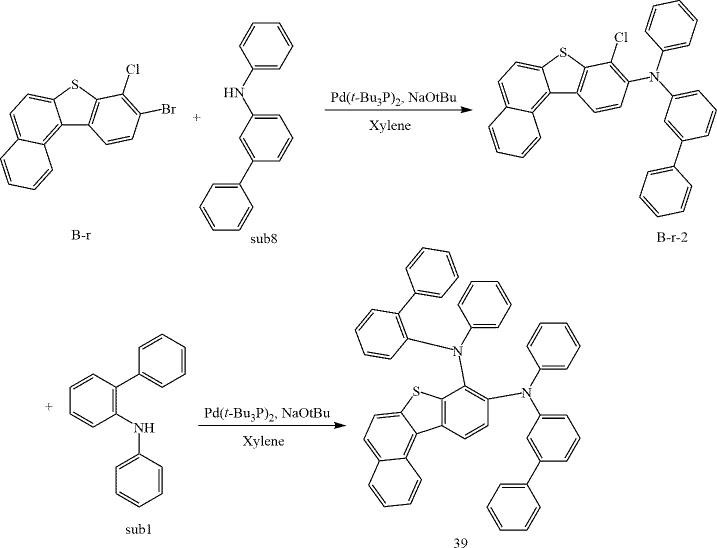 [SMILES: CC1(C)c2ccccc2-c2ccc(-n3c4ccc(-c5ccc(N(c6ccccc6)c6ccccc6)cc5)cc4c4cc(-c5ccc(N(c6ccccc6)c6ccccc6)cc5)ccc43)cc21]

Compound B-r (10 g, 28.8 mmol), sub8 (7.1 g, 29.1 mmol), and sodium tert-butoxide (3.3 g, 34.5 mmol) were added to 200 ml of xylene under a nitrogen atmosphere, and the mixture was stirred and refluxed. Then, bis(tri-tert-butylphosphine)palladium(0) (0.1 g, 0.3 mmol) was added thereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material was purified by silica gel column chromatography to produce 9.3 g of Compound B-r-2. (yield 63%, MS: [M+H]⁺= 512).

Compound B-r-2 (10 g, 19.5 mmol), sub1 (4.9 g, 19.9 mmol), sodium tert-butoxide (2.4 g, 25.4 mmol) were added to 200 ml of xylene under a nitrogen atmosphere, and the mixture was stirred and refluxed. Then, bis(tri-tert-butylphosphine)palladium(0) (0.1 g, 0.2 mmol) was added thereto. After reacting for 5 hours, the reaction mixture was cooled to room temperature, the solvent removed under reduced pressure. This was again dissolved in chloroform, washed twice with water, and then the organic layer was separated, anhydrous magnesium sulfate was added thereto, stirred, and then filtered, and the filtrate was distilled under reduced pressure. The concentrated material was purified by silica gel column chromatography to produce 8 g of Compound 39. (yield 57%, MS: [M+H]⁺= 721).

### EXAMPLES AND COMPARATIVE EXAMPLES

### Example 1

A glass substrate on which a thin film of ITO (indium tin oxide) was coated in a thickness of 1,000 Å was put into distilled water containing a detergent dissolved therein and ultrasonically washed. In this case, the detergent used was a product commercially available from Fisher Co. and the distilled water was one which had been twice filtered by using a filter commercially available from Millipore Co. The ITO was washed for 30 minutes, and ultrasonic washing was then repeated twice for 10 minutes by using distilled water. After the washing with distilled water was completed, the substrate was ultrasonically washed with isopropyl alcohol, acetone, and methanol solvent, and dried, after which it was transported to a plasma cleaner. Then, the substrate was cleaned with oxygen plasma for 5 minutes, and then transferred to a vacuum evaporator.

On the ITO transparent electrode thus prepared, the following Compound HI-1 was formed to a thickness of 1150 Å as a hole injection layer, but the following Compound A-1 was p-doped at a concentration of 1.5 wt.%. The following Compound HT-1 was vacuum deposited to a film thickness of 800 Å on the hole injection layer to form a hole transport layer. Then, the following Compound EB-1 was vacuum deposited to a film thickness of 150 Å on the hole transport layer to form an electron blocking layer. Then, the following Compound RH-1, the Compound 1 prepared in Synthesis Example 1 as a host, and the following Compound Dp-7 as a dopant were vacuum deposited in a weight ratio of 49:49:2 on the EB-1-deposited layer to form a red light emitting layer with a thickness of 400 Å. The following Compound HB-1 was vacuum deposited to a film thickness of 30 Å on the light emitting layer to form a hole blocking layer. The following Compound ET-1 and the following Compound LiQ were vacuum deposited in a ratio of 2:1 on the hole blocking layer to form an electron injection and transport layer with a film thickness of 300 Å. Lithium fluoride (LiF) and aluminum were sequentially deposited to have a thickness of 12 Å and 1,000 Å, respectively, on the electron injection and transport layer, thereby forming a cathode.

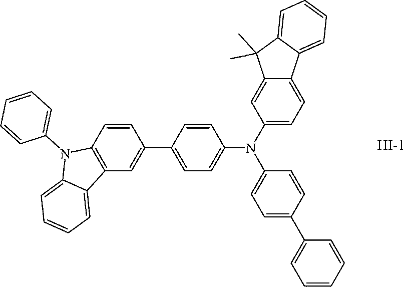 [SMILES: CC1(C)c2ccccc2-c2ccc(N(c3ccc(-c4ccccc4)cc3)c3cccc4c3-c3ccccc3C43c4ccccc4-c4ccccc43)cc21]

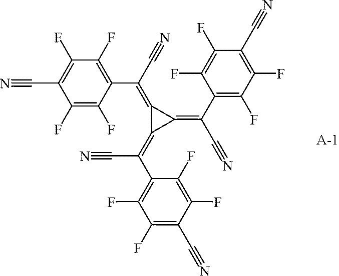 [SMILES: c1ccc(-c2nc(-c3ccccc3)nc(-n3c4ccccc4c4cc(-c5ccc6c(c5)c5ccccc5n6-c5ccccc5)ccc43)n2)cc1]

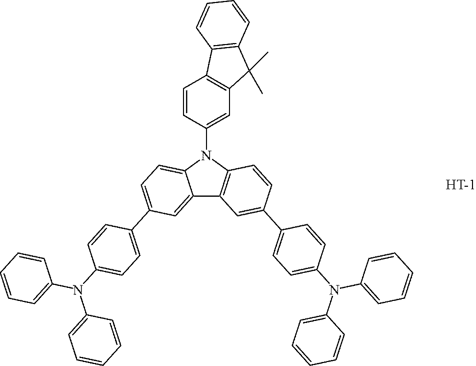

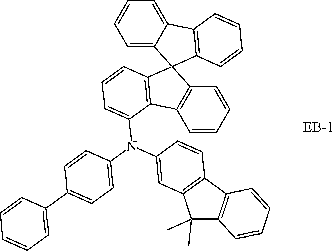 [SMILES: c1ccc(-c2nc(-c3ccccc3)nc(-c3ccc(-c4c(-c5ccc6c7ccccc7c7ccccc7c6c5)ccc5ccccc45)cc3)n2)cc1]

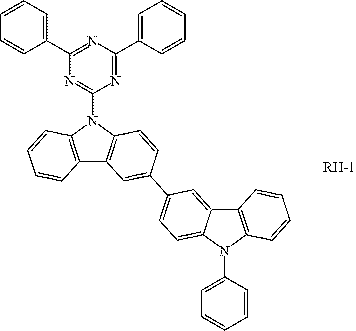 [SMILES: c1ccc(-c2nc(-c3ccccc3)nc(-c3ccc(-c4ccc5ccc(-c6ccc(-c7nc(-c8ccccc8)nc(-c8ccccc8)n7)cc6)cc5c4)cc3)n2)cc1]

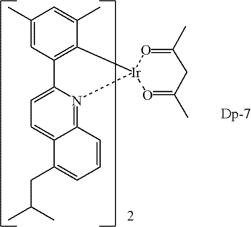

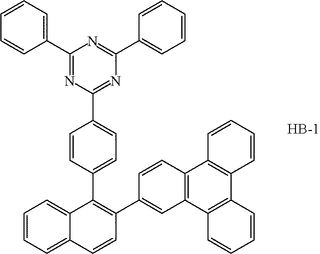 [SMILES: c1ccc(N(c2ccccc2)c2ccc3oc4cc(N(c5ccccc5)c5ccc6c(c5)sc5ccccc56)c5ccccc5c4c3c2)cc1]

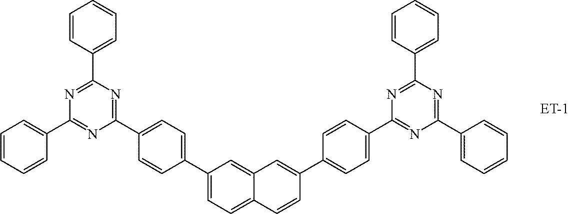 [SMILES: c1ccc(N(c2ccc3c(c2)oc2c4ccccc4c(N(c4ccccc4)c4ccccc4)cc32)c2ccc3sc4ccccc4c3c2)cc1]

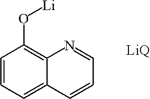 [SMILES: c1ccc(-c2cccc(N(c3ccc4cc5c(cc4c3)oc3cc(N(c4cccc(-c6ccccc6)c4)c4ccc6c(c4)oc4ccccc46)ccc35)c3ccc4c(c3)oc3ccccc34)c2)cc1]

In the above-mentioned processes, the deposition rates of the organic materials were maintained at 0.4 to 0.7 Å/sec, the deposition rates of lithium fluoride and the aluminum of the cathode were maintained at 0.3 Å/sec and 2 Å/sec, respectively, and the degree of vacuum during the deposition was maintained at 2×10⁻⁷ to 5×10⁻⁶ torr, thereby manufacturing an organic light emitting device.

### Example 2 to Example 39

The organic light emitting devices were manufactured in the same manner as in Example 1, except that the compounds shown in Table 1 below were used instead of Compound 1 in the organic light emitting device of Example 1.

### Comparative Examples 1 to 8

The organic light emitting devices were manufactured in the same manner as in Example 1, except that the compounds (C1 to C8) shown in Table 1 below were used instead of Compound 1 in the organic light emitting device of Example 1. The compounds used in the Comparative Examples 1 to 8 are as follows:

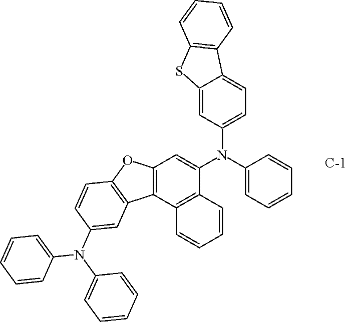 [SMILES: CC1(C)c2ccccc2-c2cccc(N(c3ccccc3)c3cc(-c4ccc5c(c4)oc4c6ccccc6ccc54)cc(N(c4ccccc4)c4ccccc4)c3)c21]

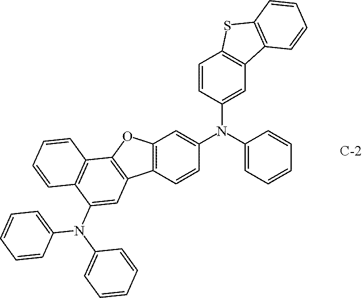 [SMILES: c1ccc(N(c2ccccc2)c2c3ccccc3c(N(c3ccccc3)c3ccccc3)c3c2oc2ccccc23)cc1]

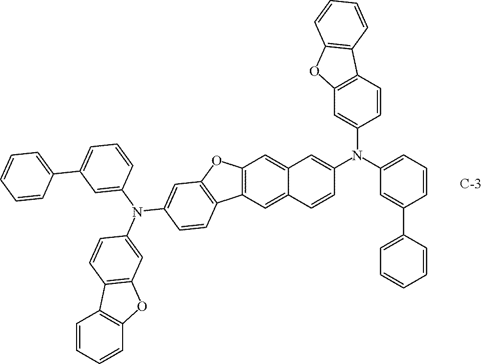 [SMILES: c1ccc(N(c2ccccc2)c2cc(-c3ccc4sc5cc(-c6cc(N(c7ccccc7)c7ccccc7)cc7c6sc6ccccc67)ccc5c4c3)c3oc4c5ccccc5ccc4c3c2)cc1]

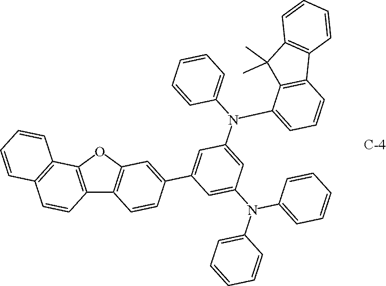 [SMILES: c1ccc(N(c2ccc3ccccc3c2)c2cc(N(c3ccccc3)c3ccc4ccccc4c3)c3oc4c5ccccc5c(-c5ccc6c(c5)c5cc7ccccc7cc5n6-c5ccccc5)cc4c3c2)cc1]

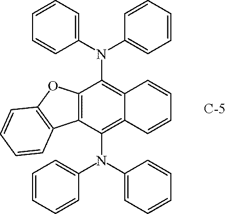 [SMILES: c1ccc(N(c2cc(N(c3ccccc3)c3ccc4sc5ccccc5c4c3)c3oc4c5ccccc5c(-c5cc6c7ccccc7n(-c7ccccc7)c6c6ccccc56)cc4c3c2)c2cc3ccccc3c3ccccc23)cc1]

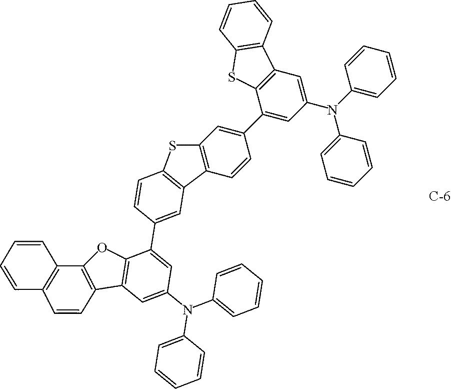

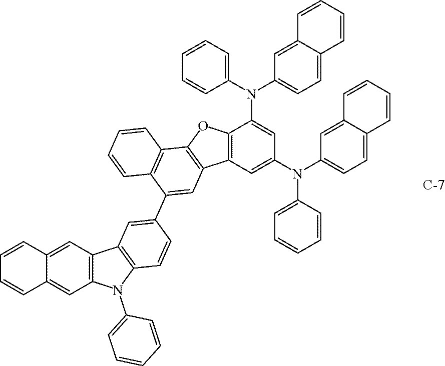

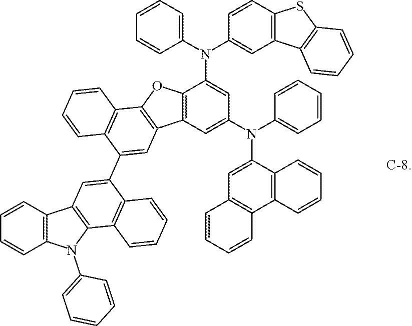

### Experimental Example

The driving voltage and efficiency were measured by applying a current of 15 mA/cm² to the organic light emitting devices manufactured in the Examples 1 to 39 and Comparative Examples 1 to 8, and the results are shown in Table 1 below. T95 means the time required for the luminance to be reduced to 95% of the initial luminance (6000 nit).

TABLE 1

| Category | Material | Driving voltage(V) | Efficiency(cd/A) | Lifetime T95(hr) | Luminescent color |
|---|---|---|---|---|---|
| Example 1 | Compound 1 | 3.85 | 18.79 | 220 | Red |
| Example 2 | Compound 2 | 3.78 | 19.00 | 217 | Red |
| Example 3 | Compound 3 | 3.76 | 18.93 | 222 | Red |
| Example 4 | Compound 4 | 3.80 | 18.19 | 214 | Red |
| Example 5 | Compound 5 | 3.87 | 19.06 | 226 | Red |
| Example 6 | Compound 6 | 3.83 | 19.25 | 218 | Red |
| Example 7 | Compound 7 | 3.86 | 18.65 | 227 | Red |
| Example 8 | Compound 8 | 3.76 | 19.00 | 214 | Red |
| Example 9 | Compound 9 | 3.68 | 20.43 | 224 | Red |
| Example 10 | Compound 10 | 3.68 | 20.40 | 223 | Red |
| Example 11 | Compound 11 | 3.73 | 19.35 | 226 | Red |
| Example 12 | Compound 12 | 3.69 | 20.14 | 233 | Red |
| Example 13 | Compound 13 | 3.76 | 19.23 | 221 | Red |
| Example 14 | Compound 14 | 3.70 | 19.57 | 231 | Red |
| Example 15 | Compound 15 | 3.76 | 19.76 | 226 | Red |
| Example 16 | Compound 16 | 3.56 | 22.93 | 186 | Red |
| Example 17 | Compound 17 | 3.65 | 20.63 | 194 | Red |
| Example 18 | Compound 18 | 3.60 | 19.55 | 180 | Red |
| Example 19 | Compound 19 | 3.60 | 22.37 | 183 | Red |
| Example 20 | Compound 20 | 3.60 | 21.44 | 199 | Red |
| Example 21 | Compound 21 | 3.65 | 22.57 | 183 | Red |
| Example 22 | Compound 22 | 3.60 | 22.76 | 195 | Red |
| Example 23 | Compound 23 | 3.55 | 20.94 | 286 | Red |
| Example 24 | Compound 24 | 3.58 | 22.20 | 310 | Red |
| Example 25 | Compound 25 | 3.64 | 22.91 | 294 | Red |
| Example 26 | Compound 26 | 3.64 | 19.82 | 306 | Red |
| Example 27 | Compound 27 | 3.55 | 22.30 | 310 | Red |
| Example 28 | Compound 28 | 3.60 | 21.72 | 301 | Red |
| Example 29 | Compound 29 | 3.80 | 19.09 | 215 | Red |
| Example 30 | Compound 30 | 3.89 | 18.62 | 222 | Red |
| Example 31 | Compound 31 | 3.75 | 19.45 | 212 | Red |
| Example 32 | Compound 32 | 3.78 | 19.09 | 221 | Red |
| Example 33 | Compound 33 | 3.87 | 18.41 | 214 | Red |
| Example 34 | Compound 34 | 3.57 | 17.76 | 190 | Red |
| Example 35 | Compound 35 | 3.63 | 17.97 | 182 | Red |
| Example 36 | Compound 36 | 3.61 | 17.07 | 194 | Red |
| Example 37 | Compound 37 | 3.57 | 18.31 | 194 | Red |
| Example 38 | Compound 38 | 3.65 | 17.91 | 180 | Red |
| Example 39 | Compound 39 | 3.60 | 18.32 | 189 | Red |
| Comparative Example 1 | C-1 | 4.11 | 13.20 | 103 | Red |
| Comparative Example 2 | C-2 | 3.91 | 16.35 | 148 | Red |
| Comparative Example 3 | C-3 | 3.97 | 16.07 | 160 | Red |
| Comparative Example 4 | C-4 | 4.05 | 13.80 | 117 | Red |
| Comparative Example 5 | C-5 | 4.04 | 10.03 | 78 | Red |
| Comparative Example 6 | C-6 | 4.02 | 12.62 | 124 | Red |
| Comparative Example 7 | C-7 | 4.13 | 11.02 | 120 | Red |
| Comparative Example 8 | C-8 | 4.28 | 10.43 | 109 | Red |

As shown in Table 1, the organic light emitting device of the example uses the compound of Formula 1 and the compound RH-1 as a host compound of the light emitting layer, and using compound Dp-7 as a dopant, it exhibits a low driving voltage, excellent luminous efficiency, and remarkably improved lifetime.

The organic light emitting device of the comparative example uses C-1 to C-8 instead of the compound of Chemical Formula 1 as a host compound of the light emitting layer, and it exhibits a high driving voltage, reduced luminous efficiency, and lifetime compared to the examples containing the compounds of Chemical Formula 1.

As a result of the experiment, it can be seen that when the compounds of Chemical Formula 1 are used as the host compound of the light emitting layer of the organic light emitting devices, energy transfer from the host to the red dopant was well performed. In addition, the organic light emitting devices of the Examples exhibited greatly improved lifetime characteristics while maintaining high efficiency, which is considered to be because the compounds of the present disclosure have higher stability to electrons and holes than the compounds used in the Comparative Examples.

Description of Symbols

| | |
|---|---|
| 1: substrate | 2: anode |
| 3: hole transport layer | 4: light emitting layer |
| 5: electron injection and transport layer | 6: cathode |
| 7: hole injection layer | 8: electron blocking layer |
| 9: hole blocking layer | |

1. A compound of Chemical Formula 1: 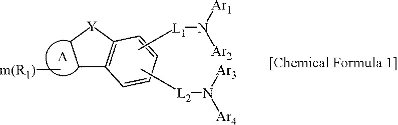
wherein, in Chemical Formula 1:
Y is S or O;
A is naphthalene ring,
L₁ and L₂ are each independently a direct bond or a substituted or unsubstituted C₆₋₆₀ arylene;
Ar₁, Ar₂, Ar₃ and Ar₄ are each independently a substituted or unsubstituted C₆₋₆₀ aryl or a substituted or unsubstituted C₂₋₆₀ heteroaryl containing at least one of N, O, and S;
R₁ is each independently hydrogen, deuterium, a substituted or unsubstituted C₁₋₆₀ alkyl or a substituted or unsubstituted C₆₋₆₀ aryl; and
m is an integer of 0 to 6.

2. The compound according to claim 1, wherein the Chemical Formula 1 is any one of the following Chemical Formula 1-1 to 1-3: 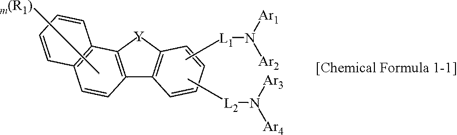 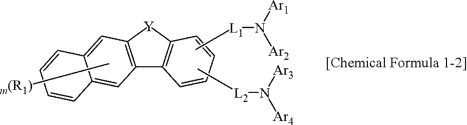 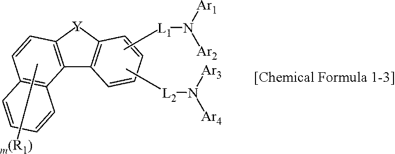
wherein, in Chemical Formula 1-1 to 1-3, Y, L
₁, L₂, Ar₁, Ar₂, Ar₃, Ar₄, R₁ and m are as defined in claim 1.

3. The compound according to claim 1, wherein the Chemical Formula 1 is any one of the following Chemical Formula 2-1 to 2-6: 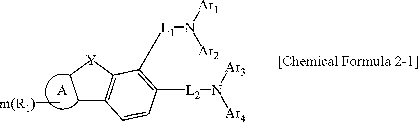 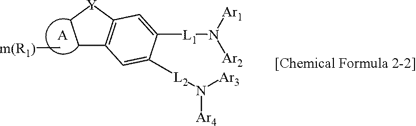 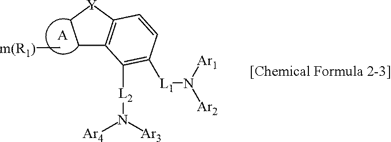 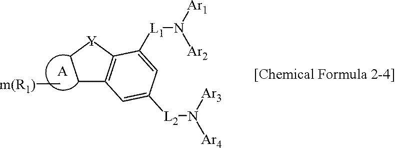 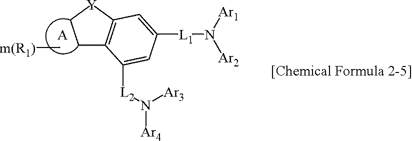 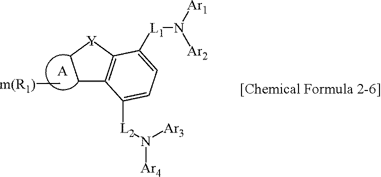
wherein, in Chemical Formula 2-1 to 2-6, Y, L
₁, L₂, Ar₁, Ar₂, Ar₃, Ar₄, R₁ and m are as defined in claim 1.

4. The compound according to claim 1, wherein L₁ and L₂ are each independently a direct bond, phenylene, biphenylylene or naphthylene.

5. The compound according to claim 1, wherein Ar₁, Ar₂, Ar₃ and Ar₄ are each independently biphenylyl, terphenylyl, naphthyl, naphthylphenyl, phenylnaphthyl, phenanthrenyl, triphenylenyl, dimethylfluorenyl, diphenylfluorenyl, dibenzofuranyl, dibenzothiophenyl, carbazol-9-yl, or 9-phenyl-9H-carbazolyl.

6. The compound according to claim 1, wherein R₁ is each independently hydrogen, deuterium, C₁₋₁₀alkyl or phenyl.

7. The compound according to claim 1, wherein m is an integer of 0 to 2.

8. The compound according to claim 1, wherein the compound of Chemical Formula 1 is any one compound selected from the group consisting of the following compounds: 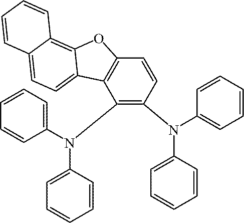 [SMILES: c1ccc(N(c2ccccc2)c2ccc3oc4cc5ccccc5cc4c3c2N(c2ccccc2)c2ccccc2)cc1] 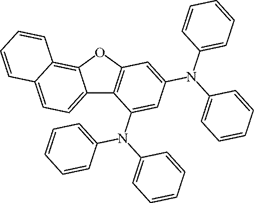 [SMILES: c1ccc(N(c2ccccc2)c2cc(N(c3ccccc3)c3ccccc3)c3c(c2)oc2cc4ccccc4cc23)cc1] 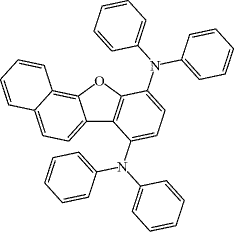 [SMILES: c1ccc(N(c2ccccc2)c2ccc(N(c3ccccc3)c3ccccc3)c3c2oc2cc4ccccc4cc23)cc1] 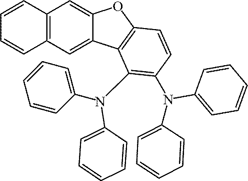 [SMILES: c1ccc(N(c2ccccc2)c2ccc3oc4ccc5ccccc5c4c3c2N(c2ccccc2)c2ccccc2)cc1] 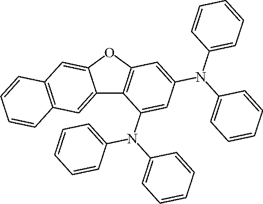 [SMILES: c1ccc(N(c2ccccc2)c2cc(N(c3ccccc3)c3ccccc3)c3c(c2)oc2ccc4ccccc4c23)cc1] 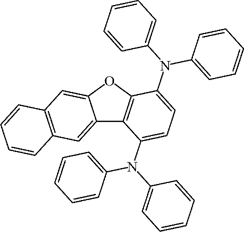 [SMILES: c1ccc(N(c2ccccc2)c2ccc(N(c3ccccc3)c3ccccc3)c3c2oc2ccc4ccccc4c23)cc1] 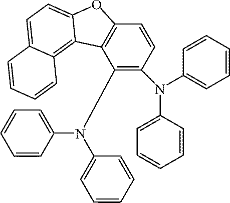 [SMILES: c1ccc(N(c2ccccc2)c2cc3oc4c5ccccc5ccc4c3cc2N(c2ccccc2)c2ccccc2)cc1] 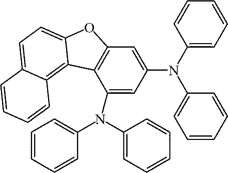 [SMILES: c1ccc(N(c2ccccc2)c2cc(N(c3ccccc3)c3ccccc3)c3oc4c5ccccc5ccc4c3c2)cc1] 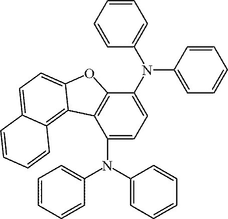 [SMILES: c1ccc(N(c2ccccc2)c2ccc3c(oc4c5ccccc5ccc34)c2N(c2ccccc2)c2ccccc2)cc1] 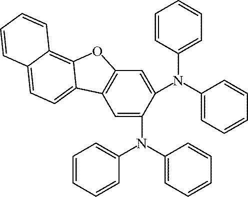 [SMILES: c1ccc(N(c2ccccc2)c2cc3oc4cc5ccccc5cc4c3cc2N(c2ccccc2)c2ccccc2)cc1] 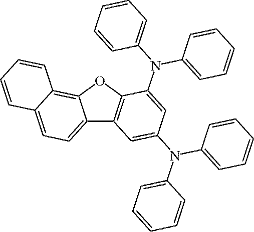 [SMILES: c1ccc(N(c2ccccc2)c2cc(N(c3ccccc3)c3ccccc3)c3oc4cc5ccccc5cc4c3c2)cc1] 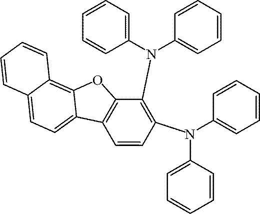 [SMILES: c1ccc(N(c2ccccc2)c2ccc3c(oc4cc5ccccc5cc43)c2N(c2ccccc2)c2ccccc2)cc1] 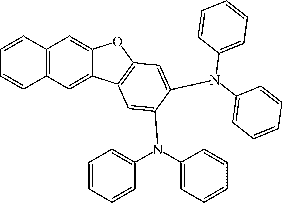 [SMILES: c1ccc(N(c2ccccc2)c2cc3oc4ccc5ccccc5c4c3cc2N(c2ccccc2)c2ccccc2)cc1] 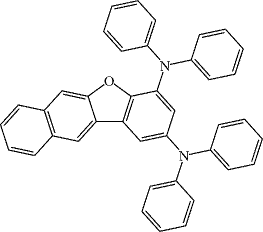 [SMILES: c1ccc(N(c2ccccc2)c2cc(N(c3ccccc3)c3ccccc3)c3oc4ccc5ccccc5c4c3c2)cc1] 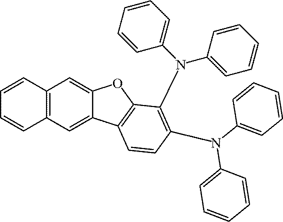 [SMILES: c1ccc(N(c2ccccc2)c2ccc3c(oc4ccc5ccccc5c43)c2N(c2ccccc2)c2ccccc2)cc1] 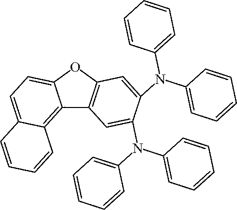 [SMILES: c1ccc(-c2ccccc2N(c2ccccc2)c2ccc3oc4c5ccccc5ccc4c3c2N(c2ccccc2)c2ccccc2-c2ccccc2)cc1] 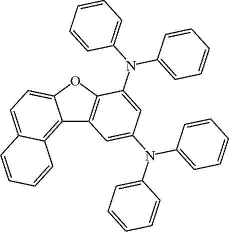 [SMILES: c1ccc(-c2ccccc2N(c2ccccc2)c2cc(N(c3ccccc3)c3ccccc3-c3ccccc3)c3c(c2)oc2c4ccccc4ccc23)cc1] 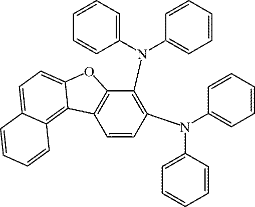 [SMILES: c1ccc(-c2ccccc2N(c2ccccc2)c2ccc(N(c3ccccc3)c3ccccc3-c3ccccc3)c3c2oc2c4ccccc4ccc23)cc1] 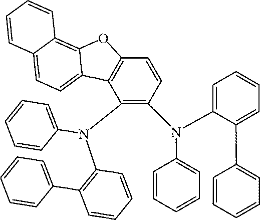 [SMILES: c1ccc(-c2ccccc2N(c2ccccc2)c2ccc3oc4cc5ccccc5cc4c3c2N(c2ccccc2)c2ccccc2-c2ccccc2)cc1] 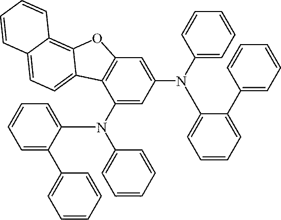 [SMILES: c1ccc(-c2ccccc2N(c2ccccc2)c2cc(N(c3ccccc3)c3ccccc3-c3ccccc3)c3c(c2)oc2cc4ccccc4cc23)cc1] 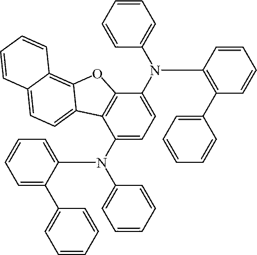 [SMILES: c1ccc(-c2ccccc2N(c2ccccc2)c2ccc(N(c3ccccc3)c3ccccc3-c3ccccc3)c3c2oc2cc4ccccc4cc23)cc1] 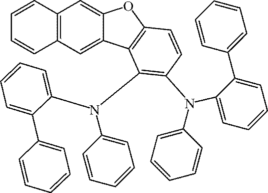 [SMILES: c1ccc(-c2ccccc2N(c2ccccc2)c2ccc3oc4ccc5ccccc5c4c3c2N(c2ccccc2)c2ccccc2-c2ccccc2)cc1] 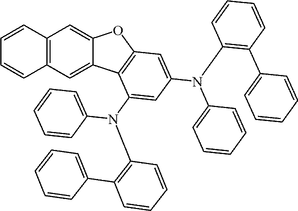 [SMILES: c1ccc(-c2ccccc2N(c2ccccc2)c2cc(N(c3ccccc3)c3ccccc3-c3ccccc3)c3c(c2)oc2ccc4ccccc4c23)cc1] 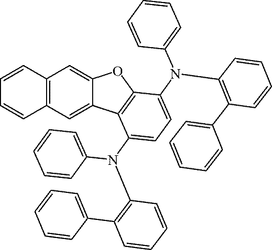 [SMILES: c1ccc(-c2ccccc2N(c2ccccc2)c2ccc(N(c3ccccc3)c3ccccc3-c3ccccc3)c3c2oc2ccc4ccccc4c23)cc1] 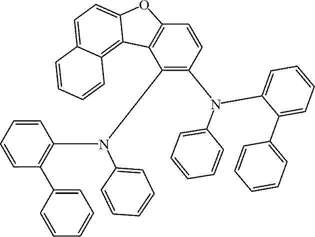 [SMILES: c1ccc(-c2ccccc2N(c2ccccc2)c2cc3oc4c5ccccc5ccc4c3cc2N(c2ccccc2)c2ccccc2-c2ccccc2)cc1] 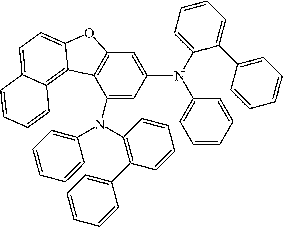 [SMILES: c1ccc(-c2ccccc2N(c2ccccc2)c2cc(N(c3ccccc3)c3ccccc3-c3ccccc3)c3oc4c5ccccc5ccc4c3c2)cc1] 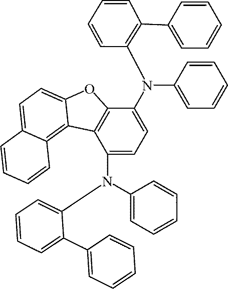 [SMILES: c1ccc(-c2ccccc2N(c2ccccc2)c2ccc3c(oc4c5ccccc5ccc34)c2N(c2ccccc2)c2ccccc2-c2ccccc2)cc1] 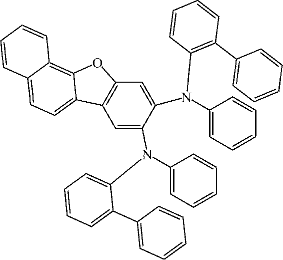 [SMILES: c1ccc(-c2ccccc2N(c2ccccc2)c2cc3oc4cc5ccccc5cc4c3cc2N(c2ccccc2)c2ccccc2-c2ccccc2)cc1] 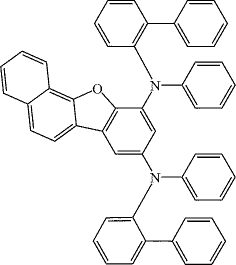 [SMILES: c1ccc(-c2ccccc2N(c2ccccc2)c2cc(N(c3ccccc3)c3ccccc3-c3ccccc3)c3oc4cc5ccccc5cc4c3c2)cc1] 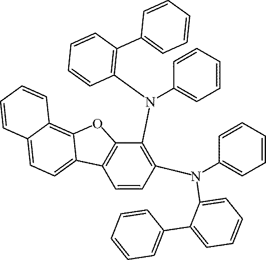 [SMILES: c1ccc(-c2ccccc2N(c2ccccc2)c2ccc3c(oc4cc5ccccc5cc43)c2N(c2ccccc2)c2ccccc2-c2ccccc2)cc1] 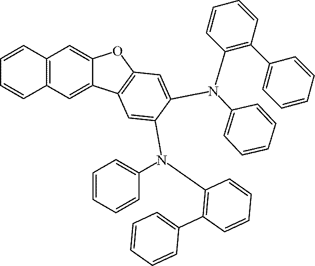 [SMILES: c1ccc(-c2ccccc2N(c2ccccc2)c2cc3oc4ccc5ccccc5c4c3cc2N(c2ccccc2)c2ccccc2-c2ccccc2)cc1] 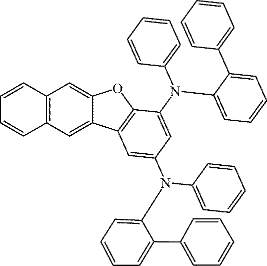 [SMILES: c1ccc(-c2ccccc2N(c2ccccc2)c2cc(N(c3ccccc3)c3ccccc3-c3ccccc3)c3oc4ccc5ccccc5c4c3c2)cc1] 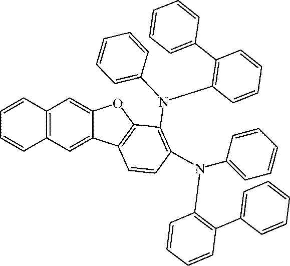 [SMILES: c1ccc(-c2ccccc2N(c2ccccc2)c2ccc3c(oc4ccc5ccccc5c43)c2N(c2ccccc2)c2ccccc2-c2ccccc2)cc1] 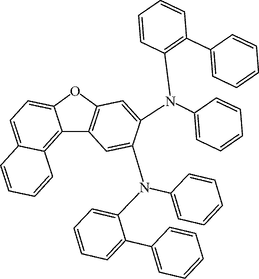 [SMILES: c1ccc(-c2cccc(N(c3ccccc3)c3ccc4oc5c6ccccc6ccc5c4c3N(c3ccccc3)c3ccccc3)c2)cc1] 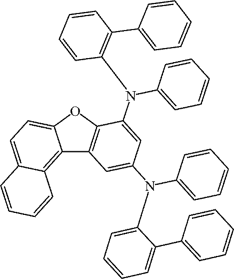 [SMILES: c1ccc(-c2cccc(N(c3ccccc3)c3cc(N(c4ccccc4)c4ccccc4)c4c(c3)oc3c5ccccc5ccc34)c2)cc1] 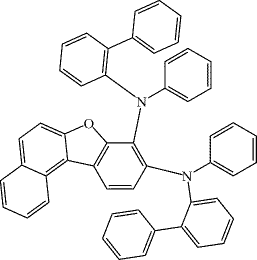 [SMILES: c1ccc(-c2cccc(N(c3ccccc3)c3ccc(N(c4ccccc4)c4ccccc4)c4oc5c6ccccc6ccc5c34)c2)cc1] 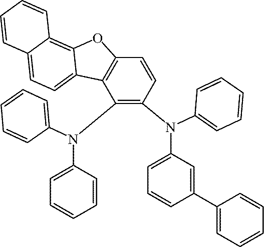 [SMILES: c1ccc(-c2cccc(N(c3ccccc3)c3ccc4oc5cc6ccccc6cc5c4c3N(c3ccccc3)c3ccccc3)c2)cc1] 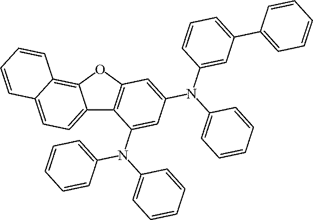 [SMILES: c1ccc(-c2ccc(N(c3ccccc3)c3cc(N(c4ccccc4)c4ccccc4)cc4oc5cc6ccccc6cc5c34)cc2)cc1] 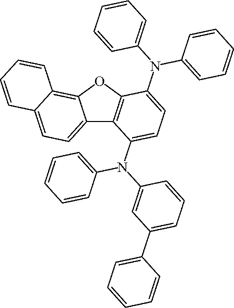 [SMILES: c1ccc(-c2cccc(N(c3ccccc3)c3ccc(N(c4ccccc4)c4ccccc4)c4c3oc3cc5ccccc5cc34)c2)cc1] 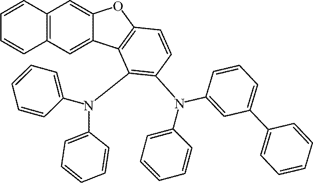 [SMILES: c1ccc(-c2cccc(N(c3ccccc3)c3ccc4oc5ccc6ccccc6c5c4c3N(c3ccccc3)c3ccccc3)c2)cc1] 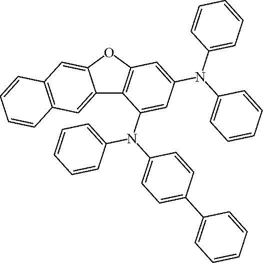 [SMILES: c1ccc(-c2cccc(N(c3ccccc3)c3cc(N(c4ccccc4)c4ccccc4)c4c(c3)oc3ccc5ccccc5c34)c2)cc1] 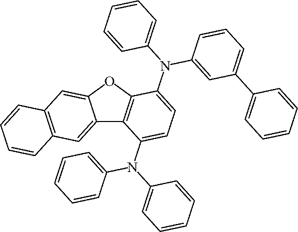 [SMILES: c1ccc(-c2cccc(N(c3ccccc3)c3ccc(N(c4ccccc4)c4ccccc4)c4c3oc3ccc5ccccc5c34)c2)cc1] 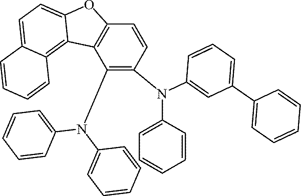 [SMILES: c1ccc(-c2cccc(N(c3ccccc3)c3cc4oc5c6ccccc6ccc5c4cc3N(c3ccccc3)c3ccccc3)c2)cc1] 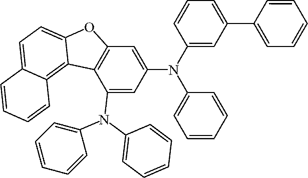 [SMILES: c1ccc(-c2cccc(N(c3ccccc3)c3cc(N(c4ccccc4)c4ccccc4)cc4c3oc3c5ccccc5ccc43)c2)cc1] 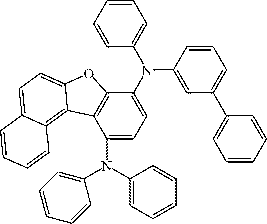 [SMILES: c1ccc(-c2cccc(N(c3ccccc3)c3ccc4c(oc5c6ccccc6ccc45)c3N(c3ccccc3)c3ccccc3)c2)cc1] 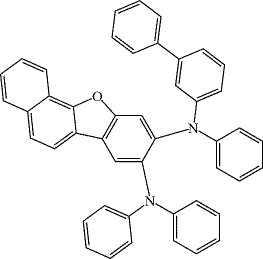 [SMILES: c1ccc(-c2cccc(N(c3ccccc3)c3cc4c(cc3N(c3ccccc3)c3ccccc3)oc3cc5ccccc5cc34)c2)cc1] 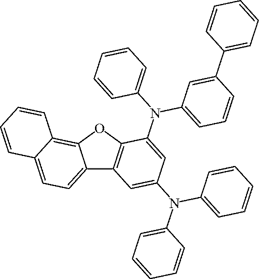 [SMILES: c1ccc(-c2cccc(N(c3ccccc3)c3cc(N(c4ccccc4)c4ccccc4)c4oc5cc6ccccc6cc5c4c3)c2)cc1] 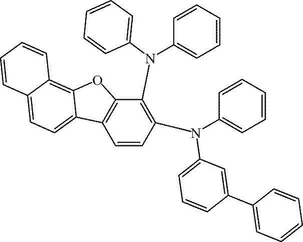 [SMILES: c1ccc(-c2cccc(N(c3ccccc3)c3ccc4c(oc5cc6ccccc6cc54)c3N(c3ccccc3)c3ccccc3)c2)cc1] 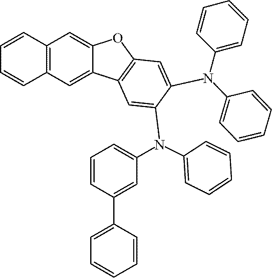 [SMILES: c1ccc(-c2cccc(N(c3ccccc3)c3cc4c(cc3N(c3ccccc3)c3ccccc3)oc3ccc5ccccc5c34)c2)cc1] 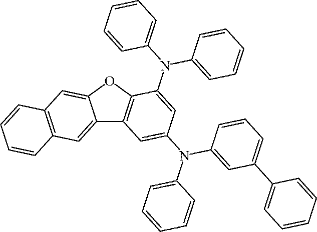 [SMILES: c1ccc(-c2cccc(N(c3ccccc3)c3cc(N(c4ccccc4)c4ccccc4)cc4c3oc3ccc5ccccc5c34)c2)cc1] 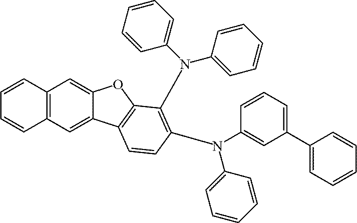 [SMILES: c1ccc(-c2cccc(N(c3ccccc3)c3ccc4c(oc5ccc6ccccc6c54)c3N(c3ccccc3)c3ccccc3)c2)cc1] 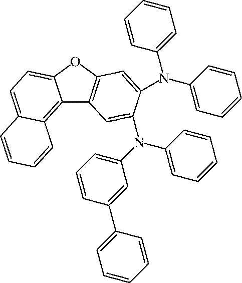 [SMILES: c1ccc(-c2cccc(N(c3ccccc3)c3ccc4oc5c6ccccc6ccc5c4c3N(c3ccccc3)c3cccc(-c4ccccc4)c3)c2)cc1] 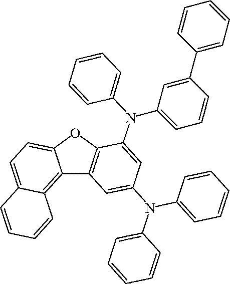 [SMILES: c1ccc(-c2cccc(N(c3ccccc3)c3cc(N(c4ccccc4)c4cccc(-c5ccccc5)c4)c4c(c3)oc3c5ccccc5ccc34)c2)cc1] 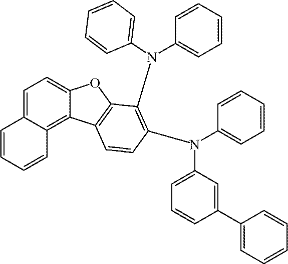 [SMILES: c1ccc(-c2cccc(N(c3ccccc3)c3ccc(N(c4ccccc4)c4cccc(-c5ccccc5)c4)c4c3oc3c5ccccc5ccc34)c2)cc1] 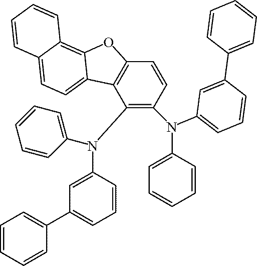 [SMILES: c1ccc(-c2cccc(N(c3ccccc3)c3ccc4oc5cc6ccccc6cc5c4c3N(c3ccccc3)c3cccc(-c4ccccc4)c3)c2)cc1] 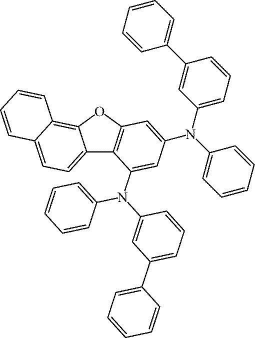 [SMILES: c1ccc(-c2cccc(N(c3ccccc3)c3cc(N(c4ccccc4)c4cccc(-c5ccccc5)c4)c4c(c3)oc3cc5ccccc5cc34)c2)cc1] 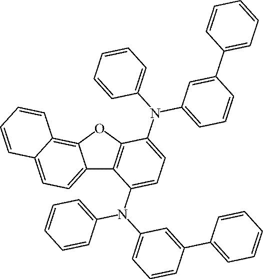 [SMILES: c1ccc(-c2cccc(N(c3ccccc3)c3ccc(N(c4ccccc4)c4cccc(-c5ccccc5)c4)c4c3oc3cc5ccccc5cc34)c2)cc1] 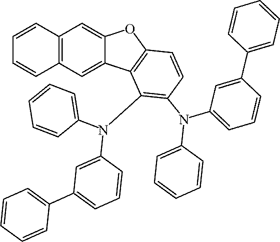 [SMILES: c1ccc(-c2cccc(N(c3ccccc3)c3ccc4oc5ccc6ccccc6c5c4c3N(c3ccccc3)c3cccc(-c4ccccc4)c3)c2)cc1] 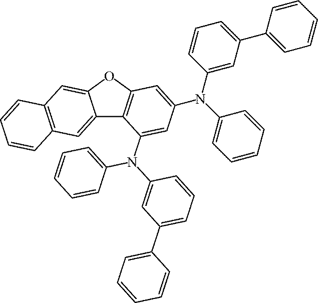 [SMILES: c1ccc(-c2cccc(N(c3ccccc3)c3cc(N(c4ccccc4)c4cccc(-c5ccccc5)c4)c4c(c3)oc3ccc5ccccc5c34)c2)cc1] 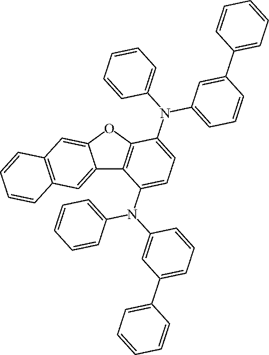 [SMILES: c1ccc(-c2cccc(N(c3ccccc3)c3ccc(N(c4ccccc4)c4cccc(-c5ccccc5)c4)c4c3oc3ccc5ccccc5c34)c2)cc1] 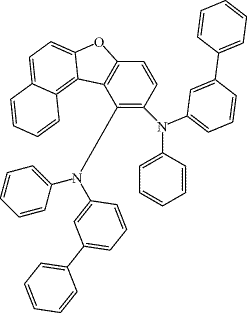 [SMILES: c1ccc(-c2cccc(N(c3ccccc3)c3cc4oc5c6ccccc6ccc5c4cc3N(c3ccccc3)c3cccc(-c4ccccc4)c3)c2)cc1] 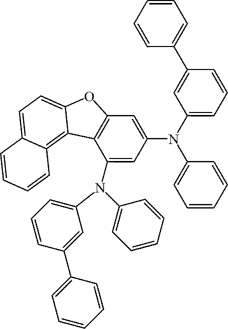 [SMILES: c1ccc(-c2cccc(N(c3ccccc3)c3cc(N(c4ccccc4)c4cccc(-c5ccccc5)c4)c4oc5c6ccccc6ccc5c4c3)c2)cc1] 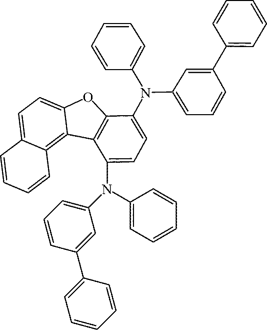 [SMILES: c1ccc(-c2cccc(N(c3ccccc3)c3ccc4c(oc5c6ccccc6ccc45)c3N(c3ccccc3)c3cccc(-c4ccccc4)c3)c2)cc1] 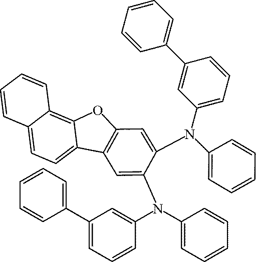 [SMILES: c1ccc(-c2cccc(N(c3ccccc3)c3cc4oc5cc6ccccc6cc5c4cc3N(c3ccccc3)c3cccc(-c4ccccc4)c3)c2)cc1] 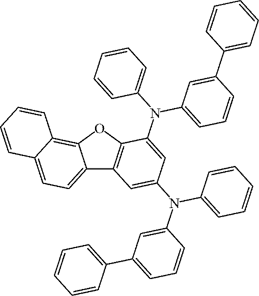 [SMILES: c1ccc(-c2cccc(N(c3ccccc3)c3cc(N(c4ccccc4)c4cccc(-c5ccccc5)c4)c4oc5cc6ccccc6cc5c4c3)c2)cc1] 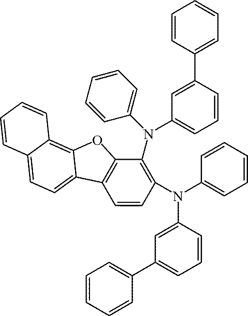 [SMILES: c1ccc(-c2cccc(N(c3ccccc3)c3ccc4c(oc5cc6ccccc6cc54)c3N(c3ccccc3)c3cccc(-c4ccccc4)c3)c2)cc1] 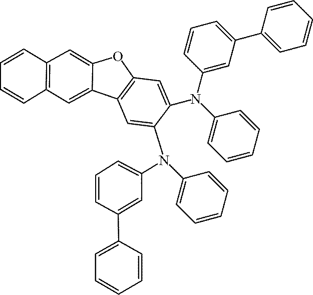 [SMILES: c1ccc(-c2cccc(N(c3ccccc3)c3cc4oc5ccc6ccccc6c5c4cc3N(c3ccccc3)c3cccc(-c4ccccc4)c3)c2)cc1] 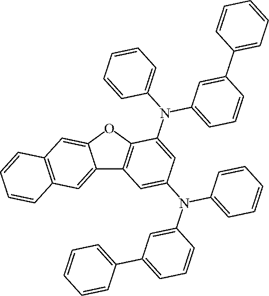 [SMILES: c1ccc(-c2cccc(N(c3ccccc3)c3cc(N(c4ccccc4)c4cccc(-c5ccccc5)c4)c4oc5ccc6ccccc6c5c4c3)c2)cc1] 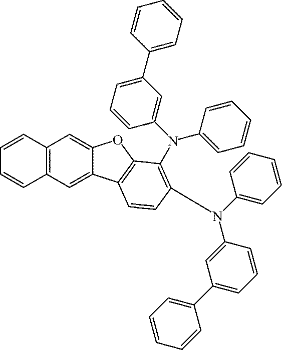 [SMILES: c1ccc(-c2cccc(N(c3ccccc3)c3ccc4c(oc5ccc6ccccc6c54)c3N(c3ccccc3)c3cccc(-c4ccccc4)c3)c2)cc1] 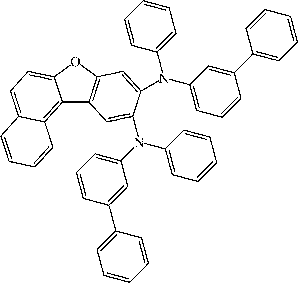 [SMILES: c1ccc(N(c2ccccc2)c2ccc3oc4c5ccccc5ccc4c3c2N(c2ccccc2)c2ccc3c(c2)oc2ccccc23)cc1] 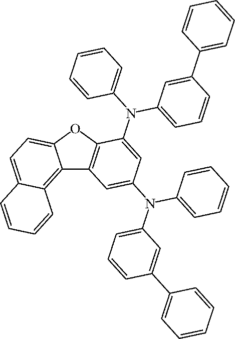 [SMILES: c1ccc(N(c2ccccc2)c2cc(N(c3ccccc3)c3ccc4c(c3)oc3ccccc34)c3c(c2)oc2c4ccccc4ccc23)cc1] 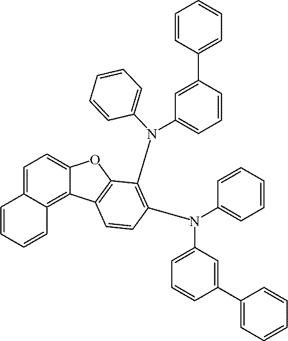 [SMILES: c1ccc(N(c2ccccc2)c2ccc(N(c3ccccc3)c3ccc4c(c3)oc3ccccc34)c3c2oc2c4ccccc4ccc23)cc1] 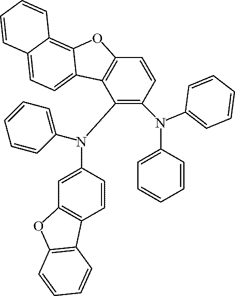 [SMILES: c1ccc(N(c2ccccc2)c2ccc3oc4cc5ccccc5cc4c3c2N(c2ccccc2)c2ccc3c(c2)sc2ccccc23)cc1] 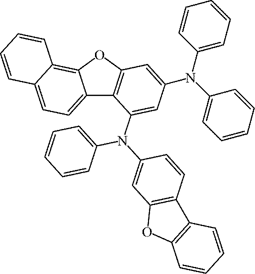 [SMILES: c1ccc(N(c2cc(N(c3ccccc3)c3ccccc3)c3c(c2)oc2cc4ccccc4cc23)c2ccc3oc4ccccc4c3c2)cc1] 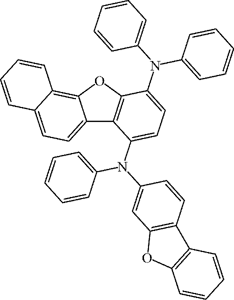 [SMILES: c1ccc(N(c2ccc3c(c2)oc2ccccc23)c2ccc(N(c3ccccc3)c3ccccc3)c3c2oc2cc4ccccc4cc23)cc1] 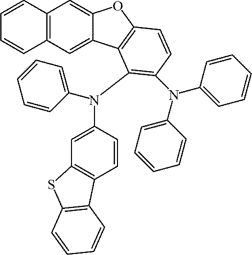 [SMILES: c1ccc(N(c2ccccc2)c2ccc3oc4ccc5ccccc5c4c3c2N(c2ccccc2)c2ccc3c(c2)sc2ccccc23)cc1] 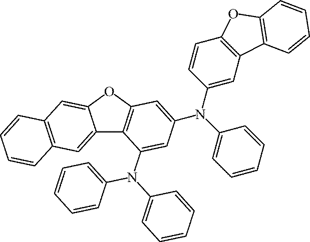 [SMILES: c1ccc(N(c2ccccc2)c2cc(N(c3ccccc3)c3ccc4c(c3)sc3ccccc34)c3c(c2)oc2ccc4ccccc4c23)cc1] 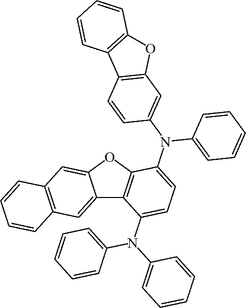 [SMILES: c1ccc(N(c2ccccc2)c2ccc(N(c3ccccc3)c3ccc4oc5ccccc5c4c3)c3c2oc2ccc4ccccc4c23)cc1] 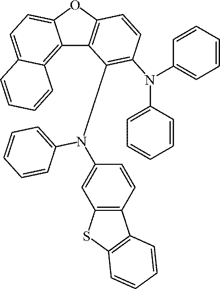 [SMILES: c1ccc(N(c2ccccc2)c2cc3oc4c5ccccc5ccc4c3cc2N(c2ccccc2)c2ccc3sc4ccccc4c3c2)cc1] 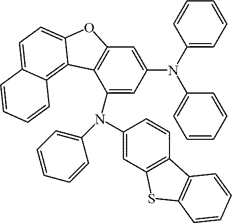 [SMILES: c1ccc(N(c2ccc3sc4ccccc4c3c2)c2cc(N(c3ccccc3)c3ccccc3)c3oc4c5ccccc5ccc4c3c2)cc1] 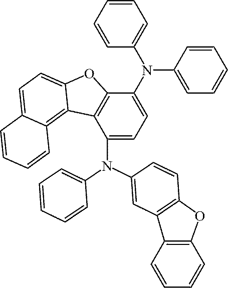 [SMILES: c1ccc(N(c2ccccc2)c2ccc3c(oc4c5ccccc5ccc34)c2N(c2ccccc2)c2ccc3c(c2)oc2ccccc23)cc1] 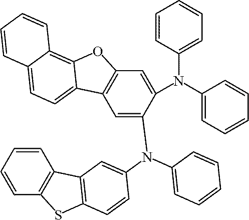 [SMILES: c1ccc(N(c2ccc3sc4ccccc4c3c2)c2cc3oc4cc5ccccc5cc4c3cc2N(c2ccccc2)c2ccccc2)cc1] 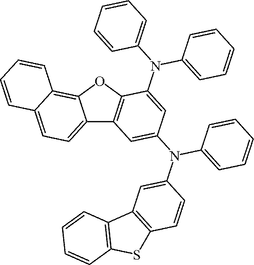 [SMILES: c1ccc(N(c2ccccc2)c2cc(N(c3ccccc3)c3ccc4c(c3)oc3ccccc34)c3oc4cc5ccccc5cc4c3c2)cc1] 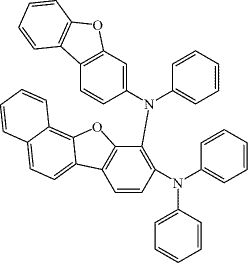 [SMILES: c1ccc(N(c2ccccc2)c2ccc3c(oc4cc5ccccc5cc43)c2N(c2ccccc2)c2ccc3sc4ccccc4c3c2)cc1] 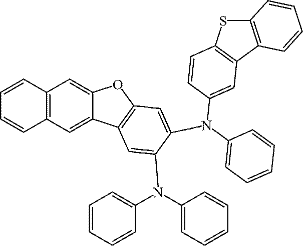 [SMILES: c1ccc(N(c2ccc3c(c2)oc2ccccc23)c2cc3oc4ccc5ccccc5c4c3cc2N(c2ccccc2)c2ccccc2)cc1] 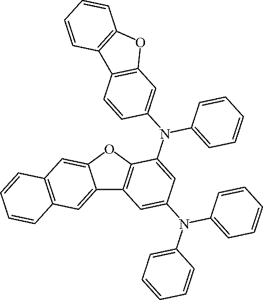 [SMILES: c1ccc(N(c2ccccc2)c2cc(N(c3ccccc3)c3ccc4c(c3)sc3ccccc34)c3oc4ccc5ccccc5c4c3c2)cc1] 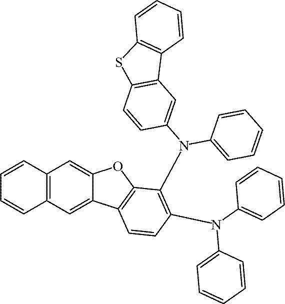 [SMILES: c1ccc(N(c2ccccc2)c2ccc3c(oc4ccc5ccccc5c43)c2N(c2ccccc2)c2ccc3sc4ccccc4c3c2)cc1] 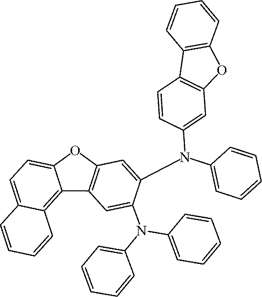 [SMILES: c1ccc(N(c2ccc3oc4c5ccccc5ccc4c3c2N(c2ccccc2)c2ccccc2)c2cccc3c2sc2ccccc23)cc1] 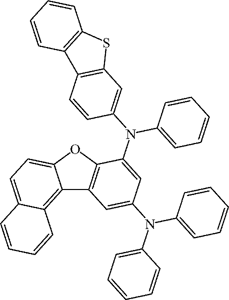 [SMILES: c1ccc(N(c2cc(N(c3ccccc3)c3ccccc3)c3c(c2)oc2c4ccccc4ccc23)c2cccc3oc4ccccc4c23)cc1] 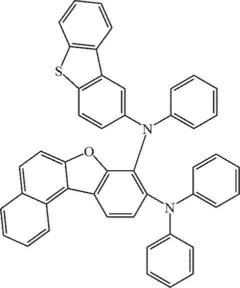 [SMILES: c1ccc(N(c2ccc(N(c3ccccc3)c3ccccc3)c3c2oc2c4ccccc4ccc23)c2cccc3oc4ccccc4c23)cc1] 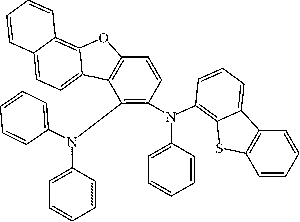 [SMILES: c1ccc(N(c2ccc3oc4cc5ccccc5cc4c3c2N(c2ccccc2)c2ccccc2)c2cccc3c2sc2ccccc23)cc1] 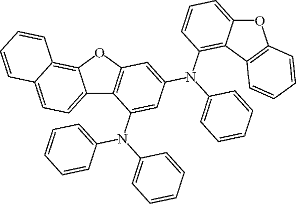 [SMILES: c1ccc(N(c2cc(N(c3ccccc3)c3ccccc3)c3c(c2)oc2cc4ccccc4cc23)c2cccc3oc4ccccc4c23)cc1] 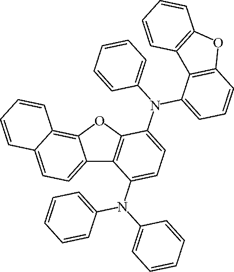 [SMILES: c1ccc(N(c2ccccc2)c2ccc(N(c3ccccc3)c3cccc4oc5ccccc5c34)c3c2oc2cc4ccccc4cc23)cc1] 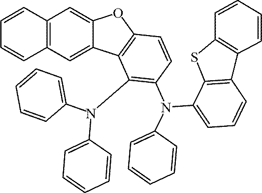 [SMILES: c1ccc(N(c2ccc3oc4ccc5ccccc5c4c3c2N(c2ccccc2)c2ccccc2)c2cccc3c2sc2ccccc23)cc1] 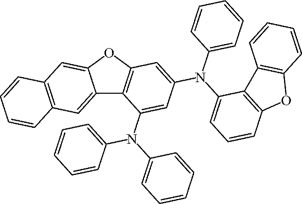 [SMILES: c1ccc(N(c2cc(N(c3ccccc3)c3ccccc3)c3c(c2)oc2ccc4ccccc4c23)c2cccc3c2sc2ccccc23)cc1] 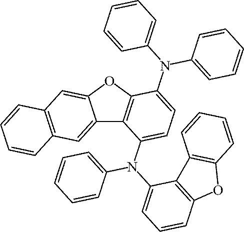 [SMILES: c1ccc(N(c2ccc(N(c3ccccc3)c3ccccc3)c3c2oc2ccc4ccccc4c23)c2cccc3c2sc2ccccc23)cc1] 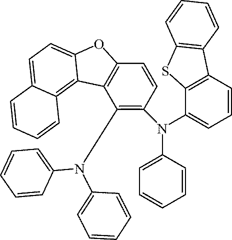 [SMILES: c1ccc(N(c2ccccc2)c2cc3oc4c5ccccc5ccc4c3cc2N(c2ccccc2)c2cccc3c2sc2ccccc23)cc1] 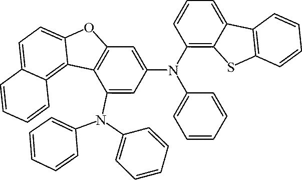 [SMILES: c1ccc(N(c2ccccc2)c2cc(N(c3ccccc3)c3cccc4oc5ccccc5c34)c3oc4c5ccccc5ccc4c3c2)cc1] 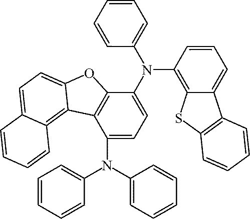 [SMILES: c1ccc(N(c2ccccc2)c2c(N(c3ccccc3)c3cccc4c3sc3ccccc34)ccc3c2oc2c4ccccc4ccc32)cc1] 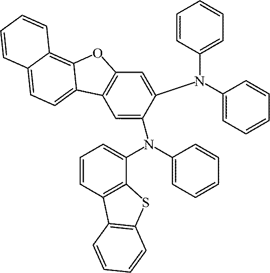 [SMILES: c1ccc(N(c2ccccc2)c2cc3c(cc2N(c2ccccc2)c2cccc4c2sc2ccccc24)oc2cc4ccccc4cc23)cc1] 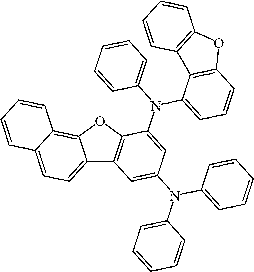 [SMILES: c1ccc(N(c2ccccc2)c2cc(N(c3ccccc3)c3cccc4oc5ccccc5c34)c3oc4cc5ccccc5cc4c3c2)cc1] 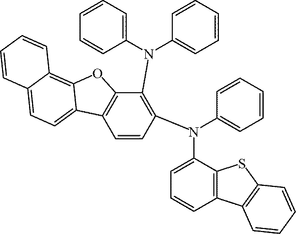 [SMILES: c1ccc(N(c2ccccc2)c2ccc3c(oc4cc5ccccc5cc43)c2N(c2ccccc2)c2cccc3c2sc2ccccc23)cc1] 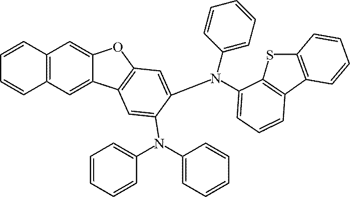 [SMILES: c1ccc(N(c2ccccc2)c2cc3c(cc2N(c2ccccc2)c2cccc4c2oc2ccccc24)oc2ccc4ccccc4c23)cc1] 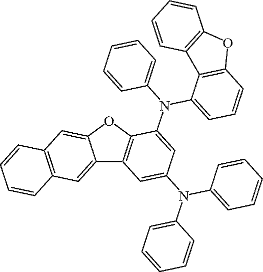 [SMILES: c1ccc(N(c2ccccc2)c2cc(N(c3ccccc3)c3cccc4oc5ccccc5c34)cc3c2oc2ccc4ccccc4c23)cc1] 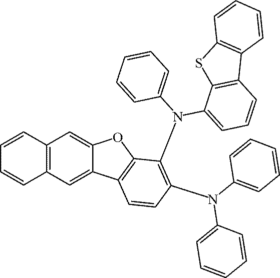 [SMILES: c1ccc(N(c2ccccc2)c2c(N(c3ccccc3)c3cccc4sc5ccccc5c34)ccc3c2oc2ccc4ccccc4c23)cc1] 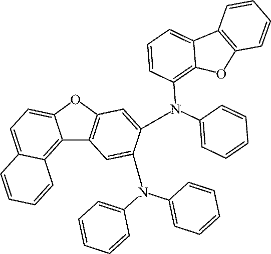 [SMILES: c1ccc(-c2ccc(N(c3ccccc3)c3ccc4oc5c6ccccc6ccc5c4c3N(c3ccccc3)c3ccc(-c4ccccc4)cc3)cc2)cc1] 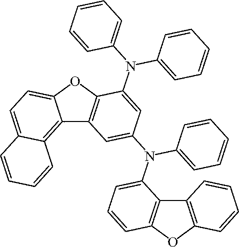 [SMILES: c1ccc(-c2ccc(N(c3ccccc3)c3cc(N(c4ccccc4)c4ccc(-c5ccccc5)cc4)c4c(c3)oc3c5ccccc5ccc34)cc2)cc1] 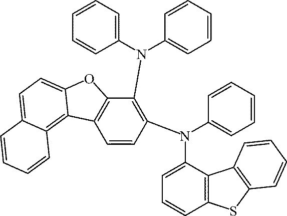 [SMILES: c1ccc(-c2ccc(N(c3ccccc3)c3ccc(N(c4ccccc4)c4ccc(-c5ccccc5)cc4)c4c3oc3c5ccccc5ccc34)cc2)cc1] 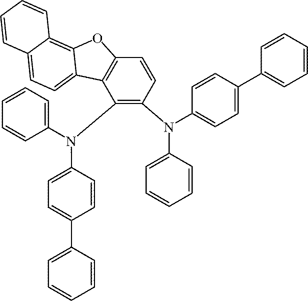 [SMILES: c1ccc(-c2ccc(N(c3ccccc3)c3ccc4oc5cc6ccccc6cc5c4c3N(c3ccccc3)c3ccc(-c4ccccc4)cc3)cc2)cc1] 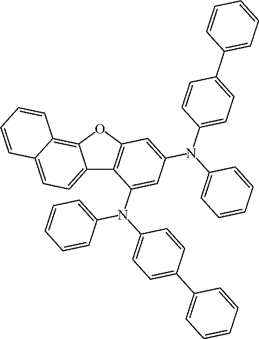 [SMILES: c1ccc(-c2ccc(N(c3ccccc3)c3cc(N(c4ccccc4)c4ccc(-c5ccccc5)cc4)c4c(c3)oc3cc5ccccc5cc34)cc2)cc1] 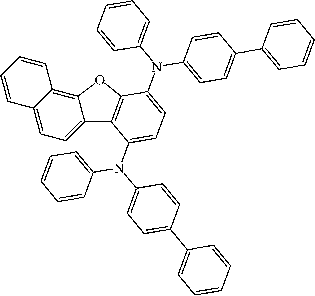 [SMILES: c1ccc(-c2ccc(N(c3ccccc3)c3ccc(N(c4ccccc4)c4ccc(-c5ccccc5)cc4)c4c3oc3cc5ccccc5cc34)cc2)cc1] 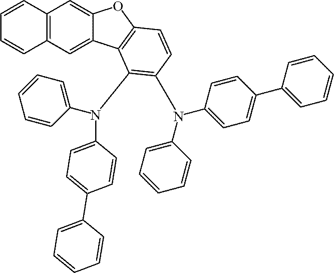 [SMILES: c1ccc(-c2ccc(N(c3ccccc3)c3ccc4oc5ccc6ccccc6c5c4c3N(c3ccccc3)c3ccc(-c4ccccc4)cc3)cc2)cc1] 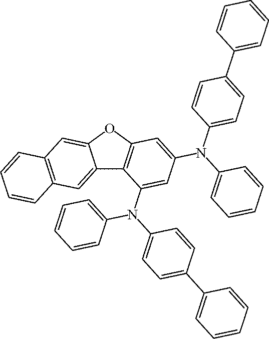 [SMILES: c1ccc(-c2ccc(N(c3ccccc3)c3cc(N(c4ccccc4)c4ccc(-c5ccccc5)cc4)c4c(c3)oc3ccc5ccccc5c34)cc2)cc1] 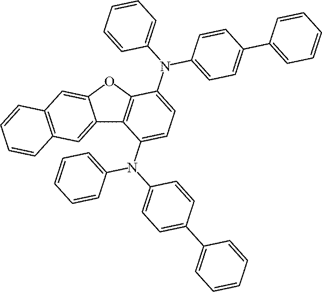 [SMILES: c1ccc(-c2ccc(N(c3ccccc3)c3ccc(N(c4ccccc4)c4ccc(-c5ccccc5)cc4)c4c3oc3ccc5ccccc5c34)cc2)cc1] 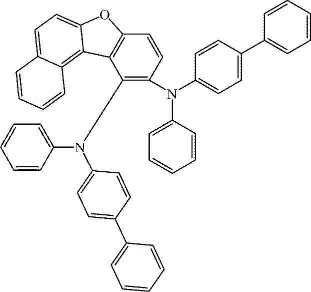 [SMILES: c1ccc(-c2ccc(N(c3ccccc3)c3cc4oc5c6ccccc6ccc5c4cc3N(c3ccccc3)c3ccc(-c4ccccc4)cc3)cc2)cc1] 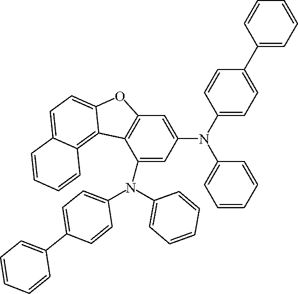 [SMILES: c1ccc(-c2ccc(N(c3ccccc3)c3cc(N(c4ccccc4)c4ccc(-c5ccccc5)cc4)c4oc5c6ccccc6ccc5c4c3)cc2)cc1] 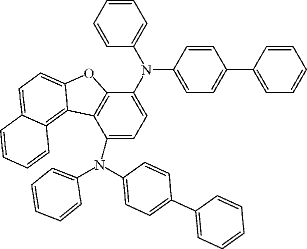 [SMILES: c1ccc(-c2ccc(N(c3ccccc3)c3ccc4c(oc5c6ccccc6ccc45)c3N(c3ccccc3)c3ccc(-c4ccccc4)cc3)cc2)cc1] 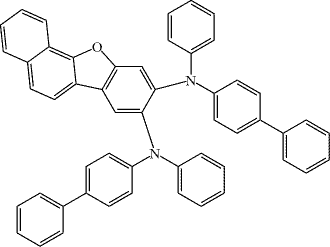 [SMILES: c1ccc(-c2ccc(N(c3ccccc3)c3cc4oc5cc6ccccc6cc5c4cc3N(c3ccccc3)c3ccc(-c4ccccc4)cc3)cc2)cc1] 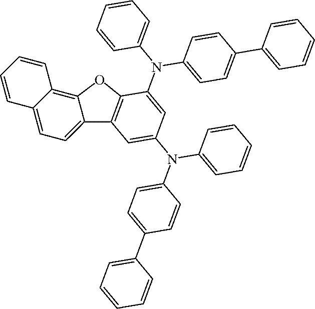 [SMILES: c1ccc(-c2ccc(N(c3ccccc3)c3cc(N(c4ccccc4)c4ccc(-c5ccccc5)cc4)c4oc5cc6ccccc6cc5c4c3)cc2)cc1] 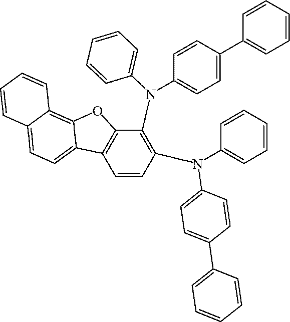 [SMILES: c1ccc(-c2ccc(N(c3ccccc3)c3ccc4c(oc5cc6ccccc6cc54)c3N(c3ccccc3)c3ccc(-c4ccccc4)cc3)cc2)cc1] 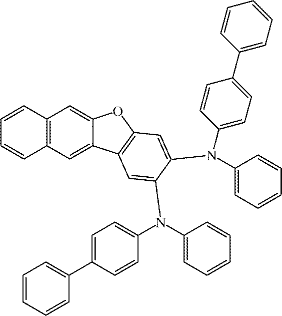 [SMILES: c1ccc(-c2ccc(N(c3ccccc3)c3cc4oc5ccc6ccccc6c5c4cc3N(c3ccccc3)c3ccc(-c4ccccc4)cc3)cc2)cc1] 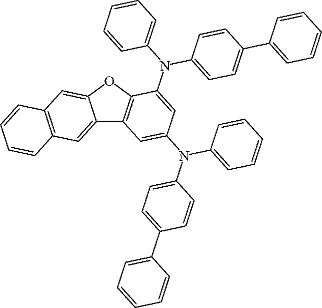 [SMILES: c1ccc(-c2ccc(N(c3ccccc3)c3cc(N(c4ccccc4)c4ccc(-c5ccccc5)cc4)c4oc5ccc6ccccc6c5c4c3)cc2)cc1] 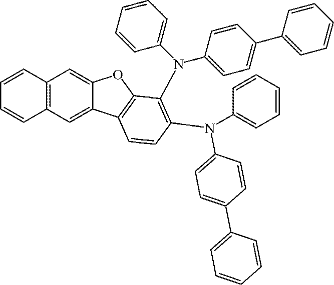 [SMILES: c1ccc(-c2ccc(N(c3ccccc3)c3ccc4c(oc5ccc6ccccc6c54)c3N(c3ccccc3)c3ccc(-c4ccccc4)cc3)cc2)cc1] 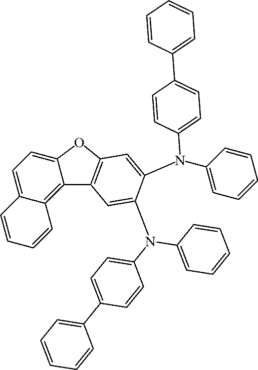 [SMILES: c1ccc(-c2ccccc2N(c2ccccc2)c2c(N(c3ccccc3)c3ccccc3)ccc3oc4c5ccccc5ccc4c23)cc1] 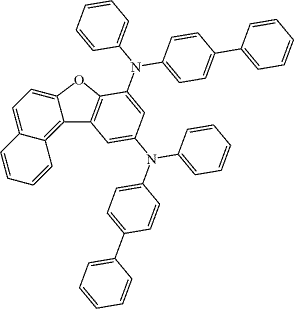 [SMILES: c1ccc(-c2ccccc2N(c2ccccc2)c2cc(N(c3ccccc3)c3ccccc3)cc3oc4c5ccccc5ccc4c23)cc1] 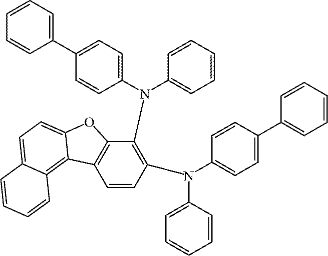 [SMILES: c1ccc(-c2ccccc2N(c2ccccc2)c2ccc(N(c3ccccc3)c3ccccc3)c3oc4c5ccccc5ccc4c23)cc1] 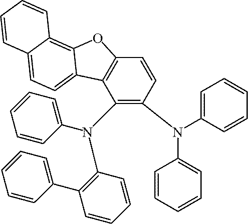 [SMILES: c1ccc(-c2ccccc2N(c2ccccc2)c2c(N(c3ccccc3)c3ccccc3)ccc3oc4cc5ccccc5cc4c23)cc1] 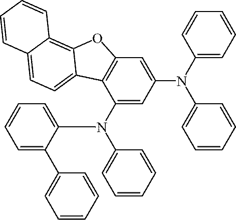 [SMILES: c1ccc(-c2ccccc2N(c2ccccc2)c2cc(N(c3ccccc3)c3ccccc3)cc3oc4cc5ccccc5cc4c23)cc1] 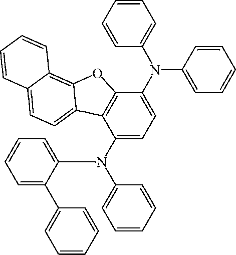 [SMILES: c1ccc(-c2ccccc2N(c2ccccc2)c2ccc(N(c3ccccc3)c3ccccc3)c3c2oc2cc4ccccc4cc23)cc1] 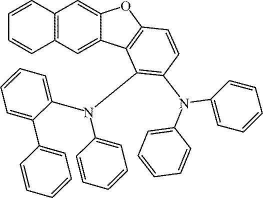 [SMILES: c1ccc(-c2ccccc2N(c2ccccc2)c2c(N(c3ccccc3)c3ccccc3)ccc3oc4ccc5ccccc5c4c23)cc1] 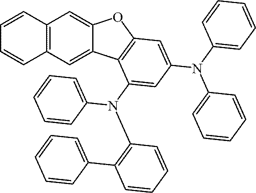 [SMILES: c1ccc(-c2ccccc2N(c2ccccc2)c2cc(N(c3ccccc3)c3ccccc3)c3c(c2)oc2ccc4ccccc4c23)cc1] 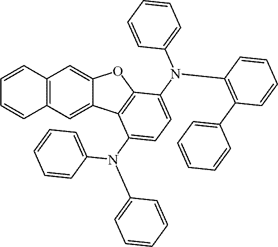 [SMILES: c1ccc(-c2ccccc2N(c2ccccc2)c2ccc(N(c3ccccc3)c3ccccc3)c3oc4ccc5ccccc5c4c23)cc1] 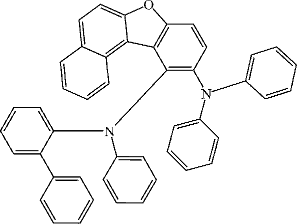 [SMILES: c1ccc(-c2ccccc2N(c2ccccc2)c2cc3c(cc2N(c2ccccc2)c2ccccc2)oc2c4ccccc4ccc32)cc1] 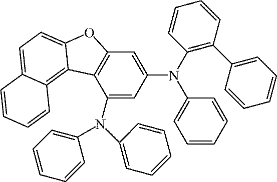 [SMILES: c1ccc(-c2ccccc2N(c2ccccc2)c2cc(N(c3ccccc3)c3ccccc3)c3oc4c5ccccc5ccc4c3c2)cc1] 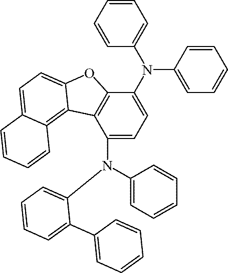 [SMILES: c1ccc(-c2ccccc2N(c2ccccc2)c2ccc3c(oc4c5ccccc5ccc34)c2N(c2ccccc2)c2ccccc2)cc1] 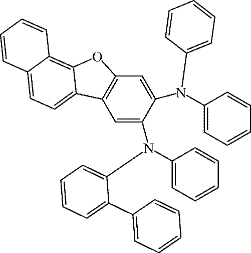 [SMILES: c1ccc(-c2ccccc2N(c2ccccc2)c2cc3c(cc2N(c2ccccc2)c2ccccc2)oc2cc4ccccc4cc23)cc1] 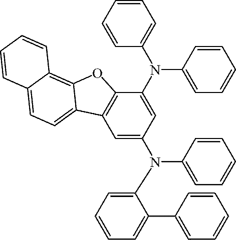 [SMILES: c1ccc(-c2ccccc2N(c2ccccc2)c2cc(N(c3ccccc3)c3ccccc3)cc3c2oc2cc4ccccc4cc23)cc1] 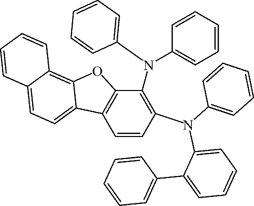 [SMILES: c1ccc(-c2ccccc2N(c2ccccc2)c2ccc3c(oc4cc5ccccc5cc43)c2N(c2ccccc2)c2ccccc2)cc1] 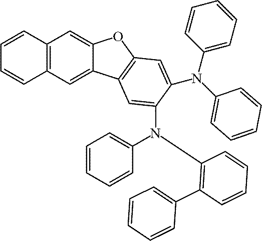 [SMILES: c1ccc(-c2ccccc2N(c2ccccc2)c2cc3c(cc2N(c2ccccc2)c2ccccc2)oc2ccc4ccccc4c23)cc1] 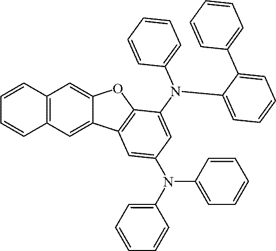 [SMILES: c1ccc(-c2ccccc2N(c2ccccc2)c2cc(N(c3ccccc3)c3ccccc3)cc3c2oc2ccc4ccccc4c23)cc1] 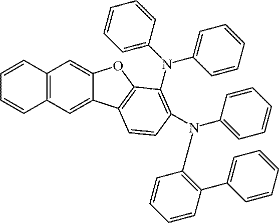 [SMILES: c1ccc(-c2ccccc2N(c2ccccc2)c2c(N(c3ccccc3)c3ccccc3)ccc3c2oc2ccc4ccccc4c23)cc1] 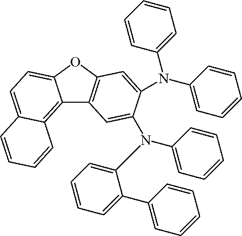 [SMILES: c1ccc(N(c2ccc3ccccc3c2)c2ccc3oc4c5ccccc5ccc4c3c2N(c2ccccc2)c2ccc3ccccc3c2)cc1] 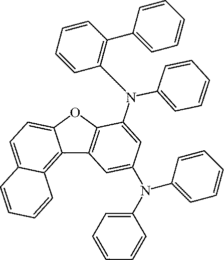 [SMILES: c1ccc(N(c2ccc3ccccc3c2)c2cc(N(c3ccccc3)c3ccc4ccccc4c3)c3c(c2)oc2c4ccccc4ccc23)cc1] 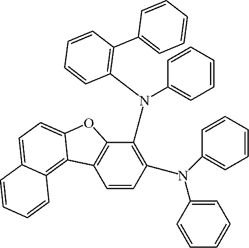 [SMILES: c1ccc(N(c2ccc3ccccc3c2)c2ccc(N(c3ccccc3)c3ccc4ccccc4c3)c3c2oc2c4ccccc4ccc23)cc1] 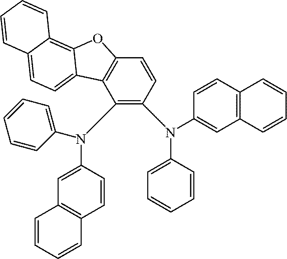 [SMILES: c1ccc(N(c2ccc3ccccc3c2)c2ccc3oc4cc5ccccc5cc4c3c2N(c2ccccc2)c2ccc3ccccc3c2)cc1] 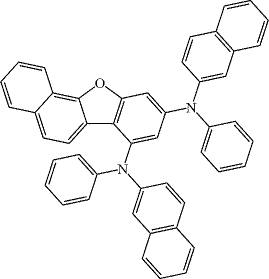 [SMILES: c1ccc(N(c2ccc3ccccc3c2)c2cc(N(c3ccccc3)c3ccc4ccccc4c3)c3c(c2)oc2cc4ccccc4cc23)cc1] 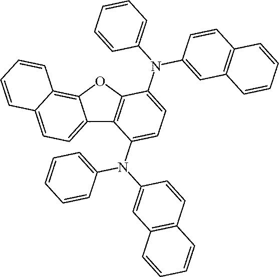 [SMILES: c1ccc(N(c2ccc3ccccc3c2)c2ccc(N(c3ccccc3)c3ccc4ccccc4c3)c3c2oc2cc4ccccc4cc23)cc1] 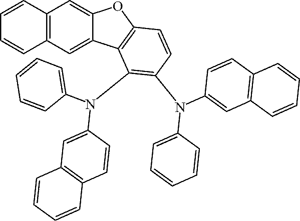 [SMILES: c1ccc(N(c2ccc3ccccc3c2)c2ccc3oc4ccc5ccccc5c4c3c2N(c2ccccc2)c2ccc3ccccc3c2)cc1] 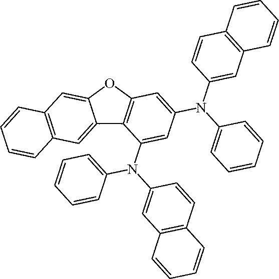 [SMILES: c1ccc(N(c2ccc3ccccc3c2)c2cc(N(c3ccccc3)c3ccc4ccccc4c3)c3c(c2)oc2ccc4ccccc4c23)cc1] 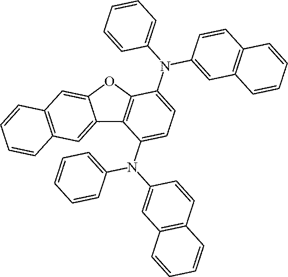 [SMILES: c1ccc(N(c2ccc3ccccc3c2)c2ccc(N(c3ccccc3)c3ccc4ccccc4c3)c3c2oc2ccc4ccccc4c23)cc1] 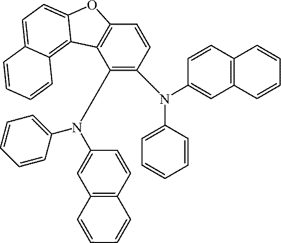 [SMILES: c1ccc(N(c2ccc3ccccc3c2)c2cc3oc4c5ccccc5ccc4c3cc2N(c2ccccc2)c2ccc3ccccc3c2)cc1] 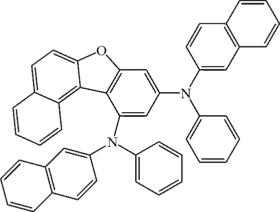 [SMILES: c1ccc(N(c2ccc3ccccc3c2)c2cc(N(c3ccccc3)c3ccc4ccccc4c3)c3oc4c5ccccc5ccc4c3c2)cc1] 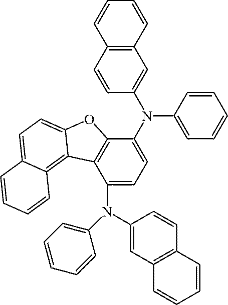 [SMILES: c1ccc(N(c2ccc3ccccc3c2)c2ccc3c(oc4c5ccccc5ccc34)c2N(c2ccccc2)c2ccc3ccccc3c2)cc1] 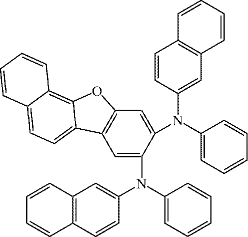 [SMILES: c1ccc(N(c2ccc3ccccc3c2)c2cc3oc4cc5ccccc5cc4c3cc2N(c2ccccc2)c2ccc3ccccc3c2)cc1] 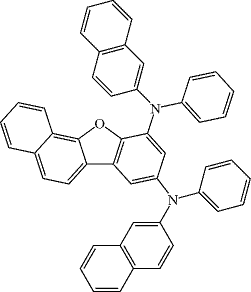 [SMILES: c1ccc(N(c2ccc3ccccc3c2)c2cc(N(c3ccccc3)c3ccc4ccccc4c3)c3oc4cc5ccccc5cc4c3c2)cc1] 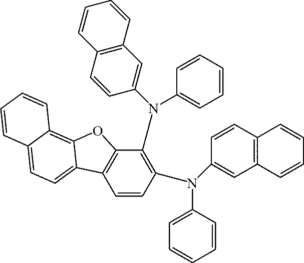 [SMILES: c1ccc(N(c2ccc3ccccc3c2)c2ccc3c(oc4cc5ccccc5cc43)c2N(c2ccccc2)c2ccc3ccccc3c2)cc1] 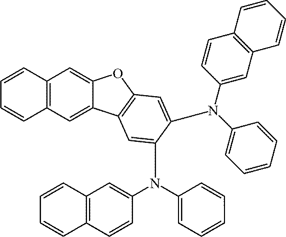 [SMILES: c1ccc(N(c2ccc3ccccc3c2)c2cc3oc4ccc5ccccc5c4c3cc2N(c2ccccc2)c2ccc3ccccc3c2)cc1] 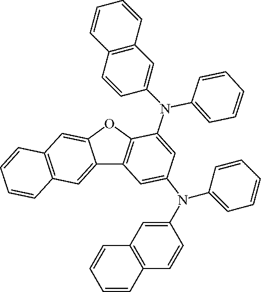 [SMILES: c1ccc(N(c2ccc3ccccc3c2)c2cc(N(c3ccccc3)c3ccc4ccccc4c3)c3oc4ccc5ccccc5c4c3c2)cc1] 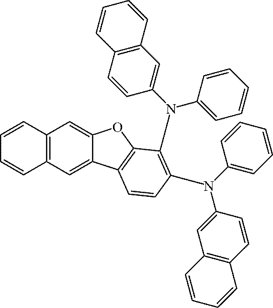 [SMILES: c1ccc(N(c2ccc3ccccc3c2)c2ccc3c(oc4ccc5ccccc5c43)c2N(c2ccccc2)c2ccc3ccccc3c2)cc1] 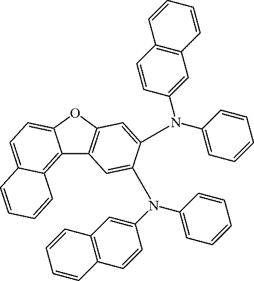 [SMILES: c1ccc(-c2cccc(N(c3ccc4ccccc4c3)c3ccc4oc5c6ccccc6ccc5c4c3N(c3ccccc3)c3ccc4ccccc4c3)c2)cc1] 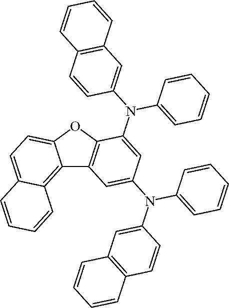 [SMILES: c1ccc(-c2cccc(N(c3ccc4ccccc4c3)c3cc(N(c4ccccc4)c4ccc5ccccc5c4)cc4oc5c6ccccc6ccc5c34)c2)cc1] 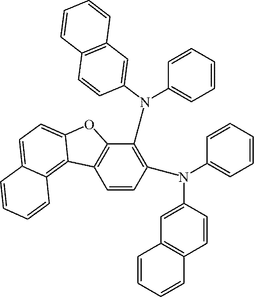 [SMILES: c1ccc(-c2ccc(N(c3ccc4ccccc4c3)c3ccc(N(c4ccccc4)c4ccc5ccccc5c4)c4c3oc3c5ccccc5ccc34)cc2)cc1] 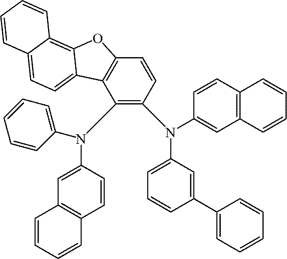 [SMILES: c1ccc(-c2cccc(N(c3ccc4ccccc4c3)c3ccc4oc5cc6ccccc6cc5c4c3N(c3ccccc3)c3ccc4ccccc4c3)c2)cc1] 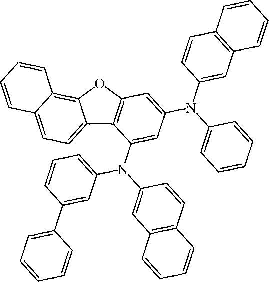 [SMILES: c1ccc(-c2ccc(N(c3ccc4ccccc4c3)c3cc(N(c4ccccc4)c4ccc5ccccc5c4)c4c(c3)oc3cc5ccccc5cc34)cc2)cc1] 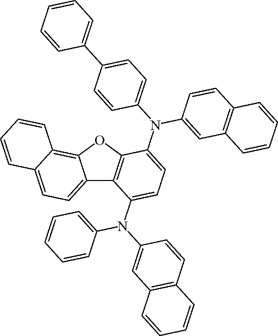 [SMILES: c1ccc(-c2cccc(N(c3ccc4ccccc4c3)c3ccc(N(c4ccccc4)c4ccc5ccccc5c4)c4oc5cc6ccccc6cc5c34)c2)cc1] 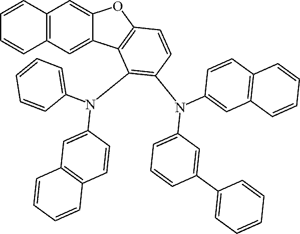 [SMILES: c1ccc(-c2ccc(N(c3ccc4ccccc4c3)c3c(N(c4ccccc4)c4ccc5ccccc5c4)ccc4oc5ccc6ccccc6c5c34)cc2)cc1] 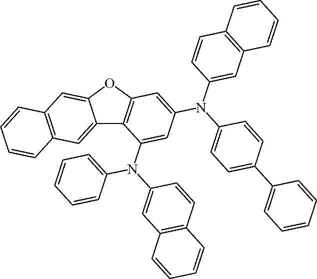 [SMILES: c1ccc(-c2cccc(N(c3ccc4ccccc4c3)c3cc(N(c4ccccc4)c4ccc5ccccc5c4)c4c(c3)oc3ccc5ccccc5c34)c2)cc1] 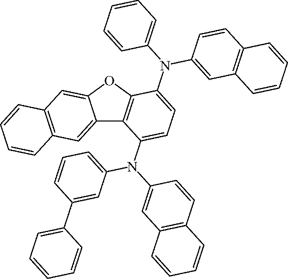 [SMILES: c1ccc(-c2cccc(N(c3ccc4ccccc4c3)c3ccc(N(c4ccccc4)c4ccc5ccccc5c4)c4c3oc3ccc5ccccc5c34)c2)cc1] 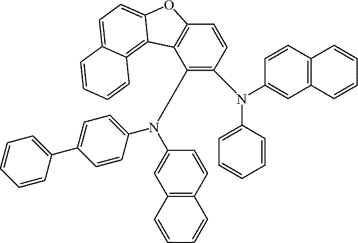 [SMILES: c1ccc(-c2cccc(N(c3ccc4ccccc4c3)c3cc4c(cc3N(c3ccccc3)c3ccc5ccccc5c3)oc3c5ccccc5ccc43)c2)cc1] 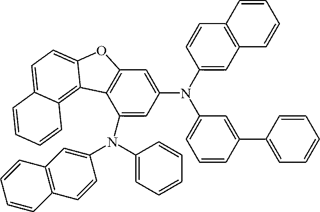 [SMILES: c1ccc(-c2ccc(N(c3ccc4ccccc4c3)c3cc(N(c4ccccc4)c4ccc5ccccc5c4)c4oc5c6ccccc6ccc5c4c3)cc2)cc1] 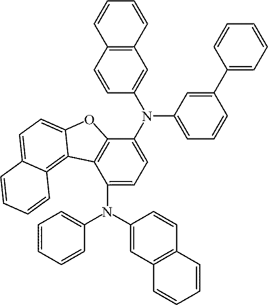 [SMILES: c1ccc(-c2cccc(N(c3ccc4ccccc4c3)c3c(N(c4ccccc4)c4ccc5ccccc5c4)ccc4c3oc3c5ccccc5ccc43)c2)cc1] 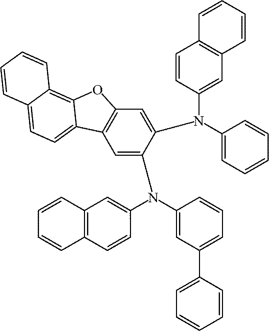 [SMILES: c1ccc(-c2cccc(N(c3ccc4ccccc4c3)c3cc4c(cc3N(c3ccccc3)c3ccc5ccccc5c3)oc3cc5ccccc5cc34)c2)cc1] 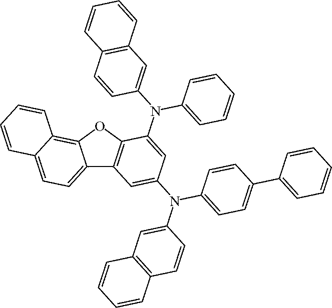 [SMILES: c1ccc(-c2cccc(N(c3ccc4ccccc4c3)c3cc(N(c4ccccc4)c4ccc5ccccc5c4)cc4c3oc3cc5ccccc5cc34)c2)cc1] 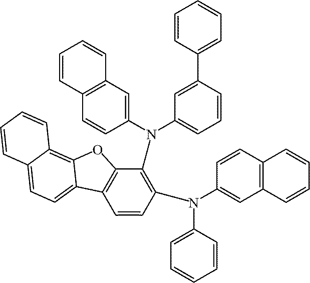 [SMILES: c1ccc(-c2ccc(N(c3ccc4ccccc4c3)c3c(N(c4ccccc4)c4ccc5ccccc5c4)ccc4c3oc3cc5ccccc5cc34)cc2)cc1] 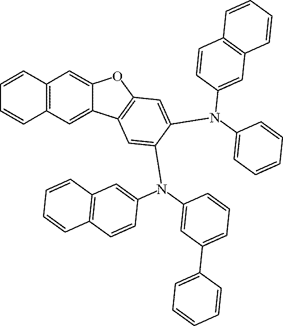 [SMILES: c1ccc(-c2cccc(N(c3ccc4ccccc4c3)c3cc4c(cc3N(c3ccccc3)c3ccc5ccccc5c3)oc3ccc5ccccc5c34)c2)cc1] 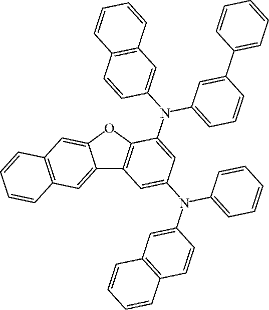 [SMILES: c1ccc(-c2ccc(N(c3ccc4ccccc4c3)c3cc(N(c4ccccc4)c4ccc5ccccc5c4)cc4c3oc3ccc5ccccc5c34)cc2)cc1] 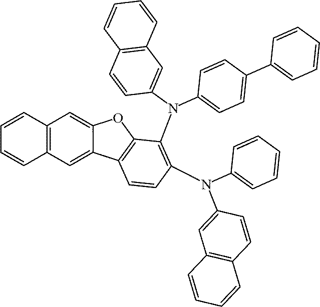 [SMILES: c1ccc(-c2ccc(N(c3ccc4ccccc4c3)c3c(N(c4ccccc4)c4ccc5ccccc5c4)ccc4c3oc3ccc5ccccc5c34)cc2)cc1] 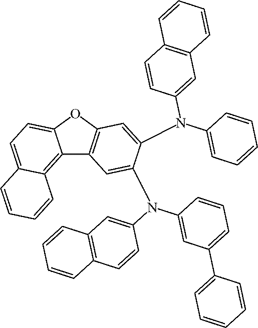 [SMILES: c1ccc(-c2ccc(N(c3ccccc3)c3ccc4oc5c6ccccc6ccc5c4c3N(c3ccccc3)c3ccc4c(c3)oc3ccccc34)cc2)cc1] 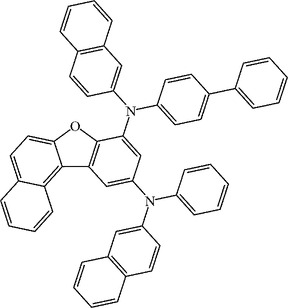 [SMILES: c1ccc(-c2ccc(N(c3ccc4c(c3)oc3ccccc34)c3cc(N(c4ccccc4)c4ccccc4)cc4oc5c6ccccc6ccc5c34)cc2)cc1] 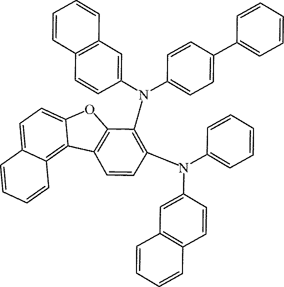 [SMILES: c1ccc(-c2cccc(N(c3ccccc3)c3ccc(N(c4cccc(-c5ccccc5)c4)c4ccc5c(c4)oc4ccccc45)c4c3oc3c5ccccc5ccc34)c2)cc1] 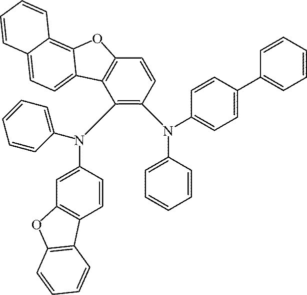 [SMILES: c1ccc(-c2ccc(N(c3ccccc3)c3ccc4oc5cc6ccccc6cc5c4c3N(c3ccccc3)c3ccc4c(c3)sc3ccccc34)c3ccccc23)cc1] 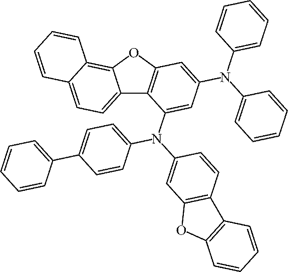 [SMILES: c1ccc(-c2ccc(N(c3ccccc3)c3cc(N(c4ccccc4)c4ccc5oc6ccccc6c5c4)cc4oc5cc6ccccc6cc5c34)cc2)cc1] 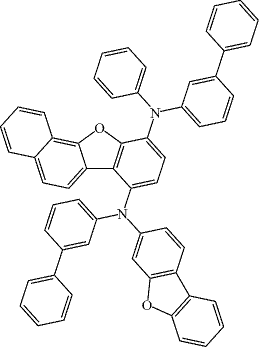 [SMILES: c1ccc(-c2cccc(N(c3ccccc3)c3ccc(N(c4ccccc4)c4ccc5c(c4)oc4ccccc45)c4oc5cc6ccccc6cc5c34)c2)cc1] 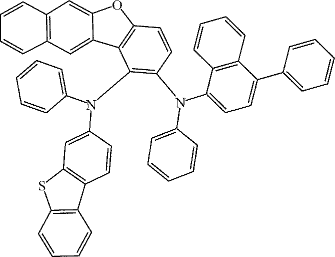 [SMILES: c1ccc(-c2ccc(N(c3ccccc3)c3ccc4oc5ccc6ccccc6c5c4c3N(c3ccccc3)c3ccc4c(c3)sc3ccccc34)c3ccccc23)cc1] 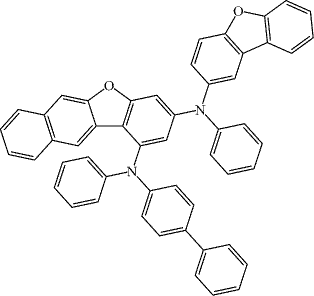 [SMILES: c1ccc(-c2ccc(N(c3ccc4c(c3)sc3ccccc34)c3cc(N(c4ccccc4)c4ccccc4)cc4oc5ccc6ccccc6c5c34)cc2)cc1] 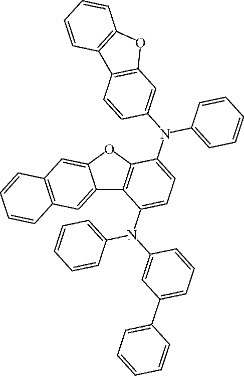 [SMILES: c1ccc(N(c2cc3ccccc3c3ccccc23)c2ccc(N(c3ccc4ccccc4c3)c3ccc4oc5ccccc5c4c3)c3c2oc2ccc4ccccc4c23)cc1] 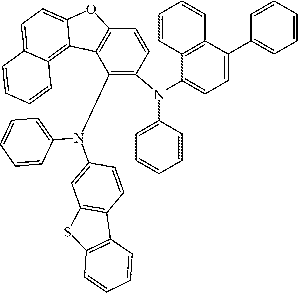 [SMILES: c1ccc(-c2ccc(N(c3ccccc3)c3cc4oc5c6ccccc6ccc5c4cc3N(c3ccc4ccccc4c3)c3ccc4sc5ccccc5c4c3)cc2)cc1] 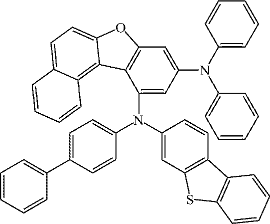 [SMILES: c1ccc(-c2ccc(N(c3ccccc3)c3cc(N(c4ccccc4)c4ccc5sc6ccccc6c5c4)cc4c3oc3c5ccccc5ccc43)cc2)cc1] 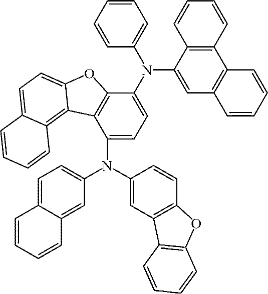 [SMILES: c1ccc(-c2cccc(N(c3ccc4c(c3)oc3ccccc34)c3c(N(c4ccccc4)c4ccccc4)ccc4c3oc3c5ccccc5ccc43)c2)cc1] 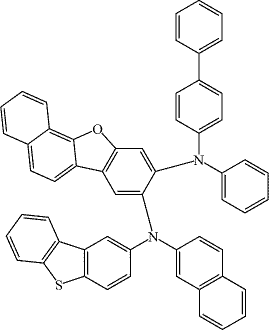 [SMILES: c1ccc(-c2ccc(N(c3ccccc3)c3cc4c(cc3N(c3ccccc3)c3ccc5sc6ccccc6c5c3)oc3cc5ccccc5cc34)c3ccccc23)cc1] 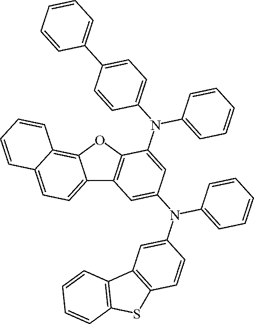 [SMILES: c1ccc(-c2ccc(N(c3ccccc3)c3cc(N(c4ccccc4)c4ccc5c(c4)oc4ccccc45)c4oc5cc6ccccc6cc5c4c3)cc2)cc1] 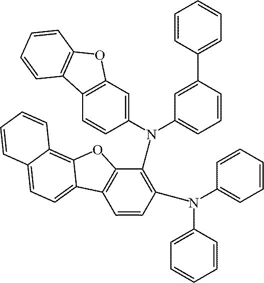 [SMILES: c1ccc(N(c2cccc(-c3ccc4ccccc4c3)c2)c2ccc3c(oc4cc5ccccc5cc43)c2N(c2ccccc2)c2ccc3sc4ccccc4c3c2)cc1] 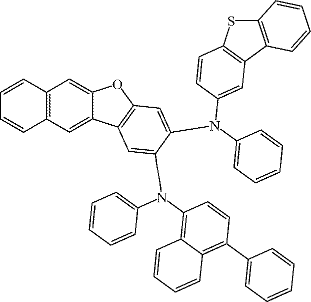 [SMILES: c1ccc(-c2ccc(N(c3ccccc3)c3cc4c(cc3N(c3ccc5ccccc5c3)c3ccc5c(c3)oc3ccccc35)oc3ccc5ccccc5c34)cc2)cc1] 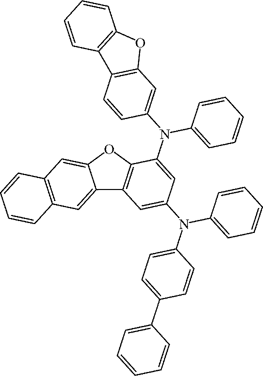 [SMILES: c1ccc(N(c2ccccc2)c2cc(N(c3ccc4ccccc4c3)c3ccc4c(c3)sc3ccccc34)c3oc4ccc5ccccc5c4c3c2)cc1] 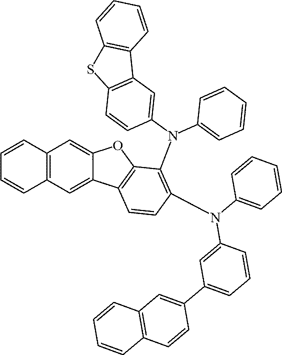 [SMILES: c1ccc(N(c2ccc3ccccc3c2)c2ccc3c(oc4ccc5ccccc5c43)c2N(c2ccc3ccccc3c2)c2ccc3sc4ccccc4c3c2)cc1] 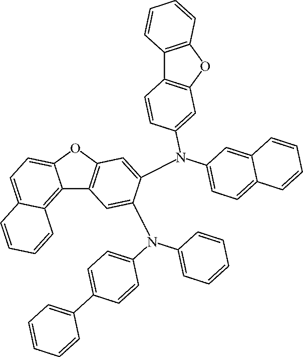 [SMILES: c1ccc(N(c2ccc3oc4c5ccccc5ccc4c3c2N(c2ccccc2)c2cc3ccccc3c3ccccc23)c2cccc3c2sc2ccccc23)cc1] 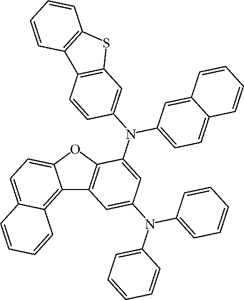 [SMILES: c1ccc(N(c2cc(N(c3ccccc3)c3ccc4c(ccc5ccccc54)c3)c3c(c2)oc2c4ccccc4ccc23)c2cccc3oc4ccccc4c23)cc1] 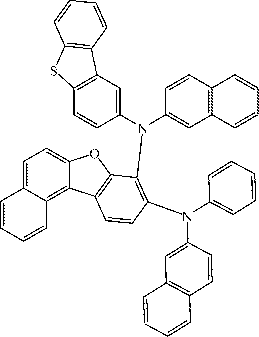 [SMILES: C1=C(N(c2ccccc2)c2ccc(N(c3ccccc3)c3cccc(-c4ccccc4)c3)c3c2oc2c4ccccc4ccc23)c2c(oc3ccccc23)CC1] 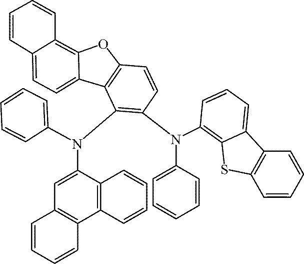 [SMILES: c1ccc(N(c2ccc3oc4cc5ccccc5cc4c3c2N(c2ccccc2)c2ccc3ccc4ccccc4c3c2)c2cccc3c2sc2ccccc23)cc1] 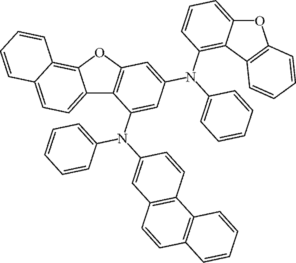 [SMILES: c1ccc(-c2ccc(N(c3ccccc3)c3cc(N(c4ccccc4)c4cccc5oc6ccccc6c45)cc4oc5cc6ccccc6cc5c34)cc2)cc1] 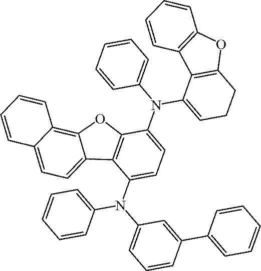 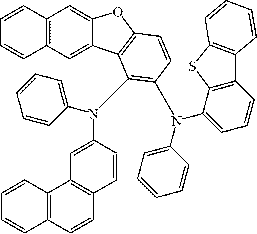 [SMILES: c1ccc(-c2cccc(N(c3ccc4oc5ccc6ccccc6c5c4c3N(c3ccccc3)c3ccc4ccccc4c3)c3cccc4c3sc3ccccc34)c2)cc1] 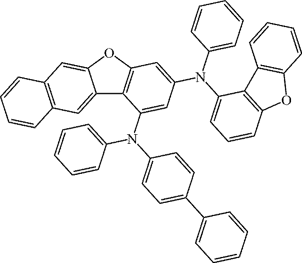 [SMILES: c1ccc(-c2ccc(N(c3cc(N(c4ccccc4)c4ccc(-c5ccccc5)cc4)c4c(c3)oc3ccc5ccccc5c34)c3cccc4c3sc3ccccc34)cc2)cc1] 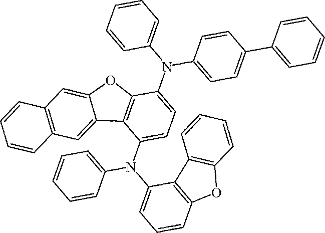 [SMILES: c1ccc(-c2cccc(N(c3ccc(N(c4ccccc4)c4cccc(-c5ccccc5)c4)c4c3oc3ccc5ccccc5c34)c3cccc4c3sc3ccccc34)c2)cc1] 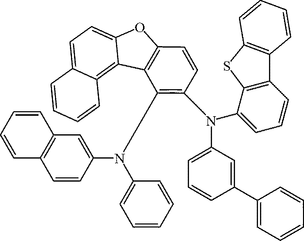 [SMILES: c1ccc(N(c2ccc(-c3cccc4ccccc34)cc2)c2cc3oc4c5ccccc5ccc4c3cc2N(c2ccccc2)c2cccc3c2sc2ccccc23)cc1] 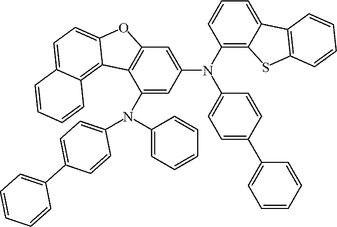 [SMILES: c1ccc(-c2cccc(N(c3ccccc3)c3cc(N(c4ccccc4)c4cccc5oc6ccccc6c45)c4oc5c6ccccc6ccc5c4c3)c2)cc1] 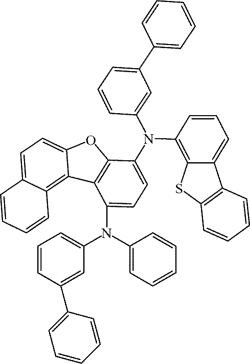 [SMILES: c1ccc(N(c2ccc(-c3ccc4ccccc4c3)cc2)c2c(N(c3ccccc3)c3cccc4c3sc3ccccc34)ccc3c2oc2c4ccccc4ccc32)cc1] 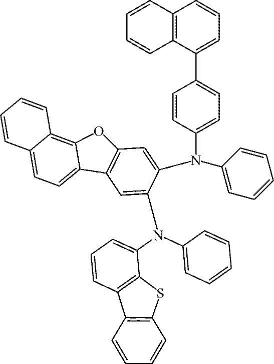 [SMILES: c1ccc(N(c2cc3oc4cc5ccccc5cc4c3cc2N(c2ccc3ccccc3c2)c2ccc3ccccc3c2)c2cccc3c2sc2ccccc23)cc1] 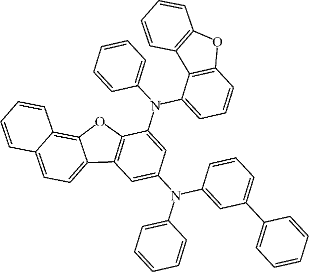 [SMILES: c1ccc(-c2ccc(N(c3ccccc3)c3cc(N(c4ccccc4)c4cccc5oc6ccccc6c45)c4oc5cc6ccccc6cc5c4c3)cc2)cc1] 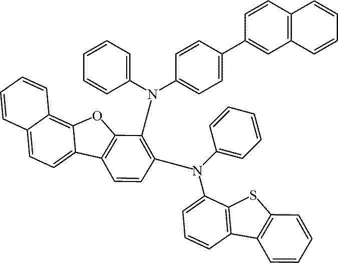 [SMILES: c1ccc(-c2ccc(N(c3c(N(c4ccccc4)c4ccc5ccccc5c4)ccc4c3oc3cc5ccccc5cc34)c3cccc4c3sc3ccccc34)cc2)cc1] 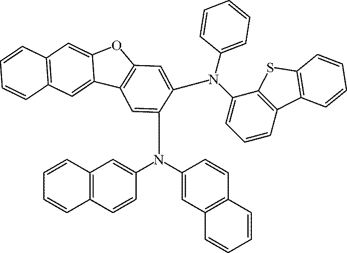 [SMILES: c1ccc(-c2cccc(N(c3cccc(-c4ccccc4)c3)c3cc4c(cc3N(c3ccc5ccccc5c3)c3cccc5c3oc3ccccc35)oc3ccc5ccccc5c34)c2)cc1] 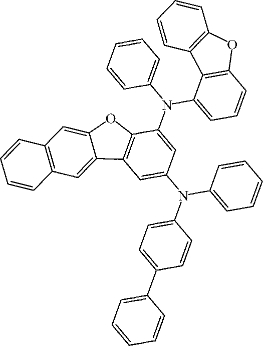 [SMILES: c1ccc(-c2ccc(N(c3ccc(-c4ccccc4)cc3)c3cc(N(c4ccccc4)c4cccc5oc6ccccc6c45)cc4c3oc3ccc5ccccc5c34)cc2)cc1] 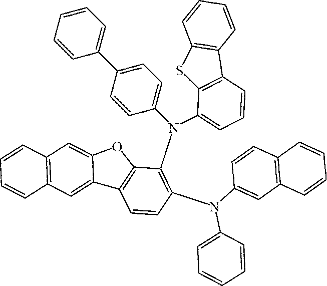 [SMILES: c1ccc(-c2ccc(N(c3ccc4ccccc4c3)c3c(N(c4ccccc4)c4cccc5sc6ccccc6c45)ccc4c3oc3ccc5ccccc5c34)cc2)cc1] 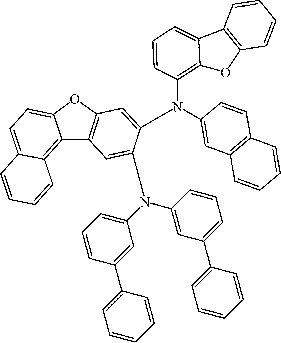 [SMILES: c1ccc(-c2ccc(N(c3ccccc3)c3ccc4oc5c6ccccc6ccc5c4c3N(c3ccc(-c4ccccc4)cc3)c3cccc(-c4ccccc4)c3)cc2)cc1] 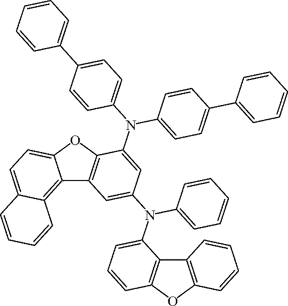 [SMILES: c1ccc(-c2ccc(N(c3cccc(-c4ccccc4)c3)c3cc(N(c4ccccc4)c4ccc(-c5ccccc5)cc4)c4c(c3)oc3c5ccccc5ccc34)cc2)cc1] 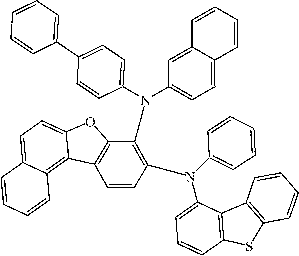 [SMILES: c1ccc(-c2ccc(N(c3ccccc3)c3ccc(N(c4ccc(-c5ccccc5)cc4)c4cccc(-c5ccccc5)c4)c4c3oc3c5ccccc5ccc34)cc2)cc1] 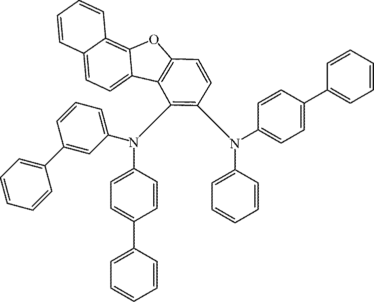 [SMILES: c1ccc(-c2ccc(N(c3cccc(-c4ccccc4)c3)c3ccc4oc5cc6ccccc6cc5c4c3N(c3ccccc3)c3ccc(-c4ccccc4)cc3)cc2)cc1] 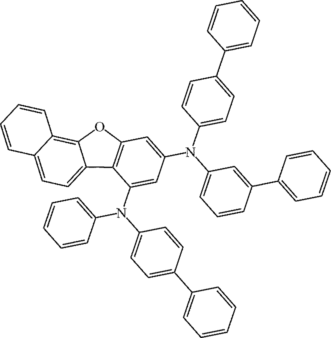 [SMILES: c1ccc(-c2ccc(N(c3cccc(-c4ccccc4)c3)c3cc(N(c4ccccc4)c4ccc(-c5ccccc5)cc4)c4c(c3)oc3cc5ccccc5cc34)cc2)cc1] 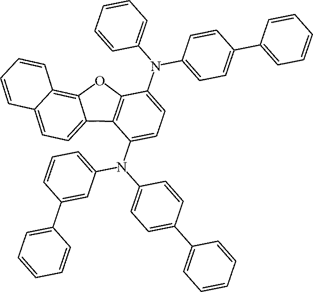 [SMILES: c1ccc(-c2ccc(N(c3ccccc3)c3ccc(N(c4ccc(-c5ccccc5)cc4)c4cccc(-c5ccccc5)c4)c4c3oc3cc5ccccc5cc34)cc2)cc1] 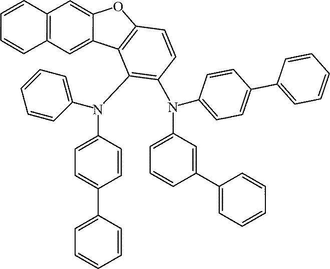 [SMILES: c1ccc(-c2ccc(N(c3ccccc3)c3ccc4oc5ccc6ccccc6c5c4c3N(c3ccc(-c4ccccc4)cc3)c3cccc(-c4ccccc4)c3)cc2)cc1] 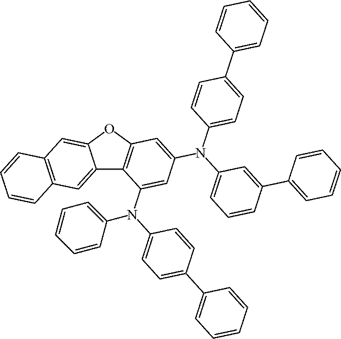 [SMILES: c1ccc(-c2ccc(N(c3cccc(-c4ccccc4)c3)c3cc(N(c4ccccc4)c4ccc(-c5ccccc5)cc4)c4c(c3)oc3ccc5ccccc5c34)cc2)cc1] 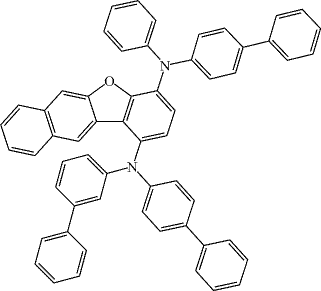 [SMILES: c1ccc(-c2ccc(N(c3ccccc3)c3ccc(N(c4ccc(-c5ccccc5)cc4)c4cccc(-c5ccccc5)c4)c4c3oc3ccc5ccccc5c34)cc2)cc1] 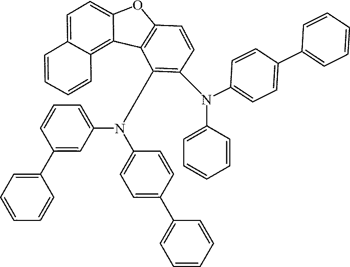 [SMILES: c1ccc(-c2ccc(N(c3ccccc3)c3cc4oc5c6ccccc6ccc5c4cc3N(c3ccc(-c4ccccc4)cc3)c3cccc(-c4ccccc4)c3)cc2)cc1] 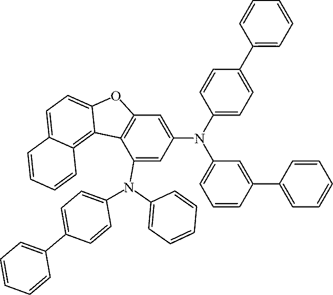 [SMILES: c1ccc(-c2ccc(N(c3cccc(-c4ccccc4)c3)c3cc(N(c4ccccc4)c4ccc(-c5ccccc5)cc4)c4oc5c6ccccc6ccc5c4c3)cc2)cc1] 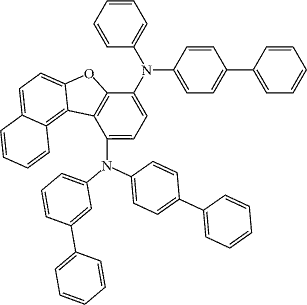 [SMILES: c1ccc(-c2ccc(N(c3cccc(-c4ccccc4)c3)c3ccc4c(oc5c6ccccc6ccc45)c3N(c3ccccc3)c3ccc(-c4ccccc4)cc3)cc2)cc1] 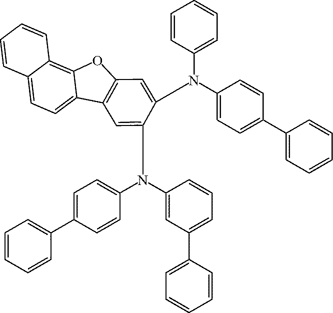 [SMILES: c1ccc(-c2ccc(N(c3ccccc3)c3cc4oc5cc6ccccc6cc5c4cc3N(c3ccc(-c4ccccc4)cc3)c3cccc(-c4ccccc4)c3)cc2)cc1] 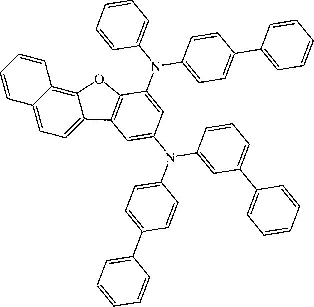 [SMILES: c1ccc(-c2ccc(N(c3ccccc3)c3cc(N(c4ccc(-c5ccccc5)cc4)c4cccc(-c5ccccc5)c4)c4oc5cc6ccccc6cc5c4c3)cc2)cc1] 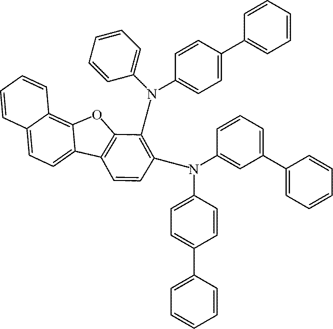 [SMILES: c1ccc(-c2ccc(N(c3ccccc3)c3ccc4c(oc5cc6ccccc6cc54)c3N(c3ccc(-c4ccccc4)cc3)c3cccc(-c4ccccc4)c3)cc2)cc1] 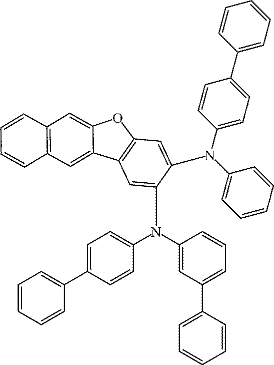 [SMILES: c1ccc(-c2ccc(N(c3cccc(-c4ccccc4)c3)c3cc4oc5ccc6ccccc6c5c4cc3N(c3ccccc3)c3ccc(-c4ccccc4)cc3)cc2)cc1] 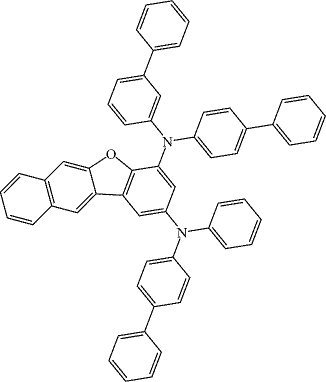 [SMILES: c1ccc(-c2ccc(N(c3cccc(-c4ccccc4)c3)c3cc(N(c4ccccc4)c4ccc(-c5ccccc5)cc4)c4oc5ccc6ccccc6c5c4c3)cc2)cc1] 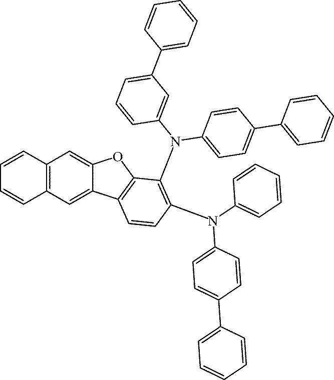 [SMILES: c1ccc(-c2ccc(N(c3ccccc3)c3ccc4c(oc5ccc6ccccc6c54)c3N(c3ccc(-c4ccccc4)cc3)c3cccc(-c4ccccc4)c3)cc2)cc1] 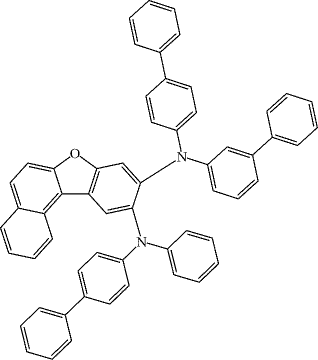 [SMILES: c1ccc(-c2ccc(N(c3ccccc3)c3ccc4oc5c6ccccc6ccc5c4c3N(c3ccccc3)c3ccccc3-c3ccccc3)cc2)cc1] 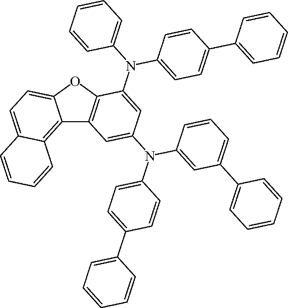 [SMILES: c1ccc(-c2ccc(N(c3ccccc3)c3cc(N(c4ccccc4)c4ccccc4-c4ccccc4)c4c(c3)oc3c5ccccc5ccc34)cc2)cc1] 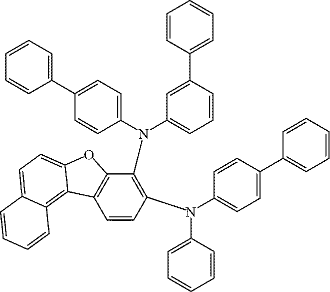 [SMILES: c1ccc(-c2ccc(N(c3ccccc3)c3ccc(N(c4ccccc4)c4ccccc4-c4ccccc4)c4c3oc3c5ccccc5ccc34)cc2)cc1] 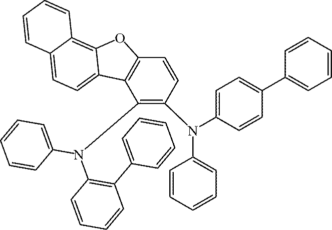 [SMILES: c1ccc(-c2ccc(N(c3ccccc3)c3ccc4oc5cc6ccccc6cc5c4c3N(c3ccccc3)c3ccccc3-c3ccccc3)cc2)cc1] 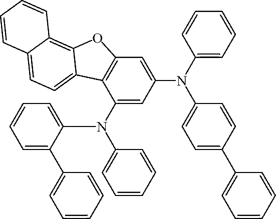 [SMILES: c1ccc(-c2ccc(N(c3ccccc3)c3cc(N(c4ccccc4)c4ccccc4-c4ccccc4)c4c(c3)oc3cc5ccccc5cc34)cc2)cc1] 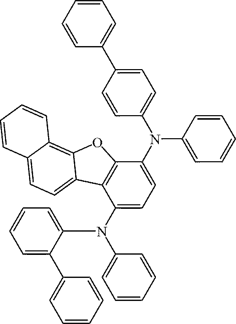 [SMILES: c1ccc(-c2ccc(N(c3ccccc3)c3ccc(N(c4ccccc4)c4ccccc4-c4ccccc4)c4oc5cc6ccccc6cc5c34)cc2)cc1] 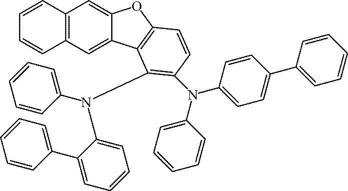 [SMILES: c1ccc(-c2ccc(N(c3ccccc3)c3ccc4oc5ccc6ccccc6c5c4c3N(c3ccccc3)c3ccccc3-c3ccccc3)cc2)cc1] 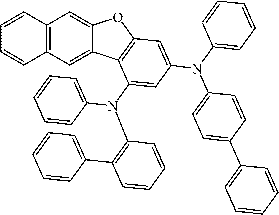 [SMILES: c1ccc(-c2ccc(N(c3ccccc3)c3cc(N(c4ccccc4)c4ccccc4-c4ccccc4)cc4oc5ccc6ccccc6c5c34)cc2)cc1] 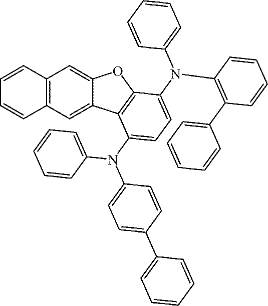 [SMILES: c1ccc(-c2ccc(N(c3ccccc3)c3ccc(N(c4ccccc4)c4ccccc4-c4ccccc4)c4c3oc3ccc5ccccc5c34)cc2)cc1] 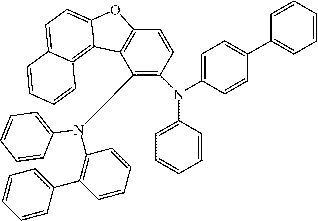 [SMILES: c1ccc(-c2ccc(N(c3ccccc3)c3cc4oc5c6ccccc6ccc5c4cc3N(c3ccccc3)c3ccccc3-c3ccccc3)cc2)cc1] 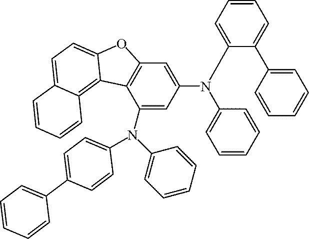 [SMILES: c1ccc(-c2ccc(N(c3ccccc3)c3cc(N(c4ccccc4)c4ccccc4-c4ccccc4)cc4c3oc3c5ccccc5ccc43)cc2)cc1] 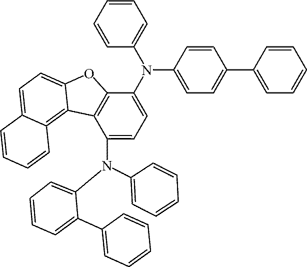 [SMILES: c1ccc(-c2ccc(N(c3ccccc3)c3c(N(c4ccccc4)c4ccccc4-c4ccccc4)ccc4c3oc3c5ccccc5ccc43)cc2)cc1] 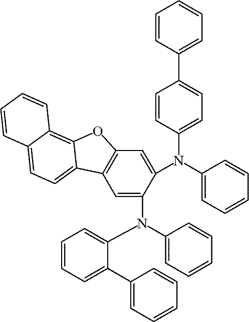 [SMILES: c1ccc(-c2ccc(N(c3ccccc3)c3cc4oc5cc6ccccc6cc5c4cc3N(c3ccccc3)c3ccccc3-c3ccccc3)cc2)cc1] 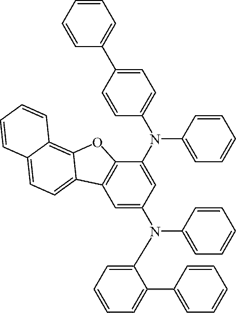 [SMILES: c1ccc(-c2ccc(N(c3ccccc3)c3cc(N(c4ccccc4)c4ccccc4-c4ccccc4)c4oc5cc6ccccc6cc5c4c3)cc2)cc1] 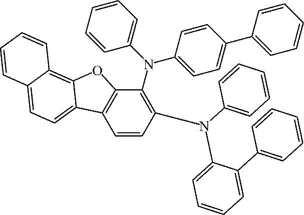 [SMILES: c1ccc(-c2ccc(N(c3ccccc3)c3c(N(c4ccccc4)c4ccccc4-c4ccccc4)ccc4c3oc3cc5ccccc5cc34)cc2)cc1] 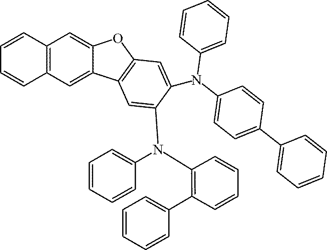 [SMILES: c1ccc(-c2ccc(N(c3ccccc3)c3cc4oc5ccc6ccccc6c5c4cc3N(c3ccccc3)c3ccccc3-c3ccccc3)cc2)cc1] 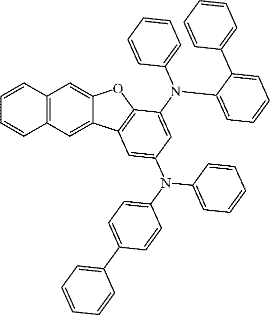 [SMILES: c1ccc(-c2ccccc2N(c2ccccc2)c2cc(N(c3ccccc3)c3ccc4ccccc4c3)cc3c2oc2ccc4ccccc4c23)cc1] 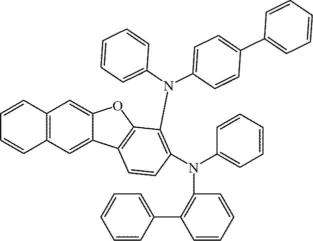 [SMILES: c1ccc(-c2ccc(N(c3ccccc3)c3ccc4c(oc5ccc6ccccc6c54)c3N(c3ccccc3)c3ccccc3-c3ccccc3)cc2)cc1] 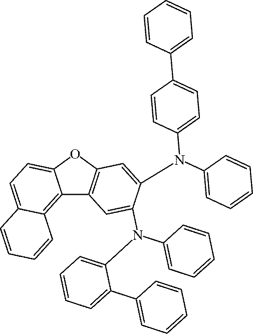 [SMILES: c1ccc(-c2ccc(N(c3ccccc3)c3ccc4oc5c6ccccc6ccc5c4c3N(c3ccccc3)c3ccc(-c4ccccc4)cc3)cc2)cc1] 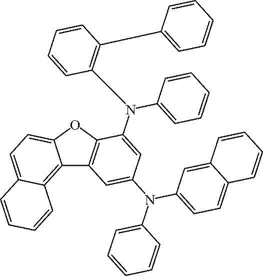 [SMILES: c1ccc(-c2ccc(N(c3ccccc3)c3cc(N(c4ccccc4)c4ccc(-c5ccccc5)cc4)c4c(c3)oc3c5ccccc5ccc34)cc2)cc1] 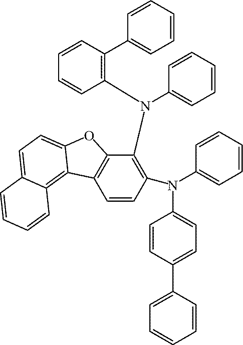 [SMILES: c1ccc(-c2ccc(N(c3ccccc3)c3ccc(N(c4ccccc4)c4ccc(-c5ccccc5)cc4)c4c3oc3c5ccccc5ccc34)cc2)cc1] 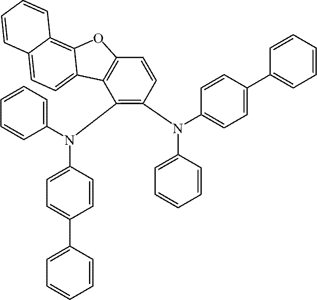 [SMILES: c1ccc(-c2ccc(N(c3ccccc3)c3ccc4oc5cc6ccccc6cc5c4c3N(c3ccccc3)c3ccc(-c4ccccc4)cc3)cc2)cc1] 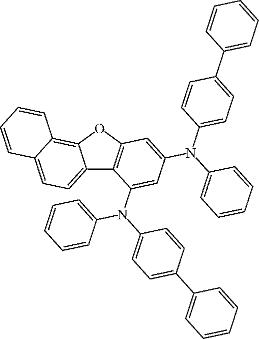 [SMILES: c1ccc(-c2ccc(N(c3ccccc3)c3cc(N(c4ccccc4)c4ccc(-c5ccccc5)cc4)c4c(c3)oc3cc5ccccc5cc34)cc2)cc1] 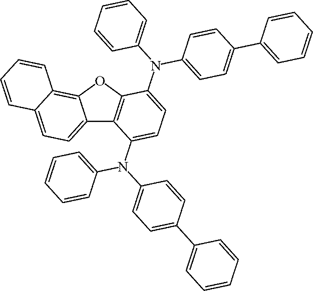 [SMILES: c1ccc(-c2ccc(N(c3ccccc3)c3ccc(N(c4ccccc4)c4ccc(-c5ccccc5)cc4)c4c3oc3cc5ccccc5cc34)cc2)cc1] 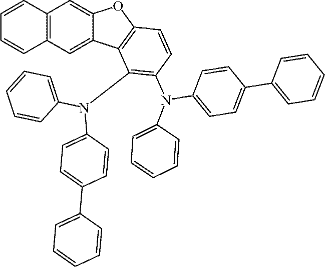 [SMILES: c1ccc(-c2ccc(N(c3ccccc3)c3ccc4oc5ccc6ccccc6c5c4c3N(c3ccccc3)c3ccc(-c4ccccc4)cc3)cc2)cc1] 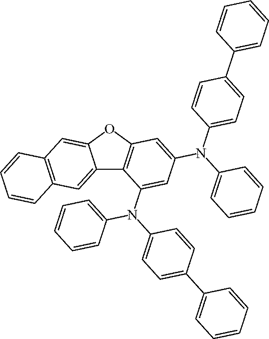 [SMILES: c1ccc(-c2ccc(N(c3ccccc3)c3cc(N(c4ccccc4)c4ccc(-c5ccccc5)cc4)c4c(c3)oc3ccc5ccccc5c34)cc2)cc1] 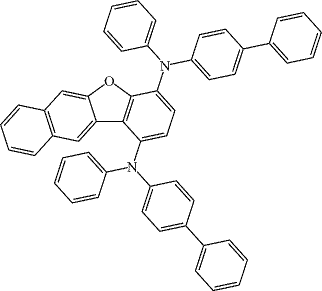 [SMILES: c1ccc(-c2ccc(N(c3ccccc3)c3ccc(N(c4ccccc4)c4ccc(-c5ccccc5)cc4)c4c3oc3ccc5ccccc5c34)cc2)cc1] 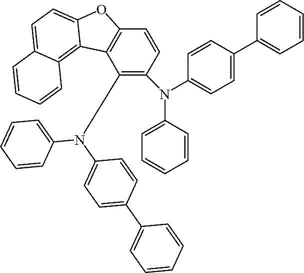 [SMILES: c1ccc(-c2ccc(N(c3ccccc3)c3cc4oc5c6ccccc6ccc5c4cc3N(c3ccccc3)c3ccc(-c4ccccc4)cc3)cc2)cc1] 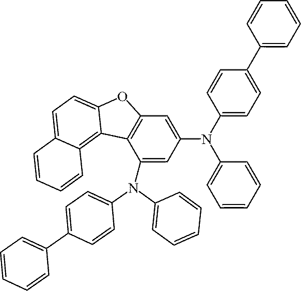 [SMILES: c1ccc(-c2ccc(N(c3ccccc3)c3cc(N(c4ccccc4)c4ccc(-c5ccccc5)cc4)c4oc5c6ccccc6ccc5c4c3)cc2)cc1] 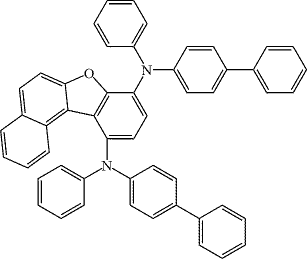 [SMILES: c1ccc(-c2ccc(N(c3ccccc3)c3ccc4c(oc5c6ccccc6ccc45)c3N(c3ccccc3)c3ccc(-c4ccccc4)cc3)cc2)cc1] 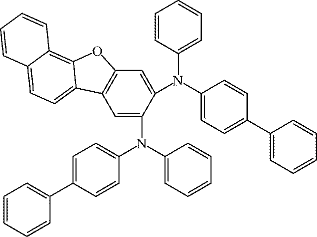 [SMILES: c1ccc(-c2ccc(N(c3ccccc3)c3cc4oc5cc6ccccc6cc5c4cc3N(c3ccccc3)c3ccc(-c4ccccc4)cc3)cc2)cc1] 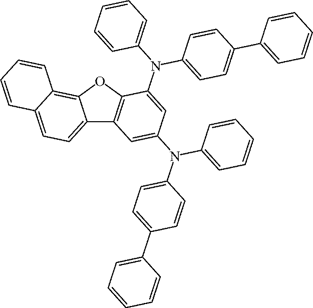 [SMILES: c1ccc(-c2ccc(N(c3ccccc3)c3cc(N(c4ccccc4)c4ccc(-c5ccccc5)cc4)c4oc5cc6ccccc6cc5c4c3)cc2)cc1] 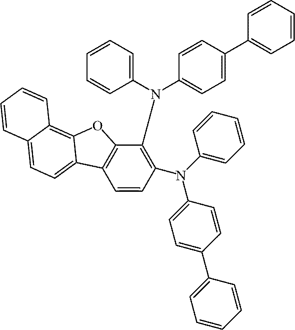 [SMILES: c1ccc(-c2ccc(N(c3ccccc3)c3ccc4c(oc5cc6ccccc6cc54)c3N(c3ccccc3)c3ccc(-c4ccccc4)cc3)cc2)cc1] 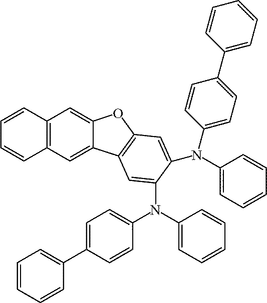 [SMILES: c1ccc(-c2ccc(N(c3ccccc3)c3cc4oc5ccc6ccccc6c5c4cc3N(c3ccccc3)c3ccc(-c4ccccc4)cc3)cc2)cc1] 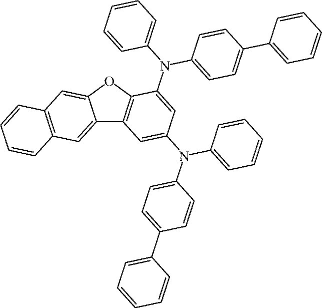 [SMILES: c1ccc(-c2ccc(N(c3ccccc3)c3cc(N(c4ccccc4)c4ccc(-c5ccccc5)cc4)c4oc5ccc6ccccc6c5c4c3)cc2)cc1] 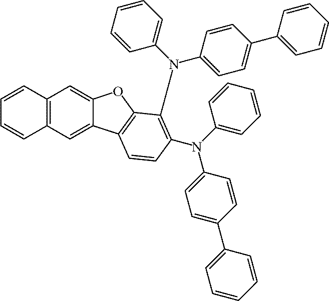 [SMILES: c1ccc(-c2ccc(N(c3ccccc3)c3ccc4c(oc5ccc6ccccc6c54)c3N(c3ccccc3)c3ccc(-c4ccccc4)cc3)cc2)cc1] 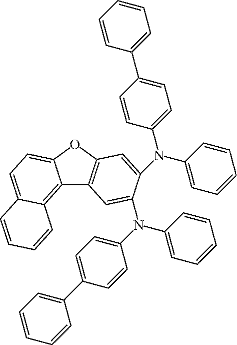 [SMILES: c1ccc(-c2cccc(N(c3ccccc3)c3ccc4oc5c6ccccc6ccc5c4c3N(c3ccccc3)c3ccccc3-c3ccccc3)c2)cc1] 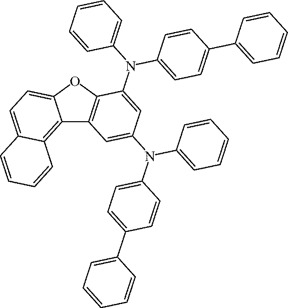 [SMILES: c1ccc(-c2cccc(N(c3ccccc3)c3cc(N(c4ccccc4)c4ccccc4-c4ccccc4)c4c(c3)oc3c5ccccc5ccc34)c2)cc1] 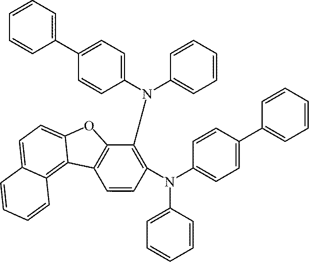 [SMILES: c1ccc(-c2cccc(N(c3ccccc3)c3ccc(N(c4ccccc4)c4ccccc4-c4ccccc4)c4c3oc3c5ccccc5ccc34)c2)cc1] 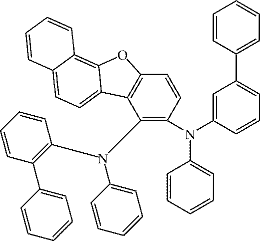 [SMILES: c1ccc(-c2cccc(N(c3ccccc3)c3ccc4oc5cc6ccccc6cc5c4c3N(c3ccccc3)c3ccccc3-c3ccccc3)c2)cc1] 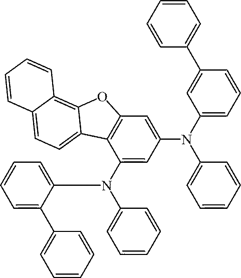 [SMILES: c1ccc(-c2cccc(N(c3ccccc3)c3cc(N(c4ccccc4)c4ccccc4-c4ccccc4)c4c(c3)oc3cc5ccccc5cc34)c2)cc1] 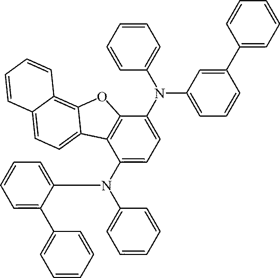 [SMILES: c1ccc(-c2cccc(N(c3ccccc3-c3ccccc3)c3ccc(N(c4ccccc4)c4ccccc4)c4c3oc3cc5ccccc5cc34)c2)cc1] 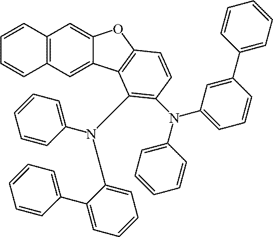 [SMILES: c1ccc(-c2cccc(N(c3ccccc3)c3ccc4oc5ccc6ccccc6c5c4c3N(c3ccccc3)c3ccccc3-c3ccccc3)c2)cc1] 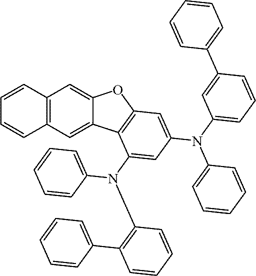 [SMILES: c1ccc(-c2cccc(N(c3ccccc3)c3cc(N(c4ccccc4)c4ccccc4-c4ccccc4)cc4oc5ccc6ccccc6c5c34)c2)cc1] 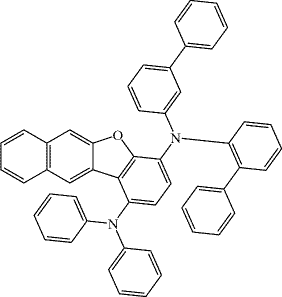 [SMILES: c1ccc(-c2cccc(N(c3ccccc3)c3ccc(N(c4ccccc4)c4ccccc4-c4ccccc4)c4c3oc3ccc5ccccc5c34)c2)cc1] 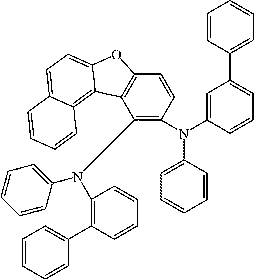 [SMILES: c1ccc(-c2cccc(N(c3ccccc3)c3cc4oc5c6ccccc6ccc5c4cc3N(c3ccccc3)c3ccccc3-c3ccccc3)c2)cc1] 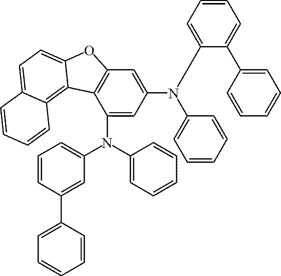 [SMILES: c1ccc(-c2cccc(N(c3ccccc3)c3cc(N(c4ccccc4)c4ccccc4-c4ccccc4)cc4c3oc3c5ccccc5ccc43)c2)cc1] 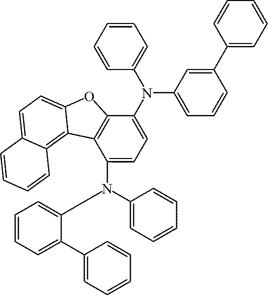 [SMILES: c1ccc(-c2cccc(N(c3ccccc3)c3c(N(c4ccccc4)c4ccccc4-c4ccccc4)ccc4c3oc3c5ccccc5ccc43)c2)cc1] 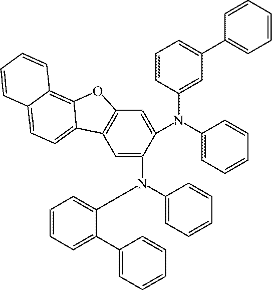 [SMILES: c1ccc(-c2cccc(N(c3ccccc3)c3cc4oc5cc6ccccc6cc5c4cc3N(c3ccccc3)c3ccccc3-c3ccccc3)c2)cc1] 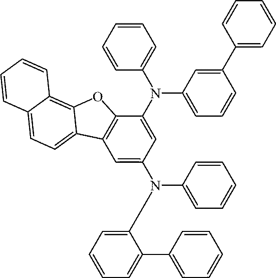 [SMILES: c1ccc(-c2cccc(N(c3ccccc3)c3cc(N(c4ccccc4)c4ccccc4-c4ccccc4)c4oc5cc6ccccc6cc5c4c3)c2)cc1] 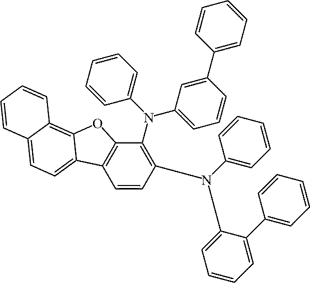 [SMILES: c1ccc(-c2cccc(N(c3ccccc3)c3c(N(c4ccccc4)c4ccccc4-c4ccccc4)ccc4c3oc3cc5ccccc5cc34)c2)cc1] 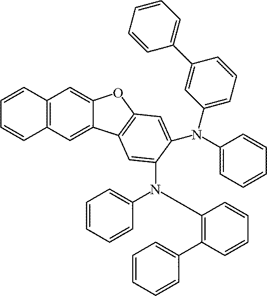 [SMILES: c1ccc(-c2cccc(N(c3ccccc3)c3cc4oc5ccc6ccccc6c5c4cc3N(c3ccccc3)c3ccccc3-c3ccccc3)c2)cc1] 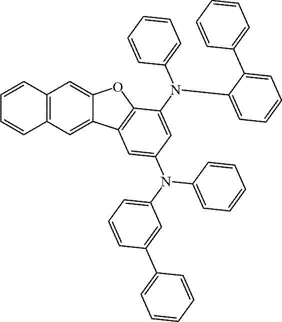 [SMILES: c1ccc(-c2cccc(N(c3ccccc3)c3cc(N(c4ccccc4)c4ccccc4-c4ccccc4)c4oc5ccc6ccccc6c5c4c3)c2)cc1] 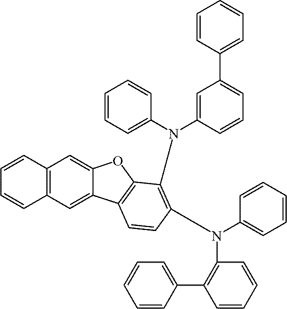 [SMILES: c1ccc(-c2cccc(N(c3ccccc3)c3ccc4c(oc5ccc6ccccc6c54)c3N(c3ccccc3)c3ccccc3-c3ccccc3)c2)cc1] 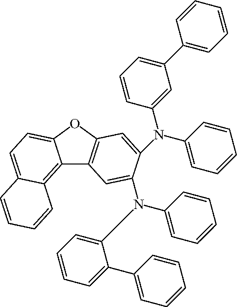 [SMILES: c1ccc(N(c2ccc3oc4ccccc4c3c2)c2ccc3oc4c5ccccc5ccc4c3c2N(c2ccccc2)c2ccc3c(c2)oc2ccccc23)cc1] 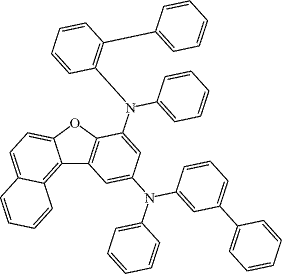 [SMILES: c1ccc(N(c2ccc3c(c2)oc2ccccc23)c2cc(N(c3ccccc3)c3ccc4c(c3)oc3ccccc34)c3c(c2)oc2c4ccccc4ccc23)cc1] 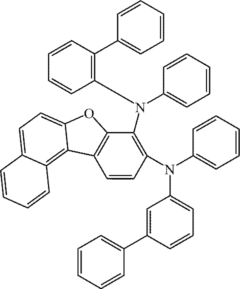 [SMILES: c1ccc(N(c2ccc3c(c2)oc2ccccc23)c2ccc(N(c3ccccc3)c3ccc4c(c3)oc3ccccc34)c3c2oc2c4ccccc4ccc23)cc1] 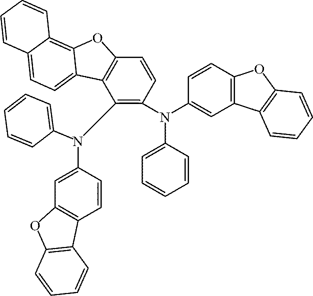 [SMILES: c1ccc(N(c2ccc3c(c2)sc2ccccc23)c2ccc3oc4cc5ccccc5cc4c3c2N(c2ccccc2)c2ccc3c(c2)sc2ccccc23)cc1] 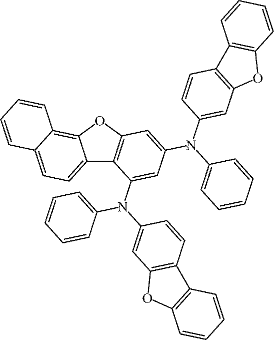 [SMILES: c1ccc(N(c2cc(N(c3ccccc3)c3ccc4oc5ccccc5c4c3)c3c(c2)oc2cc4ccccc4cc23)c2ccc3oc4ccccc4c3c2)cc1] 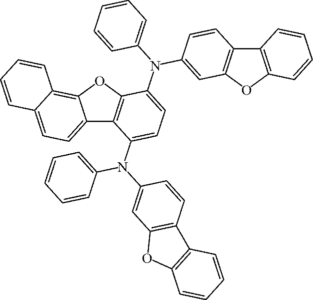 [SMILES: c1ccc(N(c2ccc3c(c2)oc2ccccc23)c2ccc(N(c3ccccc3)c3ccc4c(c3)oc3ccccc34)c3c2oc2cc4ccccc4cc23)cc1] 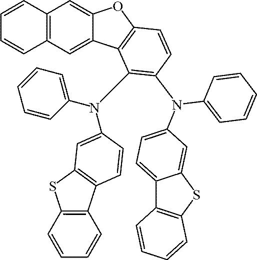 [SMILES: c1ccc(N(c2ccc3oc4ccc5ccccc5c4c3c2N(c2ccccc2)c2ccc3c(c2)sc2ccccc23)c2cccc3c2sc2ccccc23)cc1] 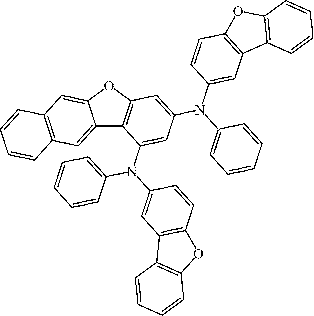 [SMILES: c1ccc(N(c2cc(N(c3ccccc3)c3ccc4c(c3)sc3ccccc34)c3c(c2)oc2ccc4ccccc4c23)c2ccc3sc4ccccc4c3c2)cc1] 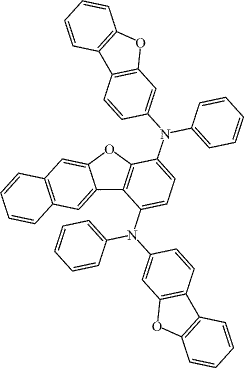 [SMILES: c1ccc(N(c2ccccc2)c2ccc(N(c3ccc4c(c3)oc3ccccc34)c3ccc4oc5ccccc5c4c3)c3c2oc2ccc4ccccc4c23)cc1] 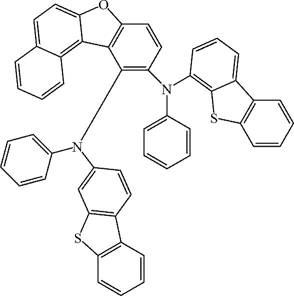 [SMILES: c1ccc(N(c2ccc3oc4ccccc4c3c2)c2cc3oc4c5ccccc5ccc4c3cc2N(c2ccccc2)c2ccc3sc4ccccc4c3c2)cc1] 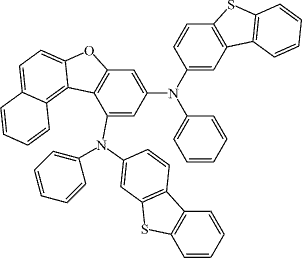 [SMILES: c1ccc(N(c2ccc3sc4ccccc4c3c2)c2cc(N(c3ccccc3)c3ccc4sc5ccccc5c4c3)c3oc4c5ccccc5ccc4c3c2)cc1] 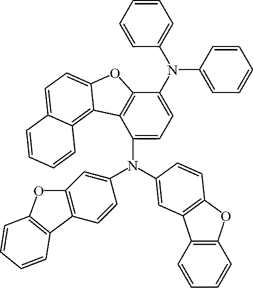 [SMILES: c1ccc(N(c2ccccc2)c2ccc3c(oc4c5ccccc5ccc34)c2N(c2ccc3c(c2)oc2ccccc23)c2ccc3c(c2)oc2ccccc23)cc1] 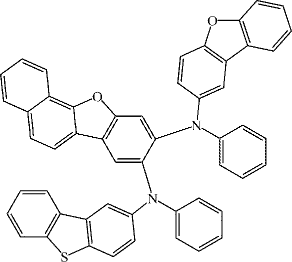 [SMILES: c1ccc(N(c2ccc3sc4ccccc4c3c2)c2cc3oc4cc5ccccc5cc4c3cc2N(c2ccccc2)c2cccc3oc4ccccc4c23)cc1] 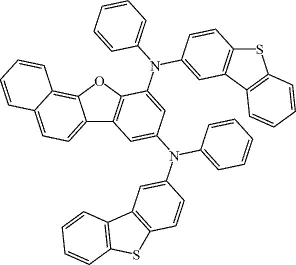 [SMILES: c1ccc(N(c2ccc3c(c2)oc2ccccc23)c2cc(N(c3ccccc3)c3ccc4c(c3)oc3ccccc34)c3oc4cc5ccccc5cc4c3c2)cc1] 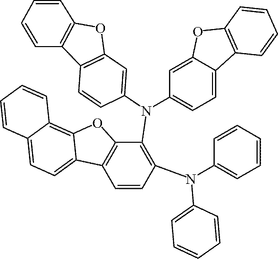 [SMILES: c1ccc(N(c2ccc3sc4ccccc4c3c2)c2c(N(c3ccccc3)c3cccc4c3sc3ccccc34)ccc3c2oc2cc4ccccc4cc23)cc1] 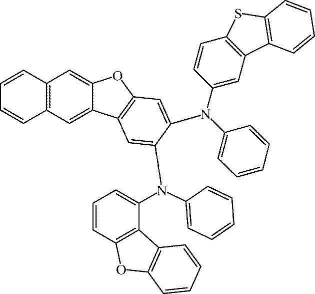 [SMILES: c1ccc(N(c2ccc3c(c2)oc2ccccc23)c2cc3oc4ccc5ccccc5c4c3cc2N(c2ccccc2)c2cccc3c2sc2ccccc23)cc1] 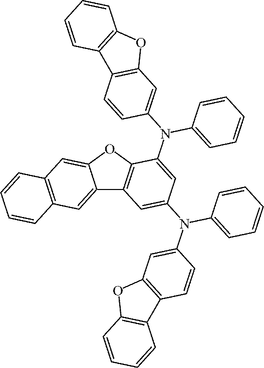 [SMILES: c1ccc(N(c2ccc3sc4ccccc4c3c2)c2cc(N(c3ccccc3)c3ccc4c(c3)sc3ccccc34)c3oc4ccc5ccccc5c4c3c2)cc1] 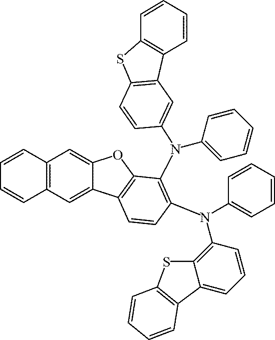 [SMILES: c1ccc(N(c2ccc3sc4ccccc4c3c2)c2c(N(c3ccccc3)c3cccc4c3sc3ccccc34)ccc3c2oc2ccc4ccccc4c23)cc1] 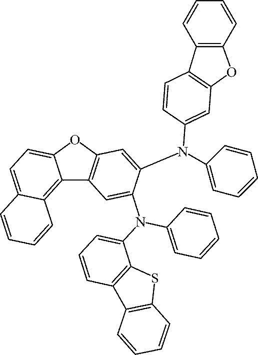 [SMILES: c1ccc(N(c2ccc3oc4c5ccccc5ccc4c3c2N(c2ccccc2)c2ccc3oc4ccccc4c3c2)c2cccc3c2sc2ccccc23)cc1] 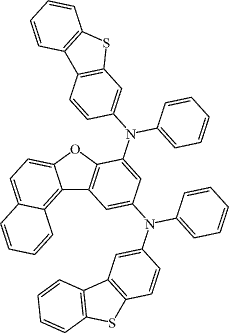 [SMILES: c1ccc(N(c2cc(N(c3ccccc3)c3cccc4oc5ccccc5c34)c3c(c2)oc2c4ccccc4ccc23)c2cccc3oc4ccccc4c23)cc1] 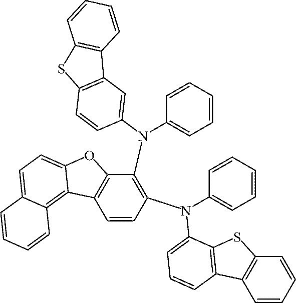 [SMILES: c1ccc(N(c2ccc(N(c3ccccc3)c3cccc4oc5ccccc5c34)c3c2oc2c4ccccc4ccc23)c2cccc3oc4ccccc4c23)cc1] 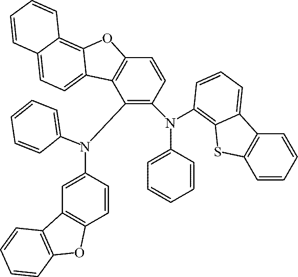 [SMILES: c1ccc(N(c2ccccc2)c2c(N(c3cccc4c3sc3ccccc34)c3cccc4c3sc3ccccc34)ccc3oc4cc5ccccc5cc4c23)cc1] 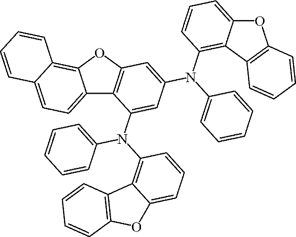 [SMILES: c1ccc(N(c2cc(N(c3ccccc3)c3cccc4oc5ccccc5c34)c3c(c2)oc2cc4ccccc4cc23)c2cccc3oc4ccccc4c23)cc1] 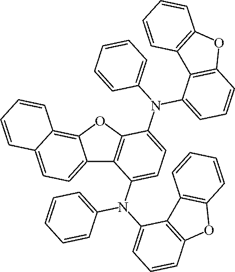 [SMILES: c1ccc(N(c2ccc(N(c3ccccc3)c3cccc4oc5ccccc5c34)c3c2oc2cc4ccccc4cc23)c2cccc3oc4ccccc4c23)cc1] 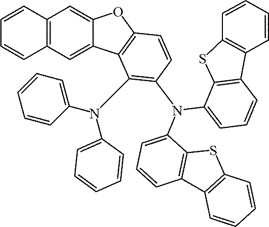 [SMILES: c1ccc(N(c2ccc3oc4ccc5ccccc5c4c3c2N(c2ccccc2)c2ccc3sc4ccccc4c3c2)c2cccc3c2sc2ccccc23)cc1] 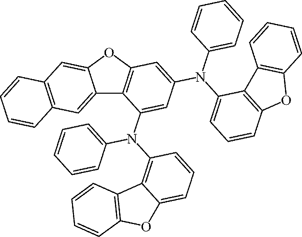 [SMILES: c1ccc(N(c2cc(N(c3ccccc3)c3ccc4c(c3)sc3ccccc34)c3c(c2)oc2ccc4ccccc4c23)c2cccc3c2sc2ccccc23)cc1] 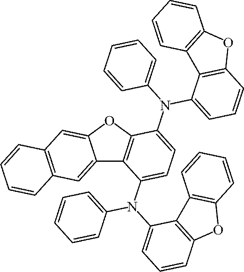 [SMILES: c1ccc(C(c2cccc3c2oc2ccccc23)c2ccc(N(c3ccccc3)c3cccc4c3sc3ccccc34)c3oc4ccc5ccccc5c4c23)cc1] 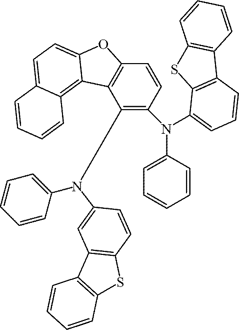 [SMILES: c1ccc(N(c2ccc3oc4ccccc4c3c2)c2cc3oc4c5ccccc5ccc4c3cc2N(c2ccccc2)c2cccc3c2sc2ccccc23)cc1] 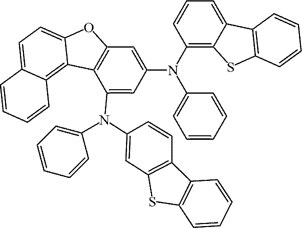 [SMILES: c1ccc(N(c2cc(N(c3ccccc3)c3cccc4oc5ccccc5c34)c3oc4c5ccccc5ccc4c3c2)c2cccc3oc4ccccc4c23)cc1] 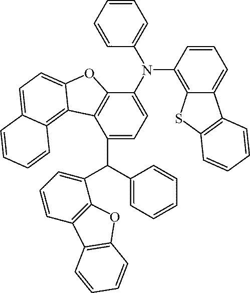 [SMILES: c1ccc(N(c2ccc3oc4ccccc4c3c2)c2c(N(c3ccccc3)c3cccc4c3sc3ccccc34)ccc3c2oc2c4ccccc4ccc32)cc1] 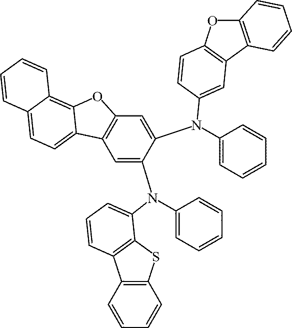 [SMILES: c1ccc(N(c2ccc3c(c2)oc2ccccc23)c2cc3c(cc2N(c2ccccc2)c2cccc4c2sc2ccccc24)oc2cc4ccccc4cc23)cc1] 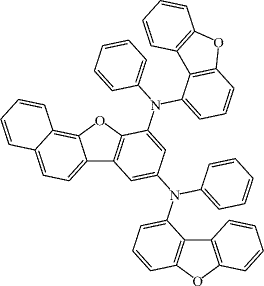 [SMILES: c1ccc(N(c2cc(N(c3ccccc3)c3cccc4oc5ccccc5c34)c3oc4cc5ccccc5cc4c3c2)c2cccc3oc4ccccc4c23)cc1] 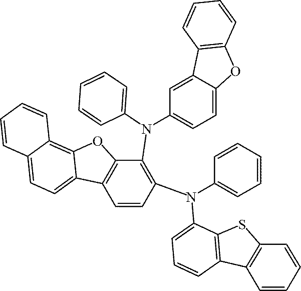 [SMILES: c1ccc(N(c2ccc3sc4ccccc4c3c2)c2ccc3c(oc4cc5ccccc5cc43)c2N(c2ccccc2)c2cccc3c2sc2ccccc23)cc1] 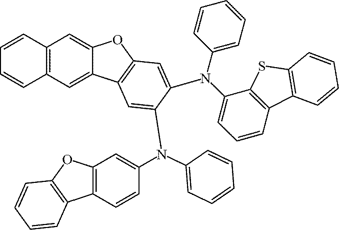 [SMILES: c1ccc(N(c2cc3oc4ccc5ccccc5c4c3cc2N(c2ccccc2)c2cccc3c2sc2ccccc23)c2cccc3c2oc2ccccc23)cc1] 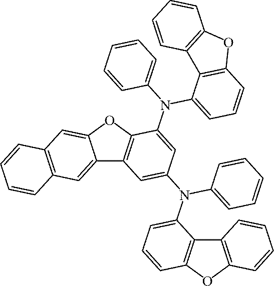 [SMILES: c1ccc(N(c2cccc3c2oc2ccccc23)c2cc(N(c3ccccc3)c3cccc4oc5ccccc5c34)cc3c2oc2ccc4ccccc4c23)cc1] 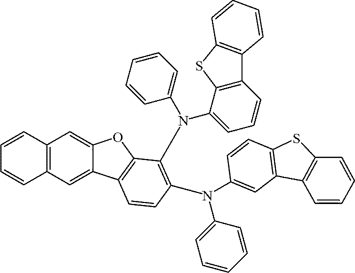 [SMILES: c1ccc(C(c2ccc3c(c2)sc2ccccc23)c2c(N(c3ccccc3)c3cccc4sc5ccccc5c34)ccc3c2oc2ccc4ccccc4c23)cc1] 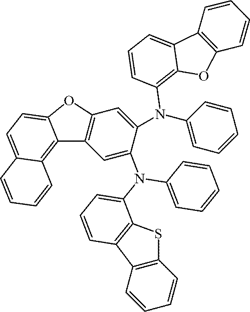 [SMILES: c1ccc(N(c2ccccc2)c2ccc3sc4c5ccccc5ccc4c3c2N(c2ccccc2)c2ccccc2)cc1] 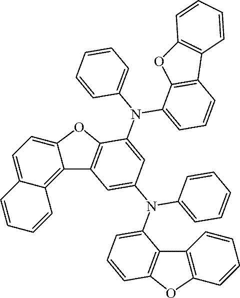 [SMILES: c1ccc(N(c2ccccc2)c2cc(N(c3ccccc3)c3ccccc3)c3c(c2)sc2c4ccccc4ccc23)cc1] 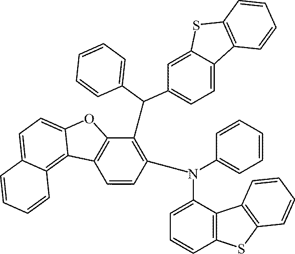 [SMILES: c1ccc(N(c2ccccc2)c2ccc(N(c3ccccc3)c3ccccc3)c3c2sc2c4ccccc4ccc23)cc1] 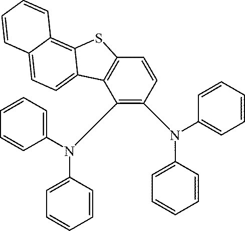 [SMILES: c1ccc(N(c2ccccc2)c2ccc3sc4cc5ccccc5cc4c3c2N(c2ccccc2)c2ccccc2)cc1] 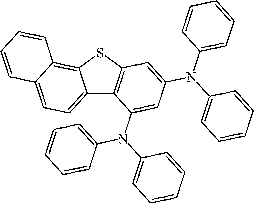 [SMILES: c1ccc(N(c2ccccc2)c2cc(N(c3ccccc3)c3ccccc3)c3c(c2)sc2cc4ccccc4cc23)cc1] 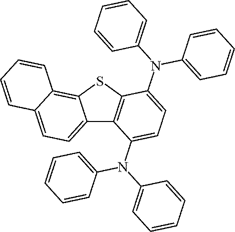 [SMILES: c1ccc(N(c2ccccc2)c2ccc(N(c3ccccc3)c3ccccc3)c3c2sc2cc4ccccc4cc23)cc1] 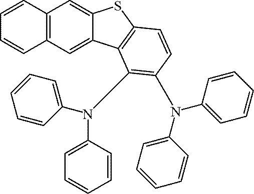 [SMILES: c1ccc(N(c2ccccc2)c2ccc3sc4ccc5ccccc5c4c3c2N(c2ccccc2)c2ccccc2)cc1] 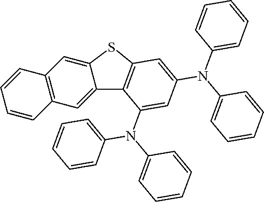 [SMILES: c1ccc(N(c2ccccc2)c2cc(N(c3ccccc3)c3ccccc3)c3c(c2)sc2ccc4ccccc4c23)cc1] 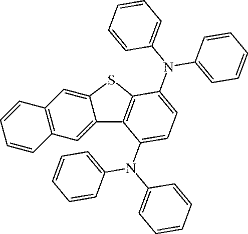 [SMILES: c1ccc(N(c2ccccc2)c2ccc(N(c3ccccc3)c3ccccc3)c3c2sc2ccc4ccccc4c23)cc1] 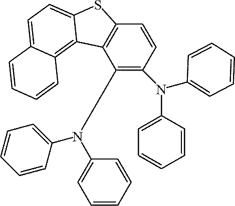 [SMILES: c1ccc(-c2ccccc2N(c2ccccc2)c2ccc3sc4c5ccccc5ccc4c3c2N(c2ccccc2)c2ccccc2-c2ccccc2)cc1] 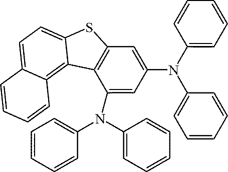 [SMILES: c1ccc(-c2ccccc2N(c2ccccc2)c2cc(N(c3ccccc3)c3ccccc3-c3ccccc3)c3c(c2)sc2c4ccccc4ccc23)cc1] 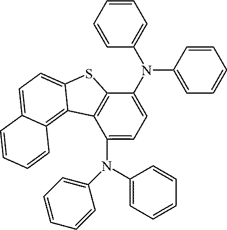 [SMILES: c1ccc(-c2ccccc2N(c2ccccc2)c2ccc(N(c3ccccc3)c3ccccc3-c3ccccc3)c3c2sc2c4ccccc4ccc23)cc1] 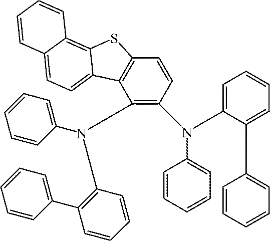 [SMILES: c1ccc(-c2ccccc2N(c2ccccc2)c2ccc3sc4cc5ccccc5cc4c3c2N(c2ccccc2)c2ccccc2-c2ccccc2)cc1] 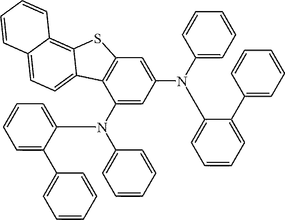 [SMILES: c1ccc(-c2ccccc2N(c2ccccc2)c2cc(N(c3ccccc3)c3ccccc3-c3ccccc3)c3c(c2)sc2cc4ccccc4cc23)cc1] 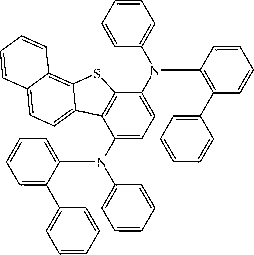 [SMILES: c1ccc(-c2ccccc2N(c2ccccc2)c2ccc(N(c3ccccc3)c3ccccc3-c3ccccc3)c3c2sc2cc4ccccc4cc23)cc1] 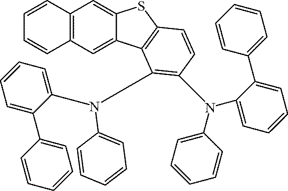 [SMILES: c1ccc(-c2ccccc2N(c2ccccc2)c2ccc3sc4ccc5ccccc5c4c3c2N(c2ccccc2)c2ccccc2-c2ccccc2)cc1] 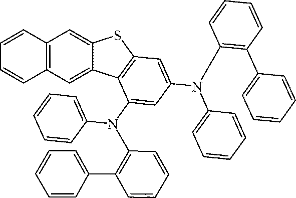 [SMILES: c1ccc(-c2ccccc2N(c2ccccc2)c2cc(N(c3ccccc3)c3ccccc3-c3ccccc3)c3c(c2)sc2ccc4ccccc4c23)cc1] 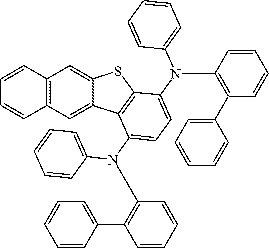 [SMILES: c1ccc(-c2ccccc2N(c2ccccc2)c2ccc(N(c3ccccc3)c3ccccc3-c3ccccc3)c3c2sc2ccc4ccccc4c23)cc1] 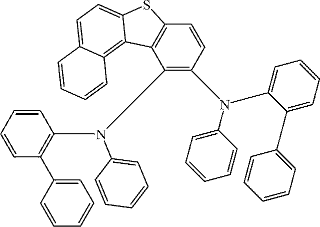 [SMILES: c1ccc(-c2cccc(N(c3ccccc3)c3ccc4sc5c6ccccc6ccc5c4c3N(c3ccccc3)c3ccccc3)c2)cc1] 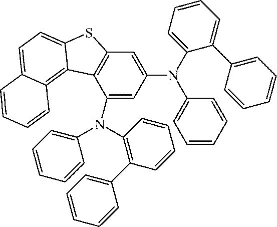 [SMILES: c1ccc(-c2cccc(N(c3ccccc3)c3cc(N(c4ccccc4)c4ccccc4)c4c(c3)sc3c5ccccc5ccc34)c2)cc1] 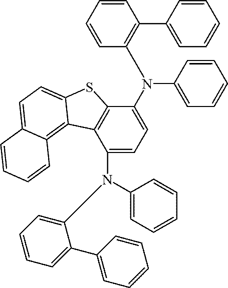 [SMILES: c1ccc(-c2cccc(N(c3ccccc3)c3ccc(N(c4ccccc4)c4ccccc4)c4sc5c6ccccc6ccc5c34)c2)cc1] 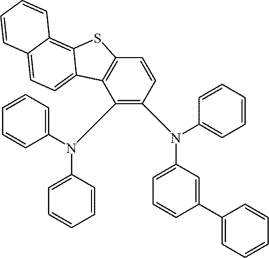 [SMILES: c1ccc(-c2cccc(N(c3ccccc3)c3ccc4sc5cc6ccccc6cc5c4c3N(c3ccccc3)c3ccccc3)c2)cc1] 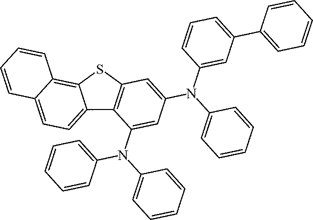 [SMILES: c1ccc(-c2ccc(N(c3ccccc3)c3cc(N(c4ccccc4)c4ccccc4)cc4sc5cc6ccccc6cc5c34)cc2)cc1] 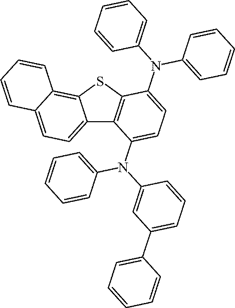 [SMILES: c1ccc(-c2cccc(N(c3ccccc3)c3ccc(N(c4ccccc4)c4ccccc4)c4c3sc3cc5ccccc5cc34)c2)cc1] 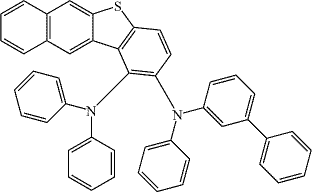 [SMILES: c1ccc(-c2cccc(N(c3ccccc3)c3ccc4sc5ccc6ccccc6c5c4c3N(c3ccccc3)c3ccccc3)c2)cc1] 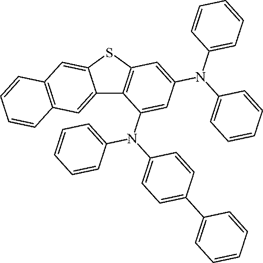 [SMILES: c1ccc(-c2cccc(N(c3ccccc3)c3cc(N(c4ccccc4)c4ccccc4)c4c(c3)sc3ccc5ccccc5c34)c2)cc1] 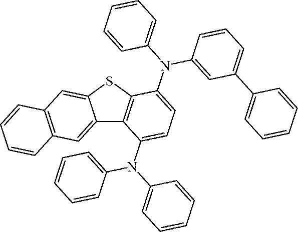 [SMILES: c1ccc(-c2cccc(N(c3ccccc3)c3ccc(N(c4ccccc4)c4ccccc4)c4c3sc3ccc5ccccc5c34)c2)cc1] 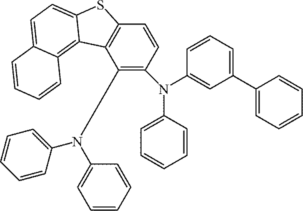 [SMILES: c1ccc(-c2cccc(N(c3ccccc3)c3ccc4sc5c6ccccc6ccc5c4c3N(c3ccccc3)c3cccc(-c4ccccc4)c3)c2)cc1] 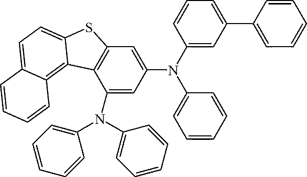 [SMILES: c1ccc(-c2cccc(N(c3ccccc3)c3cc(N(c4ccccc4)c4cccc(-c5ccccc5)c4)c4c(c3)sc3c5ccccc5ccc34)c2)cc1] 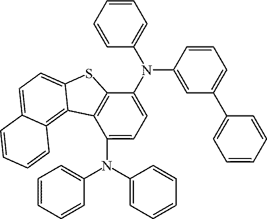 [SMILES: c1ccc(-c2cccc(N(c3ccccc3)c3ccc(N(c4ccccc4)c4cccc(-c5ccccc5)c4)c4c3sc3c5ccccc5ccc34)c2)cc1] 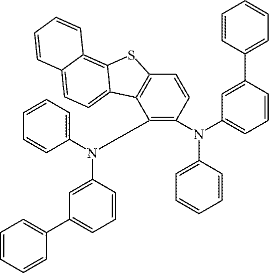 [SMILES: c1ccc(-c2cccc(N(c3ccccc3)c3ccc4sc5cc6ccccc6cc5c4c3N(c3ccccc3)c3cccc(-c4ccccc4)c3)c2)cc1] 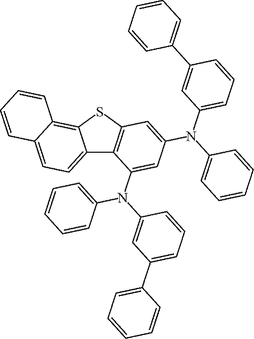 [SMILES: c1ccc(-c2cccc(N(c3ccccc3)c3cc(N(c4ccccc4)c4cccc(-c5ccccc5)c4)c4c(c3)sc3cc5ccccc5cc34)c2)cc1] 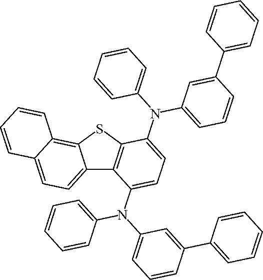 [SMILES: c1ccc(-c2cccc(N(c3ccccc3)c3ccc(N(c4ccccc4)c4cccc(-c5ccccc5)c4)c4c3sc3cc5ccccc5cc34)c2)cc1] 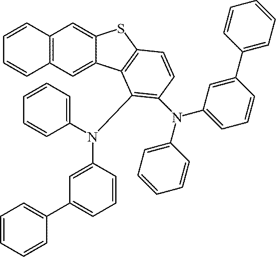 [SMILES: c1ccc(-c2cccc(N(c3ccccc3)c3ccc4sc5ccc6ccccc6c5c4c3N(c3ccccc3)c3cccc(-c4ccccc4)c3)c2)cc1] 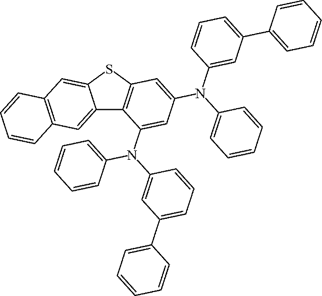 [SMILES: c1ccc(-c2cccc(N(c3ccccc3)c3cc(N(c4ccccc4)c4cccc(-c5ccccc5)c4)c4c(c3)sc3ccc5ccccc5c34)c2)cc1] 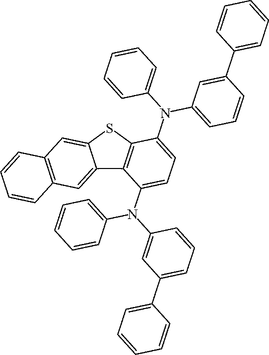 [SMILES: c1ccc(-c2cccc(N(c3ccccc3)c3ccc(N(c4ccccc4)c4cccc(-c5ccccc5)c4)c4c3sc3ccc5ccccc5c34)c2)cc1] 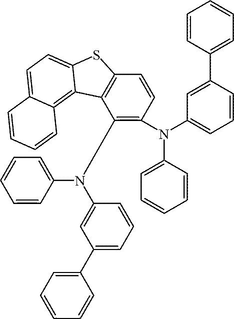 [SMILES: c1ccc(N(c2ccccc2)c2ccc3sc4c5ccccc5ccc4c3c2N(c2ccccc2)c2ccc3c(c2)oc2ccccc23)cc1] 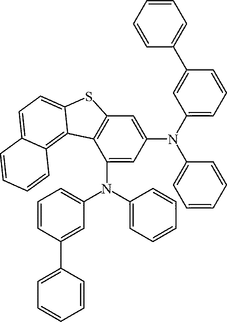 [SMILES: c1ccc(N(c2ccccc2)c2cc(N(c3ccccc3)c3ccc4c(c3)oc3ccccc34)c3c(c2)sc2c4ccccc4ccc23)cc1] 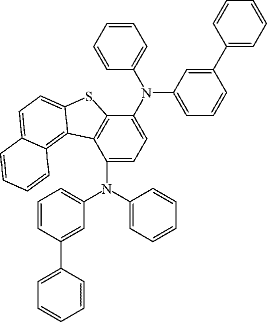 [SMILES: c1ccc(N(c2ccccc2)c2ccc(N(c3ccccc3)c3ccc4c(c3)oc3ccccc34)c3c2sc2c4ccccc4ccc23)cc1] 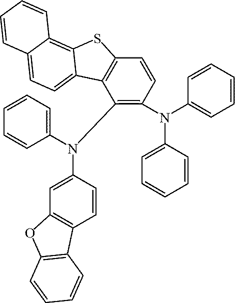 [SMILES: c1ccc(N(c2ccccc2)c2ccc3sc4cc5ccccc5cc4c3c2N(c2ccccc2)c2ccc3c(c2)sc2ccccc23)cc1] 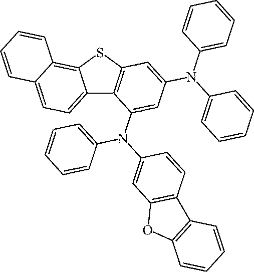 [SMILES: c1ccc(N(c2cc(N(c3ccccc3)c3ccccc3)c3c(c2)sc2cc4ccccc4cc23)c2ccc3oc4ccccc4c3c2)cc1] 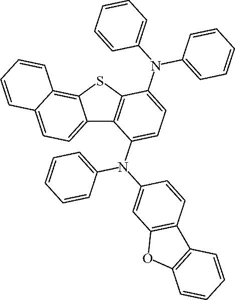 [SMILES: c1ccc(N(c2ccc3c(c2)oc2ccccc23)c2ccc(N(c3ccccc3)c3ccccc3)c3c2sc2cc4ccccc4cc23)cc1] 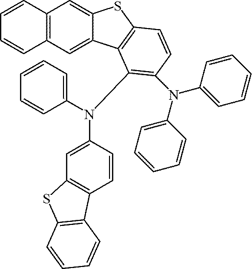 [SMILES: c1ccc(N(c2ccccc2)c2ccc3sc4ccc5ccccc5c4c3c2N(c2ccccc2)c2ccc3c(c2)sc2ccccc23)cc1] 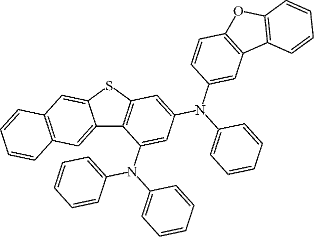 [SMILES: c1ccc(N(c2ccccc2)c2cc(N(c3ccccc3)c3ccc4c(c3)sc3ccccc34)c3c(c2)sc2ccc4ccccc4c23)cc1] 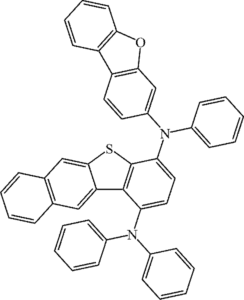 [SMILES: c1ccc(N(c2ccccc2)c2ccc(N(c3ccccc3)c3ccc4oc5ccccc5c4c3)c3c2sc2ccc4ccccc4c23)cc1] 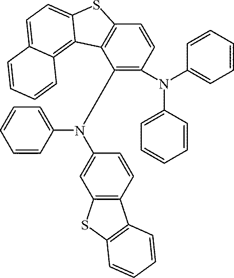 [SMILES: c1ccc(N(c2ccc3sc4c5ccccc5ccc4c3c2N(c2ccccc2)c2ccccc2)c2cccc3c2sc2ccccc23)cc1] 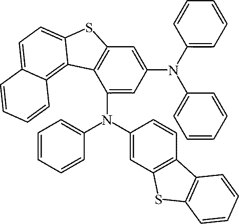 [SMILES: c1ccc(N(c2cc(N(c3ccccc3)c3ccccc3)c3c(c2)sc2c4ccccc4ccc23)c2cccc3oc4ccccc4c23)cc1] 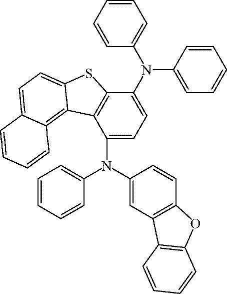 [SMILES: c1ccc(N(c2ccc(N(c3ccccc3)c3ccccc3)c3c2sc2c4ccccc4ccc23)c2cccc3oc4ccccc4c23)cc1] 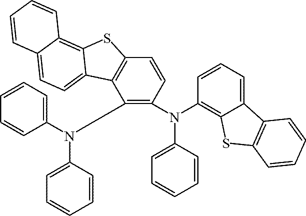 [SMILES: c1ccc(N(c2ccc3sc4cc5ccccc5cc4c3c2N(c2ccccc2)c2ccccc2)c2cccc3c2sc2ccccc23)cc1] 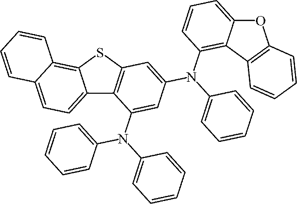 [SMILES: c1ccc(N(c2cc(N(c3ccccc3)c3ccccc3)c3c(c2)sc2cc4ccccc4cc23)c2cccc3oc4ccccc4c23)cc1] 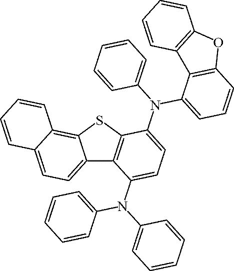 [SMILES: c1ccc(N(c2ccccc2)c2ccc(N(c3ccccc3)c3cccc4oc5ccccc5c34)c3c2sc2cc4ccccc4cc23)cc1] 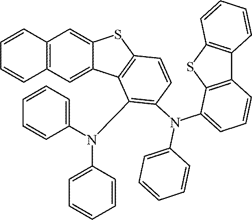 [SMILES: c1ccc(N(c2ccc3sc4ccc5ccccc5c4c3c2N(c2ccccc2)c2ccccc2)c2cccc3c2sc2ccccc23)cc1] 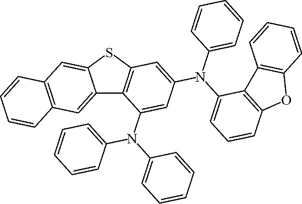 [SMILES: c1ccc(N(c2cc(N(c3ccccc3)c3ccccc3)c3c(c2)sc2ccc4ccccc4c23)c2cccc3c2sc2ccccc23)cc1] 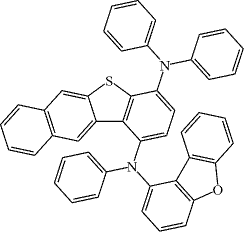 [SMILES: c1ccc(N(c2cccc3c2sc2ccccc23)c2ccc(N(c3ccccc3)c3ccccc3)c3c2sc2ccc4ccccc4c23)cc1] 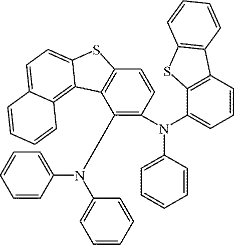 [SMILES: c1ccc(N(c2ccccc2)c2cc3sc4c5ccccc5ccc4c3cc2N(c2ccccc2)c2ccccc2)cc1] 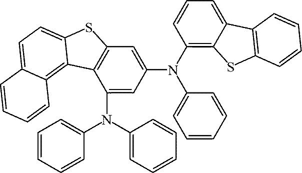 [SMILES: c1ccc(N(c2ccccc2)c2cc(N(c3ccccc3)c3ccccc3)c3sc4c5ccccc5ccc4c3c2)cc1] 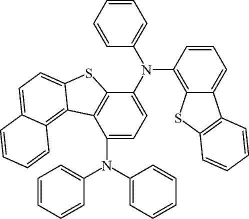 [SMILES: c1ccc(N(c2ccccc2)c2ccc3c(sc4c5ccccc5ccc34)c2N(c2ccccc2)c2ccccc2)cc1] 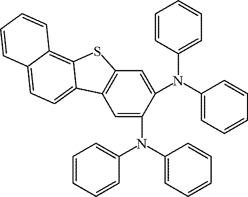 [SMILES: c1ccc(N(c2ccccc2)c2cc3sc4cc5ccccc5cc4c3cc2N(c2ccccc2)c2ccccc2)cc1] 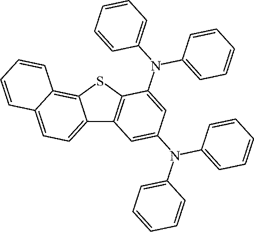 [SMILES: c1ccc(N(c2ccccc2)c2cc(N(c3ccccc3)c3ccccc3)c3sc4cc5ccccc5cc4c3c2)cc1] 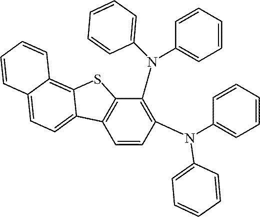 [SMILES: c1ccc(N(c2ccccc2)c2ccc3c(sc4cc5ccccc5cc43)c2N(c2ccccc2)c2ccccc2)cc1] 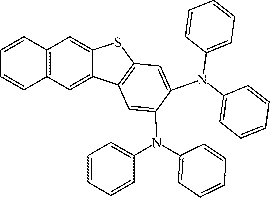 [SMILES: c1ccc(N(c2ccccc2)c2cc3sc4ccc5ccccc5c4c3cc2N(c2ccccc2)c2ccccc2)cc1] 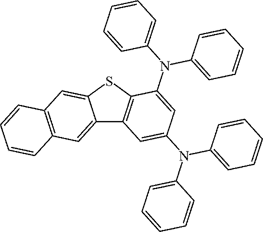 [SMILES: c1ccc(N(c2ccccc2)c2cc(N(c3ccccc3)c3ccccc3)c3sc4ccc5ccccc5c4c3c2)cc1] 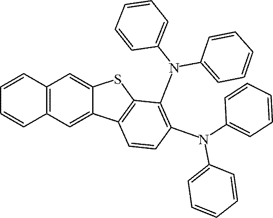 [SMILES: c1ccc(N(c2ccccc2)c2ccc3c(sc4ccc5ccccc5c43)c2N(c2ccccc2)c2ccccc2)cc1] 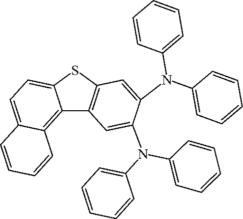 [SMILES: c1ccc(-c2ccccc2N(c2ccccc2)c2cc3sc4c5ccccc5ccc4c3cc2N(c2ccccc2)c2ccccc2-c2ccccc2)cc1] 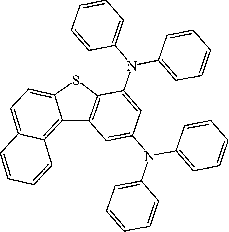 [SMILES: c1ccc(-c2ccccc2N(c2ccccc2)c2cc(N(c3ccccc3)c3ccccc3-c3ccccc3)c3sc4c5ccccc5ccc4c3c2)cc1] 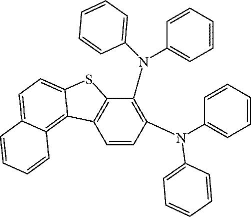 [SMILES: c1ccc(-c2ccccc2N(c2ccccc2)c2ccc3c(sc4c5ccccc5ccc34)c2N(c2ccccc2)c2ccccc2-c2ccccc2)cc1] 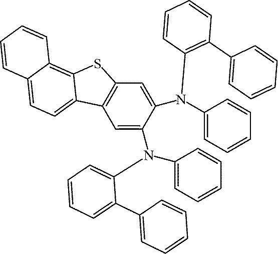 [SMILES: c1ccc(-c2ccccc2N(c2ccccc2)c2cc3sc4cc5ccccc5cc4c3cc2N(c2ccccc2)c2ccccc2-c2ccccc2)cc1] 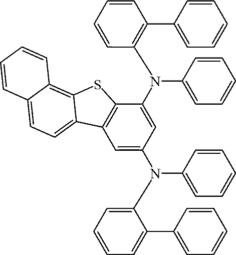 [SMILES: c1ccc(-c2ccccc2N(c2ccccc2)c2cc(N(c3ccccc3)c3ccccc3-c3ccccc3)c3sc4cc5ccccc5cc4c3c2)cc1] 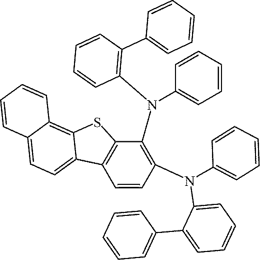 [SMILES: c1ccc(-c2ccccc2N(c2ccccc2)c2ccc3c(sc4cc5ccccc5cc43)c2N(c2ccccc2)c2ccccc2-c2ccccc2)cc1] 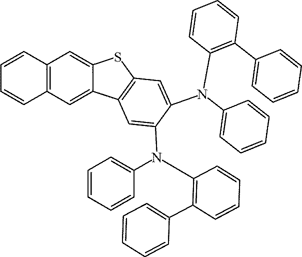 [SMILES: c1ccc(-c2ccccc2N(c2ccccc2)c2cc3sc4ccc5ccccc5c4c3cc2N(c2ccccc2)c2ccccc2-c2ccccc2)cc1] 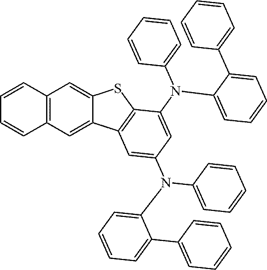 [SMILES: c1ccc(-c2ccccc2N(c2ccccc2)c2cc(N(c3ccccc3)c3ccccc3-c3ccccc3)c3sc4ccc5ccccc5c4c3c2)cc1] 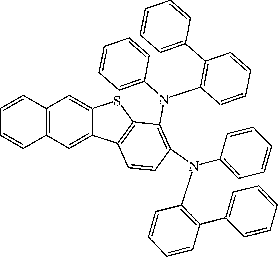 [SMILES: c1ccc(-c2ccccc2N(c2ccccc2)c2ccc3c(sc4ccc5ccccc5c43)c2N(c2ccccc2)c2ccccc2-c2ccccc2)cc1] 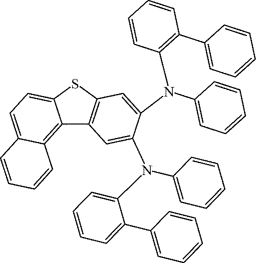 [SMILES: c1ccc(-c2cccc(N(c3ccccc3)c3cc4sc5c6ccccc6ccc5c4cc3N(c3ccccc3)c3ccccc3)c2)cc1] 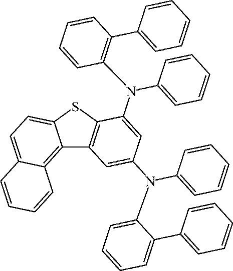 [SMILES: c1ccc(-c2cccc(N(c3ccccc3)c3cc(N(c4ccccc4)c4ccccc4)cc4c3sc3c5ccccc5ccc43)c2)cc1] 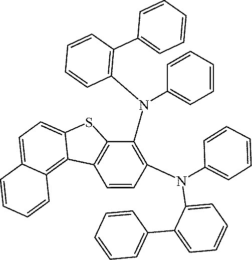 [SMILES: c1ccc(-c2cccc(N(c3ccccc3)c3ccc4c(sc5c6ccccc6ccc45)c3N(c3ccccc3)c3ccccc3)c2)cc1] 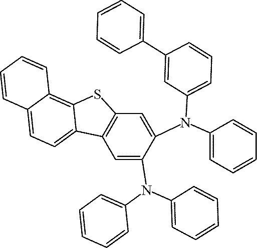 [SMILES: c1ccc(-c2cccc(N(c3ccccc3)c3cc4c(cc3N(c3ccccc3)c3ccccc3)sc3cc5ccccc5cc34)c2)cc1] 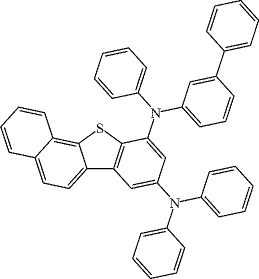 [SMILES: c1ccc(-c2cccc(N(c3ccccc3)c3cc(N(c4ccccc4)c4ccccc4)c4sc5cc6ccccc6cc5c4c3)c2)cc1] 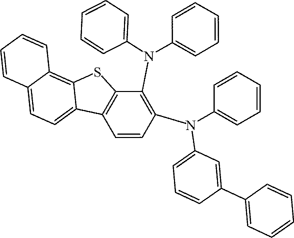 [SMILES: c1ccc(-c2cccc(N(c3ccccc3)c3ccc4c(sc5cc6ccccc6cc54)c3N(c3ccccc3)c3ccccc3)c2)cc1] 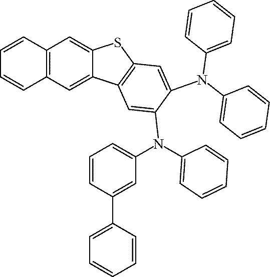 [SMILES: c1ccc(-c2cccc(N(c3ccccc3)c3cc4c(cc3N(c3ccccc3)c3ccccc3)sc3ccc5ccccc5c34)c2)cc1] 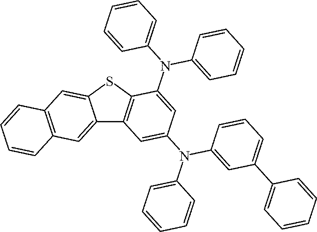 [SMILES: c1ccc(-c2cccc(N(c3ccccc3)c3cc(N(c4ccccc4)c4ccccc4)cc4c3sc3ccc5ccccc5c34)c2)cc1] 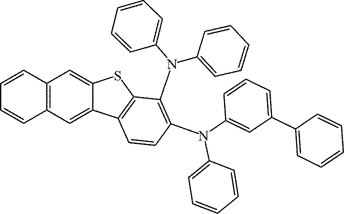 [SMILES: c1ccc(-c2cccc(N(c3ccccc3)c3ccc4c(sc5ccc6ccccc6c54)c3N(c3ccccc3)c3ccccc3)c2)cc1] 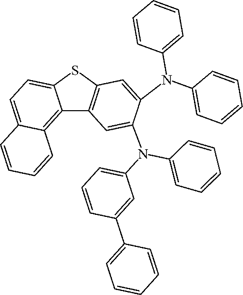 [SMILES: c1ccc(-c2cccc(N(c3ccccc3)c3cc4sc5c6ccccc6ccc5c4cc3N(c3ccccc3)c3cccc(-c4ccccc4)c3)c2)cc1] 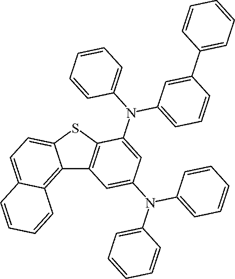 [SMILES: c1ccc(-c2cccc(N(c3ccccc3)c3cc(N(c4ccccc4)c4cccc(-c5ccccc5)c4)c4sc5c6ccccc6ccc5c4c3)c2)cc1] 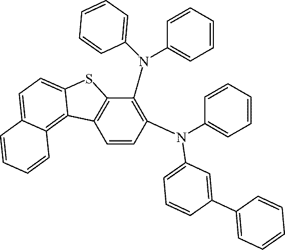 [SMILES: c1ccc(-c2cccc(N(c3ccccc3)c3ccc4c(sc5c6ccccc6ccc45)c3N(c3ccccc3)c3cccc(-c4ccccc4)c3)c2)cc1] 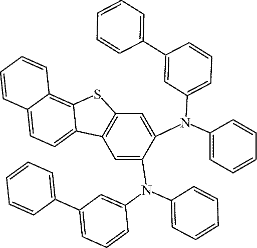 [SMILES: c1ccc(-c2cccc(N(c3ccccc3)c3cc4sc5cc6ccccc6cc5c4cc3N(c3ccccc3)c3cccc(-c4ccccc4)c3)c2)cc1] 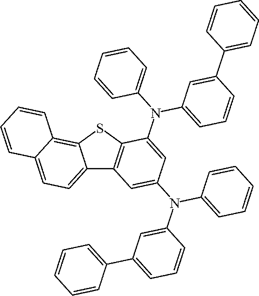 [SMILES: c1ccc(-c2cccc(N(c3ccccc3)c3cc(N(c4ccccc4)c4cccc(-c5ccccc5)c4)c4sc5cc6ccccc6cc5c4c3)c2)cc1] 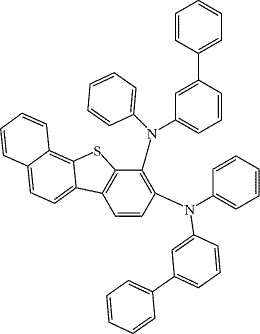 [SMILES: c1ccc(-c2cccc(N(c3ccccc3)c3ccc4c(sc5cc6ccccc6cc54)c3N(c3ccccc3)c3cccc(-c4ccccc4)c3)c2)cc1] 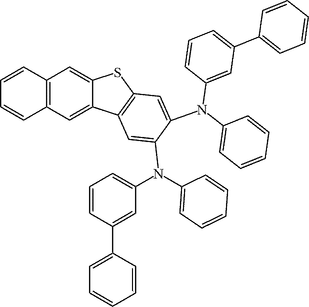 [SMILES: c1ccc(-c2cccc(N(c3ccccc3)c3cc4sc5ccc6ccccc6c5c4cc3N(c3ccccc3)c3cccc(-c4ccccc4)c3)c2)cc1] 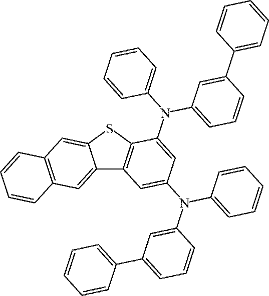 [SMILES: c1ccc(-c2cccc(N(c3ccccc3)c3cc(N(c4ccccc4)c4cccc(-c5ccccc5)c4)c4sc5ccc6ccccc6c5c4c3)c2)cc1] 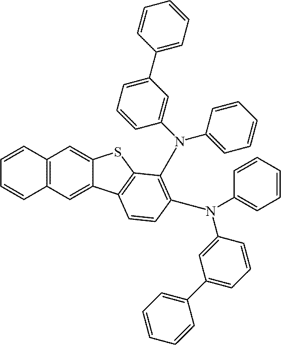 [SMILES: c1ccc(-c2cccc(N(c3ccccc3)c3ccc4c(sc5ccc6ccccc6c54)c3N(c3ccccc3)c3cccc(-c4ccccc4)c3)c2)cc1] 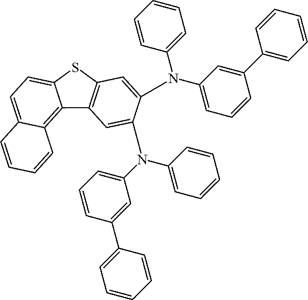 [SMILES: c1ccc(N(c2ccccc2)c2cc3sc4c5ccccc5ccc4c3cc2N(c2ccccc2)c2ccc3sc4ccccc4c3c2)cc1] 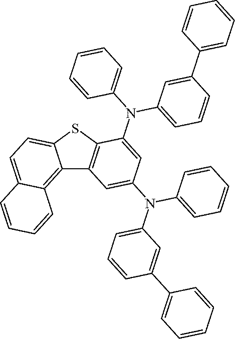 [SMILES: c1ccc(N(c2ccc3sc4ccccc4c3c2)c2cc(N(c3ccccc3)c3ccccc3)c3sc4c5ccccc5ccc4c3c2)cc1] 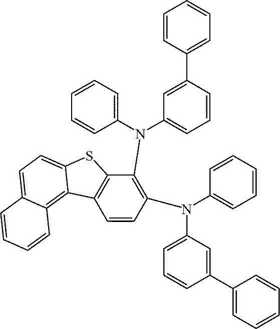 [SMILES: c1ccc(N(c2ccccc2)c2ccc3c(sc4c5ccccc5ccc34)c2N(c2ccccc2)c2ccc3c(c2)oc2ccccc23)cc1] 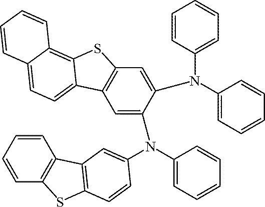 [SMILES: c1ccc(N(c2ccc3sc4ccccc4c3c2)c2cc3sc4cc5ccccc5cc4c3cc2N(c2ccccc2)c2ccccc2)cc1] 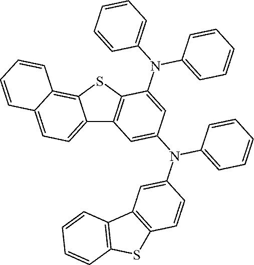 [SMILES: c1ccc(N(c2ccccc2)c2cc(N(c3ccccc3)c3ccc4c(c3)oc3ccccc34)c3sc4cc5ccccc5cc4c3c2)cc1] 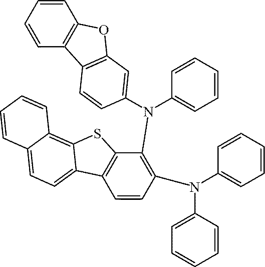 [SMILES: c1ccc(N(c2ccccc2)c2ccc3c(sc4cc5ccccc5cc43)c2N(c2ccccc2)c2ccc3sc4ccccc4c3c2)cc1] 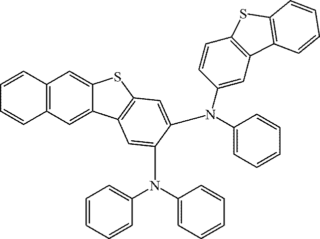 [SMILES: c1ccc(N(c2ccc3c(c2)oc2ccccc23)c2cc3sc4ccc5ccccc5c4c3cc2N(c2ccccc2)c2ccccc2)cc1] 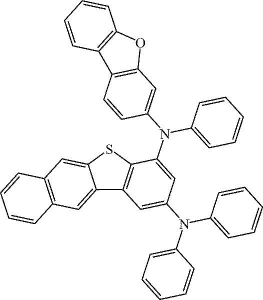 [SMILES: c1ccc(N(c2ccccc2)c2cc(N(c3ccccc3)c3ccc4c(c3)sc3ccccc34)c3sc4ccc5ccccc5c4c3c2)cc1] 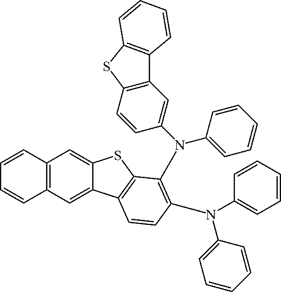 [SMILES: c1ccc(N(c2ccccc2)c2ccc3c(sc4ccc5ccccc5c43)c2N(c2ccccc2)c2ccc3sc4ccccc4c3c2)cc1] 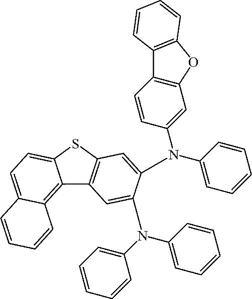 [SMILES: c1ccc(N(Cc2cc3c(cc2N(c2ccccc2)c2ccccc2)sc2c4ccccc4ccc32)c2cccc3c2sc2ccccc23)cc1] 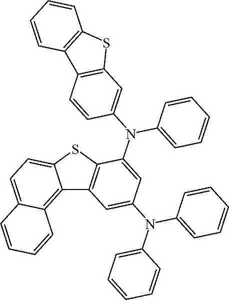 [SMILES: c1ccc(N(c2ccccc2)c2cc(N(c3ccccc3)c3cccc4oc5ccccc5c34)c3sc4c5ccccc5ccc4c3c2)cc1] 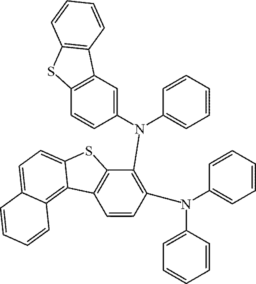 [SMILES: c1ccc(N(c2ccc3c(sc4c5ccccc5ccc34)c2N(c2ccccc2)c2ccccc2)c2cccc3c2sc2ccccc23)cc1] 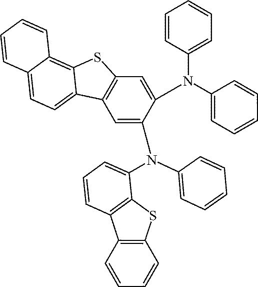 [SMILES: c1ccc(N(c2ccccc2)c2cc3c(cc2N(c2ccccc2)c2cccc4c2sc2ccccc24)sc2cc4ccccc4cc23)cc1] 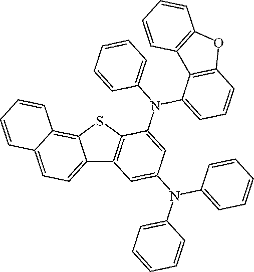 [SMILES: c1ccc(N(c2ccccc2)c2cc(N(c3ccccc3)c3cccc4oc5ccccc5c34)c3sc4cc5ccccc5cc4c3c2)cc1] 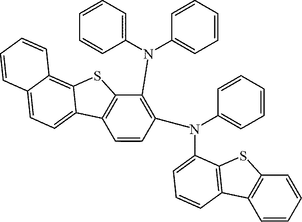 [SMILES: c1ccc(N(c2ccccc2)c2ccc3c(sc4cc5ccccc5cc43)c2N(c2ccccc2)c2cccc3c2sc2ccccc23)cc1] 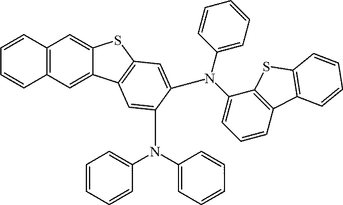 [SMILES: c1ccc(N(c2ccccc2)c2cc3c(cc2N(c2ccccc2)c2cccc4c2oc2ccccc24)sc2ccc4ccccc4c23)cc1] 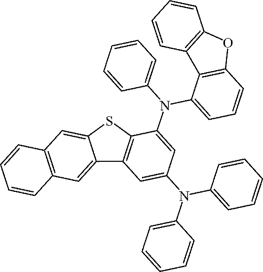 [SMILES: c1ccc(N(c2ccccc2)c2cc(N(c3ccccc3)c3cccc4oc5ccccc5c34)cc3c2sc2ccc4ccccc4c23)cc1] 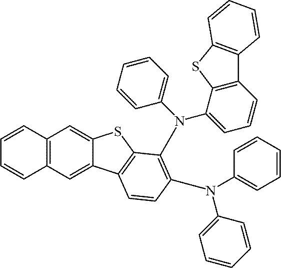 [SMILES: c1ccc(N(c2ccccc2)c2c(N(c3ccccc3)c3cccc4sc5ccccc5c34)ccc3c2sc2ccc4ccccc4c23)cc1] 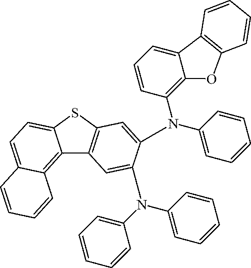 [SMILES: c1ccc(-c2ccc(N(c3ccccc3)c3ccc4sc5c6ccccc6ccc5c4c3N(c3ccccc3)c3ccc(-c4ccccc4)cc3)cc2)cc1] 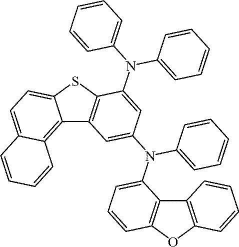 [SMILES: c1ccc(-c2ccc(N(c3ccccc3)c3cc(N(c4ccccc4)c4ccc(-c5ccccc5)cc4)c4c(c3)sc3c5ccccc5ccc34)cc2)cc1] 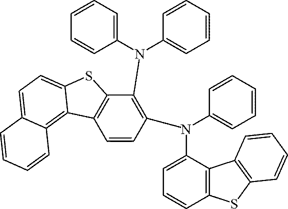 [SMILES: c1ccc(-c2ccc(N(c3ccccc3)c3ccc(N(c4ccccc4)c4ccc(-c5ccccc5)cc4)c4c3sc3c5ccccc5ccc34)cc2)cc1] 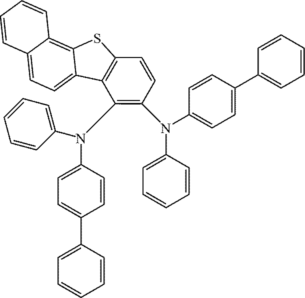 [SMILES: c1ccc(-c2ccc(N(c3ccccc3)c3ccc4sc5cc6ccccc6cc5c4c3N(c3ccccc3)c3ccc(-c4ccccc4)cc3)cc2)cc1] 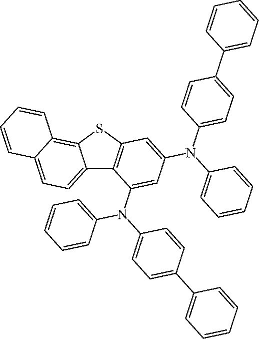 [SMILES: c1ccc(-c2ccc(N(c3ccccc3)c3cc(N(c4ccccc4)c4ccc(-c5ccccc5)cc4)c4c(c3)sc3cc5ccccc5cc34)cc2)cc1] 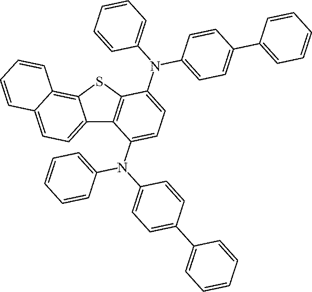 [SMILES: c1ccc(-c2ccc(N(c3ccccc3)c3ccc(N(c4ccccc4)c4ccc(-c5ccccc5)cc4)c4c3sc3cc5ccccc5cc34)cc2)cc1] 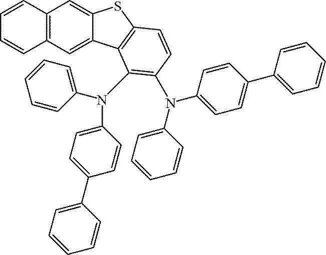 [SMILES: c1ccc(-c2ccc(N(c3ccccc3)c3ccc4sc5ccc6ccccc6c5c4c3N(c3ccccc3)c3ccc(-c4ccccc4)cc3)cc2)cc1] 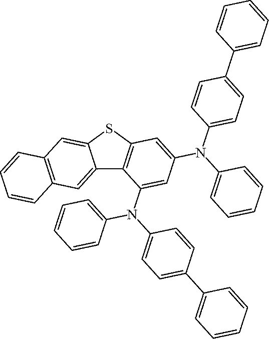 [SMILES: c1ccc(-c2ccc(N(c3ccccc3)c3cc(N(c4ccccc4)c4ccc(-c5ccccc5)cc4)c4c(c3)sc3ccc5ccccc5c34)cc2)cc1] 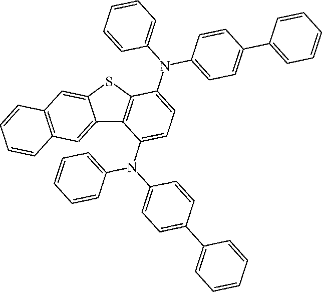 [SMILES: c1ccc(-c2ccc(N(c3ccccc3)c3ccc(N(c4ccccc4)c4ccc(-c5ccccc5)cc4)c4c3sc3ccc5ccccc5c34)cc2)cc1] 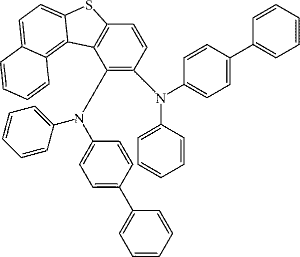 [SMILES: c1ccc(-c2ccccc2N(c2ccccc2)c2c(N(c3ccccc3)c3ccccc3)ccc3sc4c5ccccc5ccc4c23)cc1] 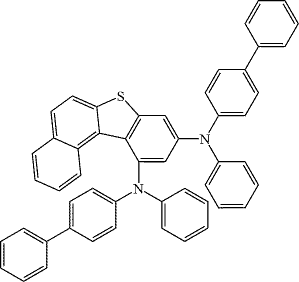 [SMILES: c1ccc(-c2ccccc2C(c2ccccc2)c2cc(N(c3ccccc3)c3ccccc3)cc3sc4c5ccccc5ccc4c23)cc1] 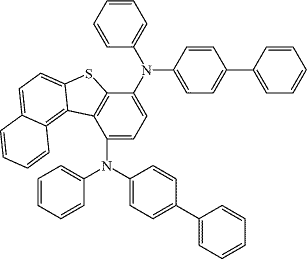 [SMILES: c1ccc(-c2ccccc2N(c2ccccc2)c2ccc(N(c3ccccc3)c3ccccc3)c3sc4c5ccccc5ccc4c23)cc1] 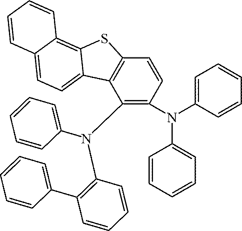 [SMILES: c1ccc(-c2ccccc2N(c2ccccc2)c2c(N(c3ccccc3)c3ccccc3)ccc3sc4cc5ccccc5cc4c23)cc1] 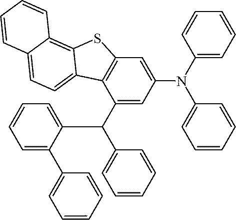 [SMILES: c1ccc(-c2ccccc2N(c2ccccc2)c2cc(N(c3ccccc3)c3ccccc3)cc3sc4cc5ccccc5cc4c23)cc1] 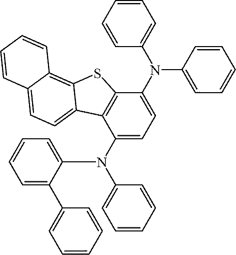 [SMILES: c1ccc(-c2ccccc2N(c2ccccc2)c2ccc(N(c3ccccc3)c3ccccc3)c3c2sc2cc4ccccc4cc23)cc1] 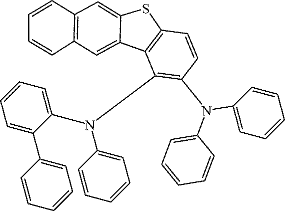 [SMILES: c1ccc(-c2ccccc2N(c2ccccc2)c2c(N(c3ccccc3)c3ccccc3)ccc3sc4ccc5ccccc5c4c23)cc1] 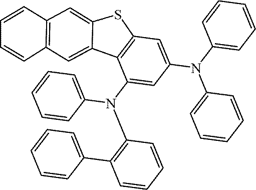 [SMILES: c1ccc(-c2ccccc2N(c2ccccc2)c2cc(N(c3ccccc3)c3ccccc3)c3c(c2)sc2ccc4ccccc4c23)cc1] 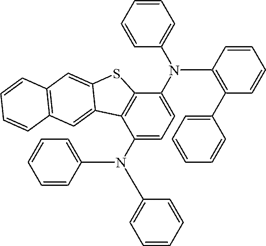 [SMILES: c1ccc(-c2ccccc2N(c2ccccc2)c2ccc(N(c3ccccc3)c3ccccc3)c3sc4ccc5ccccc5c4c23)cc1] 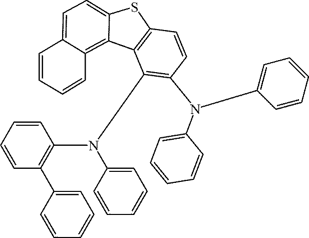 [SMILES: c1ccc(N(c2ccc3ccccc3c2)c2ccc3sc4c5ccccc5ccc4c3c2N(c2ccccc2)c2ccc3ccccc3c2)cc1] 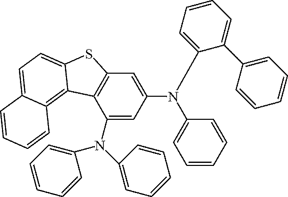 [SMILES: c1ccc(N(c2ccc3ccccc3c2)c2cc(N(c3ccccc3)c3ccc4ccccc4c3)c3c(c2)sc2c4ccccc4ccc23)cc1] 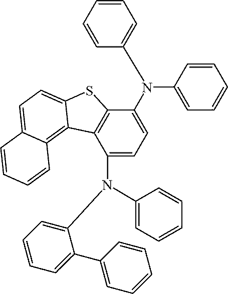 [SMILES: c1ccc(N(c2ccc3ccccc3c2)c2ccc(N(c3ccccc3)c3ccc4ccccc4c3)c3c2sc2c4ccccc4ccc23)cc1] 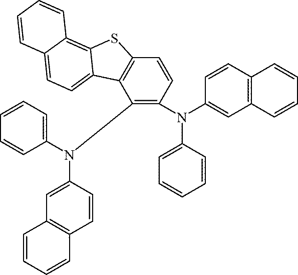 [SMILES: c1ccc(N(c2ccc3ccccc3c2)c2ccc3sc4cc5ccccc5cc4c3c2N(c2ccccc2)c2ccc3ccccc3c2)cc1] 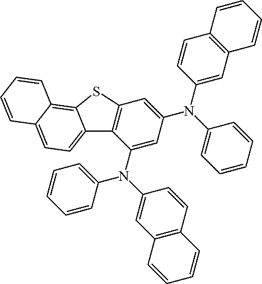 [SMILES: c1ccc(N(c2ccc3ccccc3c2)c2cc(N(c3ccccc3)c3ccc4ccccc4c3)c3c(c2)sc2cc4ccccc4cc23)cc1] 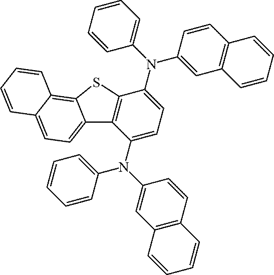 [SMILES: c1ccc(N(c2ccc3ccccc3c2)c2ccc(N(c3ccccc3)c3ccc4ccccc4c3)c3c2sc2cc4ccccc4cc23)cc1] 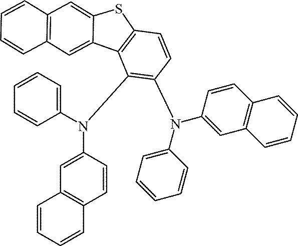 [SMILES: c1ccc(N(c2ccc3ccccc3c2)c2ccc3sc4ccc5ccccc5c4c3c2N(c2ccccc2)c2ccc3ccccc3c2)cc1] 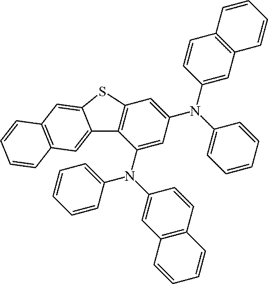 [SMILES: c1ccc(N(c2ccc3ccccc3c2)c2cc(N(c3ccccc3)c3ccc4ccccc4c3)c3c(c2)sc2ccc4ccccc4c23)cc1] 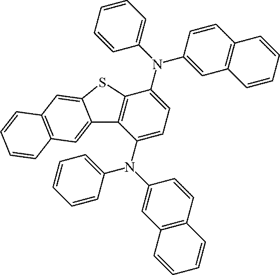 [SMILES: c1ccc(N(c2ccc3ccccc3c2)c2ccc(N(c3ccccc3)c3ccc4ccccc4c3)c3c2sc2ccc4ccccc4c23)cc1] 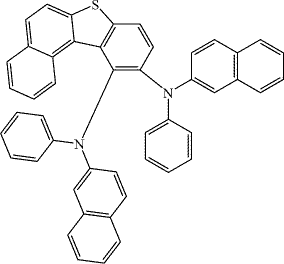 [SMILES: c1ccc(-c2cccc(N(c3ccc4ccccc4c3)c3ccc4sc5c6ccccc6ccc5c4c3N(c3ccccc3)c3ccc4ccccc4c3)c2)cc1] 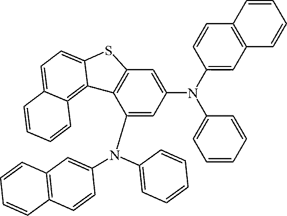 [SMILES: c1ccc(-c2cccc(N(c3ccc4ccccc4c3)c3cc(N(c4ccccc4)c4ccc5ccccc5c4)cc4sc5c6ccccc6ccc5c34)c2)cc1] 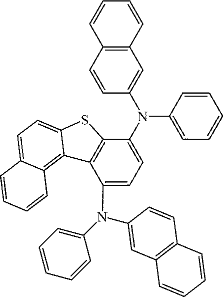 [SMILES: c1ccc(-c2ccc(N(c3ccc4ccccc4c3)c3ccc(N(c4ccccc4)c4ccc5ccccc5c4)c4c3sc3c5ccccc5ccc34)cc2)cc1] 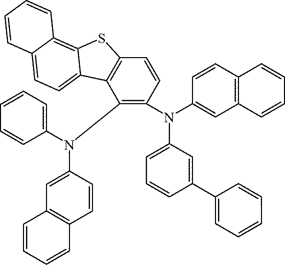 [SMILES: c1ccc(-c2cccc(N(c3ccc4ccccc4c3)c3ccc4sc5cc6ccccc6cc5c4c3N(c3ccccc3)c3ccc4ccccc4c3)c2)cc1] 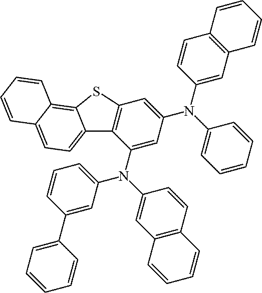 [SMILES: c1ccc(-c2ccc(N(c3ccc4ccccc4c3)c3cc(N(c4ccccc4)c4ccc5ccccc5c4)c4c(c3)sc3cc5ccccc5cc34)cc2)cc1] 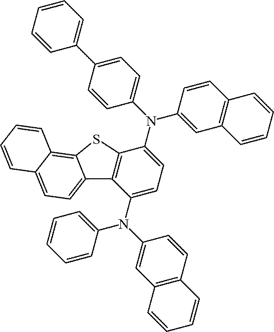 [SMILES: c1ccc(-c2cccc(N(c3ccc4ccccc4c3)c3ccc(N(c4ccccc4)c4ccc5ccccc5c4)c4sc5cc6ccccc6cc5c34)c2)cc1] 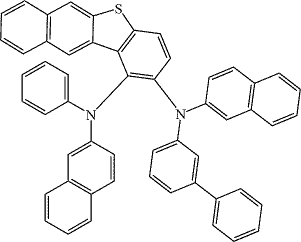 [SMILES: c1ccc(-c2ccc(N(c3ccc4ccccc4c3)c3c(N(c4ccccc4)c4ccc5ccccc5c4)ccc4sc5ccc6ccccc6c5c34)cc2)cc1] 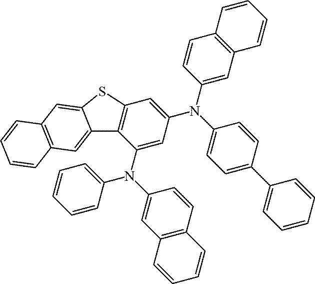 [SMILES: c1ccc(-c2cccc(N(c3ccc4ccccc4c3)c3cc(N(c4ccccc4)c4ccc5ccccc5c4)c4c(c3)sc3ccc5ccccc5c34)c2)cc1] 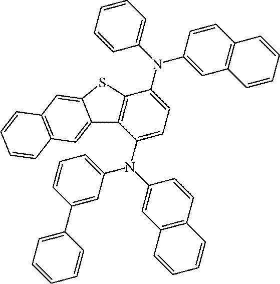 [SMILES: c1ccc(-c2cccc(N(c3ccc4ccccc4c3)c3ccc(N(c4ccccc4)c4ccc5ccccc5c4)c4c3sc3ccc5ccccc5c34)c2)cc1] 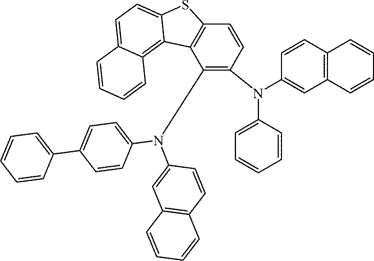 [SMILES: c1ccc(-c2ccc(N(c3ccccc3)c3ccc4sc5c6ccccc6ccc5c4c3N(c3ccccc3)c3ccc4c(c3)oc3ccccc34)cc2)cc1] 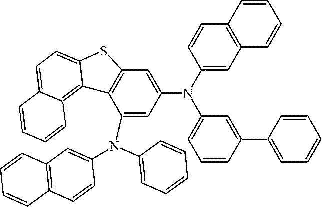 [SMILES: c1ccc(-c2ccc(N(c3ccc4c(c3)oc3ccccc34)c3cc(N(c4ccccc4)c4ccccc4)cc4sc5c6ccccc6ccc5c34)cc2)cc1] 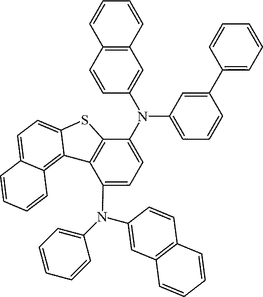 [SMILES: c1ccc(-c2cccc(N(c3ccccc3)c3ccc(N(c4cccc(-c5ccccc5)c4)c4ccc5c(c4)oc4ccccc45)c4c3sc3c5ccccc5ccc34)c2)cc1] 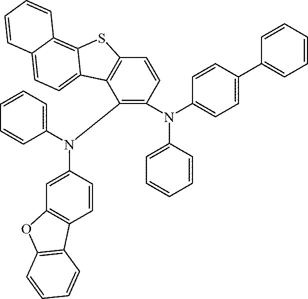 [SMILES: c1ccc(-c2ccc(N(c3ccccc3)c3ccc4sc5cc6ccccc6cc5c4c3N(c3ccccc3)c3ccc4c(c3)sc3ccccc34)c3ccccc23)cc1] 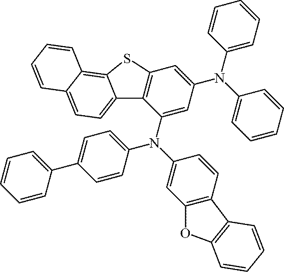 [SMILES: c1ccc(-c2ccc(N(c3ccccc3)c3cc(N(c4ccccc4)c4ccc5oc6ccccc6c5c4)cc4sc5cc6ccccc6cc5c34)cc2)cc1] 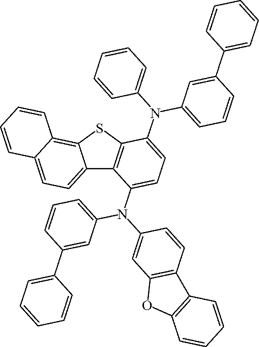 [SMILES: c1ccc(-c2cccc(N(c3ccccc3)c3ccc(N(c4ccccc4)c4ccc5c(c4)oc4ccccc45)c4sc5cc6ccccc6cc5c34)c2)cc1] 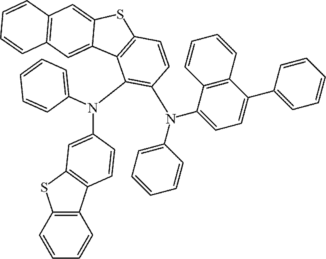 [SMILES: c1ccc(-c2ccc(N(c3ccccc3)c3ccc4sc5ccc6ccccc6c5c4c3N(c3ccccc3)c3ccc4c(c3)sc3ccccc34)c3ccccc23)cc1] 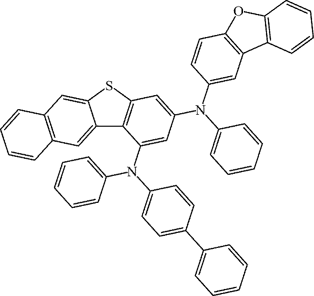 [SMILES: c1ccc(-c2ccc(N(c3ccc4c(c3)sc3ccccc34)c3cc(N(c4ccccc4)c4ccccc4)cc4sc5ccc6ccccc6c5c34)cc2)cc1] 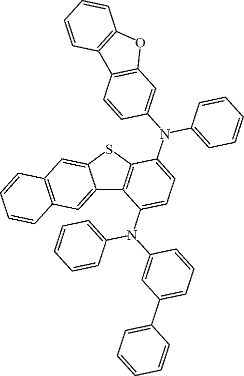 [SMILES: c1ccc(N(c2cc3ccccc3c3ccccc23)c2ccc(N(c3ccc4ccccc4c3)c3ccc4oc5ccccc5c4c3)c3c2sc2ccc4ccccc4c23)cc1] 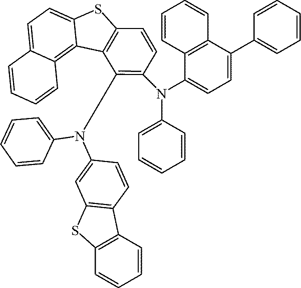 [SMILES: c1ccc(N(c2ccc3sc4c5ccccc5ccc4c3c2N(c2ccccc2)c2cc3ccccc3c3ccccc23)c2cccc3c2sc2ccccc23)cc1] 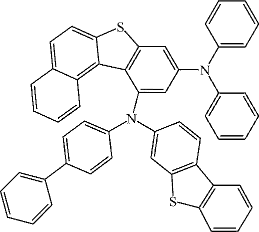 [SMILES: c1ccc(N(c2cc(N(c3ccccc3)c3ccc4c(ccc5ccccc54)c3)c3c(c2)sc2c4ccccc4ccc23)c2cccc3oc4ccccc4c23)cc1] 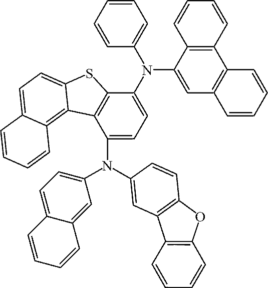 [SMILES: c1ccc(-c2cccc(N(c3ccccc3)c3ccc(N(c4ccccc4)c4cccc5oc6ccccc6c45)c4sc5c6ccccc6ccc5c34)c2)cc1] 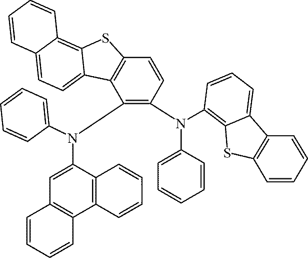 [SMILES: c1ccc(N(c2ccc3sc4cc5ccccc5cc4c3c2N(c2ccccc2)c2ccc3ccc4ccccc4c3c2)c2cccc3c2sc2ccccc23)cc1] 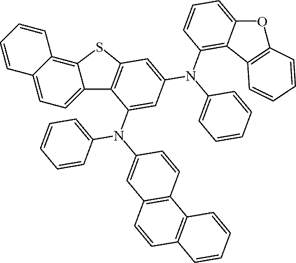 [SMILES: c1ccc(-c2ccc(N(c3ccccc3)c3cc(N(c4ccccc4)c4cccc5oc6ccccc6c45)cc4sc5cc6ccccc6cc5c34)cc2)cc1] 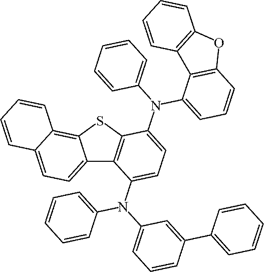 [SMILES: c1ccc(-c2ccc(N(c3ccccc3)c3ccc(N(c4ccccc4)c4cccc5oc6ccccc6c45)c4c3sc3cc5ccccc5cc34)cc2)cc1] 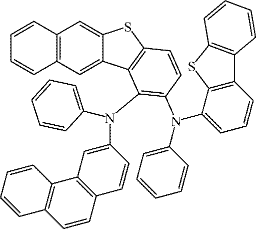 [SMILES: c1ccc(-c2cccc(N(c3ccc4sc5ccc6ccccc6c5c4c3N(c3ccccc3)c3ccc4ccccc4c3)c3cccc4c3sc3ccccc34)c2)cc1] 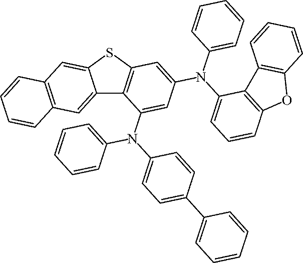 [SMILES: c1ccc(-c2ccc(N(c3cc(N(c4ccccc4)c4ccc(-c5ccccc5)cc4)c4c(c3)sc3ccc5ccccc5c34)c3cccc4c3sc3ccccc34)cc2)cc1] 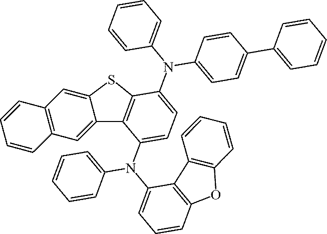 [SMILES: c1ccc(-c2cccc(N(c3cccc4c3sc3ccccc34)c3ccc(N(c4ccccc4)c4cccc(-c5ccccc5)c4)c4c3sc3ccc5ccccc5c34)c2)cc1] 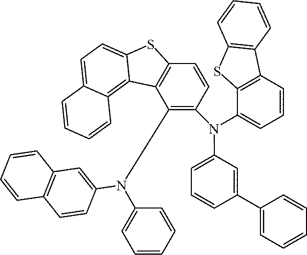 [SMILES: c1ccc(-c2ccc(N(c3ccccc3)c3cc4sc5c6ccccc6ccc5c4cc3N(c3ccccc3)c3ccc(-c4ccccc4)cc3)cc2)cc1] 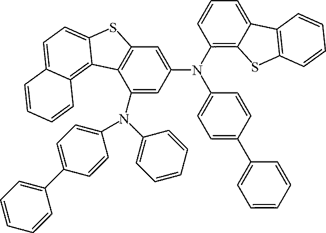 [SMILES: c1ccc(-c2ccc(N(c3ccccc3)c3cc(N(c4ccccc4)c4ccc(-c5ccccc5)cc4)c4sc5c6ccccc6ccc5c4c3)cc2)cc1] 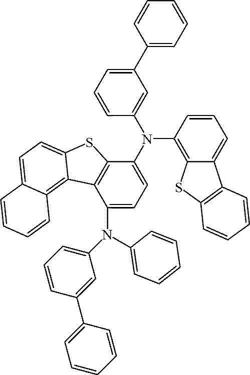 [SMILES: c1ccc(-c2ccc(N(c3ccccc3)c3ccc4c(sc5c6ccccc6ccc45)c3N(c3ccccc3)c3ccc(-c4ccccc4)cc3)cc2)cc1] 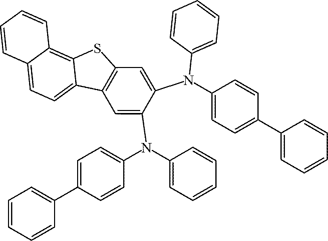 [SMILES: c1ccc(-c2ccc(N(c3ccccc3)c3cc4sc5cc6ccccc6cc5c4cc3N(c3ccccc3)c3ccc(-c4ccccc4)cc3)cc2)cc1] 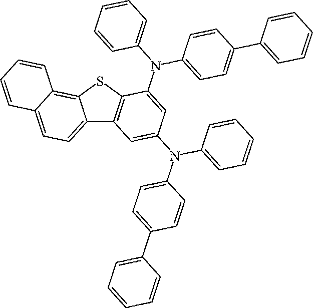 [SMILES: c1ccc(-c2ccc(N(c3ccccc3)c3cc(N(c4ccccc4)c4ccc(-c5ccccc5)cc4)c4sc5cc6ccccc6cc5c4c3)cc2)cc1] 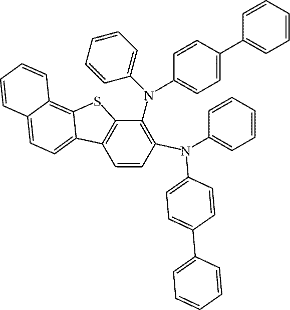 [SMILES: c1ccc(-c2ccc(N(c3ccccc3)c3ccc4c(sc5cc6ccccc6cc54)c3N(c3ccccc3)c3ccc(-c4ccccc4)cc3)cc2)cc1] 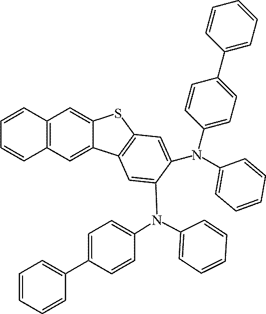 [SMILES: c1ccc(-c2ccc(N(c3ccccc3)c3cc4sc5ccc6ccccc6c5c4cc3N(c3ccccc3)c3ccc(-c4ccccc4)cc3)cc2)cc1] 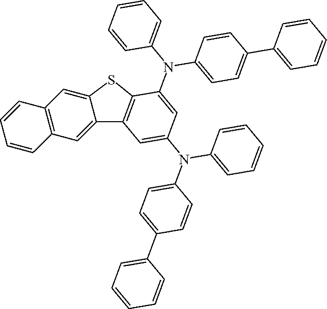 [SMILES: c1ccc(-c2ccc(N(c3ccccc3)c3cc(N(c4ccccc4)c4ccc(-c5ccccc5)cc4)c4sc5ccc6ccccc6c5c4c3)cc2)cc1] 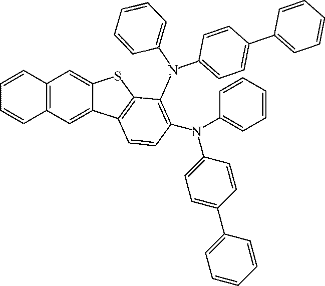 [SMILES: c1ccc(-c2ccc(N(c3ccccc3)c3ccc4c(sc5ccc6ccccc6c54)c3N(c3ccccc3)c3ccc(-c4ccccc4)cc3)cc2)cc1] 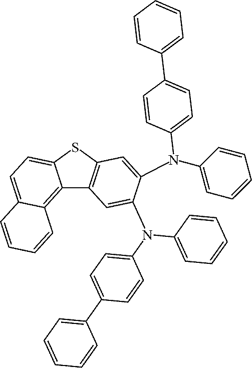 [SMILES: c1ccc(-c2ccccc2N(c2ccccc2)c2cc3c(cc2N(c2ccccc2)c2ccccc2)sc2c4ccccc4ccc32)cc1] 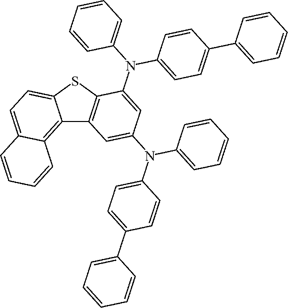 [SMILES: c1ccc(-c2ccccc2N(c2ccccc2)c2cc(N(c3ccccc3)c3ccccc3)c3sc4c5ccccc5ccc4c3c2)cc1] 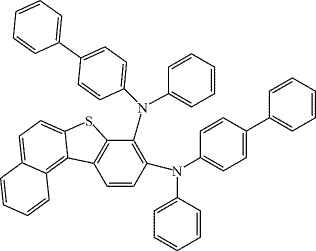 [SMILES: c1ccc(-c2ccccc2N(c2ccccc2)c2ccc3c(sc4c5ccccc5ccc34)c2N(c2ccccc2)c2ccccc2)cc1] 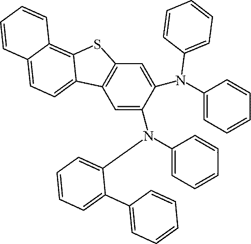 [SMILES: c1ccc(-c2ccccc2N(c2ccccc2)c2cc3c(cc2N(c2ccccc2)c2ccccc2)sc2cc4ccccc4cc23)cc1] 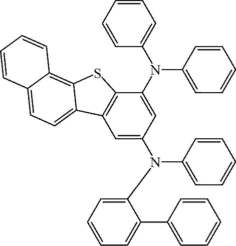 [SMILES: c1ccc(-c2ccccc2N(c2ccccc2)c2cc(N(c3ccccc3)c3ccccc3)cc3c2sc2cc4ccccc4cc23)cc1] 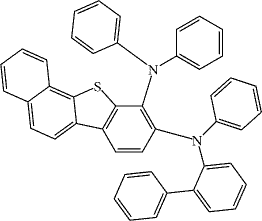 [SMILES: c1ccc(-c2ccccc2N(c2ccccc2)c2ccc3c(sc4cc5ccccc5cc43)c2N(c2ccccc2)c2ccccc2)cc1] 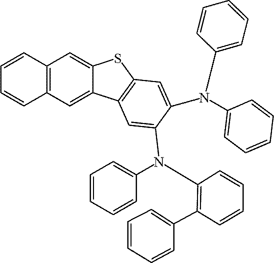 [SMILES: c1ccc(-c2ccccc2N(c2ccccc2)c2cc3c(cc2N(c2ccccc2)c2ccccc2)sc2ccc4ccccc4c23)cc1] 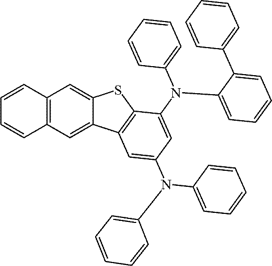 [SMILES: c1ccc(-c2ccccc2N(c2ccccc2)c2cc(N(c3ccccc3)c3ccccc3)cc3c2sc2ccc4ccccc4c23)cc1] 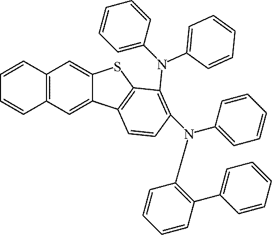 [SMILES: c1ccc(-c2ccccc2N(c2ccccc2)c2c(N(c3ccccc3)c3ccccc3)ccc3c2sc2ccc4ccccc4c23)cc1] 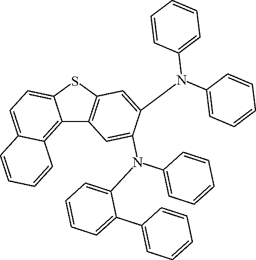 [SMILES: c1ccc(N(c2ccc3ccccc3c2)c2cc3sc4c5ccccc5ccc4c3cc2N(c2ccccc2)c2ccc3ccccc3c2)cc1] 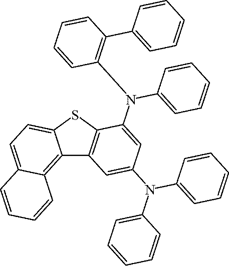 [SMILES: c1ccc(N(c2ccc3ccccc3c2)c2cc(N(c3ccccc3)c3ccc4ccccc4c3)c3sc4c5ccccc5ccc4c3c2)cc1] 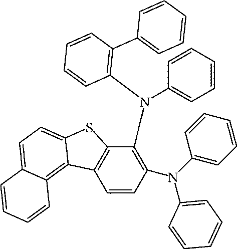 [SMILES: c1ccc(N(c2ccc3ccccc3c2)c2ccc3c(sc4c5ccccc5ccc34)c2N(c2ccccc2)c2ccc3ccccc3c2)cc1] 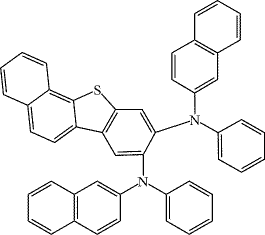 [SMILES: c1ccc(N(c2ccc3ccccc3c2)c2cc3sc4cc5ccccc5cc4c3cc2N(c2ccccc2)c2ccc3ccccc3c2)cc1] 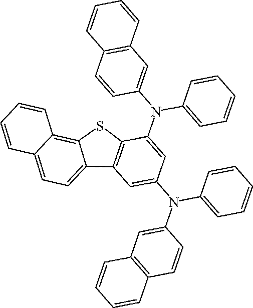 [SMILES: c1ccc(N(c2ccc3ccccc3c2)c2cc(N(c3ccccc3)c3ccc4ccccc4c3)c3sc4cc5ccccc5cc4c3c2)cc1] 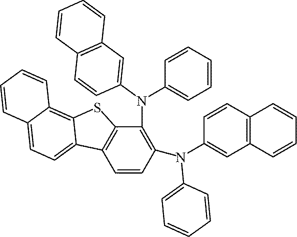 [SMILES: c1ccc(N(c2ccc3ccccc3c2)c2ccc3c(sc4cc5ccccc5cc43)c2N(c2ccccc2)c2ccc3ccccc3c2)cc1] 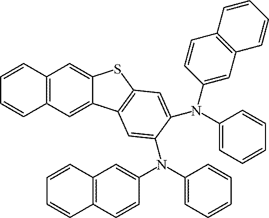 [SMILES: c1ccc(N(c2ccc3ccccc3c2)c2cc3sc4ccc5ccccc5c4c3cc2N(c2ccccc2)c2ccc3ccccc3c2)cc1] 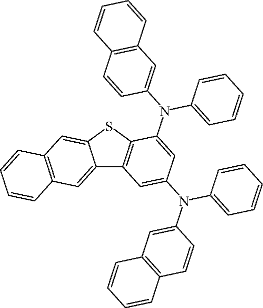 [SMILES: c1ccc(N(c2ccc3ccccc3c2)c2cc(N(c3ccccc3)c3ccc4ccccc4c3)c3sc4ccc5ccccc5c4c3c2)cc1] 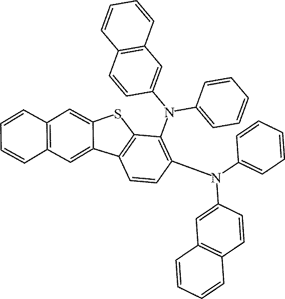 [SMILES: c1ccc(N(c2ccc3ccccc3c2)c2ccc3c(sc4ccc5ccccc5c43)c2N(c2ccccc2)c2ccc3ccccc3c2)cc1] 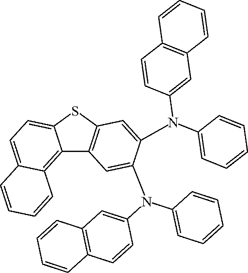 [SMILES: c1ccc(-c2cccc(N(c3ccc4ccccc4c3)c3cc4c(cc3N(c3ccccc3)c3ccc5ccccc5c3)sc3c5ccccc5ccc43)c2)cc1] 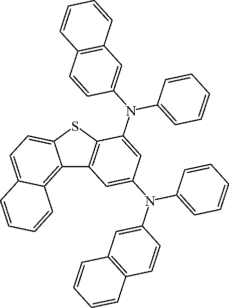 [SMILES: c1ccc(-c2ccc(N(c3ccc4ccccc4c3)c3cc(N(c4ccccc4)c4ccc5ccccc5c4)c4sc5c6ccccc6ccc5c4c3)cc2)cc1] 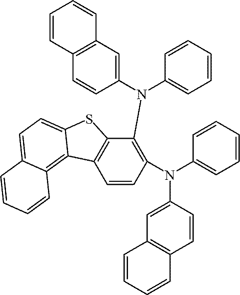 [SMILES: c1ccc(-c2cccc(N(c3ccc4ccccc4c3)c3c(N(c4ccccc4)c4ccc5ccccc5c4)ccc4c3sc3c5ccccc5ccc43)c2)cc1] 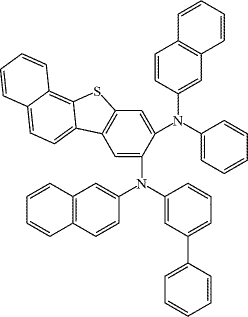 [SMILES: c1ccc(-c2cccc(N(c3ccc4ccccc4c3)c3cc4c(cc3N(c3ccccc3)c3ccc5ccccc5c3)sc3cc5ccccc5cc34)c2)cc1] 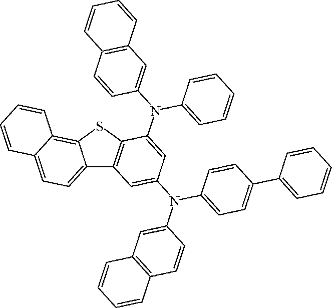 [SMILES: c1ccc(-c2cccc(N(c3ccc4ccccc4c3)c3cc(N(c4ccccc4)c4ccc5ccccc5c4)cc4c3sc3cc5ccccc5cc34)c2)cc1] 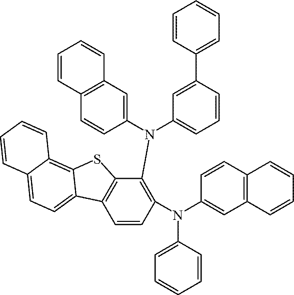 [SMILES: c1ccc(-c2ccc(N(c3ccc4ccccc4c3)c3c(N(c4ccccc4)c4ccc5ccccc5c4)ccc4c3sc3cc5ccccc5cc34)cc2)cc1] 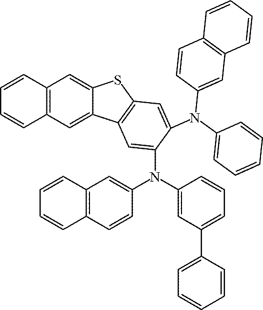 [SMILES: c1ccc(-c2cccc(N(c3ccc4ccccc4c3)c3cc4c(cc3N(c3ccccc3)c3ccc5ccccc5c3)sc3ccc5ccccc5c34)c2)cc1] 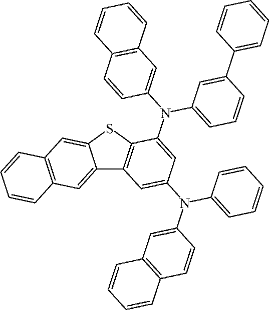 [SMILES: c1ccc(-c2ccc(N(c3ccc4ccccc4c3)c3cc(N(c4ccccc4)c4ccc5ccccc5c4)cc4c3sc3ccc5ccccc5c34)cc2)cc1] 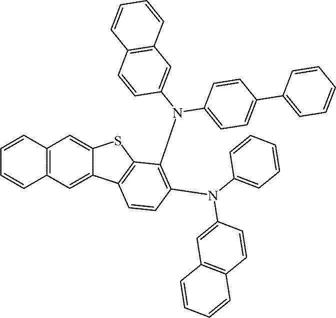 [SMILES: c1ccc(-c2ccc(N(c3ccc4ccccc4c3)c3c(N(c4ccccc4)c4ccc5ccccc5c4)ccc4c3sc3ccc5ccccc5c34)cc2)cc1] 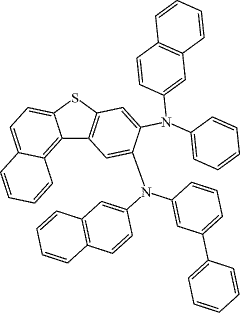 [SMILES: c1ccc(-c2ccc(N(c3ccccc3)c3cc4sc5c6ccccc6ccc5c4cc3N(c3ccc4ccccc4c3)c3ccc4sc5ccccc5c4c3)cc2)cc1] 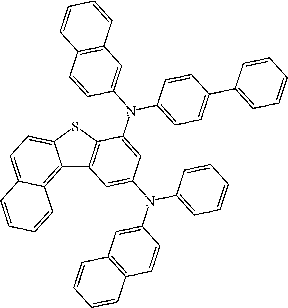 [SMILES: c1ccc(-c2ccc(N(c3ccccc3)c3cc(N(c4ccccc4)c4ccc5sc6ccccc6c5c4)cc4c3sc3c5ccccc5ccc43)cc2)cc1] 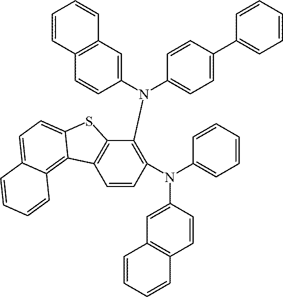 [SMILES: c1ccc(-c2cccc(N(c3ccc4c(c3)oc3ccccc34)c3c(N(c4ccccc4)c4ccccc4)ccc4c3sc3c5ccccc5ccc43)c2)cc1] 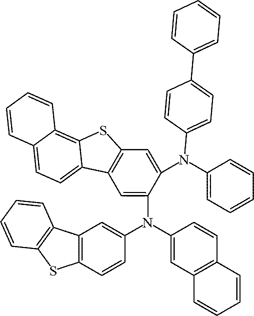 [SMILES: c1ccc(-c2ccc(N(c3ccccc3)c3cc4c(cc3N(c3ccccc3)c3ccc5sc6ccccc6c5c3)sc3cc5ccccc5cc34)c3ccccc23)cc1] 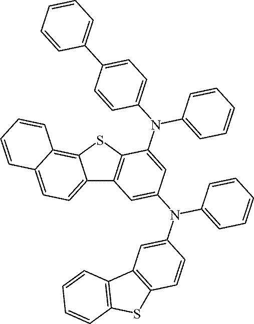 [SMILES: c1ccc(-c2ccc(N(c3ccccc3)c3cc(N(c4ccccc4)c4ccc5c(c4)oc4ccccc45)c4sc5cc6ccccc6cc5c4c3)cc2)cc1] 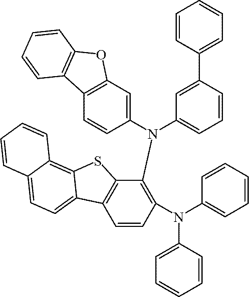 [SMILES: c1ccc(N(c2cccc(-c3ccc4ccccc4c3)c2)c2ccc3c(sc4cc5ccccc5cc43)c2N(c2ccccc2)c2ccc3sc4ccccc4c3c2)cc1] 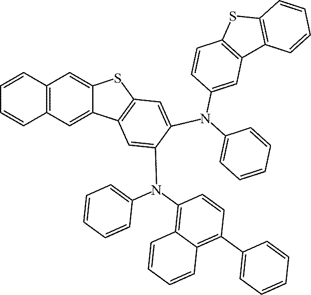 [SMILES: c1ccc(-c2ccc(N(c3ccccc3)c3cc4c(cc3N(c3ccc5ccccc5c3)c3ccc5c(c3)oc3ccccc35)sc3ccc5ccccc5c34)cc2)cc1] 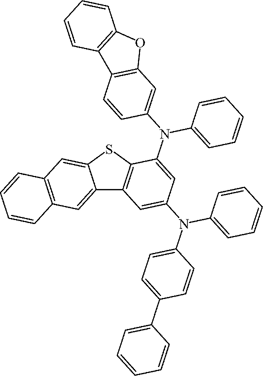 [SMILES: c1ccc(N(c2ccccc2)c2cc(N(c3ccc4ccccc4c3)c3ccc4c(c3)sc3ccccc34)c3sc4ccc5ccccc5c4c3c2)cc1] 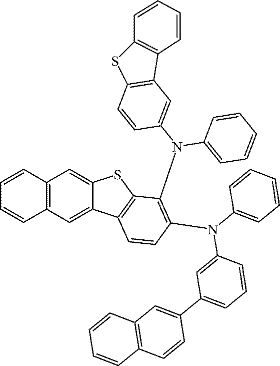 [SMILES: c1ccc(N(c2ccc3ccccc3c2)c2ccc3c(sc4ccc5ccccc5c43)c2N(c2ccc3ccccc3c2)c2ccc3sc4ccccc4c3c2)cc1] 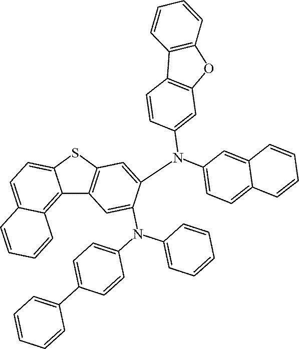 [SMILES: c1ccc(N(c2ccc(-c3cccc4ccccc34)cc2)c2cc3sc4c5ccccc5ccc4c3cc2N(c2ccccc2)c2cccc3c2sc2ccccc23)cc1] 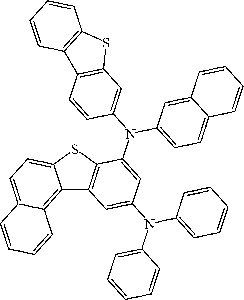 [SMILES: c1ccc(-c2cccc(N(c3ccccc3)c3cc(N(c4ccccc4)c4cccc5oc6ccccc6c45)c4sc5c6ccccc6ccc5c4c3)c2)cc1] 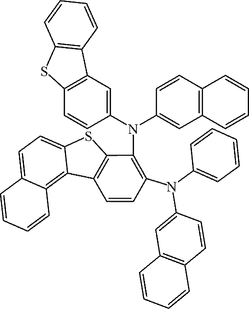 [SMILES: c1ccc(N(c2ccc3c(sc4c5ccccc5ccc34)c2N(c2ccccc2)c2ccc(-c3ccc4ccccc4c3)cc2)c2cccc3c2sc2ccccc23)cc1] 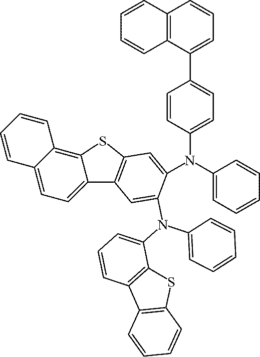 [SMILES: c1ccc(N(c2cc3sc4cc5ccccc5cc4c3cc2N(c2ccc3ccccc3c2)c2ccc3ccccc3c2)c2cccc3c2sc2ccccc23)cc1] 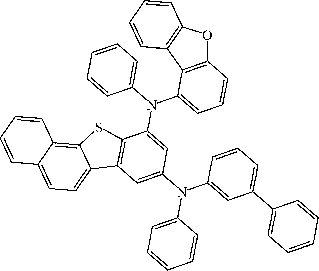 [SMILES: c1ccc(-c2ccc(N(c3ccccc3)c3cc(N(c4ccccc4)c4cccc5oc6ccccc6c45)c4sc5cc6ccccc6cc5c4c3)cc2)cc1] 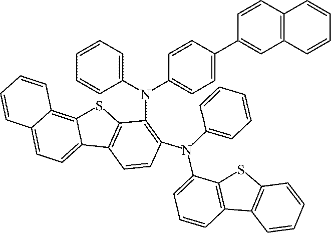 [SMILES: c1ccc(-c2ccc(N(c3cccc4c3sc3ccccc34)c3c(N(c4ccccc4)c4ccc5ccccc5c4)ccc4c3sc3cc5ccccc5cc34)cc2)cc1] 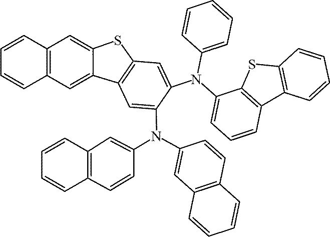 [SMILES: c1ccc(-c2cccc(N(c3cccc(-c4ccccc4)c3)c3cc4c(cc3N(c3ccc5ccccc5c3)c3cccc5c3oc3ccccc35)sc3ccc5ccccc5c34)c2)cc1] 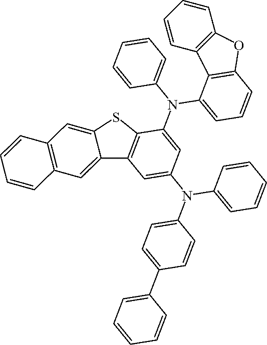 [SMILES: c1ccc(-c2ccc(N(c3ccc(-c4ccccc4)cc3)c3cc(N(c4ccccc4)c4cccc5oc6ccccc6c45)cc4c3sc3ccc5ccccc5c34)cc2)cc1] 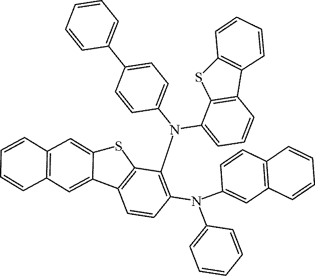 [SMILES: c1ccc(-c2ccc(N(c3ccc4ccccc4c3)c3c(N(c4ccccc4)c4cccc5sc6ccccc6c45)ccc4c3sc3ccc5ccccc5c34)cc2)cc1] 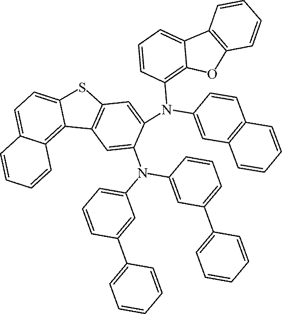 [SMILES: c1ccc(-c2ccc(N(c3ccccc3)c3ccc4sc5c6ccccc6ccc5c4c3N(c3ccc(-c4ccccc4)cc3)c3cccc(-c4ccccc4)c3)cc2)cc1] 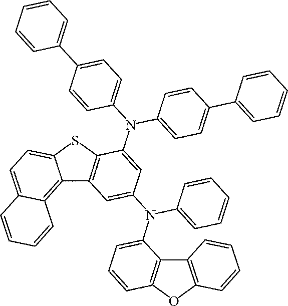 [SMILES: c1ccc(-c2ccc(N(c3cccc(-c4ccccc4)c3)c3cc(N(c4ccccc4)c4ccc(-c5ccccc5)cc4)c4c(c3)sc3c5ccccc5ccc34)cc2)cc1] 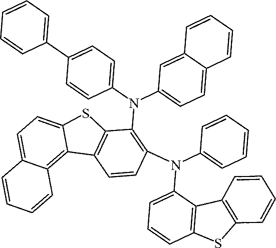 [SMILES: c1ccc(-c2ccc(N(c3ccccc3)c3ccc(N(c4ccc(-c5ccccc5)cc4)c4cccc(-c5ccccc5)c4)c4c3sc3c5ccccc5ccc34)cc2)cc1] 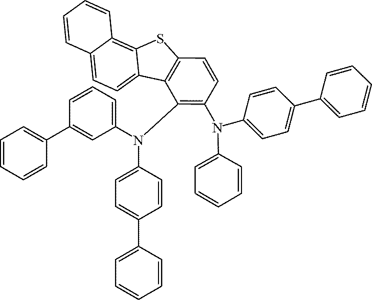 [SMILES: c1ccc(-c2ccc(N(c3cccc(-c4ccccc4)c3)c3ccc4sc5cc6ccccc6cc5c4c3N(c3ccccc3)c3ccc(-c4ccccc4)cc3)cc2)cc1] 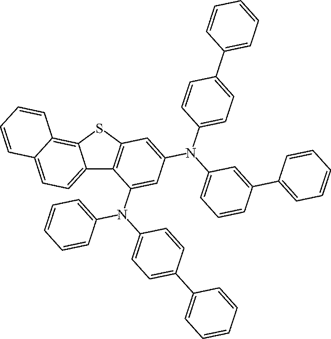 [SMILES: c1ccc(-c2ccc(N(c3cccc(-c4ccccc4)c3)c3cc(N(c4ccccc4)c4ccc(-c5ccccc5)cc4)c4c(c3)sc3cc5ccccc5cc34)cc2)cc1] 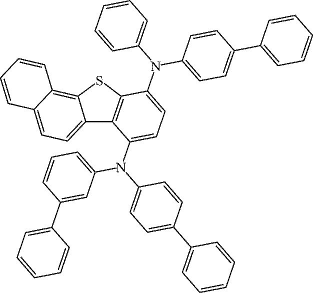 [SMILES: c1ccc(-c2ccc(N(c3ccccc3)c3ccc(N(c4ccc(-c5ccccc5)cc4)c4cccc(-c5ccccc5)c4)c4c3sc3cc5ccccc5cc34)cc2)cc1] 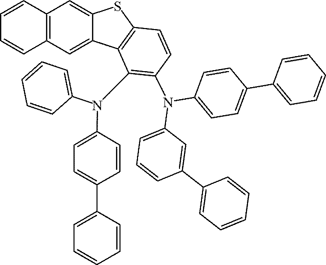 [SMILES: c1ccc(-c2ccc(N(c3ccccc3)c3ccc4sc5ccc6ccccc6c5c4c3N(c3ccc(-c4ccccc4)cc3)c3cccc(-c4ccccc4)c3)cc2)cc1] 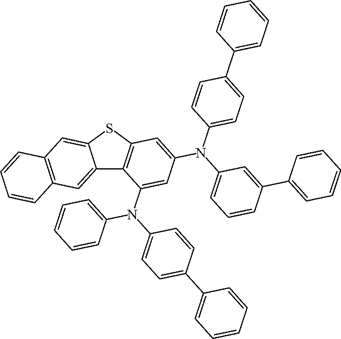 [SMILES: c1ccc(-c2ccc(N(c3cccc(-c4ccccc4)c3)c3cc(N(c4ccccc4)c4ccc(-c5ccccc5)cc4)c4c(c3)sc3ccc5ccccc5c34)cc2)cc1] 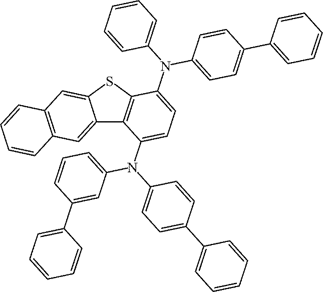 [SMILES: c1ccc(-c2ccc(N(c3ccccc3)c3ccc(N(c4ccc(-c5ccccc5)cc4)c4cccc(-c5ccccc5)c4)c4c3sc3ccc5ccccc5c34)cc2)cc1] 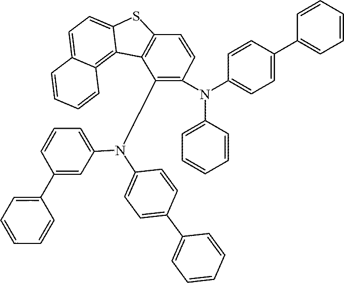 [SMILES: c1ccc(-c2ccc(N(c3ccccc3)c3ccc4sc5c6ccccc6ccc5c4c3N(c3ccccc3)c3ccccc3-c3ccccc3)cc2)cc1] 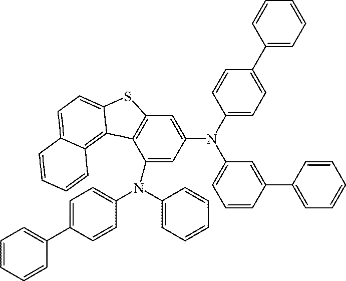 [SMILES: c1ccc(-c2ccc(N(c3ccccc3)c3cc(N(c4ccccc4)c4ccccc4-c4ccccc4)c4c(c3)sc3c5ccccc5ccc34)cc2)cc1] 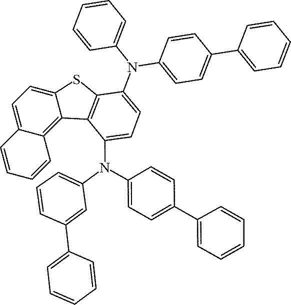 [SMILES: c1ccc(-c2ccc(N(c3ccccc3)c3ccc(N(c4ccccc4)c4ccccc4-c4ccccc4)c4c3sc3c5ccccc5ccc34)cc2)cc1] 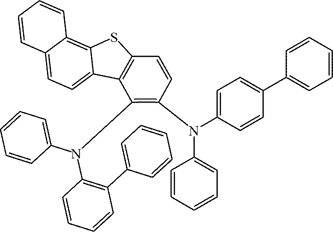 [SMILES: c1ccc(-c2ccc(N(c3ccccc3)c3ccc4sc5cc6ccccc6cc5c4c3N(c3ccccc3)c3ccccc3-c3ccccc3)cc2)cc1] 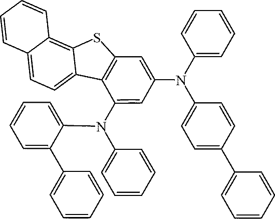 [SMILES: c1ccc(-c2ccc(N(c3ccccc3)c3cc(N(c4ccccc4)c4ccccc4-c4ccccc4)c4c(c3)sc3cc5ccccc5cc34)cc2)cc1] 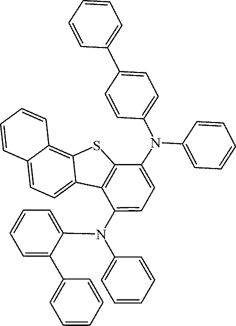 [SMILES: c1ccc(-c2ccc(N(c3ccccc3)c3ccc(N(c4ccccc4)c4ccccc4-c4ccccc4)c4sc5cc6ccccc6cc5c34)cc2)cc1] 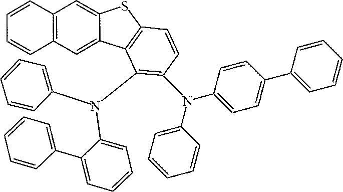 [SMILES: c1ccc(-c2ccc(N(c3ccccc3)c3ccc4sc5ccc6ccccc6c5c4c3N(c3ccccc3)c3ccccc3-c3ccccc3)cc2)cc1] 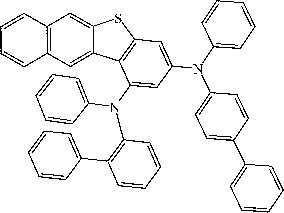 [SMILES: c1ccc(-c2ccc(N(c3ccccc3)c3cc(N(c4ccccc4)c4ccccc4-c4ccccc4)cc4sc5ccc6ccccc6c5c34)cc2)cc1] 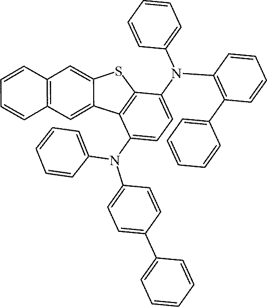 [SMILES: c1ccc(-c2ccc(N(c3ccccc3)c3ccc(N(c4ccccc4)c4ccccc4-c4ccccc4)c4c3sc3ccc5ccccc5c34)cc2)cc1] 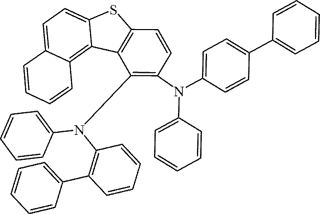 [SMILES: c1ccc(-c2ccc(N(c3ccccc3)c3ccc4sc5c6ccccc6ccc5c4c3N(c3ccccc3)c3ccc(-c4ccccc4)cc3)cc2)cc1] 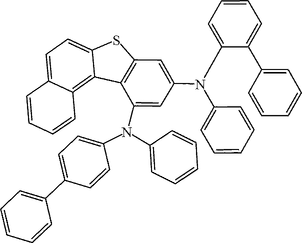 [SMILES: c1ccc(-c2ccc(N(c3ccccc3)c3cc(N(c4ccccc4)c4ccc(-c5ccccc5)cc4)c4c(c3)sc3c5ccccc5ccc34)cc2)cc1] 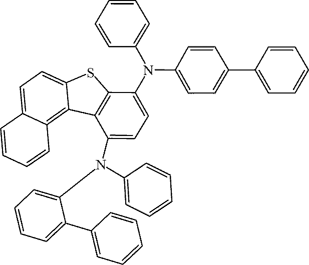 [SMILES: c1ccc(-c2ccc(N(c3ccccc3)c3ccc(N(c4ccccc4)c4ccc(-c5ccccc5)cc4)c4c3sc3c5ccccc5ccc34)cc2)cc1] 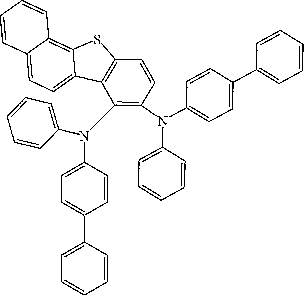 [SMILES: c1ccc(-c2ccc(N(c3ccccc3)c3ccc4sc5cc6ccccc6cc5c4c3N(c3ccccc3)c3ccc(-c4ccccc4)cc3)cc2)cc1] 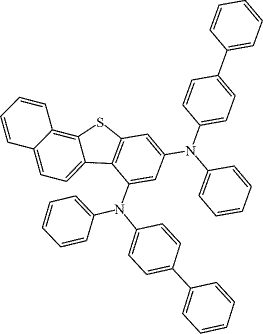 [SMILES: c1ccc(-c2ccc(N(c3ccccc3)c3cc(N(c4ccccc4)c4ccc(-c5ccccc5)cc4)c4c(c3)sc3cc5ccccc5cc34)cc2)cc1] 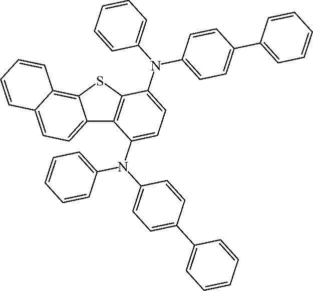 [SMILES: c1ccc(-c2ccc(N(c3ccccc3)c3ccc(N(c4ccccc4)c4ccc(-c5ccccc5)cc4)c4c3sc3cc5ccccc5cc34)cc2)cc1] 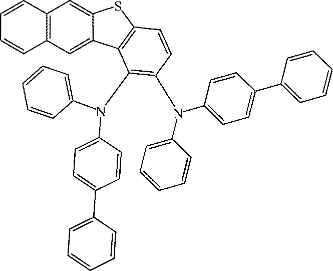 [SMILES: c1ccc(-c2ccc(N(c3ccccc3)c3ccc4sc5ccc6ccccc6c5c4c3N(c3ccccc3)c3ccc(-c4ccccc4)cc3)cc2)cc1] 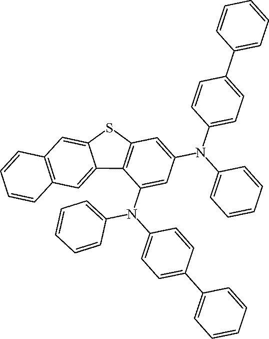 [SMILES: c1ccc(-c2ccc(N(c3ccccc3)c3cc(N(c4ccccc4)c4ccc(-c5ccccc5)cc4)c4c(c3)sc3ccc5ccccc5c34)cc2)cc1] 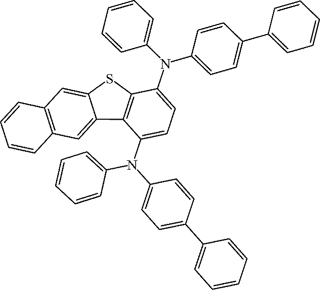 [SMILES: c1ccc(-c2ccc(N(c3ccccc3)c3ccc(N(c4ccccc4)c4ccc(-c5ccccc5)cc4)c4c3sc3ccc5ccccc5c34)cc2)cc1] 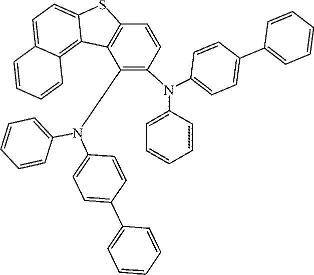 [SMILES: c1ccc(-c2cccc(N(c3ccccc3)c3ccc4sc5c6ccccc6ccc5c4c3N(c3ccccc3)c3ccccc3-c3ccccc3)c2)cc1] 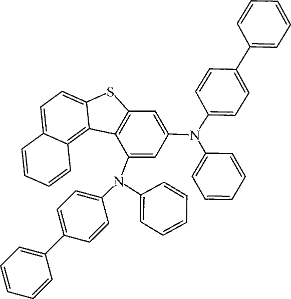 [SMILES: c1ccc(-c2cccc(N(c3ccccc3)c3cc(N(c4ccccc4)c4ccccc4-c4ccccc4)c4c(c3)sc3c5ccccc5ccc34)c2)cc1] 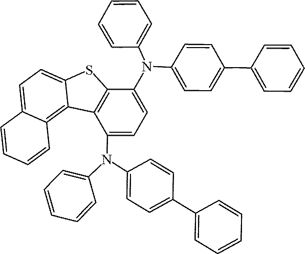 [SMILES: c1ccc(-c2cccc(N(c3ccccc3)c3ccc(N(c4ccccc4)c4ccccc4-c4ccccc4)c4c3sc3c5ccccc5ccc34)c2)cc1] 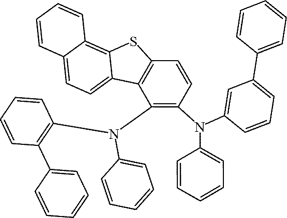 [SMILES: c1ccc(-c2cccc(N(c3ccccc3)c3ccc4sc5cc6ccccc6cc5c4c3N(c3ccccc3)c3ccccc3-c3ccccc3)c2)cc1] 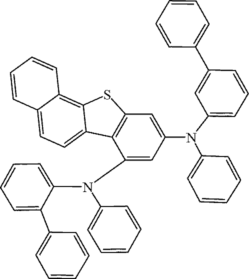 [SMILES: c1ccc(-c2cccc(N(c3ccccc3)c3cc(N(c4ccccc4)c4ccccc4-c4ccccc4)c4c(c3)sc3cc5ccccc5cc34)c2)cc1] 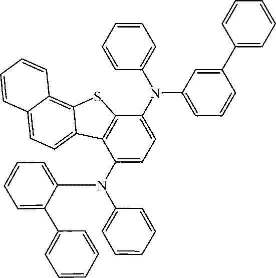 [SMILES: c1ccc(-c2cccc(N(c3ccccc3-c3ccccc3)c3ccc(N(c4ccccc4)c4ccccc4)c4c3sc3cc5ccccc5cc34)c2)cc1] 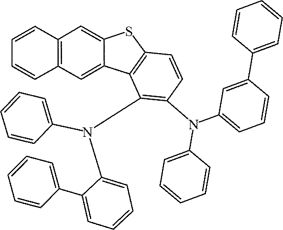 [SMILES: c1ccc(-c2cccc(N(c3ccccc3)c3ccc4sc5ccc6ccccc6c5c4c3N(c3ccccc3)c3ccccc3-c3ccccc3)c2)cc1] 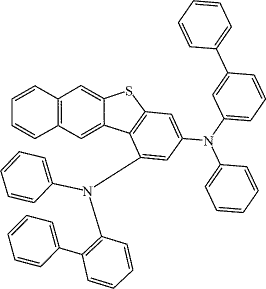 [SMILES: c1ccc(-c2cccc(N(c3ccccc3)c3cc(N(c4ccccc4)c4ccccc4-c4ccccc4)cc4sc5ccc6ccccc6c5c34)c2)cc1] 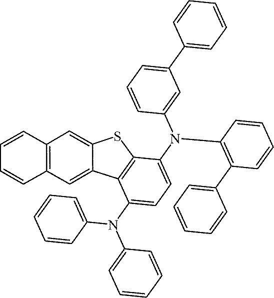 [SMILES: c1ccc(-c2cccc(N(c3ccccc3)c3ccc(N(c4ccccc4)c4ccccc4-c4ccccc4)c4c3sc3ccc5ccccc5c34)c2)cc1] 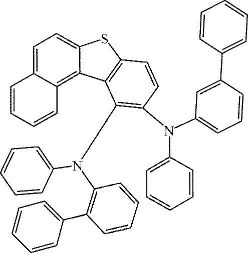 [SMILES: c1ccc(N(c2ccc3oc4ccccc4c3c2)c2ccc3sc4c5ccccc5ccc4c3c2N(c2ccccc2)c2ccc3c(c2)oc2ccccc23)cc1] 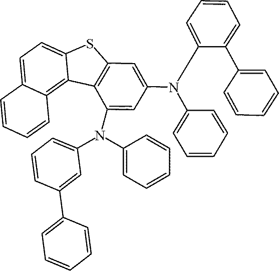 [SMILES: c1ccc(N(c2ccc3c(c2)oc2ccccc23)c2cc(N(c3ccccc3)c3ccc4c(c3)oc3ccccc34)c3c(c2)sc2c4ccccc4ccc23)cc1] 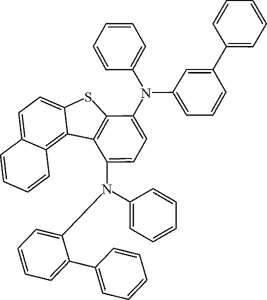 [SMILES: c1ccc(N(c2ccc3c(c2)oc2ccccc23)c2ccc(N(c3ccccc3)c3ccc4c(c3)oc3ccccc34)c3c2sc2c4ccccc4ccc23)cc1] 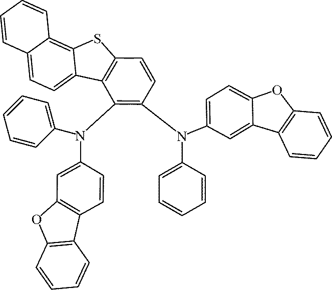 [SMILES: c1ccc(N(c2ccc3c(c2)sc2ccccc23)c2ccc3sc4cc5ccccc5cc4c3c2N(c2ccccc2)c2ccc3c(c2)sc2ccccc23)cc1] 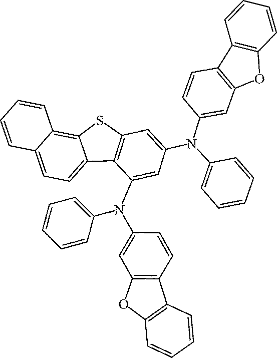 [SMILES: c1ccc(N(c2cc(N(c3ccccc3)c3ccc4oc5ccccc5c4c3)c3c(c2)sc2cc4ccccc4cc23)c2ccc3oc4ccccc4c3c2)cc1] 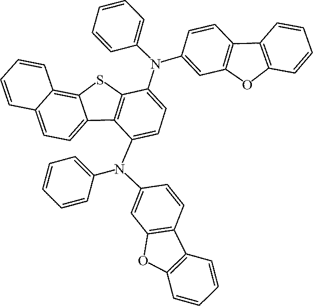 [SMILES: c1ccc(N(c2ccc3c(c2)oc2ccccc23)c2ccc(N(c3ccccc3)c3ccc4c(c3)oc3ccccc34)c3c2sc2cc4ccccc4cc23)cc1] 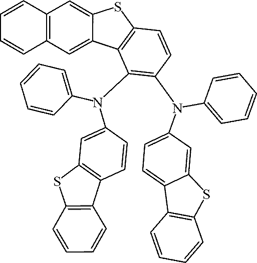 [SMILES: c1ccc(N(c2ccc3sc4ccc5ccccc5c4c3c2N(c2ccccc2)c2ccc3c(c2)sc2ccccc23)c2cccc3c2sc2ccccc23)cc1] 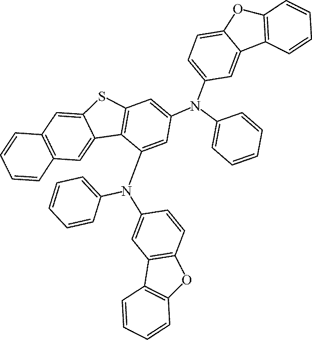 [SMILES: c1ccc(N(c2cc(N(c3ccccc3)c3ccc4c(c3)sc3ccccc34)c3c(c2)sc2ccc4ccccc4c23)c2ccc3sc4ccccc4c3c2)cc1] 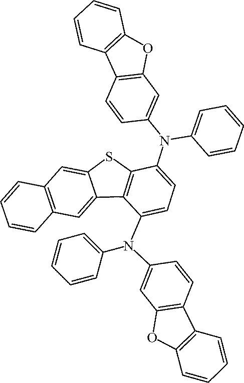 [SMILES: c1ccc(N(c2ccccc2)c2ccc(N(c3ccc4c(c3)oc3ccccc34)c3ccc4oc5ccccc5c4c3)c3c2sc2ccc4ccccc4c23)cc1] 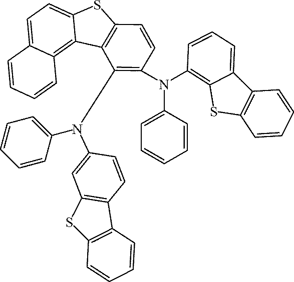 [SMILES: c1ccc(N(c2ccc3sc4c5ccccc5ccc4c3c2N(c2ccccc2)c2ccc3oc4ccccc4c3c2)c2cccc3c2sc2ccccc23)cc1] 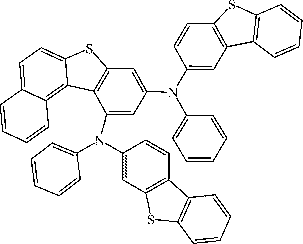 [SMILES: c1ccc(N(c2cc(N(c3ccccc3)c3cccc4oc5ccccc5c34)c3c(c2)sc2c4ccccc4ccc23)c2cccc3oc4ccccc4c23)cc1] 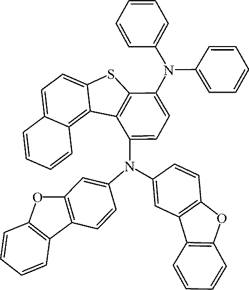 [SMILES: c1ccc(N(c2ccc(N(c3ccccc3)c3cccc4oc5ccccc5c34)c3c2sc2c4ccccc4ccc23)c2cccc3oc4ccccc4c23)cc1] 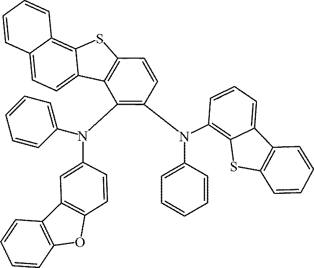 [SMILES: c1ccc(N(c2ccccc2)c2c(N(c3cccc4c3sc3ccccc34)c3cccc4c3sc3ccccc34)ccc3sc4cc5ccccc5cc4c23)cc1] 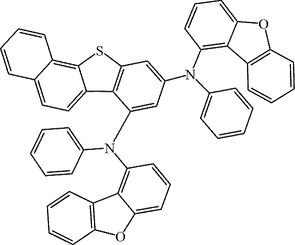 [SMILES: c1ccc(N(c2cc(N(c3ccccc3)c3cccc4oc5ccccc5c34)c3c(c2)sc2cc4ccccc4cc23)c2cccc3oc4ccccc4c23)cc1] 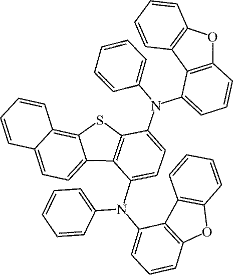 [SMILES: c1ccc(N(c2ccc(N(c3ccccc3)c3cccc4oc5ccccc5c34)c3c2sc2cc4ccccc4cc23)c2cccc3oc4ccccc4c23)cc1] 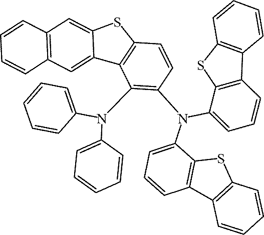 [SMILES: c1ccc(N(c2ccc3sc4ccc5ccccc5c4c3c2N(c2ccccc2)c2ccc3sc4ccccc4c3c2)c2cccc3c2sc2ccccc23)cc1] 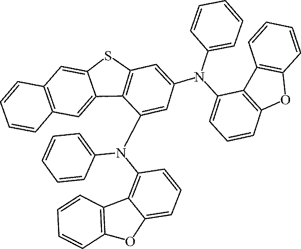 [SMILES: c1ccc(N(c2cc(N(c3ccccc3)c3ccc4c(c3)sc3ccccc34)c3c(c2)sc2ccc4ccccc4c23)c2cccc3c2sc2ccccc23)cc1] 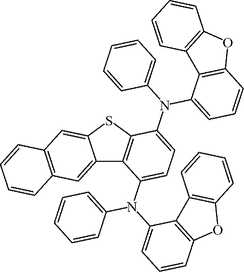 [SMILES: c1ccc(N(c2cccc3c2sc2ccccc23)c2ccc(N(c3ccccc3)c3cccc4c3oc3ccccc34)c3c2sc2ccc4ccccc4c23)cc1] 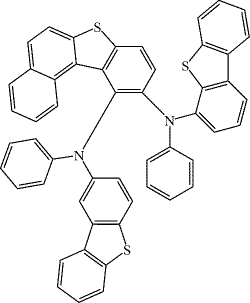 [SMILES: c1ccc(-c2ccc(N(c3ccccc3)c3cc4sc5c6ccccc6ccc5c4cc3N(c3ccc(-c4ccccc4)cc3)c3cccc(-c4ccccc4)c3)cc2)cc1] 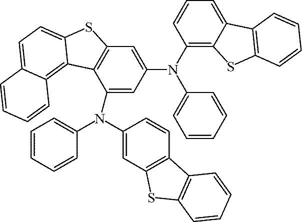 [SMILES: c1ccc(-c2ccc(N(c3cccc(-c4ccccc4)c3)c3cc(N(c4ccccc4)c4ccc(-c5ccccc5)cc4)c4sc5c6ccccc6ccc5c4c3)cc2)cc1] 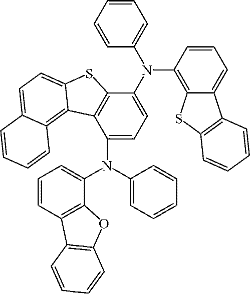 [SMILES: c1ccc(-c2ccc(N(c3cccc(-c4ccccc4)c3)c3ccc4c(sc5c6ccccc6ccc45)c3N(c3ccccc3)c3ccc(-c4ccccc4)cc3)cc2)cc1] 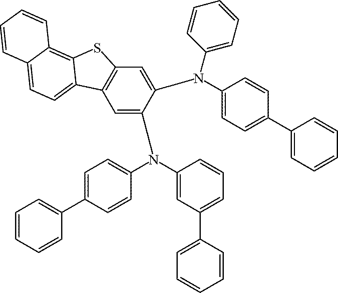 [SMILES: c1ccc(-c2ccc(N(c3ccccc3)c3cc4sc5cc6ccccc6cc5c4cc3N(c3ccc(-c4ccccc4)cc3)c3cccc(-c4ccccc4)c3)cc2)cc1] 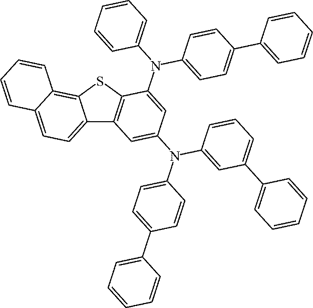 [SMILES: c1ccc(-c2ccc(N(c3ccccc3)c3cc(N(c4ccc(-c5ccccc5)cc4)c4cccc(-c5ccccc5)c4)c4sc5cc6ccccc6cc5c4c3)cc2)cc1] 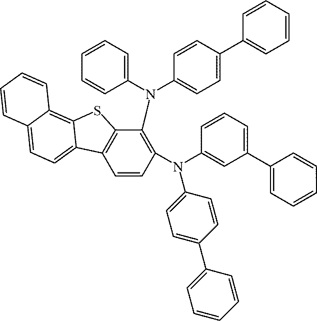 [SMILES: c1ccc(-c2ccc(N(c3ccccc3)c3ccc4c(sc5cc6ccccc6cc54)c3N(c3ccc(-c4ccccc4)cc3)c3cccc(-c4ccccc4)c3)cc2)cc1] 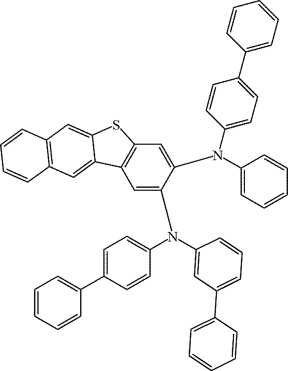 [SMILES: c1ccc(-c2ccc(N(c3cccc(-c4ccccc4)c3)c3cc4sc5ccc6ccccc6c5c4cc3N(c3ccccc3)c3ccc(-c4ccccc4)cc3)cc2)cc1] 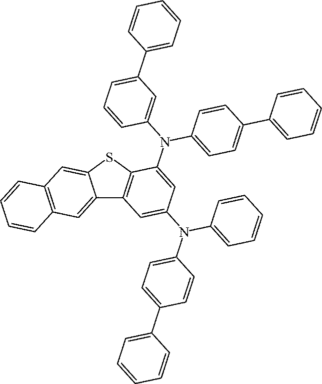 [SMILES: c1ccc(-c2ccc(N(c3cccc(-c4ccccc4)c3)c3cc(N(c4ccccc4)c4ccc(-c5ccccc5)cc4)c4sc5ccc6ccccc6c5c4c3)cc2)cc1] 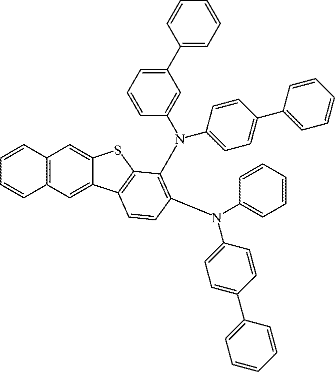 [SMILES: c1ccc(-c2ccc(N(c3ccccc3)c3ccc4c(sc5ccc6ccccc6c54)c3N(c3ccc(-c4ccccc4)cc3)c3cccc(-c4ccccc4)c3)cc2)cc1] 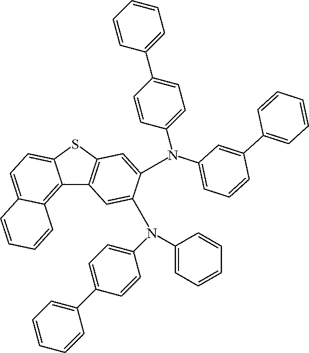 [SMILES: c1ccc(-c2ccc(N(c3ccccc3)c3cc4sc5c6ccccc6ccc5c4cc3N(c3ccccc3)c3ccccc3-c3ccccc3)cc2)cc1] 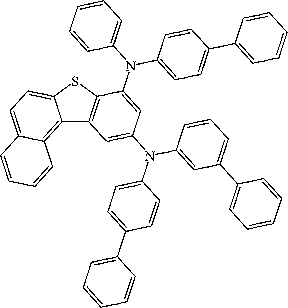 [SMILES: c1ccc(-c2ccc(N(c3ccccc3)c3cc(N(c4ccccc4)c4ccccc4-c4ccccc4)cc4c3sc3c5ccccc5ccc43)cc2)cc1] 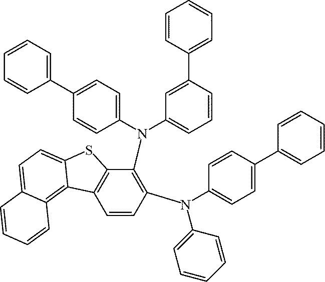 [SMILES: c1ccc(-c2ccc(N(c3ccccc3)c3c(N(c4ccccc4)c4ccccc4-c4ccccc4)ccc4c3sc3c5ccccc5ccc43)cc2)cc1] 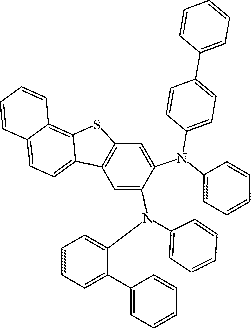 [SMILES: c1ccc(-c2ccc(N(c3ccccc3)c3cc4sc5cc6ccccc6cc5c4cc3N(c3ccccc3)c3ccccc3-c3ccccc3)cc2)cc1] 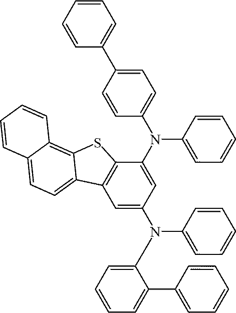 [SMILES: c1ccc(-c2ccc(N(c3ccccc3)c3cc(N(c4ccccc4)c4ccccc4-c4ccccc4)c4sc5cc6ccccc6cc5c4c3)cc2)cc1] 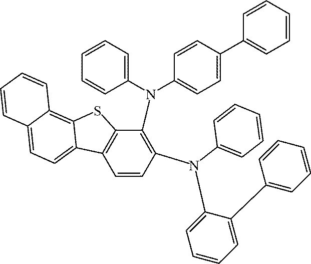 [SMILES: c1ccc(-c2ccc(N(c3ccccc3)c3c(N(c4ccccc4)c4ccccc4-c4ccccc4)ccc4c3sc3cc5ccccc5cc34)cc2)cc1] 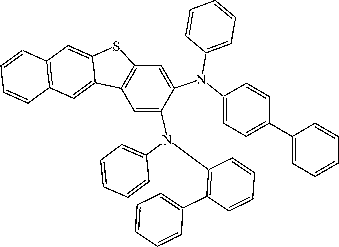 [SMILES: c1ccc(-c2ccc(N(c3ccccc3)c3cc4sc5ccc6ccccc6c5c4cc3N(c3ccccc3)c3ccccc3-c3ccccc3)cc2)cc1] 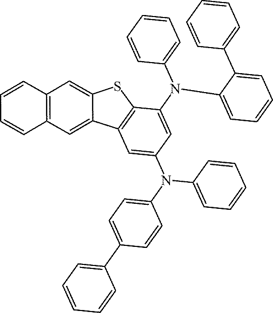 [SMILES: c1ccc(-c2ccccc2N(c2ccccc2)c2cc(N(c3ccccc3)c3ccc4ccccc4c3)cc3c2sc2ccc4ccccc4c23)cc1] 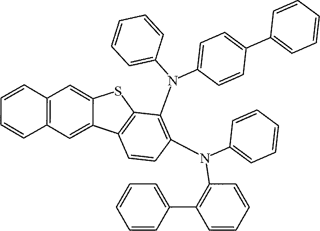 [SMILES: c1ccc(-c2ccc(N(c3ccccc3)c3ccc4c(sc5ccc6ccccc6c54)c3N(c3ccccc3)c3ccccc3-c3ccccc3)cc2)cc1] 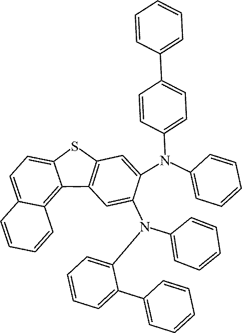 [SMILES: c1ccc(-c2ccc(N(c3ccccc3)c3cc4sc5c6ccccc6ccc5c4cc3N(c3ccccc3)c3ccc(-c4ccccc4)cc3)cc2)cc1] 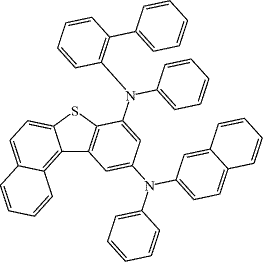 [SMILES: c1ccc(-c2ccc(N(c3ccccc3)c3cc(N(c4ccccc4)c4ccc(-c5ccccc5)cc4)c4sc5c6ccccc6ccc5c4c3)cc2)cc1] 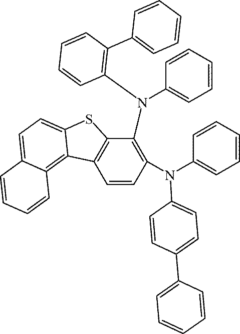 [SMILES: c1ccc(-c2ccc(N(c3ccccc3)c3ccc4c(sc5c6ccccc6ccc45)c3N(c3ccccc3)c3ccc(-c4ccccc4)cc3)cc2)cc1] 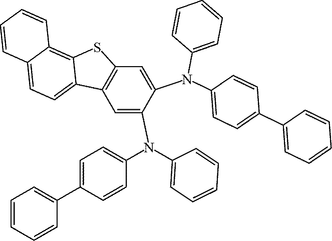 [SMILES: c1ccc(-c2ccc(N(c3ccccc3)c3cc4sc5cc6ccccc6cc5c4cc3N(c3ccccc3)c3ccc(-c4ccccc4)cc3)cc2)cc1] 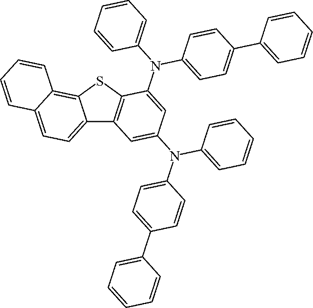 [SMILES: c1ccc(-c2ccc(N(c3ccccc3)c3cc(N(c4ccccc4)c4ccc(-c5ccccc5)cc4)c4sc5cc6ccccc6cc5c4c3)cc2)cc1] 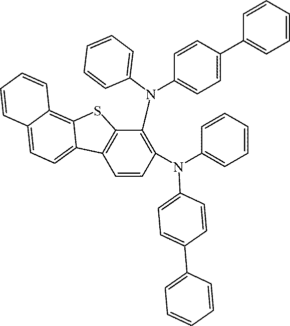 [SMILES: c1ccc(-c2ccc(N(c3ccccc3)c3ccc4c(sc5cc6ccccc6cc54)c3N(c3ccccc3)c3ccc(-c4ccccc4)cc3)cc2)cc1] 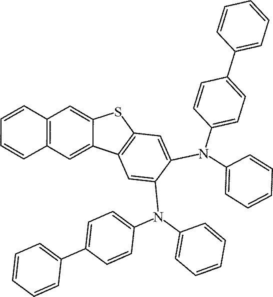 [SMILES: c1ccc(-c2ccc(N(c3ccccc3)c3cc4sc5ccc6ccccc6c5c4cc3N(c3ccccc3)c3ccc(-c4ccccc4)cc3)cc2)cc1] 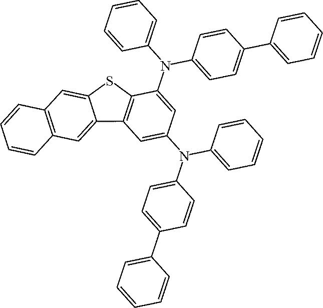 [SMILES: c1ccc(-c2ccc(N(c3ccccc3)c3cc(N(c4ccccc4)c4ccc(-c5ccccc5)cc4)c4sc5ccc6ccccc6c5c4c3)cc2)cc1] 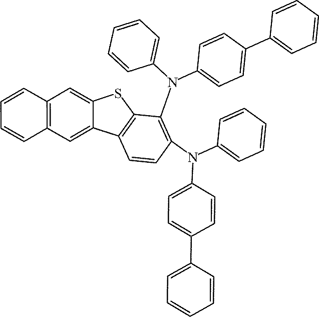 [SMILES: c1ccc(-c2ccc(N(c3ccccc3)c3ccc4c(sc5ccc6ccccc6c54)c3N(c3ccccc3)c3ccc(-c4ccccc4)cc3)cc2)cc1] 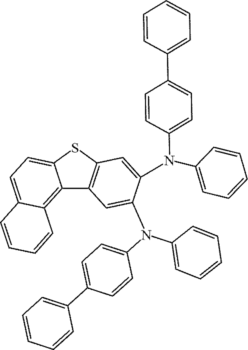 [SMILES: c1ccc(-c2cccc(N(c3ccccc3)c3cc4sc5c6ccccc6ccc5c4cc3N(c3ccccc3)c3ccccc3-c3ccccc3)c2)cc1] 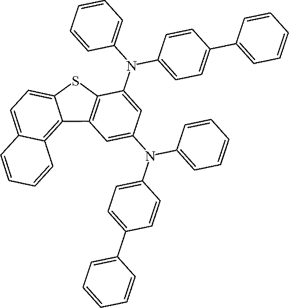 [SMILES: c1ccc(-c2cccc(N(c3ccccc3)c3cc(N(c4ccccc4)c4ccccc4-c4ccccc4)cc4c3sc3c5ccccc5ccc43)c2)cc1] 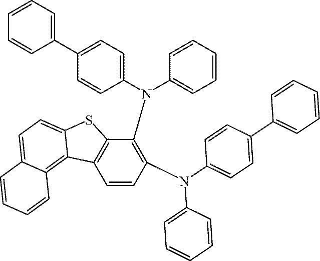 [SMILES: c1ccc(-c2cccc(N(c3ccccc3)c3c(N(c4ccccc4)c4ccccc4-c4ccccc4)ccc4c3sc3c5ccccc5ccc43)c2)cc1] 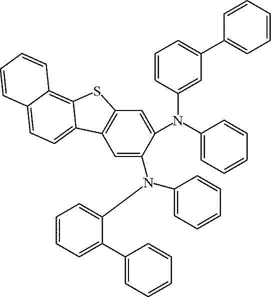 [SMILES: c1ccc(-c2cccc(N(c3ccccc3)c3cc4sc5cc6ccccc6cc5c4cc3N(c3ccccc3)c3ccccc3-c3ccccc3)c2)cc1] 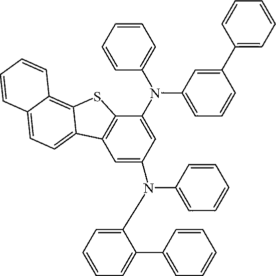 [SMILES: c1ccc(-c2cccc(N(c3ccccc3)c3cc(N(c4ccccc4)c4ccccc4-c4ccccc4)c4sc5cc6ccccc6cc5c4c3)c2)cc1] 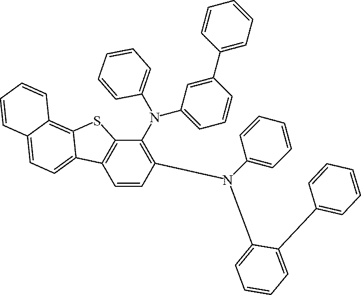 [SMILES: c1ccc(-c2cccc(N(c3ccccc3)c3c(N(c4ccccc4)c4ccccc4-c4ccccc4)ccc4c3sc3cc5ccccc5cc34)c2)cc1] 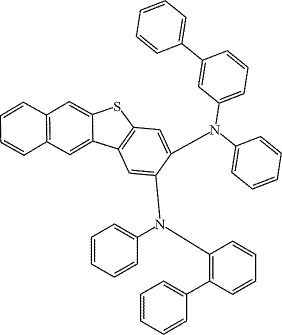 [SMILES: c1ccc(-c2cccc(N(c3ccccc3)c3cc4sc5ccc6ccccc6c5c4cc3N(c3ccccc3)c3ccccc3-c3ccccc3)c2)cc1] 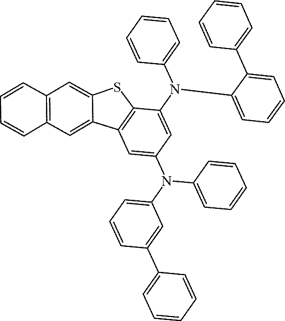 [SMILES: c1ccc(-c2cccc(N(c3ccccc3)c3cc(N(c4ccccc4)c4ccccc4-c4ccccc4)c4sc5ccc6ccccc6c5c4c3)c2)cc1] 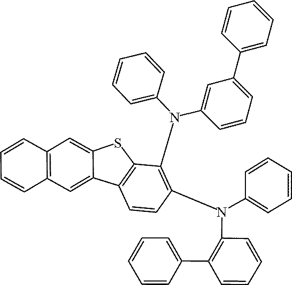 [SMILES: c1ccc(-c2cccc(N(c3ccccc3)c3ccc4c(sc5ccc6ccccc6c54)c3N(c3ccccc3)c3ccccc3-c3ccccc3)c2)cc1] 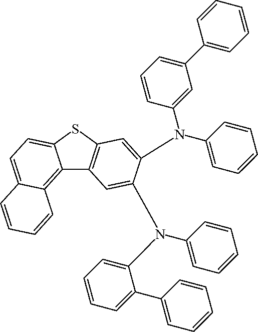 [SMILES: c1ccc(N(c2ccc3oc4ccccc4c3c2)c2cc3sc4c5ccccc5ccc4c3cc2N(c2ccccc2)c2ccc3sc4ccccc4c3c2)cc1] 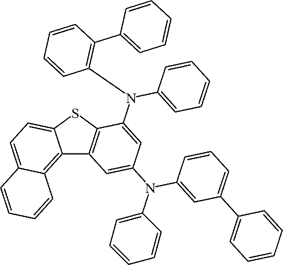 [SMILES: c1ccc(N(c2ccc3sc4ccccc4c3c2)c2cc(N(c3ccccc3)c3ccc4sc5ccccc5c4c3)c3sc4c5ccccc5ccc4c3c2)cc1] 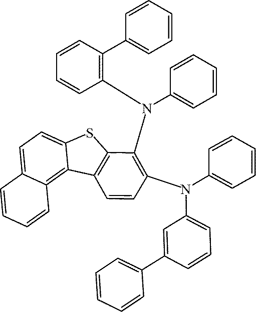 [SMILES: c1ccc(N(c2ccccc2)c2ccc3c(sc4c5ccccc5ccc34)c2N(c2ccc3c(c2)oc2ccccc23)c2ccc3c(c2)oc2ccccc23)cc1] 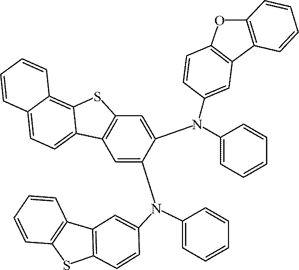 [SMILES: c1ccc(N(c2ccc3sc4ccccc4c3c2)c2cc3sc4cc5ccccc5cc4c3cc2N(c2ccccc2)c2cccc3oc4ccccc4c23)cc1] 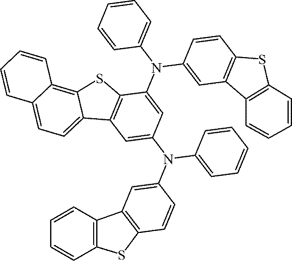 [SMILES: c1ccc(N(c2ccc3c(c2)oc2ccccc23)c2cc(N(c3ccccc3)c3ccc4c(c3)oc3ccccc34)c3sc4cc5ccccc5cc4c3c2)cc1] 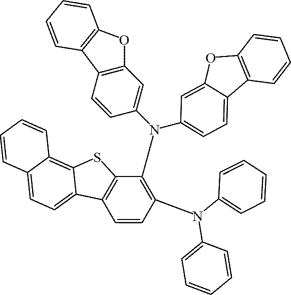 [SMILES: c1ccc(N(c2ccc3c(sc4cc5ccccc5cc43)c2N(c2ccccc2)c2ccc3sc4ccccc4c3c2)c2cccc3c2sc2ccccc23)cc1] 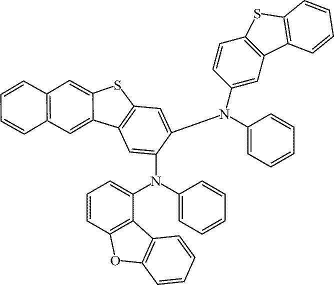 [SMILES: c1ccc(N(c2ccc3c(c2)oc2ccccc23)c2cc3sc4ccc5ccccc5c4c3cc2N(c2ccccc2)c2cccc3c2sc2ccccc23)cc1] 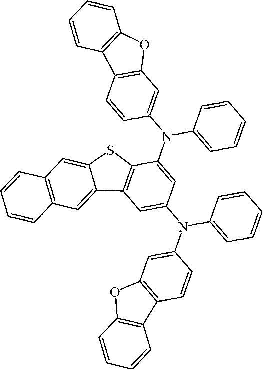 [SMILES: c1ccc(N(c2ccc3sc4ccccc4c3c2)c2cc(N(c3ccccc3)c3ccc4c(c3)sc3ccccc34)c3sc4ccc5ccccc5c4c3c2)cc1] 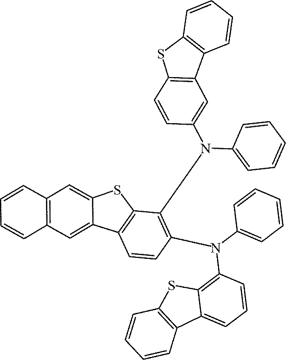 [SMILES: c1ccc(N(c2ccc3c(sc4ccc5ccccc5c43)c2N(c2ccccc2)c2ccc3sc4ccccc4c3c2)c2cccc3c2sc2ccccc23)cc1] 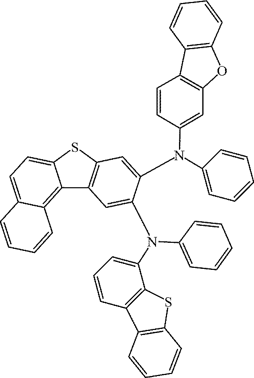 [SMILES: c1ccc(N(c2ccc3oc4ccccc4c3c2)c2cc3sc4c5ccccc5ccc4c3cc2N(c2ccccc2)c2cccc3c2sc2ccccc23)cc1] 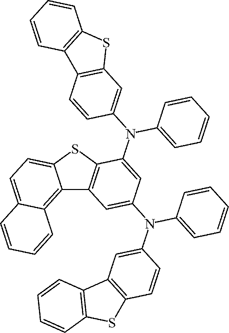 [SMILES: c1ccc(N(c2cc(N(c3ccccc3)c3cccc4oc5ccccc5c34)c3sc4c5ccccc5ccc4c3c2)c2cccc3oc4ccccc4c23)cc1] 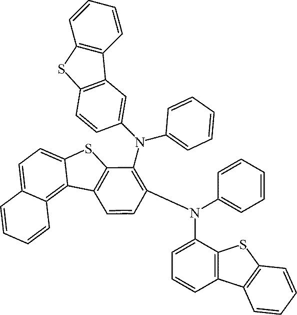 [SMILES: c1ccc(N(c2ccc3c(sc4c5ccccc5ccc34)c2N(c2ccccc2)c2ccc3oc4ccccc4c3c2)c2cccc3c2sc2ccccc23)cc1] 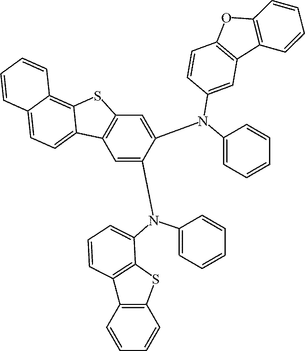 [SMILES: c1ccc(N(c2ccc3c(c2)oc2ccccc23)c2cc3c(cc2N(c2ccccc2)c2cccc4c2sc2ccccc24)sc2cc4ccccc4cc23)cc1] 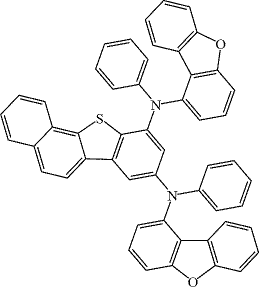 [SMILES: c1ccc(N(c2cc(N(c3ccccc3)c3cccc4oc5ccccc5c34)c3sc4cc5ccccc5cc4c3c2)c2cccc3oc4ccccc4c23)cc1] 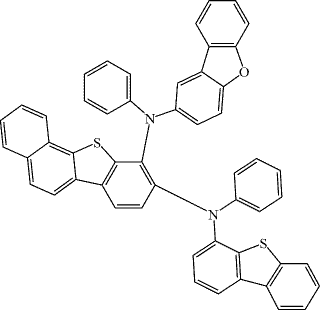 [SMILES: c1ccc(N(c2ccc3sc4ccccc4c3c2)c2ccc3c(sc4cc5ccccc5cc43)c2N(c2ccccc2)c2cccc3c2sc2ccccc23)cc1] 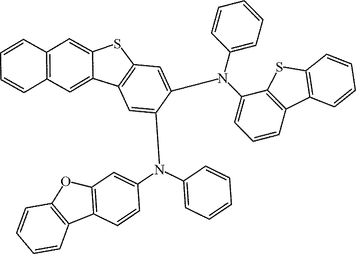 [SMILES: c1ccc(N(c2cc3sc4ccc5ccccc5c4c3cc2N(c2ccccc2)c2cccc3c2sc2ccccc23)c2cccc3c2oc2ccccc23)cc1] 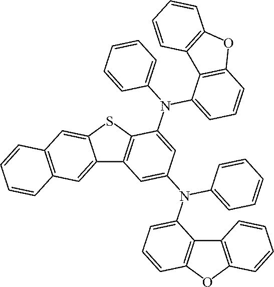 [SMILES: c1ccc(N(c2cccc3c2oc2ccccc23)c2cc(N(c3ccccc3)c3cccc4oc5ccccc5c34)cc3c2sc2ccc4ccccc4c23)cc1] 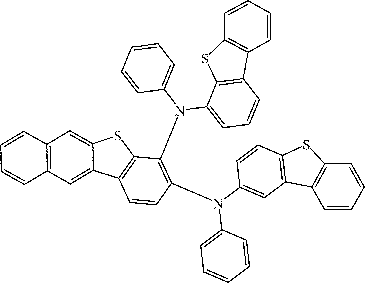 [SMILES: c1ccc(N(c2ccc3c(c2)sc2ccccc23)c2c(N(c3ccccc3)c3cccc4sc5ccccc5c34)ccc3c2sc2ccc4ccccc4c23)cc1] 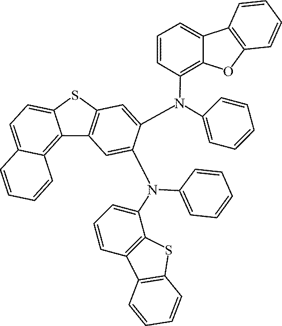 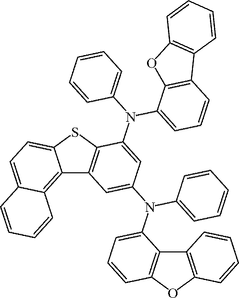 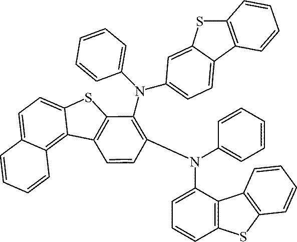
.

9. An organic light emitting device comprising: a first electrode; a second electrode that is provided opposite to the first electrode; and one or more organic material layers that are provided between the first electrode and the second electrode, wherein one or more layers of the organic material layers comprises the compound of claim 1.

10. The organic light emitting device according to claim 9, wherein, one of the one or more organic material layers including the compound is a light emitting layer.
